(19) United States
(12) Patent Application Publication
(10) Pub. No.: US 2006/0078901 A1
(43) Pub. Date: Apr. 13, 2006

(54) Listeria monocytogenes genome, polypeptides and uses

(76) Inventors: Carmen Buchrieser, Paris (FR); Lionel Frangeul, Saint Germain En Laye (FR); Elisabeth Couve, Rambouillet (FR); Christophe Rusniok, Le Plessis Robinson (FR); Hafida Fsihi, Paris (FR); Pierre Dehoux, Paris (FR); Olivier Dussurget, Paris (FR); Farid Chetouani, Cachan (FR); Hafed Nedjari, Puteaux (FR); Philippe Glaser, Paris (FR); Franck Kunst, Ivry Sur Seine (FR); Pascale Cossart, Paris (FR); Justin Daniels, Coulsdon (GB); Werner Goebei, Gerbrunn (DE); Jurgen Kreft, Gerbrunn (DE); Michael Kuhn, Westheim (DE); Eva Yee Wah Ng, Cambridge, MA (US); Jose Antonio Vazquez-Boland, Madrid (ES); Gustavo Dominguez-Bernal, Madrid (ES); Patricia Garrido-Garcia, Madrid (ES); Alberto Tierrez-Martinez, Madrid (ES); Alexandra Amend, Braunfels (DE); Trinad Chakraborty, Giessen (DE); Eugen Domann, Biebertal (DE); Thorsten Hain, Giessen (DE); Patrick Berche, Garches (FR); Alain Charbit, Paris (FR); Lionel Durant, Bievres (FR); Jose-Claudio Perez-Diaz, Madrid (ES); Fernando Baquero, Madrid (ES); Francisco Garcia Del Portillo, Madrid (ES); Nuria Gomez-Lopez, Madrid (ES); Encarna Maduenio, Madrid (ES); Betriz de Pablos, Madrid (ES); Jurgen Wehland, Braunschweig (DE); Uwe Karst, Wolfenbuttel (DE); Karl-Dieter Entian, Oberursel (DE); Jorg Hauf, Wolfskehlen (DE); Matthias Rose, Neu-Anspach (DE); Hamut Voss, Heidelberg (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/045,004

(22) Filed: Jan. 28, 2005

Related U.S. Application Data
(63) Continuation of application No. 10/637,657, filed on Aug. 11, 2003, now abandoned.
Application No. 10/637,657 is a continuation of application No. 10/257,023, now abandoned.

(30) Foreign Application Priority Data
Apr. 11, 2000 (FR) 00/04,629

(51) Int. Cl. C12Q 1/68; C07H 21/04; C12N 15/74; C12N 1/21; C07K 14/195

(52) U.S. Cl. 435/6; 435/69.3; 435/252.3; 435/471; 530/350; 536/23.7

(57) ABSTRACT

The genome sequence and nucleotide sequences of Listeria monocytogenes EGD-e are provided. Polypeptide sequences, including surface or cell envelope polypeptides, and polypeptides involved in metabolism, including B12 biosynthesis, are also provided. Uses of these sequences are also provided.

The invention relates to the genomic sequence and to nucleotide sequences encoding polypeptides of Listeria monocytogenes, such as cell envelope, secreted or specific polypeptides, or polypeptides involved in metabolism, in the replication process or in virulence, and also to vectors which include said sequences and to cells or animals transformed with these vectors. The invention also relates to methods for detecting these nucleic acids or polypeptides and to kits for diagnosing Listeria monocytogenes infection. The invention is also directed toward a method for selecting compounds capable of modulating bacterial infection and a method of biosynthesis or of biodegradation of molecules of interest using said nucleotide sequences or said polypeptides. Finally, the invention comprises pharmaceutical compositions, in particular vaccinal compositions, for preventing and/or treating bacterial infections, in particular Listeria monocytogenes infections.

Listeria monocytogenes is a facultative intracellular pathogen. It is the etiological agent of listeriosis, a food-related infection which poses increasingly great public health problems, with a considerable economic impact for the European food industry. Listeriosis is the most lethal food-related infection (approximately 30% mortality). Listeria monocytogenes has the unusual property of being able to cross three barriers: the intestinal barrier, the blood-brain barrier and the placental barrier. Clinical manifestations of listeriosis include meningitis, meningo-encephalitis, abortions and septicemias. This infection is opportunistic and mainly affects pregnant women, babies, elderly individuals and immunodepressed individuals, in particular individuals suffering from AIDS. This disease apparently also affects healthy individuals and is responsible for a considerable number of epidemics due to contaminated food products. Listeria monocytogenes is also important in veterinary terms, with the main risk being for members of the ovine family (sheep) and of the bovine family. Listeria monocytogenes is particularly resistant to stress or to extreme conditions, and it is important to search for its presence with care not only for problems with food safety, but also for problems of environmental safety.

The physical map and a preliminary genetic map of the Listeria monocytogenes genome have been established for the LO28 strain. However, no fine genetic map is available for the moment. The genome of this bacterium is circular and comprises approximately 3 000 kilobases. Its GC content is approximately 38%. Studies of virulence factors have enabled the identification of a 15 kb locus, which may be considered to be a pathogenicity island insofar as it contains most of the genes whose function in virulence has been clearly identified. In addition to this locus, some other genes have been identified, in particular the invasion and motility genes and genes which encode a murein hydrolase, a superoxide dismutase, sigma factors, etc.

An important family of Listeria monocytogenes proteins is the surface protein family. Evolutionary processes have allowed the development of a number of unique mechanisms on Gram+ bacteria, by which they can immobilize proteins at their surface. The functions of these various cell wall proteins are extremely diverse. However, many proteins covalently attached to the surface of Gram+ pathogens are felt to be important for survival of the pathogen inside the infected host. For Listeria monocytogenes, the ability to penetrate into eukaryotic cells has been linked to a family of surface and/or secreted proteins, the internalins. So far, nine members of the internalin family have been identified (InlA, InlB, InlC, InlC2, INlD, InlE, InlF, InlG and InlH). It is thought that they are anchored in the cell wall by proteolytic cleavage of the T-G bond of the LP×TG motif and that the covalent bonding of the carboxylic group of the threonine to a free amino group in the peptidoglycan [lacuna]. Recent studies have shown that there are two classes of internalins; a group of proteins associated with the cell wall, of high molecular weight (>50 kDa), and a group of smaller proteins (<30 kDa) which are secreted. These two classes have similar, very homologous LRR motifs, and also the LRR flanking regions, and the N-terminal signal peptide sequence. The small internalins (s-inl) do not have the B repeat region or the sequence for anchoring in the cell wall, and are thus secreted.

The study of Listeria monocytogenes requires new approaches, in particular genetic approaches, in order to improve understanding of the various metabolic pathways of this organism.

Thus, an object of the present invention is to disclose the complete sequence of the genome of Listeria monocytogenes EGD-e deposited with the CNCM [National Collection of Microorganism Cultures] on Apr. 11, 2000, under the number I-2440, and also of all the genes contained in said genome.

In fact, knowledge of the genome of this organism makes it possible to define more clearly the interactions between the various genes, the various proteins and, by the same token, the various metabolic pathways. In fact, and unlike the disclosure of isolated sequences, the complete genomic sequence of an organism makes up a whole entity, which immediately makes it possible to obtain all the information required by this organism in order to grow and function.

The present invention therefore relates to a nucleotide sequence of Listeria monocytogenes, characterized in that it corresponds to the sequence SEQ ID NO. 1.

The present invention also relates to a nucleotide sequence of Listeria monocytogenes, characterized in that it is chosen from:
a) a nucleotide sequence comprising at least 80%, 85%, 90%, 95% or 98% identity with SEQ ID NO. 1;
b) a nucleotide sequence which hybridizes, under high stringency conditions, with SEQ ID NO. 1;
c) a nucleotide sequence complementary to SEQ ID NO. 1 or complementary to a nucleotide sequence as defined in a) or b), or a nucleotide sequence of the corresponding RNA;
d) a nucleotide sequence of a representative fragment of SEQ ID NO. 1, or of a representative fragment of a nucleotide sequence as defined in a), b) or c);
e) a nucleotide sequence comprising a sequence as defined in a), b), c) or d); and
f) a modified nucleotide sequence of a nucleotide sequence as defined in a), b), c), d) or e).

More particularly, a subject of the present invention is also the nucleotide sequences characterized in that they are derived from SEQ ID NO. 1 and in that they encode a polypeptide chosen from the polypeptides of sequence SEQ ID NO. 2 to SEQ ID NO. 2854, preferably encoding a cell envelope polypeptide or a polypeptide present at the surface of Listeria monocytogenes of sequence SEQ ID NO. 2 to SEQ ID NO. 41, or encoding a polypeptide involved in vitamin B12 bio synthesis of sequence SEQ ID NO. 42 to SEQ ID NO. 64.

More generally, the present invention also relates to the nucleotide sequences derived from SEQ ID NO.1, and encoding a polypeptide of L. monocytogenes, as may be isolated from SEQ ID NO. 1.

In addition, the nucleotide sequences characterized in that they comprise a nucleotide sequence chosen from:
a) a nucleotide sequence encoding a polypeptide chosen from the sequences SEQ ID NO. 2 to SEQ ID NO. 2854, preferably chosen from the polypeptides of sequence SEQ ID NO. 2 to SEQ ID NO. 41 or SEQ ID NO. 42 to SEQ ID NO. 64;
b) a nucleotide sequence comprising at least 80%, 85%, 90%, 95% or 98% identity with a nucleotide sequence encoding a polypeptide chosen from the sequences SEQ ID NO. 2 to SEQ ID NO. 2854, preferably chosen from the polypeptides of sequence SEQ ID NO. 2 to SEQ ID NO. 41 or SEQ ID NO. 42 to SEQID NO. 64;
c) a nucleotide sequence which hybridizes, under high stringency conditions, with a nucleotide sequence encoding a polypeptide chosen from the sequences SEQ ID NO. 2 to SEQ ID NO. 2854, preferably chosen from the polypeptides of sequence SEQ ID NO. 2 to SEQ ID NO. 41 or SEQ ID NO. 42 to SEQ ID NO. 64;
d) a complementary nucleotide sequence or an RNA sequence corresponding to a sequence as defined in a), b) or c);
e) a nucleotide sequence of a representative fragment of a sequence as defined in a), b), c) or d); and
f) a modified nucleotide sequence of a sequence as defined in a), b), c), d) or e), are also subjects of the invention.

The terms “nucleic acid”, “nucleic acid sequence”, “polynucleotide”, “oligonucleotide”, “polynucleotide sequence” and “nucleotide sequence”, which will be used indifferently in the present description, are intended to denote a precise series of nucleotides, which may or may not be modified, making it possible to define a fragment or a region of a nucleic acid, which may or may not comprise unnatural nucleotides, and which may correspond equally to a double-stranded DNA, a single-stranded DNA and products of transcription of said DNAs. Thus, the nucleic acid sequences according to the invention also encompass PNAs (peptide nucleic acids), or the like.

It should be understood that the present invention does not relate to the nucleotide sequences in their natural chromosomal environment, i.e. in the natural state. They are sequences which have been isolated and/or purified, i.e. they have been taken directly or indirectly, for example by copying, their environment having been at least partially modified. Nucleic acids obtained by chemical synthesis are also intended to be denoted.

For the purpose of the present invention, the term “percentage identity” between two nucleic acid or amino acid sequences is intended to denote a percentage of nucleotides or of amino acid residues which are identical between the two sequences to be compared, obtained after the best alignment, this percentage being purely statistical and the differences between the two sequences being distributed randomly and over their entire length. The term “best alignment” or “optimal alignment” is intended to denote the alignment for which the percentage identity determined as below is the highest. Sequence comparisons between two nucleic acid or amino acid sequences are conventionally carried out by comparing these sequences after having aligned them optimally, said comparison being made by segment or by “window of comparison” so as to identify and compare local regions of sequence similarity. The optimal alignment of the sequences for the comparison may be produced, besides manually, by means of the local homology algorithm of Smith and Waterman (1981, Ad. App. Math. 2: 482), by means of the local homology algorithm of Neddleman and Wunsch (1970, J. Mol. Biol. 48 : 443), by means of the similarity search method of Pearson and Lipman (1988, Proc. Natl. Acad. Sci. USA 85 : 2444), by means of computer programs using these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain the optimal alignment, the BLAST program is preferably used, with the BLOSUM 62 matrix. The PAM or PAM250 matrices may also be used.

The percentage identity between two nucleic acid or amino acid sequences is determined by comparing these two sequences aligned in an optimal manner in which the nucleic acid or amino acid sequence to be compared may comprise additions or deletions compared to the reference sequence for an optimal alignment between these two sequences. The percentage identity is calculated by determining the number of identical positions for which the nucleotide or the amino acid residue is identical between the two sequences, dividing this number of identical positions by the total number of positions compared and multiplying the result obtained by 100 so as to obtain the percentage identity between these two sequences.

The expression “nucleic acid sequences having a percentage identity of at least 80%, preferably 85% to 90%, more preferably 95% or even 98%, after optimal alignment with a reference sequence” is intended to denote the nucleic acid sequences having, compared to the reference nucleic acid sequence, certain modifications, such as in particular a deletion, a truncation, an extension, a chimeric fusion and/or a substitution, in particular of the point type, and in which the nucleic acid sequence exhibits at least 80%, preferably 85%, 90%, 95% or 98%, identity after optimal alignment with the reference nucleic acid sequence. They are preferably sequences whose complementary sequences are capable of hybridizing specifically with the reference sequences. Preferably, the specific or high stringency hybridization conditions will be such that they ensure at least 80%, preferably 85%, 90%, 95% or 98%, identity after optimal alignment between one of the two sequences and the sequence complementary to the other.

Hybridization after high stringency conditions means that the conditions of temperature and of ionic strength are chosen such that they allow hybridization between two complementary DNA fragments to be maintained. By way of illustration, high stringency conditions in the hybridization step for the purposes of defining the polynucleotide fragments described above are advantageously as follows.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 M NaCl+0.015 M sodium citrate), 50% of formamide, 7% of sodium dodecyl sulfate (SDS), 10×Denhardt's, 5% of dextran sulfate and 1% of salmon sperm DNA; (2) hybridization per se for 20 hours at a temperature which depends on the length of the probe (i.e. 42° C. for a probe >100 nucleotides in length), followed by 2 washes of 20 minutes at 20° C. in 2×SSC +2% SDS, and I wash of 20 minutes at 20° C. in 0.1×SSC+0.1% SDS. The final wash is carried out in 0.1 ×SSC+0.1% SDS for 30 minutes at 60° C. for a probe >100 nucleotides in length. The high stringency hybridization conditions described above for a polynucleotide of defined length may be adjusted by those skilled in the art for oligonucleotides which are longer or shorter, according to the teaching of Sambrook et al., (1989, Molecular cloning: a laboratory manual. 2nd Ed. Cold Spring Harbor).

In addition, the expression “representative fragment of sequences according to the invention” is intended to denote any nucleotide fragment having at least 15 consecutive nucleotides, preferably at least 20, 25, 30, 50, 75, 100, 150, 300 and 450 consecutive nucleotides, of the sequence from which it is derived.

The term “representative fragment” is in particular intended to mean a nucleic acid sequence encoding a biologically active fragment of a polypeptide, as defined below.

The term “representative fragment” is also intended to mean the intergenic sequences, and in particular the nucleotide sequences carrying the regulatory signals (promoters, terminators, or even enhancers, etc).

Among said representative fragments, preference is given to those having nucleotide sequences corresponding to open reading frames, named ORF sequences (ORF for open reading frame), generally included between an initiation codon and a stop codon, or between two stop codons, and encoding polypeptides, preferably of at least 100 amino acids, such as, for example, without being limited thereto, the ORF sequences which will subsequently be described.

The numbering of the ORF nucleotide sequences which will subsequently be used in the present description corresponds to the numbering of the amino acid sequences of the proteins encoded by said ORFs.

The representative fragments according to the invention may be obtained, for example, by specific amplification, such as PCR, or after digestion, with suitable restriction enzymes, of nucleotide sequences according to the invention, this method being described in particular in the work by Sambrook et al. Said representative fragments may also be obtained by chemical synthesis when they are not too long, according to methods well known to those skilled in the art.

The sequences containing sequences according to the invention, or representative fragments, are also intended to include the sequences which are naturally framed by sequences which exhibit at least 80%, 85%, 90%, 95% or 98% identity with the sequences according to the invention.

The term “modified nucleotide sequence” is intended to mean any nucleotide sequence obtained by mutagenesis according to techniques well known to those skilled in the art, and comprising modifications, preferably a maximum of 10% of modified nucleotides, compared to the normal sequences, for example mutations in the regulatory and/or promoter sequences for expression of the polypeptide, in particular leading to a modification of the level of expression or of the activity of said polypeptide.

The term “modified nucleotide sequence” is also intended to mean any nucleotide sequence encoding a modified polypeptide as defined below.

The representative fragments according to the invention may also be probes or primers, which may be used in methods for detecting, identifying, assaying or amplifying nucleic acid sequences.

For the purpose of the invention, a probe or primer is defined as being a single-stranded nucleic acid fragment or a denatured double-stranded fragment, comprising, for example, from 12 bases to a few kb, in particular from 15 bases to a few hundreds of bases, preferably from 15 to 50 or 100 bases, and possessing a specificity of hybridization under given conditions so as to form a hybridization complex with a target nucleic acid.

The probes and primers according to the invention may be labeled directly or indirectly with a radioactive or nonradioactive compound using methods well known to those skilled in the art, in order to obtain a detectable and/or quantifiable signal.

The unlabeled polynucleotide sequences according to the invention may be used directly as a probe or primer.

The sequences are generally labeled so as to obtain sequences which can be used for many applications. The primers or the probes according to the invention are labeled with radioactive elements or with nonradioactive molecules.

Among the radioactive isotopes used, mention may be made of ³²P, ³³P, ³⁵S, ³H or ¹²⁵I. The nonradioactive entities are selected from ligands, such as biotin, avidin, streptavidin or dioxigenin, haptens, dyes and luminescent agents, such as radioluminescent, chemiluminescent, bioluminescent, fluorescent or phosphorescent agents.

The polynucleotides according to the invention may thus be used as a primer and/or probe in methods using in particular the PCR (polymerase chain reaction) technique) (Rolfs et al., 1991, Berlin: Springer-Verlag). This technique requires choosing pairs of oligonucleotide primers framing the fragment which must be amplified. Reference may, for example, be made to the technique described in U.S. Pat. No. 4,683,202. The amplified fragments can be identified, for example after agarose or polyacrylamide gel electrophoresis, or after a chromatographic technique such as gel filtration or ion exchange chromatography, and then sequenced. The specificity of the amplification can be controlled using, as a primer, the nucleotide sequences of polynucleotides of the invention and, as a matrix, plasmids containing these sequences or else the derived amplification products. The amplified nucleotide fragments may be used as reagents in hybridization reactions in order to demonstrate the presence, in a biological sample, of a target nucleic acid with the sequence complementary to that of said amplified nucleotide fragments.

The invention is also directed toward the nucleic acids which can be obtained by amplification using primers according to the invention.

Other techniques for amplifying the target nucleic acid may advantageously be used as an alternative to PCR (PCR-like), using a pair of primers of nucleotide sequences according to the invention. The term “PCR-like” is intended to denote all the methods carrying out direct or indirect reproductions of nucleic acid sequences, or in which the labeling systems have been amplified; these techniques are of course well known; in general, they involve amplification of the DNA by a polymerase; when the sample of origin is an RNA a prior reverse transcription should be carried out. There are currently a large number of methods for this amplification, such as, for example, the SDA (strand displacement amplification) technique (Walker et al., 1992, Nucleic Acids Res. 20: 1691), the TAS (transcription-based amplification system) technique described by Kwoh et al. (1989, Proc. Natl. Acad. Sci. USA, 86, 1173), the 3SR (self-sustained sequence replication) technique described by Guatelli et al. (1990, Proc. Natl. Acad. Sci. USA 87: 1874), the NASBA (nucleic acid sequence based amplification) technique described by Kievitis et al. (1991, J. Virol. Methods, 35, 273), the TMA (transcription mediated amplification) technique, the LCR (ligase chain reaction) technique described by Landegren et al. (1988, Science 241, 1077), the RCR (repair chain reaction) technique described by Segev (1992, Kessler C. Springer Verlag, Berlin, New-York, 197-205), the CPR (cycling probe reaction) technique described by Duck et al. (1990, Biotechniques, 9, 142) or the Q-beta-replicase amplification technique described by Miele et al. (1983, J. Mol. Biol., 171, 281). Some of these techniques have since been improved.

When the target polynucleotide to be detected is an mRNA, an enzyme of the reverse transcriptase type is advantageously used, prior to carrying out an amplification reaction using the primers according to the invention or to carrying out a detection method using the probes of the invention, in order to obtain a cDNA from the mRNA contained in the biological sample. The cDNA obtained will then serve as a target for the primers or the probes used in the amplification or detection method according to the invention.

The probe hybridization technique may be performed in many ways (Matthews et al., 1988, Anal. Biochem., 169, 1-25). The most general method consists in immobilizing the nucleic acid extracted from the cells of various tissues, or from cells in culture, on a support (such as nitrocellulose, nylon or polystyrene) and in incubating, under well-defined conditions, the immobilized target nucleic acid with the probe. After hybridization, the excess probe is removed and the hybrid molecules formed are detected using a suitable method (measurement of the radioactivity, of the fluorescence or of the enzymatic activity linked to the probe).

According to another embodiment of the nucleic acid probes according to the invention, the latter may be used as capture probes. In this case, a probe, termed “capture probe”, is immobilized on a support and is used to capture, by specific hybridization, the target nucleic acid obtained from the biological sample to be tested, and the target nucleic acid is then detected using a second probe, termed “detection probe”, labeled with a readily detectable element.

Among the advantageous nucleic acid fragments, mention should thus in particular be made of antisense oligonucleotides, i.e. oligonucleotides with a structure which ensures, by hybridization with the target sequence, inhibition of expression of the corresponding product. Mention should also be made of the sense oligonucleotides which, by interacting with proteins involved in the regulation of expression of the corresponding product, will induce either inhibition or activation of this expression.

Preferably, the probes or primers according to the invention are covalently or noncovalently immobilized on a support. In particular, the support may be a DNA chip or a high density filter, which are also subjects of the present invention.

The term “DNA chip” or “high density filter” is intended to denote a support to which DNA sequences are attached, it being possible to pinpoint each one of these sequences via its geographical location. These chips or filters differ mainly in their size, the material of the support and, possibly, the number of DNA sequences which are attached thereto.

The probes or primers according to the first invention can be attached to solid supports, in particular DNA chips, using various production methods. In particular, in situ synthesis may be performed by photochemical addressing or by inkjet. Other techniques consist in performing ex situ synthesis and attaching the probes to the DNA chip support by mechanical or electronic addressing or by inkjet. These various methods are well known to those skilled in the art.

A nucleotide sequence (probe or primer) according to the invention therefore makes it possible to detect or amplify specific nucleic acid sequences. In particular, the detection of these said sequences is facilitated when the probe is attached to a DNA chip, or to a high density filter.

The use of DNA chips or of high density filters in fact makes it possible to determine gene expression in an organism having a genomic sequences close to L. monocytogenes EGD-e.

The genomic sequence of L. monocytogenes EGD-e, supplemented by the identification of all the genes in this organism, as presented in the present invention, serves as a basis for constructing these DNA chips or filters.

The preparation of these filters or chips consists in synthesizing oligonucleotides corresponding to the 5′ and 3′ ends of the genes. These oligonucleotides are chosen using the genomic sequence and its annotations disclosed by the present invention. The temperature for pairing of these oligonucleotides at the corresponding sites on the DNA should be approximately the same for each oligonucleotide. This makes it possible to prepare DNA fragments corresponding to each gene using suitable PCR conditions in a highly automated environment. The amplified fragments are then immobilized on filters or supports made of glass, silicon or synthetic polymers and these media are used for the hybridization.

The availability of such filters and/or chips and of the corresponding annotated genomic sequence makes it possible to study the expression of large groups, or even all, of the genes in the microorganisms associated with Listeria monocytogenes, by preparing these complementary DNAs and hybridizing them to the DNA or to the oligonucleotides immobilized on the filters or the chips. In addition, the filters and/or the chips make it possible to study the variability of strains or of species, by preparing the DNA of these organisms and hybridizing it to the DNA or to the oligonucleotides immobilized on the filters or the chips.

The differences between the genomic sequences of the various strains or species can greatly affect the strength of hybridization and, consequently, affect the interpretation of the results. It may therefore be necessary to have the precise sequence of the genes of the strains intended to be studied. The method for detecting genes described below in detail, involving determining the sequence of random fragments of a genome and organizing them according to the sequence of the complete genome of Listeria monocytogenes EGD-e disclosed in the present invention, may be very useful.

The nucleotide sequences according to the invention may be used in DNA chips to carry out mutation analysis. This analysis is based on constituting chips capable of analyzing each base of a nucleotide sequence according to the invention. For this purpose, techniques for microsequencing on a DNA chip may in particular be used. The mutations are detected by extension of immobilized primers which hybridize with the matrix of the sequences analyzed, in a position just adjacent to that of the mutated nucleotide sought. A single-stranded RNA or DNA matrix of the sequences to be analyzed will advantageously be prepared according to conventional methods, using products amplified according to techniques such as PCR. The single-stranded DNA or RNA matrices thus obtained are then deposited onto the DNA chip, under conditions which allow them to hybridize specifically to the immobilized primers. A heat-stable polymerase, for example Tth or Taq DNA polymerase, specifically extends the 3′ end of the immobilized primer with a labeled nucleotide analog complementary to the nucleotide in the position of the variable site; for example, a thermal cycle is performed in the presence of fluorescent dideoxyribonucleotides. The experimental conditions will be adjusted in particular to the chips used, to the primers immobilized, to the polymerases used and to the labeling system chosen. An advantage of microsequencing, compared to techniques based on probe hybridization, is that it makes it possible to identify all the variable nucleotides with optimum discrimination under homogeneous reaction conditions; used on DNA chips, it allows optimum resolution and specificity for the routine and industrial multiplex detection of mutations.

The use of the high density filters and/or of the chips thus makes it possible to obtain new knowledge regarding gene regulation in organisms of industrial importance, and in particular listeria propagated under various conditions. It also allows rapid identification of the differences between the genomes of the strains used in many industrial applications.

In addition, a DNA chip or a filter may be an extremely advantageous tool for determining, detecting and/or identifying a microorganism. Thus, preference is also given to the DNA chips according to the invention which also contain at least one nucleotide sequence of a microorganism other than Listeria monocytogenes, immobilized on the support of said chip. Preferably, the microorganism chosen is selected from the bacteria of the Listeria genus (hereinafter designated as L. monocytogenes-associated bacteria), or the variants of Listeria monocytogenes EGD-e.

A DNA chip or a filter according to the invention is a very useful element of certain kits or packs for detecting and/or identifying microorganisms, in particular bacteria belonging to the species Listeria monocytogenes or associated microorganisms, which are also subjects of the invention.

Moreover, the DNA chips or the filters according to the invention, containing probes or primers specific for Listeria monocytogenes, are very advantageous elements of kits or packs for detecting and/or quantifying the expression of genes of Listeria monocytogenes (or of associated microorganisms).

In fact, the control of gene expression is a critical point for optimizing the growth and yield of a strain, either by allowing the expression of one or more new genes, or by modifying the expression of genes already present in the cell. The present invention provides all the sequences naturally active in L. monocytogenes allowing gene expression. It thus makes it possible to determine all the sequences expressed in L. monocytogenes. It also provides a tool for locating the genes the expression of which follows a given scheme. To do this, the DNA of some or all of the genes of L. monocytogenes may be amplified using primers according to the invention, and then attached to a support, such as, for example, glass or nylon or a DNA chip, in order to construct a tool which makes it possible to follow the expression profile of these genes. This tool, consisting of this support containing the coding sequences, is used as a matrix for hybridization to a mixture of labeled molecules which reflect the messenger RNAs expressed in the cell (in particular the labeled probes according to the invention). By repeating this experiment at various times and combining all of these data by a suitable processing, the expression profiles of all of these genes are then obtained. It is also possible to take advantage of knowledge of the sequences which follow a given regulatory scheme in order to search, in a directed manner, for example by homology, for other sequences which follow the same regulatory scheme overall, but in a slightly different manner. In addition, it is possible to isolate each control sequence present upstream of the segments being used as probes and to follow the activity thereof using suitable means, such as a reporter gene (luciferase, β-galactosidase, GFP (for green fluorescent protein)). These isolated sequences can then be modified and assembled by metabolic engineering with sequences of interest with a view to optimal expression thereof.

Using the genomic sequence presented in the present invention, those skilled in the art will be able to identify the genes encoding proteins which regulate gene transcription in L. monocytogenes. Moreover, table I provides the list of open reading frames (ORF) identified on the Listeria monocytogenes genome (SEQ ID NO. 1), with their position on said genome, and the putative functions which may be attributed to them. However, such a list should not be considered to be limiting, a protein possibly being made to have several roles in the cell.

Modifying the structure or the integrity of these genes may make it possible to modify the expression of the target genes controlled by promoters which are targets for these regulators. Thus, those skilled in the art may choose the regulator(s) relevant for the desired application and also their target, which makes it possible to optimize the expression of genes of interest. The use of the tools described above, such as the DNA chips, also makes it possible to pinpoint all the genes the regulation of which is modified by inactivation of certain genes. It is thus possible to select a set of control sequences corresponding, to within a few slight differences, to the same type of regulation. These sequences may then be used to control the expression of genes of interest.

The invention also relates to the polypeptides encoded by a nucleotide sequence according to the invention, preferably by a representative fragment of the sequence SEQ ID NO. 1, and corresponding to an ORF sequence, as described in table I. In particular, the polypeptides of Listeria monocytogenes characterized in that they are chosen from the polypeptides of sequence SEQ ID NO. 2 to SEQ ID NO. 2854, preferably of sequence SEQ ID NO. 2 to SEQ ID NO. 41 and SEQ ID NO. 42 to SEQ ID NO. 64, are a subject of the invention.

The invention also comprises the polypeptides characterized in that they comprise a polypeptide chosen from:
a) a polypeptide according to the invention;
b) a polypeptide exhibiting at least 80%, preferably 85%, 90%, 95% and 98%, identity with a polypeptide according to the invention;
c) a fragment of at least 5 amino acids of a polypeptide according to the invention, or as defined in b);
d) a biologically active fragment of a polypeptide according to the invention, or as defined in b) or c); and
e) a modified polypeptide of a polypeptide according to the invention, or as defined in b), c) or d).

The nucleotide sequences encoding the polypeptides described above are also a subject of the invention.

In the present description, the terms “polypeptides”, “polypeptide sequences”, “peptides” and “proteins” are interchangeable.

It should be understood that the invention does not relate to the polypeptides in natural form, i.e. they are not taken in their natural environment, but it has been possible to isolate or obtain them by purification from natural sources, or else obtain them by genetic recombination or by chemical synthesis, and they may then comprise unnatural amino acids, as will be described below.

The expression “polypeptide having a certain percentage identity with another”, which will also be denoted by the term “homologous polypeptide”, is intended to denote polypeptides which, compared to natural polypeptides, have certain modifications, in particular a deletion, addition or substitution of at least one amino acid, a truncation, an extension, a chimeric solution and/or a mutation, or the polypeptides which have post-translational modifications. Among the homologous polypeptides, preference is given to those the amino acid sequence of which exhibits at least 80%, preferably 85%, 90%, 95% and 98%, homology with the amino acid sequences of the polypeptides according to the invention. In the case of a substitution, one or more consecutive or nonconsecutive amino acid(s) is (are) replaced with “equivalent” amino acids. The expression “equivalent amino acids” is herein intended to denote any amino acid which can be substituted for one of the amino acids of the basic structure without, however, essentially modifying the biological activities of the corresponding peptides, and as will be defined subsequently.

These equivalent amino acids may be determined either based on their structural homology with the amino acids for which they substitute, or based on the results of comparative assays of biological activity between the various polypeptides liable to be produced.

By way of example, mention is made of the possibilities of substitution which can be performed without resulting in a profound modification of the biological activity of the corresponding modified polypeptide. It is thus possible to replace leucine with valine or isoleucine, aspartic acid with glutamic acid, glutamine with asparagine, arginine with lysine, etc, it naturally being possible to envisage the reverse substitution under the same conditions.

The homologous polypeptides also correspond to the polypeptides encoded by the homologous or identical nucleotide sequences as previously defined, and thus comprise, in the present definition, mutated polypeptides or polypeptides corresponding to inter- or intraspecies variations, possibly existing in Listeria, and which in particular correspond to truncations, substitutions, deletions and/or additions of at least one amino acid residue.

It is understood that the percentage identity between two polypeptides is calculated in the same way as between two nucleic acid sequences. Thus, the percentage identity between two polypeptides is calculated after optimal alignment of these two sequences, on a window of maximum homology. To define said window of maximum homology, it is possible to use the same algorithms as for the nucleic acid sequences.

The expression “biologically active fragment of a polypeptide according to the invention” is intended in particular to denote a polypeptide fragment, as defined below, having at least one of the biological characteristics of the polypeptides according to the invention, in particular in that it is capable of generally exercising an activity, even a partial activity, such as, for example:
an enzymatic (metabolic) activity or an activity which may be involved in the biosynthesis or the biodegradation of organic or inorganic compounds;
a structural activity (cell envelope, chaperone molecule, ribosome);
a transport activity (transporting energy, transporting an ion); or an activity in protein secretion;
an activity in the process of replication, amplification, preparation, transcription, translation or maturation, in particular of DNA, of RNA or of proteins.

The expression “polypeptide fragment according to the invention” is intended to denote a polypeptide comprising a minimum of 5 amino acids, preferably 10, 15, 25, 50, 100 and 150 amino acids.

The polypeptide fragments may correspond to isolated or purified fragments naturally present in strains of Listeria, or to fragments which may be obtained by cleaving said polypeptide with a proteolytic enzyme such as trypsin or chymotrypsin or collagenase, or with a chemical reagent (cyanogen bromide, CNBr), or by placing said polypeptide in a very acidic environment (for example at pH=2.5). Polypeptide fragments may also be prepared by chemical synthesis, and using hosts transformed with an expression vector according to the invention, which contain a nucleic acid allowing expression of said fragment and placed under the control of the appropriate regulatory and/or expression elements.

The “modified polypeptide” of a polypeptide according to the invention is intended to denote a polypeptide obtained by genetic recombination or by chemical synthesis, as described below, which has at least one modification compared to the normal sequence, preferably at most 10% of modified amino acids compared to the normal sequence. These modifications may in particular be carried on amino acids required for the specificity or the effectiveness of the activity, or responsible for the structural conformation, for the charge or for the hydrophobicity of the polypeptide according to the invention. It is thus possible to create polypeptides with equivalent, increased or decreased activity, or with equivalent, narrower or broader specificity. Among the modified polypeptides, mention should be made of the polypeptides in which up to five amino acids may be modified, truncated at the N— or C-terminal end, or else deleted, or added.

As is indicated, the aim of the modifications of a polypeptide are in particular:
to allow the use thereof in methods of biosynthesis or biodegradation of organic or inorganic compounds,
to allow the use thereof in methods of replication, amplification, repair and regulation of transcription, translation or maturation, in particular of DNA, RNA or proteins,
to allow the improved secretion thereof,
to modify the solubility thereof, or the effectiveness or specificity of the activity thereof, or alternatively to facilitate the purification thereof.

Chemical synthesis also has the advantage of being able to use unnatural amino acids or nonpeptide bonds. Thus, it may be advantageous to use unnatural amino acids, for example in D form, or analogs of amino acids, in particular sulfur-containing forms.

The present invention provides the nucleotide sequence of the genome of Listeria monocytogenes EGD-e, and also certain polypeptide sequences. Those skilled in the art may determine the other ORFs using known methods and suitable software.

Among the genes identified in the genomic sequence of L. monocytogenes, mention may in particular be made of the genes involved in vitamin B12 biosynthesis (SEQ ID NO. 42 to SEQ ID NO. 64). This bacterium is thus capable of naturally synthesizing this vitamin, and knowledge of the genes leading to their synthesis allows those skilled in the art to optimize expression of these genes or to modify them for the purpose of increasing production of this vitamin. Thus, a subject of the present invention is also a method for producing vitamin B12, characterized in that a host cell containing the genes corresponding to SEQ ID NO. 42 to SEQ ID NO. 64 is provided with the starting substrate, in that it is cultured under conditions suitable for the production of vitamin B12, and in that said vitamin is recovered. The host cell is preferably a bacterial cell, more preferably a bacterium of the Bacillus or Listeria genus. A method for producing vitamin B12 using a nucleic acid or polypeptide sequence according to the invention, a host cell according to the invention, or an animal or plant according to the invention is also a subject of the present invention.

In general, the list of SEQ ID sequences, or their corresponding coding nucleic acid sequence, may be determined by those skilled in the art using the most probable putative functions determined for each of the SEQ ID sequences in table I hereinafter for each of the classes of activity listed hereinafter.

Thus and preferably, the invention relates to a nucleotide sequence according to the invention, characterized in that it encodes a polypeptide of Listeria monocytogenes, or a fragment thereof, involved in vitamin B12 biosynthesis. It is preferably a polypeptide sequence of SEQ ID NO. 42 to SEQ ID NO. 64.

Preferably, the invention relates to a nucleotide sequence according to the invention, characterized in that it encodes a cell envelope polypeptide or polypeptide at the surface of Listeria monocytogenes, or a fragment thereof. It is preferably a polypeptide of sequence SEQ ID NO. 2 to SEQ ID NO. 41.

Preferably, the invention relates to a nucleotide sequence according to the invention, characterized in that it encodes a polypeptide of Listeria monocytogenes, or a fragment thereof, involved in amino acid biosynthesis.

Preferably, the invention relates to a nucleotide sequence according to the invention, characterized in that it encodes a polypeptide of Listeria monocytogenes, or a fragment thereof, involved in the biosynthesis of cofactors, prosthetic groups and transporters.

Preferably, the invention relates to a nucleotide sequence according to the invention, characterized in that it encodes a polypeptide of Listeria monocytogenes, or a fragment thereof, involved in the cellular machinery.

Preferably, the invention relates to a nucleotide sequence according to the invention, characterized in that it encodes a polypeptide of Listeria monocytogenes, or a fragment thereof, involved in central intermediate metabolism.

Preferably, the invention relates to a nucleotide sequence according to the invention, characterized in that it encodes a polypeptide of Listeria monocytogenes, or a fragment thereof, involved in energetic metabolism.

Preferably, the invention relates to a nucleotide sequence according to the invention, characterized in that it encodes a polypeptide of Listeria monocytogenes, or a fragment thereof, involved in the metabolism of fatty acids and phospholipids.

Preferably, the invention relates to a nucleotide sequence according to the invention, characterized in that it encodes a polypeptide of Listeria monocytogenes, or a fragment thereof, involved in the metabolism of nucleotides, purines, pyrimidines or nucleosides.

Preferably, the invention relates to a nucleotide sequence according to the invention, characterized in that it encodes a polypeptide of Listeria monocytogenes, or a fragment thereof, involved in regulatory functions.

Preferably, the invention relates to a nucleotide sequence according to the invention, characterized in that it encodes a polypeptide of Listeria monocytogenes, or a fragment thereof, involved in the replication process.

Preferably, the invention relates to a nucleotide sequence according to the invention, characterized in that it encodes a polypeptide of Listeria monocytogenes, or a fragment thereof, involved in the transcription process.

Preferably, the invention relates to a nucleotide sequence according to the invention, characterized in that it encodes a polypeptide of Listeria monocytogenes, or a fragment thereof, involved in the translation process.

Preferably, the invention relates to a nucleotide sequence according to the invention, characterized in that it encodes a polypeptide of Listeria monocytogenes, or a fragment thereof, involved in the process of transport and binding of proteins.

Preferably, the invention relates to a nucleotide sequence according to the invention, characterized in that it encodes a polypeptide of Listeria monocytogenes, or a fragment thereof, involved in adaptation to atypical conditions.

Preferably, the invention relates to a nucleotide sequence according to the invention, characterized in that it encodes a polypeptide of Listeria monocytogenes, or a fragment thereof, involved in sensitivity to medicinal products and analogs.

Preferably, the invention relates to a nucleotide sequence according to the invention, characterized in that it encodes a polypeptide of Listeria monocytogenes, or a fragment thereof, involved in functions relating to transposons.

Preferably, the invention relates to a nucleotide sequence according to the invention, characterized in that it encodes a polypeptide specific for Listeria monocytogenes, or a fragment thereof.

In another aspect, a subject of the invention is preferably a polypeptide according to the invention, characterized in that it is a polypeptide of Listeria monocytogenes, or a fragment thereof, involved in vitamin B12 biosynthesis. It is preferably a polypeptide of sequence SEQ ID NO. 42 to SEQ ID NO. 64.

In another aspect, a subject of the invention is preferably a polypeptide according to the invention, characterized in that it is a cell envelope polypeptide or a surface polypeptide of Listeria monocytogenes, or a fragment thereof. It is preferably a polypeptide of sequence SEQ ID NO. 2 to SEQ ID NO. 41.

In another aspect, a subject of the invention is preferably a polypeptide according to the invention, characterized in that it is a polypeptide of Listeria monocytogenes, or a fragment thereof, involved in amino acid biosynthesis.

In another aspect, a subject of the invention is preferably a polypeptide according to the invention, characterized in that it is a polypeptide of Listeria monocytogenes, or a fragment thereof, involved in the biosynthesis of cofactors, prosthetic groups and transporters.

In another aspect, a subject of the invention is preferably a polypeptide according to the invention, characterized in that it is a polypeptide of Listeria monocytogenes, or a fragment thereof, involved in the cellular machinery.

In another aspect, a subject of the invention is preferably a polypeptide according to the invention, characterized in that it is a polypeptide of Listeria monocytogenes, or a fragment thereof, involved in central intermediate metabolism.

In another aspect, a subject of the invention is preferably a polypeptide according to the invention, characterized in that it is a polypeptide of Listeria monocytogenes, or a fragment thereof, involved in energetic metabolism.

In another aspect, a subject of the invention is preferably a polypeptide according to the invention, characterized in that it is a polypeptide of Listeria monocytogenes, or a fragment thereof, involved in the metabolism of fatty acids and phospholipids.

In another aspect, a subject of the invention is preferably a polypeptide according to the invention, characterized in that it is a polypeptide of Listeria monocytogenes, or a fragment thereof, involved in the metabolism of nucleotides, purines, pyrimidines or nucleosides.

In another aspect, a subject of the invention is preferably a polypeptide according to the invention, characterized in that it is a polypeptide of Listeria monocytogenes, or a fragment thereof, involved in regulatory functions.

In another aspect, a subject of the invention is preferably a polypeptide according to the invention, characterized in that it is a polypeptide of Listeria monocytogenes, or a fragment thereof, involved in the replication process.

In another aspect, a subject of the invention is preferably a polypeptide according to the invention, characterized in that it is a polypeptide of Listeria monocytogenes, or a fragment thereof, involved in the transcription process.

In another aspect, a subject of the invention is preferably a polypeptide according to the invention, characterized in that it is a polypeptide of Listeria monocytogenes, or a fragment thereof, involved in the translation process.

In another aspect, a subject of the invention is preferably a polypeptide according to the invention, characterized in that it is a polypeptide of Listeria monocytogenes, or a fragment thereof, involved in the process of transport and binding of proteins.

In another aspect, a subject of the invention is preferably a polypeptide according to the invention, characterized in that it is a polypeptide of Listeria monocytogenes, or a fragment thereof, involved in adaptation to atypical conditions.

In another aspect, a subject of the invention is preferably a polypeptide according to the invention, characterized in that it is a polypeptide of Listeria monocytogenes, or a fragment thereof, involved in sensitivity to medicinal products and analogs.

In another aspect, a subject of the invention is preferably a polypeptide according to the invention, characterized in that it is a polypeptide of Listeria monocytogenes, or a fragment thereof, involved in functions relating to transposons.

In another aspect, a subject of the invention is preferably a polypeptide according to the invention, characterized in that it is a polypeptide specific for Listeria monocytogenes, or a fragment thereof.

It is important to note, however, that a living organism is a whole entity and should be taken as such. Thus, in order to be able to develop and to exhibit its properties, any organism needs interactions between the various metabolic pathways. Thus, the classification stated above should not be considered to be limiting, a gene possibly being involved in two different metabolic pathways.

A subject of the present invention is also the nucleotide and/or polypeptide sequences according to the invention, characterized in that said sequences are recorded on a recording medium, the form and nature of which facilitate the reading, analysis and/or exploitation of said sequence(s). These media may also contain other information extracted from the present invention, in particular the analogies with already known sequences, and/or information concerning the nucleotide and/or polypeptide sequences of other microorganisms, in order to facilitate the comparative analysis and exploitation of the results obtained.

Among these said recording media, preference is given in particular to computer-readable media, such as magnetic, optical, electrical or hybrid media, in particular computer disks, CD-ROMs and computer servers. Such recording media are also a subject of the invention.

The recording media according to the invention, with the information provided, are very useful for choosing nucleotide primers or probes for determining genes in Listeria monocytogenes or strains close to this organism. Similarly, the use of these media for studying the genetic polymorphism of a strain close to Listeria monocytogenes, in particular by determining the regions of colinearity, is very useful insofar as these media provide not only the nucleotide sequence of the genome of Listeria monocytogenes egb, but also the genomic organization in said sequence. Thus, the uses of recording media according to the invention are also subjects of the invention.

The analysis of homology between various sequences is in fact advantageously performed using sequence comparison programs, such as the Blast program or the programs of the GCG package, described above.

The invention is also directed toward the cloning and/or expression vectors which contain a nucleotide sequence according to the invention. Preference is particularly given to the nucleotide sequences encoding cell envelope or surface polypeptides, or polypeptides involved in the cellular machinery, in particular secretion, central intermediate metabolism, in particular sugar production, energetic metabolism, and the processes of vitamin B12 synthesis, of transcription and translation and of polypeptide synthesis.

The vectors according to the invention preferably comprise elements which allow expression and/or secretion of the nucleotide sequences in a given host cell.

The vector should then comprise a promoter, translation initiation and determination signals, and also regions suitable for regulating transcription. It must be possible for it to be maintained stably in the host cell and it may optionally contain particular signals which specify secretion of the translated protein. These various elements are chosen and optimized by those skilled in the art depending on the cellular host used. To this effect, the nucleotide sequences according to the invention may be inserted into vectors which replicate autonomously in the host chosen, or may be vectors which integrate in the host chosen.

Such vectors are prepared by methods commonly used by those skilled in the art, and the resulting clones may be introduced into a suitable host using standard methods, such as lipofection, electroporation, heat shock or chemical methods.

The vectors according to the invention are, for example, vectors of plasmid or viral origin. They are of use in transforming host cells in order to clone or express the nucleotide sequences according to the invention.

The invention also comprises the host cells transformed with a vector according to the invention.

The cellular host may be chosen from prokaryotic or eukaryotic systems, for example bacterial cells but also yeast cells or animal cells, in particular mammalian cells. Insect cells or plant cells may also be used. The preferred host cells according to the invention are in particular prokaryotic cells, preferably bacteria belonging to the Listeria genus, to the species Listeria monocytogenes, or microorganisms associated with the species Listeria monocytogenes. The invention also relates to the animals, except humans, which comprise a transformed cell according to the invention. The transformed cells according to the invention can be used in methods for preparing recombinant polypeptides according to the invention. The methods for preparing a polypeptide according to the invention in recombinant form. characterized in that they use a vector and/or a cell transformed with a vector according to the invention, are themselves included in the present invention. Preferably, a cell transformed with a vector according to the invention is cultured under conditions which allow expression of said polypeptide, and said recombinant peptide is recovered. The host cells according to the invention may also be used for preparing food compositions, which are themselves a subject of the present invention.

As has been mentioned, the cellular host may be chosen from prokaryotic or eukaryotic systems. In particular, it is possible to identify nucleotide sequences according to the invention which facilitate secretion in such a prokaryotic or eukaryotic system. A vector according to the invention carrying such a sequence may therefore be advantageously used for producing recombinant proteins intended to be secreted. As a result, the purification of these recombinant proteins of interest will be facilitated by the fact that they are present in the cell culture supernatant rather than inside the host cells.

The polypeptides according to the invention may also be prepared by chemical synthesis. Such a method of preparation is also a subject of the invention. Those skilled in the art are aware of the methods of chemical synthesis, for example techniques using solid phases (see in particular Steward et al., 1984, Solid phase peptides synthesis, Pierce Chem. Company, Rockford, 111, 2nd ed. (1984)) or techniques using partial solid phases, by fragment condensation or by conventional synthesis in solution. The polypeptides obtained by chemical synthesis, and possibly comprising corresponding unnatural amino acids, are also included in the invention.

The invention also relates to hybrid polypeptides having at least one polypeptide, or a fragment thereof, according to the invention, and a sequence of a polypeptide capable of inducing an immune response in humans or animals.

Advantageously, the antigenic determinant is such that it is capable of inducing a humoral and/or cellular response.

Such a determinant may comprise a polypeptide, or a fragment thereof, according to the invention in a glycosylated form used for the purpose of obtaining immunogenic compositions capable of inducing the synthesis of antibodies directed against multiple epitopes. Said polypeptides, or the glycosylated fragments thereof, are also part of the invention.

These molecules may consist partly of a molecule bearing polypeptides, or fragments thereof, according to the invention, combined with an optionally immunogenic component, in particular an epitope of diphtheria toxin, tetanus toxin, a surface antigen of the hepatitis B virus (patent FR 79 21811), the VP1 antigen of the poliomyelitis virus or any other viral or bacterial antigen or toxin.

The methods for synthesizing the hybrid molecules encompass the methods used in genetic engineering for constructing hybrid nucleotide sequences encoding the desired polypeptide sequences. Reference may, for example, advantageously be made to the technique for obtaining genes encoding fusion proteins, described by Minton in 1984.

Said hybrid nucleotide sequences encoding a hybrid polypeptide and also the hybrid polypeptides according to the invention, characterized in that they are recombinant polypeptides obtained by expressing said hybrid nucleotide sequences, are also part of the invention.

The invention also comprises the vectors characterized in that they contain one of said hybrid nucleotide sequences. The host cells transformed with said vectors, the transgenic animals comprising one of said transformed cells and also the methods for preparing recombinant polypeptides using said vectors, said transformed cells and/or said transgenic animals are, of course, also part of the invention.

The coupling between a polypeptide according to the invention and an immunogenic polypeptide may be carried out chemically or biologically. Thus, according to the invention, it is possible to introduce one or more attachment elements, in particular amino acids, so as to facilitate the reactions for coupling between the polypeptide according to the invention and the immunostimulatory polypeptide, the covalent coupling of the immunostimulatory antigen possibly taking place at the N— or C-terminal end of the polypeptide according to the invention. Bifunctional reagents for this coupling are determined as a function of the end chosen for carrying out this coupling, and the coupling techniques are well known to those skilled in the art.

The conjugates derived from a peptide coupling may be prepared by genetic recombination. The hybrid peptide (conjugate) may in effect be produced by recombinant DNA techniques, by insertion or addition of a sequence encoding the antigenic, immunogenic or hapten peptide(s) into or to the DNA sequence encoding the polypeptide according to the invention. These techniques for preparing hybrid peptides by genetic recombination are well known to those skilled in the art (see, for example, Makrides, 1996, Microbiological Reviews 60, 512-538).

Preferably, said immune polypeptide is chosen from the group of peptides containing toxoids, in particular diphtheria toxoid or tetanus toxoid, streptococcus-derived proteins (such as the human serum albumin-binding protein), OMPA membrane proteins and outer membrane protein complexes, outer membrane vesicles or heat shock proteins.

The hybrid polypeptides according to the invention are very useful for obtaining monoclonal or polyclonal antibodies capable of specifically recognizing the polypeptides according to the invention. In fact, a hybrid polypeptide according to the invention allows potentiation of the immune response, against the polypeptide according to the invention coupled to the immunogenic molecule. Such monoclonal or polyclonal antibodies, fragments thereof or the chimeric antibodies, which recognize the polypeptides according to the invention, are also subjects of the invention.

The specific monoclonal antibodies may be obtained according to the conventional method of hybridoma culturing described by Köhler and Milstein (1975, Nature 256, 495).

The antibodies according to the invention are, for example, chimeric antibodies, humanized antibodies, or Fab or F(ab′)² fragments. It may also be in the form of an immunoconjugate or of an antibody which is labeled in order to obtain a detectable and/or quantifiable signal.

Thus, the antibodies according to the invention may be used in a method for depicting and/or identifying bacteria belonging to the species Listeria monocytogenes, or to an associated microorganism, in a biological sample, characterized in that it comprises the following steps:
a) bringing the biological sample into contact with an antibody according to the invention;
b) demonstrating the antigen-antibody complex possibly formed.

The antibodies according to the present invention can also be used in order to detect expression of a gene of Listeria monocytogenes or of associated microorganisms. Specifically, the presence of the expression product of a gene recognized by an antibody specific for said expression product can be detected via the presence of an antigen-antibody complex formed after the Listeria monocytogenes strain or the associated microorganism has been brought into contact with an antibody according to the invention. The bacterial strain used may have been “prepared”, i.e. centrifuged, lyzed, and placed in an appropriate reagent for constituting the medium suitable for the immunoreaction. In particular, preference is given to a method for detecting the expression in the gene corresponding to a Western blot, which may be performed after polyacrylamide gel electrophoresis of a lysate of the bacterial strain, in the presence or absence of reducing conditions (SDS-PAGE). After migration and separation of the proteins on the polyacrylamide gel, said proteins are transferred onto a suitable membrane (for example made of nylon) and the presence of the protein or of the polypeptide of interest is detected by bringing said membrane into contact with an antibody according to the invention.

Thus, the present invention also comprises the kits or packs for carrying out a method as described (for detecting the expression of a gene of Listeria monocytogenes, or an associated microorganism, or for detecting and/or identifying bacteria belonging to the species Listeria monocytogenes, or an associated microorganism), comprising the following elements:
a) a polyclonal or monoclonal antibody according to the invention;
b) optionally, the reagents for constituting the medium suitable for the immunoreaction;
c) optionally, the reagents for demonstrating the antigen-antibody complexes produced by the immunoreaction.

The polypeptides and the antibodies according to the invention may advantageously be immobilized on a support, in particular a protein chip. Such a protein chip is a subject of the invention and may also contain at least one polypeptide of a microorganism other than Listeria monocytogenes, or an antibody directed against a compound of a microorganism other than Listeria monocytogenes.

The protein chips or high density filters containing proteins according to the invention may be constructed in the same way as the DNA chips according to the invention. In practice, it is possible to carry out the synthesis of the polypeptides attached directly to the protein chip, or to carry out an ex situ synthesis followed by a step of attaching the synthesized polypeptide to said chip. The latter method is preferable when the intention is to attach proteins of considerable size to the support, which are advantageously prepared by genetic engineering. However, if the intention is to attach only peptides to the support of said chip, it may be more advantageous to synthesize said peptides directly in situ.

The protein chips according to the invention may advantageously be used in kits or packs for detecting and/or identifying bacteria associated with the species Listeria monocytogenes, or with a microorganism, or more generally in kits or packs for detecting and/or identifying microorganisms. When the polypeptides according to the invention are attached to DNA chips, the presence of antibodies in the samples tested is sought, the attachment of an antibody according to the invention to the support of the protein chip allowing identification of the protein for which said antibody is specific.

Preferably, an antibody according to the invention is attached to the support of the protein chip and the presence of the corresponding antigen, specific for Listeria monocytogenes, or for an associated microorganism, is detected.

A protein chip described above may be used for detecting gene products, in order to establish an expression profile for said genes, in addition to a DNA chip according to the invention.

The protein chips according to the invention are also extremely useful for proteomic experiments, which study interactions between the various proteins of a given microorganism. In a simplified manner, peptides representative of the various proteins of an organism are attached to a support. Said support is brought into contact with labeled proteins and, after an optional rinsing step, interactions between said labeled proteins and the peptides attached to the protein chip are detected.

Thus, the protein chips comprising a polypeptide sequence according to the invention are an antibody according to the invention are a subject of the invention, as are the kits or packs containing them.

The present invention also covers a method for detecting and/or identifying bacteria belonging to the species Listeria monocytogenes, or to an associated microorganism, in a biological sample, which uses a nucleotide sequence according to the invention.

It should be clearly understood that, in the present invention, the term “biological sample” concerns samples taken from a living organism (in particular blood, tissues, organs or other samples taken from a mammal) or a sample containing biological material, i.e. DNA. Such a biological sample therefore encompasses food compositions containing bacteria (for example cheeses, dairy products), but also food compositions containing yeast (beers, breads) or others.

The method for detection and/or identification using the nucleotide sequences according to the invention may be diverse in nature.

A method comprising the following steps is preferred:
a) optionally isolating the DNA from the biological sample to be analyzed, or obtaining a cDNA from the RNA of the biological sample;
b) specifically amplifying the DNA of bacteria belonging to the species Listeria monocytogenes, or to an associated microorganism, using at least one primer according to the invention;
c) demonstrating the amplification products.

This method is based on the specific amplification of the DNA, in particular via a polymerase chain reaction.

A method comprising the following steps is also preferred:
a) bringing a nucleotide probe according to the invention into contact with a biological sample, the nucleic acid contained in the biological sample having, where appropriate, been previously made accessible to hybridization, under conditions which allow hybridization of the probe to the nucleic acid of a bacterium belonging to the species Listeria monocytogenes, or to an associated microorganism;
b) demonstrating the hybrid possibly formed between the nucleotide probe and the DNA of the biological sample.

Such a method should not be limited to detecting the presence of the DNA contained in the biological sample in question, it may also be used to detect the RNA contained into said sample. This method in particular encompasses Southern and Northern blotting.

Another preferred method according to the invention comprises the following steps:
a) bringing a nucleotide probe immobilized on a support according to the invention into contact with a biological sample, the nucleic acid of the sample having, where appropriate, previously been made accessible to hybridization, under conditions which allow hybridization of the probe to the nucleic acid of a bacterium belonging to the species Listeria monocytogenes, or to an associated microorganism;
b) bringing the hybrid formed between the nucleotide probe immobilized on a support and the nucleic acid contained in the biological sample, where appropriate after removing the DNA of the biological sample which has not hybridized with the probe, into contact with a labeled nucleotide probe according to the invention;
c) demonstrating the new hybrid formed in step b).

This method is advantageously used with a DNA chip according to the invention, the nucleic acid being sought hybridizing with a probe present at the surface of said chip, and being detected using a labeled probe. This method is advantageously carried out by combining a prior step of amplifying the DNA or the complementary DNA optionally obtained by reverse transcription, using primers according to the invention.

Thus, the present invention also encompasses the kits or packs for detecting and/or identifying bacteria belonging to the species Listeria monocytogenes, or to an associated microorganism, characterized in that it comprises the following elements:
a) a nucleotide probe according to the invention;
b) optionally, the reagents required for carrying out a hybridization reaction;
c) optionally, at least one primer according to the invention and also the reagents required for a DNA amplification reaction.

Similarly, the present invention also encompasses the kits or packs for detecting and/or identifying bacteria belonging to the species Listeria monocytogenes, or to an associated microorganism, characterized in that it comprises the following elements:
a) a nucleotide probe, termed capture probe, according to the invention;
b) an oligonucleotide probe, termed detection probe, according to the invention;
c) optionally, at least one primer according to the invention and also the reagents required for a DNA amplification reaction.

Finally, the kits or packs for detecting and/or identifying bacteria belonging to the species Listeria monocytogenes, or to an associated microorganism, characterized in that it comprises the following elements:
a) at least one primer according to the invention;
b) optionally, the reagents required for carrying out a DNA amplification reaction;
c) optionally, a component for verifying the sequence of the amplified fragment, more particularly an oligonucleotide probe according to the invention,
are also subjects of the present invention.

Preferably, said primers and/or probes and/or polypeptides and/or antibodies according to the present invention, used in the methods and/or kits or packs according to the present invention, are chosen from the primers and/or probes and/or polypeptides and/or antibodies specific for the species Listeria monocytogenes. Preferably, these elements are chosen from the nucleotide sequences encoding a secreted protein, from the secreted polypeptides, or from the antibodies directed against secreted polypeptides of Listeria monocytogenes.

A subject of the present invention is also the strains of Listeria monocytogenes, and/or of associated microorganisms, containing one or more mutation(s) in a nucleotide sequence according to the invention, in particular an ORF sequence, or regulatory elements thereof (in particular promoters).

According to the invention, preference is given to the strains of Listeria monocytogenes having one or more mutation(s) in the nucleotide sequences encoding polypeptides involved in the cellular machinery, in particular secretion, central intermediate metabolism, energetic metabolism, and processes of amino acid synthesis, of transcription and translation, and of polypeptide synthesis.

Said mutations may lead to inactivation of this gene or, in particular when they are located in the regulatory elements of said gene, to overexpression of this gene.

The invention also relates to the use of a nucleotide sequence according to the invention, of a polypeptide according to the invention, of an antibody according to the invention, of a cell according to the invention and/or of a transformed animal according to the invention, for selecting an organic or inorganic compound capable of modulating, regulating, inducing or inhibiting gene expression, and/or of modifying cell replication in eukaryotic or prokaryotic cells, or capable of inducing, inhibiting or worsening pathological conditions linked to an infection with Listeria monocytogenes, or a microorganism associated therewith.

The invention also comprises a method for selecting compounds capable of binding to a polypeptide, or a fragment thereof, according to the invention, capable of binding to a nucleotide sequence according to the invention, or capable of recognizing an antibody according to the invention, and/or capable of modulating, regulating, inducing or inhibiting gene expression, and/or modifying the cell growth or replication in eukaryotic or prokaryotic cells, or capable of inducing, inhibiting or worsening, in an animal or human organism, pathological conditions linked to an infection with Listeria monocytogenes, or a microorganism associated therewith, characterized in that it comprises the following steps:
a) bringing said compound into contact with said polypeptide or said nucleotide sequence or with a transformed cell according to the invention and/or administering said compound to a transformed animal according to the invention;
b) determining the ability of said compound to bind to said polypeptide or said nucleotide sequence, or to modulate, regulate, induce or inhibit gene expression, or to modulate cell growth or replication, or to induce, inhibit or worsen, in said transformed animal, pathological conditions linked to an infection with Listeria monocytogenes or a microorganism associated therewith.

The transformed cells and/or animals according to the invention may advantageously serve as a model and be used in methods for studying, identifying and/or selecting compounds which may be responsible for pathological conditions induced or worsened by Listeria monocytogenes, or which may prevent and/or treat these pathological conditions, such as, for example, genital, eye or systemic diseases, in particular diseases of the lymphatic system. In particular, the transformed host cells, especially the bacteria of the Listeriae family, the transformation of which with a vector according to the invention may, for example, increase or inhibit its infectious capacity, or modulate the pathological conditions usually induced or worsened by the infection, may be used to infect animals in which the appearance of the pathological conditions will be monitored. These nontransformed animals, infected, for example, with transformed Listeriae bacteria, may serve as a study model. In the same way, the transformed animals according to the invention may be used in methods for selecting compounds capable of preventing and/or treating diseases due to Listeria. Said methods using said transformed cells and/or transformed animals are part of the invention.

The compounds liable to be selected may be organic compounds, such as polypeptides or carbohydrates, or any other already known organic or inorganic compounds, or new organic compounds developed using molecular modeling techniques and obtained by chemical or biochemical synthesis, these techniques being known to those skilled in the art.

Said selected compounds may be used for modulating cell growth and/or replication in Listeria monocytogenes, or any other associated microorganism, and also for controlling infection with these microorganisms. Said compounds according to the invention may also be used for modulating cell growth and/or replication in any eukaryotic or prokaryotic cells, in particular tumor cells and infectious microorganisms, for which said compounds will prove to be active, the methods for determining said modulations being well known to those skilled in the art.

The expression “compound capable of modulating the growth of a microorganism” is intended to denote any compound making it possible to intervene in, modify, limit and/or reduce the development, growth, rate of proliferation and/or viability of said microorganism.

This modulation may be carried out, for example, using an agent capable of binding to a protein and thus of inhibiting or potentiating its biological activity, or capable of binding to an outer surface membrane protein of a microorganism and blocking the penetration of said microorganism into the host cell or promoting the action of the immune system of the infected organism, directed against said microorganism. This modulation may also be carried out using an agent capable of binding to a nucleotide sequence of a DNA or RNA of a microorganism and blocking, for example, the expression of a polypeptide the biological or structural activity of which is necessary for the growth or for the reproduction of said microorganism.

In the present invention, the term “associated microorganism” is intended to denote any microorganism in which the gene expression may be modulated, regulated, induced or inhibited, or the cell growth or replication of which may also be modulated, by a compound of the invention. In the present invention, the term “associated microorganism” is also intended to denote any microorganism comprising nucleotide sequences or polypeptides according to the invention. These microorganisms may, in certain cases, comprise polypeptides or nucleotide sequences identical or homologous to those of the invention [lacuna] may also be detected and/or identified using the methods or kit for detection and/or identification according to the invention and may also serve as a target for the compounds of the invention.

The invention relates to the compounds which may be selected using a election method according to the invention.
The invention also relates to a pharmaceutical composition comprising a compound chosen from the following compounds:
a) a nucleotide sequence according to the invention;
b) a polypeptide according to the invention;
c) a vector according to the invention;
d) an antibody according to the invention; and
e) a compound which may be selected using a selection method according to the invention, optionally in combination with a pharmaceutically acceptable vehicle.

The term “effective amount” is intended to denote a sufficient amount of said compound or antibody, or of polypeptide of the invention, for modulating the growth of Listeria monocytogenes or of an associated microorganism.

The invention also relates to a pharmaceutical composition according to the invention, for preventing or treating an infection with a bacterium belonging to the species Listeria monocytogenes , or with an associated microorganism.

The invention is also directed toward an immunogenic and/or vaccinal composition, characterized in that it comprises one or more polypeptides according to the invention and/or one or more hybrid polypeptides according to the invention.

The invention also comprises the use of a transformed cell according to the invention, for preparing a vaccinal composition.

The invention is also directed toward a vaccinal composition, characterized in that it contains a nucleotide sequence according to the invention, a vector according to the invention and/or a transformed cell according to the invention.

The invention also relates to the vaccinal compositions according to the invention, for preventing or treating an infection with a bacterium belonging to the species Listeria monocytogenes, or with an associated microorganism.

Preferably, the immunogenic and/or vaccinal compositions according to the invention intended for the prevention and/or treatment of infection with Listeria monocytogenes, or with an associated microorganism will be chosen from the immunogenic and/or vaccinal compositions comprising a polypeptide, or a fragment thereof, corresponding to a protein, or a fragment thereof, of the cell envelope of Listeria monocytogenes. The vaccinal compositions comprising nucleotide sequences will preferably also comprise nucleotide sequences encoding a polypeptide, or a fragment thereof, corresponding to a protein, or a fragment thereof, of the cell envelope of Listeria monocytogenes.

Among these preferred immunogenic and/or vaccinal compositions, the most preferred are those comprising a polypeptide, or a fragment thereof, or a nucleotide sequence, or a fragment thereof, the sequences of which are chosen from the nucleotide or amino acid sequences identified in this functional group and listed previously.

The polypeptides of the invention, or the fragments thereof, which are part of the immunogenic compositions according to the invention may be selected using techniques known to those skilled in the art, such as, for example, the ability of said polypeptides to stimulate T cells, which, for example, causes the proliferation thereof or the secretion of interleukins, and which results in the production of antibodies directed against said polypeptides.

In mice, to which a weight dose of the vaccinal composition comparable to the dose used in humans is administered, the antibody reaction is tested by taking a serum sample and then studying the formation of a complex between the antibodies present in the serum and the antigen of the vaccinal composition, according to usual techniques.

According to the invention, said vaccinal compositions will preferably be in combination with a pharmaceutically acceptable vehicle and, where appropriate, with one or more suitable adjuvants of immunity.

Today, various types of vaccine are available for protecting humans against infectious diseases: attenuated live microorganisms (M. bovis—BCG for tuberculosis), inactive microorganisms (flu virus), acellular extracts (Bordetella pertussis for whooping cough), recombined proteins (hepatitis B virus surface antigens) and polysaccharides (pneumococci). Vaccines prepared from synthetic peptides or from genetically modified microorganisms expressing heterologous antigens are undergoing experimentation. Even more recently, recombined plasmid DNAs carrying genes encoding protective antigens have been proposed as an alternative vaccinal strategy. This type of vaccination is performed with a particular plasmid derived from an E. coli plasmid which does not replicate in vivo and which encodes only the immunizing protein. Animals have been immunized by simply injecting the naked plasmid DNA into muscle. This technique leads to the expression of the immunizing protein in situ and to an immune response of the cellular type (CTL) and of the humoral type (antibodies). This double induction of the immune response is one of the main advantages of the technique of vaccination with naked DNA.

The vaccinal compositions comprising nucleotide sequences or vectors into which said sequences are inserted are in particular described in international application No. WO 90/11092 and also in international application No. WO 95/11307.

The nucleotide sequence constituting the vaccinal composition according to the invention may be injected into the host after having been coupled to compounds which promote penetration of this polynucleotide into the cell or its transport as far as the cell nucleus. The resulting conjugates may be encapsulated in polymeric microparticles, as described in international application No. WO 94/27238 (Medisorb Technologies International).

According to another embodiment of the vaccinal composition according to the invention, the nucleotide sequence, preferably a DNA, is complexed with DEAE-dextran, with nuclear proteins or with lipids, or encapsulated in liposomes, or alternatively introduced in the form of a gel which facilitates its transfection into cells. The polynucleotide or the vector according to the invention may also be in suspension in a buffer solution or may be associated with liposomes.

Advantageously, such a vaccine will be prepared in accordance with the technique described by Tacson et al. or Huygen et al. in 1996, or else in accordance with the technique described by Davis et al. in international application No. WO 95/11307.

Such a vaccine may also be prepared in the form of a composition containing a vector according to the invention, placed under the control of regulatory elements for its expression in humans or animals. As a vector for in vivo expression of the polypeptide antigen of interest, use may, for example, be made of the plasmid pcDNA3 or the plasmid pcDNA1/neo, both marketed by Invitrogen (R & D Systems, Abi\overline{ng}don, United Kingdom). Such a vaccine will advantageously comprise, besides the recombinant vector, a saline solution, for example a sodium chloride solution.

The expression “pharmaceutically acceptable vehicle” is intended to denote a compound, or a combination of compounds, included in a pharmaceutical or vaccinal composition, which does not cause any side reactions and which makes it possible, for example, to facilitate administration of the active compound, to increase the lifetime thereof and/or the effectiveness thereof in the organism, to increase the solubility thereof in solution or else to improve the conservation thereof. These pharmaceutically acceptable vehicles are well known and will be adjusted by those skilled in the art depending on the nature and on the method of administration of the active compound chosen.

With regard to the vacinnal formulations, they may comprise suitable adjuvants of immunity which are known to those skilled in the art, such as, for example, aluminum hydroxide, a representative of the muramyl peptide family, such as one of the peptide derivatives of N-acetylmuramyl, a bacterial lyzate, or incomplete Freund's adjuvant.

Preferably, these compounds will be administered systemically, in particular intravenously, intramuscularly, intradermally or subcutaneously, or orally. More preferably, the vaccinal composition comprising polypeptides according to the invention will be administered several times, spread out over time, intradermally or subcutaneously.

The optimal methods of administration, doses and pharmaceutical forms of these compounds can be determined according to the criteria generally taken into account in establishing a suitable treatment for a patient, such as, for example, the age or body weight of the patient, the seriousness of his or her general condition, the tolerance to the treatment and the side effects noted.

The invention comprises the use of a composition according to the invention, for treating or preventing genital diseases induced or worsened by Listeria monocytogenes.

Finally, the invention comprises the use of a composition according to the invention, for treating or preventing diseases induced or worsened by the presence of Listeria monocytogenes.

Finally, the invention comprises the use of a composition according to the invention, for treating or preventing systemic diseases, in particular diseases of the lymphatic system, induced or worsened by the presence of Listeria monocytogenes.

Moreover, a subject of the present invention is also a genomic DNA library of a bacterium of the Listeria genus, preferably Listeria monocytogenes, preferably the EGD-e strain, said DNA library being cloned in bacterial artificial chromosomes (BACs). Such a genomic DNA library contains very large inserts of the Listeria genome, in particular inserts of between 50 and 200 kb in length.

One of the advantages of using the BAC system compared to a cosmid system is that there is only one or a maximum of two copies of the plasmids used per transformed cell, which decreases the potential for recombination between DNA fragments and, more importantly, which eliminates the risk of lethal overexpression of bacterial cloned genes. However, the presence of the BAC as a single copy means that the plasmid DNA must be extracted from a large volume of culture in order to obtain sufficient DNA for the sequence. In addition, the stability and fidelity with which the clones are maintained in a BAC library allows the identification of genomic differences between various Listeria strains, and the identification of these genetic differences which may be responsible for the phenotypic variations observed between the various strains.

The genomic DNA library described in the present invention, in particular the LM_baclim library deposited with the CNCM [National Collection of Microorganism Cultures] on Apr. 11, 2000, under the number I-2439, in fact covers the Listeria monocytogenes genome. However, although certain regions could not be cloned into said library, due to problems of lethality in Escherichia coli, these regions can easily be amplified and identified by those skilled in the art, using oligonucleotides specific for the sequences of the ends of the various clones which form the contigs.

The present invention also relates to the methods for isolating a polynucleotide of interest present in a strain of Listeria and absent from another strain, which use as at least one DNA library based on a BAC, containing the Listeria genome. The method according to the invention for isolating a polynucleotide of interest may comprise the following steps:
a) isolating at least one polynucleotide contained in a clone from the DNA library based on a BAC, of listeria origin;
b) isolating:
at least one genomic polynucleotide or cDNA of a listeria, said listeria belonging to a strain which is different from the strain used to construct the BAC DNA library of step a) or, alternatively,
at least one polynucleotide contained in a clone from a DNA library based on a BAC prepared from the genome of a listeria which is different from the listeria used to construct the DNA library based on the BAC of step a);
c) hybridizing the polynucleotide of step a) to the polynucleotide of step b);
d) selecting the polynucleotides of step a) which have not formed a hybridization complex with the polynucleotides of step b);
e) characterizing the polynucleotide selected.

The polynucleotide of step a) may be prepared by digesting at least one recombinant BAC clone with a suitable restriction enzyme and, optionally, amplifying the polynucleotide insert which results therefrom.

Thus, the method of the invention allows those skilled in the art to perform comparative genomic studies between the various strains or species of the listeria genus, for example between the pathogenic strains and their nonpathogenic equivalents.

In particular, it is possible to study and determine the regions of polymorphism between said strains.

### EXAMPLES

### Example 1

### Production of the BAC Library of Listeria monocytogenes DNA

Blocks containing the chromosomal DNA of Listeria monocytogenes, with an average weight of 80 mg, were prepared in agarose using methods known to those skilled in the art. They were kept in a solution of 500 mM EDTA, pH 8.0.

8 blocks are used to construct the library. These agarose blocks are washed twice for 30 min on ice in TE/PMSF (10 mM Tris-HCl, pH 8.0; 1 mM EDTA; 100 μM PMSF). The agarose blocks are then washed twice for 30 min on ice in TE (10 mM Tris-HCl, pH 8.0; 1 mM EDTA). Each agarose block is cut into eight thin slices, and sixteen slices are placed together in a single tube. The blocks are then incubated in a preincubation solution (20 μM spermidine, 5 mM DTT, 1×restriction buffer) for 40 min on ice. The preincubation buffer is changed once and incubated for a further 40 min.

The partial digestions are carried out in a digestion buffer (2 mM spermidine, 0.5 mM DTT, 0.02 μg BSA, 0.5×restriction buffer) for 30 min at 4° C., with no restriction enzyme, and then 0.1, 0.25, 0.4 or 0.5 units of EcoRI (Life Tech) per tube are added for 2 h at 37° C. The partial digestion is stopped by replacing the digestion buffer with 200 μl of 0.5M EDTA, the tubes being placed on ice.

The agarose blocks are then placed on a 1% SeaKem GTG agarose (FMC) gel containing 0.5×TBE buffer. Pulsed field gel electrophoresis is then carried out with the following conditions:
initial pulse time: 90 seconds,
final pulse time: 90 seconds,
6 V/cm,
included angle: 120°,
gel time: 16 h, 12° C.

The region between 50 and 200 kb is cut from the gel and separated into three pieces (50-100 kb, 100-150 kb, 150-200 kb). The gel for cloning should not be stained with ethidium bromide.

These agarose blocks are placed in a new 1% SeaKem GTG agarose (FMC) gel containing 0.5×TBE buffer. A further pulsed field gel electrophoresis is performed with the following conditions:
initial pulse time: 5 seconds,
final pulse time: 5 seconds,
4 V/cm,
included angle: 120°,
gel time: 15 h, 12° C.

The region between 50 and 200 kb is again cut out, without having stained the DNA with ethidium bromide, and separated into three pieces (50-100 kb, 100-150 kb, 150-200 kb).

The pieces of agarose are cut into small pieces, each of approximately 100 mg.

The agarose is incubated at 67° C. for 10 min and then cooled to 42° C., and 1 μl of beta-agarose (FMC, 1 U/μl) is added. The mixture is incubated for 30 min at 42° C., and the beta-agarose is denatured, after complete digestion of the agarose, by incubation for 10 min at 67° C. and then incubation on ice.

150 ng of DNA vectors digested with EcoRI and phosphorylated (CIP, Roche) are used to construct the BAC library. The vector pBelaBAC-Kan (Mozo et al., Mol. Gen. Genet., 1998, 258, 562-70) is used to construct said library. 150 ng of said vector are therefore incubated with 150 ng of DNA inserts, in a 1×ligation buffer with 5 units of ligase (USB 1 unit/μl) for 16 h at 12° C.

The ligation buffer is the buffer recommended by the manufacturer.

The transformation is carried out by adding 5 μl of the ligation reaction to 40 μl of DH10B electrocompetent cells, and the electroporation is performed in a Life Tech electroporator with the following conditions:
330 μF, 4 kΩ, DC volts: lowΩ, charge rate: fast, 1.5 mm electroporation cuvette.

1 mm of SOC culture medium is added to the cells, which are incubated for 45 min at 37° C. The cells are then plated out on a dish containing LB agar and which also contains 30 g/ml of kanamycin, 100 μg/ml of IPTG and 40 μg/ml of X-Gal. The recombinant cells which are white compared to the blue cells are selected, and the BAC library is characterized by preparing restriction maps for each of the clones thus obtained.

### Example 2

### Annotations and Analysis of the Genomic Sequence of Listeria monocytogenes

Bioinformatics has a key role in the three phases of a genome project: shotgun follow-up of the inserts produced using the random sequencing method, genome sequence closure phase, and annotations. The inventors have developed a complete software package which makes it possible to satisfy these three requirements: GMP-Tool-box (GMPTB).

Shotgun follow-up: During the random sequencing procedure, GMPTB extracts from the results files (Phrap format) or the characteristics required for the assembly (number of contigs, number of sequences, etc) and displays them in a table. This table may be used to create graphics which show the progression of this method and which allow rapid identification of the assembly problems. Importantly, GMPTB allows comparison between the assembly results and creates an HTML page to explain the relationship between new and old contigs (fusion, creation, etc).

Sequence closure phase: Various strategies are used by GMPTB to predict links between contigs. GMPTB in particular searches for all the clones which allow links, on the basis of the location and of the orientation of the terminal sequences. It can also indicate misassemblies. GMPTB can also predict links, on the basis of genome comparisons, by searching for similarities between the ends of the contigs and other genomic sequences (at nucleotide and amino acid level).

Annotations: GMPTB makes it possible to begin the annotation during the terminal phase. In fact, GMPTB creates an individual protein file (IPF) for each open reading frame (ORF) at the time of assembly. These are text files in a specific format which contain three categories of fields:
the minimum fields contain an identification number, a version number, location and sequences. The nucleotide sequence exported corresponds to the sequence of the open reading frame with 500 additional bases before the first stop codon and 200 additional bases after the second stop.
the automatic field contains results added to the IPF by different programs. It concerns the DNA sequence (search for ribosome binding sites, promoters or terminators, coding capacity, etc) and the predicted protein sequence (homology, domain, etc).
the manual field contains results and comments added by the users. After a new assembly, GMPTB extracts all the ORFs and creates new IPFs according to the IPF sequences derived from the previous assemblies. GMPTB recognizes the modified IPFs which are the only ones used for a new automatic analysis after each assembly.

The specificity of this strategy is that the annotation of the IPFs is independent of the assembly step, unless its sequence is modified. The IPFs are connected to a Sybases genomic databank (of the SubtiList model) and are accessible via a web server. They can be modified and annotated by different inventors during the genome project phase.

Depositing of Biological Material

The following organisms were deposited, on Apr. 11, 2000, at the Collection Nationale de Cultures de Microorganismes (CNCM) [National Collection of Microorganism Cultures], 25 rue du Docteur Roux, 75724 Paris Cedex 15, France, according to the provisions of the Treaty of Budapest:
Listeria monocytogenes strain EGD-e, number I-2440;

BAC library of Listeria DNA (145 clones), LM-baclib, number I-2439. Said BAC library (1-2439) was produced in the E. coli strain DH10B (Grant et al., PNAS, 87, 4645, 1990), constructed after partial digestion of the Listeria monocytogenes DNA with the EcoRI enzyme in the vector pBelaBAC-Kan (Mozo et al., Mol. Gen. Genet., 1998, 258, 562-70).

TABLE 1

| | | Location of nucleic acid | | |
|---|---|---|---|---|
| | | sequence of the ORF on | | |
| SEQ ID | Name | SEQ ID NO. 1 | | Function |
| SEQ ID NO. 2 | LM-1000.1 | From 589066 | to 589362 | Unknown, peptdidoglycan |
| | | | | bound protein (LPXTG |
| | | | | motif) |
| SEQ ID NO. 3 | LM-1002.1 | From 587012 | to 589033 | Unknown, similar to |
| | | | | internalin protein |
| SEQ ID NO. 4 | LM-1050.1 | From 2907153 | to 2909708 | Unknown, LPXTG protein |
| | | | | with LRR repeats |
| SEQ ID NO. 5 | LM-1179.1 | From 761552 | to 763468 | Unknown, putative |
| | | | | peptidoglycan bound protein |
| | | | | (LPXTG motif) |
| SEQ ID NO. 6 | LM-118.2 | From 2787416 | to 2788363 | Unknown, peptidoglycan |
| | | | | anchored protein (LPXTG |
| | | | | motif) |
| SEQ ID NO. 7 | LM-1235.1 | From 875721 | to 881855 | Unknown, surface protein |
| | | | | (LPXTG motif) |
| SEQ ID NO. 8 | LM-1248.1 | From 865530 | From | unknown, surface protein |
| | | | 865530 | (LPXTG motif) |
| SEQ ID NO. 9 | LM-1248.1 | From 865530 | From | unknown, surface protein |
| | | | 865530 | (LPXTG motif) |
| SEQ ID NO. 10 | LM-1305.1 | From 919020 | to 920408 | Unknown, similar to wall |
| | | | | associated protein |
| | | | | precursor (LPXTG motif) |
| SEQ ID NO. 11 | LM-1490.1 | From 649864 | to 651633 | Unknown, similar to |
| | | | | internalin proteins |
| SEQ ID NO. 12 | LM-1514.1 | From 664242 | to 668990 | Unknown, peptidoglycan |
| | | | | bound protein (LPXTG |
| | | | | motif) similar to adhesion |
| SEQ ID NO. 13 | LM-1660.3 | From 501312 | to 501617 | Unknown |
| SEQ ID NO. 14 | LM-1738.1 | From 547520 | to 549337 | Unknown, similar to |
| | | | | internalin proteins |
| SEQ ID NO. 15 | LM-1756.2 | From 2679599 | to 2681125 | Unknown, surface protein |
| | | | | (GW repeat) similar to |
| | | | | N-acetylmuramidase |
| SEQ ID NO. 16 | LM-1778.1 | From 1442368 | to 1443687 | Unknown, putative |
| | | | | peptidoglycan bound protein |
| | | | | (LPXTG motif) |
| SEQ ID NO. 17 | LM-1972.3 | From 1313654 | to 1315435 | Unknown, similar to |
| | | | | internalin proteins |
| SEQ ID NO. 18 | LM-1974.3 | From 1315767 | to 1317563 | Unknown, similar to |
| | | | | internalin proteins |
| SEQ ID NO. 19 | LM-2137.2 | From 360936 | to 366272 | Unknown, similar to |
| | | | | internalin proteins |
| SEQ ID NO. 20 | LM-229.1 | From 1170002 | to 1171621 | Unknown, putative |
| | | | | interanlin, similar to InlA |
| SEQ ID NO. 21 | LM-2323.1 | From 344850 | to 346049 | Unknown, similar to surface |
| | | | | proteins |
| SEQ ID NO. 22 | LM-2435.1 | From 159663 | to 161378 | Unknown, surface anchored |
| | | | | protein (LPXTG motif) |
| SEQ ID NO. 23 | LM-2438.1 | From 157089 | to 159470 | Unknown, surface anchored |
| | | | | protein (LPXTG motif) |
| SEQ ID NO. 24 | LM-2503.1 | From 2108500 | to 2109603 | Unknown, putative cell |
| | | | | surface protein, similar to |
| | | | | internalin proteins |
| SEQ ID NO. 25 | LM-2504.1 | From 2106329 | to 2108209 | Unknown, putative |
| | | | | peptidoglycan bound protein |
| | | | | (LPXTG motif) |
| SEQ ID NO. 26 | LM-3009.3 | From 1149887 | to 1152475 | Unknown, similar to |
| | | | | fibrinogen-binding protein |
| | | | | (LPXTG motif) |
| SEQ ID NO. 27 | LM-311.2 | From 131419 | to 133773 | Unknown, similar to 5- |
| | | | | nucleotidase (LPXTG motif) |
| SEQ ID NO. 28 | LM-3144.1 | From 351459 | to 355505 | Unknown, similar to cell |
| | | | | surface proteins (LPXTG |
| | | | | motif) |
| SEQ ID NO. 29 | LM-3369.1 | From 1869532 | to 1872243 | Unknown, putative |
| | | | | peptidoglycan bound protein |
| | | | | (LPXTG motif) |
| SEQ ID NO. 30 | LM-3418.2 | From 1717193 | to 1722328 | Unknown, peptidoglycan |
| | | | | linked protein (LPxTG) |
| SEQ ID NO. 31 | LM-3477.1 | From 828168 | to 830108 | Unknown, similar to |
| | | | | internalin |
| SEQ ID NO. 32 | LM-3609.1 | From 1106041 | to 1107759 | Unknown, similar to |
| | | | | AUTOLYSIN (EC 3.5.1.28) |
| | | | | (N-ACETYLMURAMOYL-L- |
| | | | | ALANINE AMIDASE) |
| SEQ ID NO. 33 | LM-3691.2 | From 2162323 | to 2164011 | Unknown, putative |
| | | | | peptidoglycan bound protein |
| | | | | (LPXTG motif) |
| SEQ ID NO. 34 | LM-3700.2 | From 2653211 | to 2657803 | Unknown, peptidoglycan |
| | | | | anchored protein (LPXTG |
| | | | | motif) |
| SEQ ID NO. 35 | LM-3752.3 | From 357775 | to 359676 | Unknown, similar to |
| | | | | internalin |
| SEQ ID NO. 36 | LM-757.1 | From 2544267 | to 2545433 | Unknown, similar to |
| | | | | internalin proteins |
| SEQ ID NO. 37 | LM-814.2 | From 2264772 | to 2268230 | Unknown, putative |
| | | | | peptidoglycan bound protein |
| | | | | (LPXTG motif) |
| SEQ ID NO. 38 | LM-816.2 | From 2259753 | to 2264591 | Unknown, putative |
| | | | | peptidoglycan bound protein |
| | | | | (LPXTG motif) |
| SEQ ID NO. 39 | LM-894.1 | From 2469093 | to 2471915 | Unknown, similar to |
| | | | | internalin proteins |
| SEQ ID NO. 40 | LM-966.1 | From 173309 | to 174556 | Unknown, cell wall |
| | | | | anchored protein |
| SEQ ID NO. 41 | LM-973.1 | From 169510 | to 172008 | Unknown, similar to |
| | | | | internalin proteins |
| SEQ ID NO. 42 | LM-133.1 | From 1230773 | to 1232308 | Unknown, similar to cobyric |
| | | | | acid synthase CbiP |
| SEQ ID NO. 43 | LM-134.1 | From 1229967 | to 1230773 | Unknown, similar to cobalt |
| | | | | transport ATP-binding |
| | | | | protein CbiO |
| SEQ ID NO. 44 | LM-135.1 | From 1229277 | to 1229954 | Unknown, similar to cobalt |
| | | | | transport protein Q |
| SEQ ID NO. 45 | LM-136.1 | From 1228994 | to 1229290 | Unknown, similar to putative |
| | | | | cobalt transport protein |
| | | | | CbiN |
| SEQ ID NO. 46 | LM-137.1 | From 1228263 | to 1228997 | Unknown, similar to |
| | | | | cobalamin biosynthesis |
| | | | | protein M |
| SEQ ID NO. 47 | LM-138.1 | From 1227556 | to 1228266 | Unknown, similar to |
| | | | | S-adenosyl-methionine: |
| | | | | precorrin-2 |
| | | | | methyltransferase |
| SEQ ID NO. 48 | LM-139.1 | From 1226778 | to 1227563 | unknown, similar to |
| | | | | anaerobic Cobalt Chelatase |
| | | | | In Cobalamin Biosynthesis |
| SEQ ID NO. 49 | LM-141.1 | From 1225300 | to 1226781 | Unknown, similar |
| | | | | uroporphyrinogen-III |
| | | | | methyltransferase/uroporphyrinogen- |
| | | | | III synthase |
| SEQ ID NO. 50 | LM-142.1 | From 1224548 | to 1225300 | Unknown, similar to |
| | | | | cobalamin biosynthesis J |
| | | | | protein CbiJ |
| SEQ ID NO. 51 | LM-143.1 | From 1223826 | to 1224551 | Unknown, similar to |
| | | | | precorrin methylase |
| SEQ ID NO. 52 | LM-144.1 | From 1222798 | to 1223829 | Unknown, similar to |
| | | | | cobalamin biosynthesis |
| | | | | protein G CbiG |
| SEQ ID NO. 53 | LM-145.1 | From 1222062 | to 1222811 | Unknown, similar to |
| | | | | precorrin-3 methylase |
| SEQ ID NO. 54 | LM-146.1 | From 1221487 | to 1222056 | Unknown, similar to |
| | | | | precorrin decarbocylase |
| SEQ ID NO. 55 | LM-147.1 | From 1220901 | to 1221497 | Unknown, similar to |
| | | | | precorrin methylase |
| SEQ ID NO. 56 | LM-149.1 | From 1219783 | to 1220904 | Unknown, similar to |
| | | | | cobalamin biosynthesis |
| | | | | protein CbiD |
| SEQ ID NO. 57 | LM-150.1 | From 1219135 | to 1219767 | Unknown, similar to |
| | | | | precorrin isomerase |
| SEQ ID NO. 58 | LM-151.1 | From 1218175 | to 1219122 | Unknown, similar to |
| | | | | cobalamine synthesis |
| | | | | protein CbiB |
| SEQ ID NO. 59 | LM-152.1 | From 1216963 | to 1217322 | FALSE ORF |
| SEQ ID NO. 60 | LM-181.1 | From 1196097 | to 1197182 | unknown, similar to |
| | | | | Salmonella typhimurium |
| | | | | CobD protein and to |
| | | | | histidinol-phosphate |
| | | | | aminotransferase |
| SEQ ID NO. 61 | LM-207.1 | From 1180152 | to 1181036 | Regulatory protein similar to |
| | | | | Salmonella typhimurium |
| | | | | PocR protein |
| SEQ ID NO. 62 | LM-209.1 | From 1179168 | to 1179743 | unknown, similar to alpha- |
| | | | | ribazole-5-phosphatase |
| SEQ ID NO. 63 | LM-210.1 | From 1178421 | to 1179167 | Unknown, highly similar to |
| | | | | cobalamin (5-phosphatase) |
| | | | | synthetase |
| SEQ ID NO. 64 | LM-212.1 | From 1177862 | to 1178419 | unknown, similar to |
| | | | | bifunctional cobalamin |
| | | | | biosynthesis protein CopB, |
| | | | | (cobinamide kinase; |
| | | | | cobinamide phosphatase |
| | | | | guanylyltransferase) |
| SEQ ID NO. 65 | LM-1.3 | From 2710869 | to 2712755 | Unknown, similar to NADH |
| | | | | dehydrogenase |
| SEQ ID NO. 66 | LM-10.1 | From 2718312 | to 2719055 | Unknown |
| SEQ ID NO. 67 | LM-100.1 | From 2775644 | to 2776486 | Unknown, similar to |
| | | | | aldo/keto reductase |
| SEQ ID NO. 68 | LM-1003.1 | From 586329 | to 586994 | Unknown |
| SEQ ID NO. 69 | LM-1004.1 | From 585015 | to 585962 | Unknown, similar to B. subtilis DeoR |
| | | | | transcriptional |
| | | | | regulator |
| SEQ ID NO. 70 | LM-1005.1 | From 583507 | to 584757 | Unknown, similar to putative |
| | | | | NAD(P)-dependent |
| | | | | oxidoreductase |
| SEQ ID NO. 71 | LM-1007.1 | From 583072 | to 583452 | Unknown |
| SEQ ID NO. 72 | LM-1008.1 | From 582526 | to 583047 | Unknown, similar to PTS |
| | | | | system, glucitol/sorbitol- |
| | | | | specific enzyme II CII |
| | | | | component |
| SEQ ID NO. 73 | LM-1009.1 | From 581519 | to 582505 | Unknown, similar to PTS |
| | | | | system, glucitol/sorbitol- |
| | | | | specific enzyme IIBC |
| | | | | component |
| SEQ ID NO. 74 | LM-101.1 | From 2776532 | to 2776777 | Unknown, similar to B. subtilis |
| | | | | YaaL protein |
| SEQ ID NO. 75 | LM-1010.1 | From 581150 | to 581500 | Unknown, similar to PTS |
| | | | | system, glucitol/sorbitol- |
| | | | | specific enzyme IIA |
| | | | | component |
| SEQ ID NO. 76 | LM-1011.1 | From 580119 | to 581039 | Unknown, similar to ABC |
| | | | | transporter (binding protein) |
| SEQ ID NO. 77 | LM-1012.1 | From 578886 | to 580079 | Unknown, similar to |
| | | | | penicillin-binding protein |
| SEQ ID NO. 78 | LM-1013.1 | From 577632 | to 578648 | Unknown, similar to |
| | | | | tagatose-1,6-diphosphate |
| | | | | aldolase |
| SEQ ID NO. 79 | LM-1015.1 | From 576403 | to 577584 | Unknown, similar to N-acyl- |
| | | | | L-amino acid |
| | | | | amidohydrolase |
| SEQ ID NO. 80 | LM-1017.1 | From 575166 | to 576437 | Unknown, similar to N- |
| | | | | carbamyl-L-amino acid |
| | | | | amidohydrolase |
| SEQ ID NO. 81 | LM-1018.1 | From 573740 | to 575062 | Unknown, similar to 6- |
| | | | | phospho-beta-glucosidase |
| SEQ ID NO. 82 | LM-102.1 | From 2776792 | to 2777388 | Unknown, highly similar to |
| | | | | recombination protein recR |
| SEQ ID NO. 83 | LM-1020.1 | From 572600 | to 573568 | Unknown, similar to |
| | | | | transcription regulator (Lacl |
| | | | | family) |
| SEQ ID NO. 84 | LM-1022.1 | From 571204 | to 572559 | Unknown, similar to |
| | | | | unknown proteins |
| SEQ ID NO. 85 | LM-1023.1 | From 570916 | to 571185 | Unknown, similar to |
| | | | | unknown proteins |
| SEQ ID NO. 86 | LM-1024.1 | From 570008 | to 570838 | Unknown |
| SEQ ID NO. 87 | LM-1025.1 | From 569094 | to 569948 | Unknown |
| SEQ ID NO. 88 | LM-1029.1 | From 567030 | to 569081 | Unknown |
| SEQ ID NO. 89 | LM-103.1 | From 2777497 | to 2777814 | Unknown, highly similar to |
| | | | | B. subtilis YaaK protein |
| SEQ ID NO. 90 | LM-1031.1 | From 565764 | to 567014 | Unknown, conserved |
| | | | | hypothetical protein similar |
| | | | | to putative |
| | | | | glucosaminyltransferase |
| SEQ ID NO. 91 | LM-1032.1 | From 564257 | to 565753 | Unknown, hypothetical |
| | | | | secreted protein |
| SEQ ID NO. 92 | LM-1033.1 | From 562789 | to 564264 | Unknown, transmembrane |
| | | | | protein |
| SEQ ID NO. 93 | LM-1036.1 | From 561819 | to 562559 | Unknown, similar to |
| | | | | transcription regulator (TipA |
| | | | | from Streptomyces |
| | | | | coelicolor) |
| SEQ ID NO. 94 | LM-1037.1 | From 560443 | to 561774 | Unknown |
| SEQ ID NO. 95 | LM-1040.2 | From 2915744 | to 2916097 | Unknown |
| SEQ ID NO. 96 | LM-1041.2 | From 2915219 | to 2915641 | Unknown similar to |
| | | | | transcriptional regulator |
| | | | | (MarR family) |
| SEQ ID NO. 97 | LM-1043.1 | From 2914057 | to 2915205 | Unknown, similar to efflux |
| | | | | proteins |
| SEQ ID NO. 98 | LM-1045.1 | From 2912595 | to 2913686 | Unknown, highly similar to |
| | | | | phosphoserine |
| | | | | aminotransferase |
| SEQ ID NO. 99 | LM-1047.1 | From 2911415 | to 2912602 | Unknown, similar to D-3- |
| | | | | phosphoglycerate |
| | | | | dehydrogenase |
| SEQ ID NO. 100 | LM-1048.1 | From 2910085 | to 2911326 | Unknown, similar to an |
| | | | | hypothetical protein from |
| | | | | Thermotoga maritima |
| SEQ ID NO. 101 | LM-1049.1 | From 2909753 | to 2910076 | Unknown |
| SEQ ID NO. 102 | LM-1052.1 | From 2904598 | to 2906940 | Unknown, amino-terminal |
| | | | | domain similar to |
| | | | | transcription regulators |
| SEQ ID NO. 103 | LM-1054.1 | From 2903200 | to 2904402 | Unknown, similar to |
| | | | | carboxypeptidase |
| SEQ ID NO. 104 | LM-1055.1 | From 2901800 | to 2903200 | Unknown, similar to |
| | | | | transmembrane efflux |
| | | | | protein |
| SEQ ID NO. 105 | LM-1056.1 | From 2900594 | to 2901784 | Unknown, similar to |
| | | | | peptidases |
| SEQ ID NO. 106 | LM-1057.1 | From 2899313 | to 2900506 | Unknown, similar to |
| | | | | transport protein |
| SEQ ID NO. 107 | LM-1058.1 | From 2898535 | to 2899266 | Unknown, similar to |
| | | | | reductases |
| SEQ ID NO. 108 | LM-1059.1 | From 2897847 | to 2898374 | Unknown, similar to |
| | | | | transcriptional regulator |
| SEQ ID NO. 109 | LM-1060.1 | From 2897116 | to 2897823 | Unknown |
| SEQ ID NO. 110 | LM-1061.1 | From 2896241 | to 2897059 | Unknown, similar to D- |
| | | | | alanyl-D-alanine |
| | | | | carboxypeptidase |
| SEQ ID NO. 111 | LM-1064.1 | From 2894510 | to 2895883 | Unknown, similar to |
| | | | | GTPase |
| SEQ ID NO. 112 | LM-1066.1 | From 2892596 | to 2894485 | Unknown, highly similar to |
| | | | | GidA protein |
| SEQ ID NO. 113 | LM-1067.2 | From 2892057 | to 2892437 | Unknown, hypothetical |
| | | | | secreted protein |
| SEQ ID NO. 114 | LM-1069.3 | From 434945 | to 435817 | Unknown |
| SEQ ID NO. 115 | LM-107.1 | From 2777844 | to 2779583 | Unknown, highly similar to |
| | | | | DNA polymerase III |
| | | | | (gamma and tau subunits) |
| SEQ ID NO. 116 | LM-1070.1 | From 433153 | to 434745 | Unknown, similar to |
| | | | | phosphoenolpyruvate |
| | | | | synthase (N-terminal part) |
| SEQ ID NO. 117 | LM-1074.1 | From 429630 | to 432095 | Internalin |
| SEQ ID NO. 118 | LM-1075.1 | From 428981 | to 429403 | Unknown |
| SEQ ID NO. 119 | LM-1076.1 | From 428576 | to 428968 | Unknown |
| SEQ ID NO. 120 | LM-1077.1 | From 428127 | to 428507 | Unknown, similar to B. subtilis |
| | | | | YyaH protein |
| SEQ ID NO. 121 | LM-1079.1 | From 427104 | to 428111 | Unknown, similar to |
| | | | | phosphate transport protein |
| SEQ ID NO. 122 | LM-108.1 | From 2779910 | to 2780374 | Unknown |
| SEQ ID NO. 123 | LM-1080.1 | From 426597 | to 426995 | Unknown |
| SEQ ID NO. 124 | LM-1081.1 | From 426190 | to 426606 | Unknown |
| SEQ ID NO. 125 | LM-1082.1 | From 424130 | to 426064 | Unknown, similar to |
| | | | | transcriptional |
| | | | | antiterminator (BglG family) |
| SEQ ID NO. 126 | LM-1083.1 | From 421469 | to 424096 | Unknown, highly similar to |
| | | | | E. col YbgG protein, a |
| | | | | putative sugar hydrolase |
| SEQ ID NO. 127 | LM-1084.1 | From 420335 | to 421447 | Unknown, similar to |
| | | | | fructose-specific |
| | | | | phosphotransferase |
| | | | | enzyme IIC |
| SEQ ID NO. 128 | LM-1085.1 | From 419999 | to 420331 | Unknown, similar to |
| | | | | fructose-specific |
| | | | | phosphotransferase |
| | | | | enzyme IIB |
| SEQ ID NO. 129 | LM-1088.1 | From 419544 | to 420002 | Unknown, similar to |
| | | | | phosphotransferase system |
| | | | | enzyme IIA |
| SEQ ID NO. 130 | LM-1089.1 | From 418891 | to 419373 | Unknown, similar to |
| | | | | unknown proteins |
| SEQ ID NO. 131 | LM-109.1 | From 2780933 | to 2782297 | Unknown, similar to PTS |
| | | | | system, cellobiose-specific |
| | | | | enzyme IIC |
| SEQ ID NO. 132 | LM-1091.1 | From 417963 | to 418763 | Unknown, similar to 1- |
| | | | | pyrroline-5-carboxylate |
| | | | | reductase (ProC) |
| SEQ ID NO. 133 | LM-1092.1 | From 417535 | to 417960 | Unknown, weakly similar to |
| | | | | blasticidin S- |
| | | | | acetyltransferase |
| SEQ ID NO. 134 | LM-1093.1 | From 416796 | to 417479 | Unknown, similar to L. monocytogenes |
| | | | | extracellular P60 protein |
| SEQ ID NO. 135 | LM-1094.1 | From 416203 | to 416652 | Unknown |
| SEQ ID NO. 136 | LM-1097.1 | From 415224 | to 416168 | Unknown, highly similar to |
| | | | | B. subtilis YqfA protein |
| SEQ ID NO. 137 | LM-1098.1 | From 414928 | to 415227 | Unknown |
| SEQ ID NO. 138 | LM-1099.1 | From 414095 | to 414760 | Unknown, similar to uracil- |
| | | | | DNA glycosylase |
| SEQ ID NO. 139 | LM-11.1 | From 2719056 | to 2719775 | Unknown |
| SEQ ID NO. 140 | LM-1100.1 | From 412849 | to 413964 | Unknown, low temperature |
| | | | | requirement protein A |
| SEQ ID NO. 141 | LM-1101.1 | From 412457 | to 412825 | Unknown |
| SEQ ID NO. 142 | LM-1102.1 | From 411992 | to 412363 | Unknown, similar to B. subtilis |
| | | | | YhdG protein |
| SEQ ID NO. 143 | LM-1105.1 | From 409944 | to 411860 | Unknown, similar to B. subtilis |
| | | | | IolD protein, to |
| | | | | acetolactate synthase |
| SEQ ID NO. 144 | LM-1107.1 | From 408954 | to 409931 | Unknown, similar to B. subtilis |
| | | | | IolC protein and to |
| | | | | fructokinase |
| SEQ ID NO. 145 | LM-1108.1 | From 408116 | to 408937 | Unknown, similar to B. subtilis |
| | | | | IolB protein |
| SEQ ID NO. 146 | LM-1110.2 | From 406637 | to 408103 | Unknown, highly similar to |
| | | | | B. subtilis methylmalonate- |
| | | | | semialdehyde |
| | | | | dehydrogenase IolA |
| SEQ ID NO. 147 | LM-1112.2 | From 405680 | to 406441 | Unknown, similar to B. subtilis |
| | | | | transcription |
| | | | | repressor of myo-inositol |
| | | | | catabolism operon IolR |
| SEQ ID NO. 148 | LM-1114.1 | From 405221 | to 405607 | Unknown |
| SEQ ID NO. 149 | LM-1115.3 | From 404365 | to 404994 | Unknown |
| SEQ ID NO. 150 | LM-1116.4 | From 807976 | to 810792 | Unknown, similar to |
| | | | | transcriptional regulator |
| | | | | (NifA/NtrC family) |
| SEQ ID NO. 151 | LM-1117.1 | From 807255 | to 807689 | Unknown, similar to |
| | | | | mannose-specific |
| | | | | phosphotransferase system |
| | | | | (PTS) component IIA |
| SEQ ID NO. 152 | LM-1118.1 | From 806770 | to 807255 | Unknown, similar to |
| | | | | mannose-specific |
| | | | | phosphotransferase system |
| | | | | (PTS) component IIB |
| SEQ ID NO. 153 | LM-112.1 | From 2783466 | to 2784107 | Unknown |
| SEQ ID NO. 154 | LM-1121.1 | From 805784 | to 806596 | Unknown, similar to |
| | | | | mannose-specific |
| | | | | phosphotransferase system |
| | | | | (PTS) component IIC |
| SEQ ID NO. 155 | LM-1122.1 | From 804887 | to 805765 | Unknown, similar to |
| | | | | mannose-specific |
| | | | | phosphotransferase system |
| | | | | (PTS) component IID |
| SEQ ID NO. 156 | LM-1123.1 | From 804382 | to 804729 | Unknown |
| SEQ ID NO. 157 | LM-1125.1 | From 803745 | to 804212 | Unknown |
| SEQ ID NO. 158 | LM-1126.1 | From 803201 | to 803548 | Unknown |
| SEQ ID NO. 159 | LM-1127.1 | From 802356 | to 802739 | Unknown |
| SEQ ID NO. 160 | LM-1128.1 | From 801336 | to 802202 | Unknown, similar to |
| | | | | transcription regulator |
| | | | | (repressor) |
| SEQ ID NO. 161 | LM-1129.1 | From 800965 | to 801297 | Unknown |
| SEQ ID NO. 162 | LM-113.1 | From 2784354 | to 2784674 | Unknown, similar to |
| | | | | hypothetical proteins |
| SEQ ID NO. 163 | LM-1130.1 | From 799996 | to 800925 | Unknown, conserved |
| | | | | hypothetical protein |
| SEQ ID NO. 164 | LM-1131.1 | From 798828 | to 799817 | Unknown, similar to alcohol |
| | | | | dehydrogenase |
| SEQ ID NO. 165 | LM-1132.1 | From 798141 | to 798788 | Unknown, similar to |
| | | | | transcription regulator |
| SEQ ID NO. 166 | LM-1133.1 | From 797353 | to 798105 | Unknown |
| SEQ ID NO. 167 | LM-1134.1 | From 796075 | to 797193 | Unknown, similar to |
| | | | | transcriptional regulator |
| | | | | (LacI family) |
| SEQ ID NO. 168 | LM-1135.1 | From 795034 | to 796062 | Unknown, similar to alpha- |
| | | | | 1,6-mannanase |
| SEQ ID NO. 169 | LM-1136.1 | From 793777 | to 795030 | Unknown, similar to sugar |
| | | | | ABC transporter, |
| | | | | periplasmic sugar-binding |
| | | | | protein |
| SEQ ID NO. 170 | LM-1137.1 | From 792879 | to 793754 | Unknown, similar to ABC |
| | | | | transporter, permease |
| | | | | protein |
| SEQ ID NO. 171 | LM-1138.1 | From 791977 | to 792867 | Unknown, similar to putative |
| | | | | sugar ABC transporter, |
| | | | | permease protein |
| SEQ ID NO. 172 | LM-1139.1 | From 790662 | to 791960 | Unknown |
| SEQ ID NO. 173 | LM-114.1 | From 2784805 | to 2786319 | Unknown, highly similar to |
| | | | | gluconate kinase |
| SEQ ID NO. 174 | LM-1140.1 | From 789520 | to 790509 | Unknown, similar to lipoate- |
| | | | | protein ligase |
| SEQ ID NO. 175 | LM-1141.1 | From 788642 | to 789514 | Unknown, similar to |
| | | | | unknown proteins |
| SEQ ID NO. 176 | LM-1142.1 | From 787320 | to 788576 | Unknown, similar to |
| | | | | ATP/GTP-binding protein |
| SEQ ID NO. 177 | LM-1143.1 | From 786372 | to 786992 | Unknown, similar to |
| | | | | unknown proteins |
| SEQ ID NO. 178 | LM-1144.1 | From 785754 | to 786362 | Unknown |
| SEQ ID NO. 179 | LM-1146.1 | From 784788 | to 785747 | Unknown |
| SEQ ID NO. 180 | LM-1147.1 | From 783901 | to 784788 | Unknown |
| SEQ ID NO. 181 | LM-1149.1 | From 782794 | to 783774 | Unknown, similar to |
| | | | | hypothetical proteins |
| SEQ ID NO. 182 | LM-1151.1 | From 781896 | to 782801 | Unknown; Similar to ABC |
| | | | | transporter (ATP-binding |
| | | | | protein) |
| SEQ ID NO. 183 | LM-1152.1 | From 781103 | to 781924 | Unknown, similar to |
| | | | | unknown proteins |
| SEQ ID NO. 184 | LM-1153.2 | From 780377 | to 780988 | Unknown, weakly similar to |
| | | | | a bile acid 7-alpha |
| | | | | dehydratase |
| SEQ ID NO. 185 | LM-1154.1 | From 779616 | to 780296 | Unknown, similar to |
| | | | | transcription regulator |
| | | | | Crp/Fnr family |
| SEQ ID NO. 186 | LM-1155.1 | From 778654 | to 779490 | Unknown, weakly similar to |
| | | | | a putative haloacetate |
| | | | | dehalogenase |
| SEQ ID NO. 187 | LM-1156.1 | From 778262 | to 778558 | Unknown |
| SEQ ID NO. 188 | LM-1157.1 | From 777692 | to 778207 | Unknown |
| SEQ ID NO. 189 | LM-1158.1 | From 777209 | to 777496 | Unknown |
| SEQ JD NO. 190 | LM-1159.1 | From 776761 | to 777102 | Unknown |
| SEQ ID NO. 191 | LM-1160.1 | From 776398 | to 776697 | Unknown, hypothetical |
| SEQ ID NO. 192 | LM-1161.1 | From 775744 | to 776247 | Unknown |
| SEQ ID NO. 193 | LM-1164.1 | From 773715 | to 775715 | Unknown, similar to ABC |
| | | | | transporter, ATP-binding |
| | | | | protein |
| SEQ ID NO. 194 | LM-1165.1 | From 772996 | to 773700 | Unknown |
| SEQ ID NO. 195 | LM-1166.1 | From 772310 | to 772999 | Unknown, similar to ABC |
| | | | | transporter, ATP-binding |
| | | | | protein |
| SEQ ID NO. 196 | LM-1167.1 | From 771949 | to 772326 | Unknown, similar to |
| | | | | transcriptional regulator |
| | | | | (GntR family) |
| SEQ ID NO. 197 | LM-1168.1 | From 771026 | to 771685 | Unknown, similar to putative |
| | | | | transcription regulator |
| SEQ ID NO. 198 | LM-1169.1 | From 769639 | to 771012 | Unknown, similar to 6- |
| | | | | phospho-beta-glucosidase |
| SEQ ID NO. 199 | LM-117.1 | From 2786435 | to 2787373 | Unknown, secreted protein |
| | | | | with 1 GW repeat |
| SEQ ID NO. 200 | LM-1171.1 | From 767766 | to 769619 | Unknown, similar to |
| | | | | phosphotransferase system |
| | | | | (PTS) beta-glucoside- |
| | | | | specific enzyme IIABC |
| | | | | component |
| SEQ ID NO. 201 | LM-1172.1 | From 766810 | to 767742 | Unknown |
| SEQ ID NO. 202 | LM-1173.1 | From 766351 | to 766797 | Unknown, similar to ribose |
| | | | | 5-phosphate isomerase |
| SEQ ID NO. 203 | LM-1174.1 | From 765683 | to 766354 | Unknown, similar to |
| | | | | Ribulose-5-Phosphate 3- |
| | | | | Epimerase |
| SEQ ID NO. 204 | LM-1175.1 | From 764465 | to 765469 | Unknown, similar to |
| | | | | transcriptional regulator |
| | | | | (LacI family) |
| SEQ ID NO. 205 | LM-1176.1 | From 763798 | to 764307 | Unknown, similar to |
| | | | | transcription regulator |
| SEQ ID NO. 206 | LM-1181.1 | From 760455 | to 760859 | Unknown |
| SEQ ID NO. 207 | LM-1182.2 | From 759972 | to 760310 | Unknown |
| SEQ ID NO. 208 | LM-1183.2 | From 759479 | to 759958 | Unknown |
| SEQ ID NO. 209 | LM-1184.3 | From 2916291 | to 2916890 | Unknown, similar to yeast |
| | | | | protein Frm2p involved in |
| | | | | fatty acid signaling |
| SEQ ID NO. 210 | LM-1186.1 | From 2916946 | to 2917266 | Unknown, similar to |
| | | | | thioredoxin |
| SEQ ID NO. 211 | LM-1189.1 | From 2917389 | to 2918051 | Unknown, similar to |
| | | | | phosphoglucomutase |
| SEQ ID NO. 212 | LM-119.2 | From 1239842 | to 1240201 | Unknown |
| SEQ ID NO. 213 | LM-1190.1 | From 2918048 | to 2919169 | Unknown; similar to |
| | | | | unknown proteins |
| SEQ ID NO. 214 | LM-1195.1 | From 2919166 | to 2921343 | Unknown, similar to a |
| | | | | maltose phosphorylase |
| SEQ ID NO. 215 | LM-1196.1 | From 2921340 | to 2922371 | Unknown, similar to |
| | | | | oxidoreductases |
| SEQ ID NO. 216 | LM-1198.1 | From 2922429 | to 2923223 | Unknown, highly similar to |
| | | | | an E. coli protein |
| SEQ ID NO. 217 | LM-1199.1 | From 2923236 | to 2924288 | Unknown, similar to alcohol |
| | | | | dehydrogenase |
| SEQ ID NO. 218 | LM-1201.1 | From 2924307 | to 2925158 | Unknown, similar to sugar |
| | | | | ABC transporter permease |
| | | | | protein |
| SEQ ID NO. 219 | LM-1202.1 | From 2925145 | to 2926026 | Unknown, similar to sugar |
| | | | | ABC transporter permease |
| | | | | protein |
| SEQ ID NO. 220 | LM-1203.1 | From 2926101 | to 2927393 | Unknown, similar to sugar |
| | | | | binding protein (ABC |
| | | | | transporter) |
| SEQ ID NO. 221 | LM-1205.1 | From 2927390 | to 2928631 | Unknown, similar to |
| | | | | Sucrose phosphorylase |
| SEQ ID NO. 222 | LM-1206.1 | From 2928668 | to 2929090 | Unknown, weakly similar to |
| | | | | sucrose phosphorylase |
| SEQ ID NO. 223 | LM-1208.1 | From 2929315 | to 2930340 | Unknown, similar to |
| | | | | transcriptional regulator |
| SEQ ID NO. 224 | LM-1209.1 | From 2930479 | to 2932473 | Unknown |
| SEQ ID NO. 225 | LM-121.1 | From 1239039 | to 1239797 | Unknown, similar to rRNA |
| | | | | methylase |
| SEQ ID NO. 226 | LM-1210.1 | From 2932609 | to 2933148 | Unknown, similar to |
| | | | | unknown proteins |
| SEQ ID NO. 227 | LM-1212.1 | From 2933303 | to 2934715 | Unknown, similar to |
| | | | | transmembrane efflux |
| | | | | proteins |
| SEQ ID NO. 228 | LM-1213.1 | From 2934756 | to 2935070 | Unknown, highly similar to |
| | | | | B. subtilis YuID protein |
| SEQ ID NO. 229 | LM-1214.1 | From 2935083 | to 2935904 | Unknown, highly similar to |
| | | | | rhamnulose-1-phosphate |
| | | | | aldolase |
| SEQ ID NO. 230 | LM-1216.1 | From 2935917 | to 2937179 | Unknown, highly similar to |
| | | | | L-rhamnose isomerase |
| SEQ ID NO. 231 | LM-1218.1 | From 2937192 | to 2938643 | Unknown; similar to |
| | | | | rhamnulokinase |
| SEQ ID NO. 232 | LM-122.1 | From 1237934 | to 1239013 | Unknown, similar to endo- |
| | | | | 1,4-beta-glucanase and to |
| | | | | aminopeptidase |
| SEQ ID NO. 233 | LM-1220.1 | From 2938662 | to 2939930 | Unknown, similar to sugar |
| | | | | transport proteins |
| SEQ ID NO. 234 | LM-1221.1 | From 2940053 | to 2941033 | Unknown, similar to AraC- |
| | | | | type regulatory protein |
| SEQ ID NO. 235 | LM-1222.1 | From 2941083 | to 2941544 | Unknown |
| SEQ ID NO. 236 | LM-1223.2 | From 2941599 | to 2942219 | Unknown, highly similar to |
| | | | | B. subtilis Jag protein |
| SEQ ID NO. 237 | LM-1224.3 | From 2942216 | to 2943079 | Unknown, highly similar to |
| | | | | B. subtilis SpoIIIJ protein |
| SEQ ID NO. 238 | LM-1225.2 | From 884170 | to 885270 | Unknown, similar to |
| | | | | excinuclease ABC, chain C |
| | | | | (UvrC) |
| SEQ ID NO. 239 | LM-1228.1 | From 882962 | to 884065 | Unknown, similar to B. subtilis |
| | | | | YxjH and YxjG |
| | | | | proteins |
| SEQ ID NO. 240 | LM-123.1 | From 1236836 | to 1237822 | Unknown, similar to N- |
| | | | | acetylmuramoyl-L-alanine |
| | | | | amidase (autolysin) |
| SEQ ID NO. 241 | LM-1230.1 | From 882328 | to 882705 | Unknown, conserved |
| | | | | hypothetical protein |
| SEQ ID NO. 242 | LM-1231.1 | From 881992 | to 882249 | Unknown, similar to B. subtilis |
| | | | | protein YsdA |
| SEQ ID NO. 243 | LM-1239.1 | From 872616 | to 875258 | unknown, similar to cation |
| | | | | (clacium) transporting |
| | | | | ATPase |
| SEQ ID NO. 244 | LM-1241.1 | From 871940 | to 872410 | Unknown |
| SEQ ID NO. 245 | LM-1242.1 | From 870587 | to 871807 | Unknown, similar to |
| | | | | Tetracycline resistance |
| | | | | protein |
| SEQ ID NO. 246 | LM-1243.1 | From 869095 | to 870480 | unknown, highly similar to |
| | | | | hexose phosphate transport |
| | | | | protein |
| SEQ ID NO. 247 | LM-1245.1 | From 867163 | to 868800 | Unknown, ABC transporter |
| | | | | (ATP binding protein) |
| SEQ ID NO. 248 | LM-1246.1 | From 866577 | to 866990 | Unknown, similar to B. subtilis |
| | | | | YrkR protein |
| SEQ ID NO. 249 | LM-1249.1 | From 864791 | to 865537 | Unknown |
| SEQ ID NO. 250 | LM-125.1 | From 1235670 | to 1236539 | Unknown, similar to N- |
| | | | | acetylmuramoyl-L-alanine |
| | | | | amidase (autolysin) |
| SEQ ID NO. 251 | LM-1250.1 | From 863798 | to 864688 | Unknown; similar to |
| | | | | transcriptional regulator |
| SEQ ID NO. 252 | LM-1251.1 | From 863322 | to 863606 | Unknown, similar to |
| | | | | transposase |
| SEQ ID NO. 253 | LM-1254.1 | From 861731 | to 862642 | Unknown |
| SEQ ID NO. 254 | LM-1255.1 | From 859656 | to 861617 | unknown, highly similar to |
| | | | | fructose-1,6- |
| | | | | bisphosphatase |
| SEQ ID NO. 255 | LM-1257.1 | From 855759 | to 859406 | unknown, highly similar to |
| | | | | pyruvate-flavodoxin |
| | | | | oxidoreductase |
| SEQ ID NO. 256 | LM-1258.1 | From 854745 | to 855581 | Unknown, similar to |
| | | | | transposases |
| SEQ ID NO. 257 | LM-1259.1 | From 854443 | to 854748 | Unknown, similar to |
| | | | | transposases |
| SEQ ID NO. 258 | LM-1261.1 | From 852704 | to 854338 | Unknown, similar to |
| | | | | transport protein |
| SEQ ID NO. 259 | LM-1262.1 | From 851225 | to 852505 | Unknown, similar to 3- |
| | | | | hydroxy-3-methylglutaryl- |
| | | | | coenzyme a reductase |
| SEQ ID NO. 260 | LM-1263.1 | From 850216 | to 851184 | Unknown |
| SEQ ID NO. 261 | LM-1265.1 | From 849314 | to 850138 | Unknown, similar to |
| | | | | oxydoreductases |
| SEQ ID NO. 262 | LM-1266.1 | From 848804 | to 849193 | Unknown, similar to |
| | | | | transcriptional regulators |
| SEQ ID NO. 263 | LM-1267.1 | From 848126 | to 848788 | Unknown |
| SEQ ID NO. 264 | LM-1268.1 | From 847552 | to 848109 | unknown, some similarity to |
| | | | | acetyltransferases |
| SEQ ID NO. 265 | LM-1269.1 | From 846594 | to 847442 | Unknown |
| SEQ ID NO. 266 | LM-1271.2 | From 843949 | to 846579 | unknown, similar to cation |
| | | | | transporting ATPase |
| SEQ ID NO. 267 | LM-1273.1 | From 939106 | to 939819 | Unknown, similar to |
| | | | | transcription regulator |
| | | | | (GntR family) |
| SEQ ID NO. 268 | LM-1275.2 | From 937739 | to 939025 | Unknown, similar to PTS |
| | | | | system, cellobiose-specific |
| | | | | IIC component |
| SEQ ID NO. 269 | LM-1277.2 | From 937202 | to 937594 | Unknown |
| SEQ ID NO. 270 | LM-1278.1 | From 936136 | to 936603 | Unknown, similar to B. subtilis |
| | | | | YdcK protein |
| SEQ ID NO. 271 | LM-128.1 | From 1233904 | to 1234434 | Unknown, similar to |
| | | | | unknown proteins |
| SEQ ID NO. 272 | LM-1283.1 | From 933959 | to 936136 | Unknown, conserved |
| | | | | hypothetical protein |
| SEQ ID NO. 273 | LM-1285.1 | From 932257 | to 933882 | Unknown, similar to |
| | | | | transport proteins |
| SEQ ID NO. 274 | LM-1286.1 | From 931451 | to 932050 | Indirect negative regulation |
| | | | | of sigma B dependant gene |
| | | | | expression (serine |
| | | | | phosphatase) |
| SEQ ID NO. 275 | LM-1287.1 | From 930671 | to 931450 | RNA polymerase sigma-37 |
| | | | | factor (sigma-B) |
| SEQ ID NO. 276 | LM-1288.1 | From 930220 | to 930693 | sigma-B activity negative |
| | | | | regulator RsbW |
| SEQ ID NO. 277 | LM-1289.1 | From 929892 | to 930236 | anti-anti-sigma factor |
| | | | | (antagonist of RsbW) |
| SEQ ID NO. 278 | LM-129.1 | From 1233366 | to 1233815 | Unknown, similar to |
| | | | | unknown proteins |
| SEQ ID NO. 279 | LM-1290.1 | From 928738 | to 929742 | Unknown, highly similar to |
| | | | | serine phosphatase RsbU |
| SEQ ID NO. 280 | LM-1291.1 | From 928311 | to 928721 | Unknown, highly similar to |
| | | | | positive regulation of sigma- |
| | | | | B activity |
| SEQ ID NO. 281 | LM-1293.1 | From 927952 | to 928308 | Unknown, highly similar to |
| | | | | negative regulation of |
| | | | | sigma-B activity |
| SEQ ID NO. 282 | LM-1294.1 | From 927110 | to 927946 | Unknown, highly similar to |
| | | | | positive regulator of sigma- |
| | | | | B activity |
| SEQ ID NO. 283 | LM-1295.1 | From 926511 | to 926858 | Unknown, similar to B. subtilis |
| | | | | YdcE protein |
| SEQ ID NO. 284 | LM-1296.1 | From 926229 | to 926507 | Unknown, similar to B. subtilis |
| | | | | YdcD protein |
| SEQ ID NO. 285 | LM-1297.1 | From 924917 | to 926023 | Unknown, similar to alanine |
| | | | | racemase |
| SEQ ID NO. 286 | LM-1298.1 | From 924542 | to 924898 | Unknown, similar to holo- |
| | | | | acyl-carrier protein synthase |
| SEQ ID NO. 287 | LM-13.1 | From 2719735 | to 2720484 | Unknown, similar to |
| | | | | creatinine amidohydrolase |
| SEQ ID NO. 288 | LM-130.1 | From 1232884 | to 1233351 | Unknown, similar to |
| | | | | unknown proteins |
| SEQ ID NO. 289 | LM-1300.1 | From 923161 | to 924540 | Unknown, similar to |
| | | | | protoporphyrinogen IX and |
| | | | | coproporphyrinogen III |
| | | | | oxidase (HemY) |
| SEQ ID NO. 290 | LM-1302.1 | From 921537 | to 923012 | Unknown, similar to B. subtilis |
| | | | | YbtB protein |
| SEQ ID NO. 291 | LM-1303.1 | From 921062 | to 921544 | Unknown, similar to B. subtilis |
| | | | | YdbS protein |
| SEQ ID NO. 292 | LM-1304.1 | From 920452 | to 920928 | unknown |
| SEQ ID NO. 293 | LM-1306.1 | From 918135 | to 918902 | unknown |
| SEQ ID NO. 294 | LM-1307.1 | From 917164 | to 918117 | Unknown, similar to |
| | | | | oxidoreductases |
| SEQ ID NO. 295 | LM-1309.1 | From 916436 | to 917164 | Unknown, similar to B. subtilis |
| | | | | NagB protein |
| | | | | (glucosamine-6-phosphate |
| | | | | isomerase) |
| SEQ ID NO. 296 | LM-131.1 | From 1232272 | to 1232838 | Unknown, similar to |
| | | | | unknown protein |
| SEQ ID NO. 297 | LM-1312.1 | From 915090 | to 916415 | Unknown, similar to PTS |
| | | | | system, Lichenan-specific |
| | | | | enzyme IIC component |
| SEQ ID NO. 298 | LM-1313.1 | From 914735 | to 915070 | Unknown, similar to PTS |
| | | | | system, beta-glucoside |
| | | | | enzyme IIB component |
| SEQ ID NO. 299 | LM-1314.1 | From 914405 | to 914734 | Unknown, similar to PTS |
| | | | | system enzyme IIA |
| | | | | component |
| SEQ ID NO. 300 | LM-1316.1 | From 912372 | to 914390 | Unknown; Similar to |
| | | | | transcriptional regulator |
| | | | | (antiterminator) |
| SEQ ID NO. 301 | LM-1317.1 | From 910998 | to 912164 | Unknown; similar to |
| | | | | antibiotic resistance protein |
| SEQ ID NO. 302 | LM-1318.1 | From 910517 | to 910870 | Unknown, similar to B. subtilis |
| | | | | YtcD protein |
| SEQ ID NO. 303 | LM-1319.1 | From 910173 | to 910484 | unknown |
| SEQ ID NO. 304 | LM-1320.1 | From 909159 | to 910157 | unknown |
| SEQ ID NO. 305 | LM-1321.1 | From 908499 | to 909056 | unknown |
| SEQ ID NO. 306 | LM-1322.1 | From 907979 | to 908467 | unknown |
| SEQ ID NO. 307 | LM-1325.1 | From 905962 | to 907524 | Unknown, similar to ATP- |
| | | | | dependent RNA helicase |
| SEQ ID NO. 308 | LM-1326.1 | From 903837 | to 905510 | Unknown, similar to |
| | | | | phosphomannomutase |
| SEQ ID NO. 309 | LM-1327.1 | From 902773 | to 903840 | unknown |
| SEQ ID NO. 310 | LM-1328.1 | From 901837 | to 902751 | unknown |
| SEQ ID NO. 311 | LM-1329.1 | From 900303 | to 901835 | Unknown, similar to oligo- |
| | | | | 1,6-glucosidase |
| SEQ ID NO. 312 | LM-1331.1 | From 899454 | to 900287 | Unknown, similar to sugar |
| | | | | ABC transporter, permease |
| | | | | protein- |
| SEQ ID NO. 313 | LM-1332.1 | From 898589 | to 899467 | Unknown, similar to sugar |
| | | | | ABC transporter, permease |
| | | | | protein |
| SEQ ID NO. 314 | LM-1333.3 | From 897273 | to 898592 | Unknown, similar to putative |
| | | | | sugar ABC transporter, |
| | | | | periplasmic sugar-binding |
| | | | | protein, |
| SEQ ID NO. 315 | LM-1335.3 | From 1104048 | to 1104851 | Unknown, highly similar to |
| | | | | teichoic acid translocation |
| | | | | permease protein TagG |
| SEQ ID NO. 316 | LM-1337.2 | From 1102858 | to 1103757 | Unknown, similar to metal |
| | | | | binding protein |
| SEQ ID NO. 317 | LM-1338.1 | From 1099266 | to 1102706 | Unknown, highly similar to |
| | | | | pyruvate carboxylase |
| SEQ ID NO. 318 | LM-1339.1 | From 1097786 | to 1098988 | Unknown, similar to cell- |
| | | | | division protein RodA and |
| | | | | FtsW |
| SEQ ID NO. 319 | LM-1341.1 | From 1097322 | to 1097603 | Unknown, similar to B. subtilis |
| | | | | YlaN protein |
| SEQ ID NO. 320 | LM-1342.1 | From 1096874 | to 1097116 | Unknown, similar to B. subtilis |
| | | | | YlaI protein |
| SEQ ID NO. 321 | LM-1343.2 | From 1095975 | to 1096835 | unknown |
| SEQ ID NO. 322 | LM-1349.1 | From 1093965 | to 1095803 | Unknown, similar to GTP- |
| | | | | binding ptotein TypA/BipA |
| | | | | (tyrosine phosphorylated |
| | | | | protein A) from E. coli and |
| | | | | B. subtilis (YlaG) |
| SEQ ID NO. 323 | LM-1351.1 | From 1093000 | to 1093773 | Unknown, similar to |
| | | | | extragenic suppressor |
| | | | | protein SuhB and to myoinositol- |
| | | | | 1(or 4)- |
| | | | | monophosphatase |
| SEQ ID NO. 324 | LM-1352.1 | From 1092255 | to 1092869 | Unknown, similar to B. subtilis |
| | | | | YktB protein |
| SEQ ID NO. 325 | LM-1353.1 | From 1091110 | to 1092054 | Unknown, similar to |
| | | | | membrane and transport |
| | | | | proteins |
| SEQ ID NO. 326 | LM-1354.1 | From 1090375 | to 1091043 | Unknown, similar to ABC |
| | | | | transporter (permease) |
| SEQ ID NO. 327 | LM-1357.1 | From 1088941 | to 1090362 | Unknown, conserved |
| | | | | hypothetical protein |
| SEQ ID NO. 328 | LM-1358.1 | From 1087409 | to 1088854 | Unknown, similar to sensor |
| | | | | protein histidine kinases (2 |
| | | | | components regulatory |
| | | | | systems) |
| SEQ ID NO. 329 | LM-1359.1 | From 1086772 | to 1087434 | Unknown, similar to |
| | | | | transcription response |
| | | | | regulator |
| SEQ ID NO. 330 | LM-1360.1 | From 1086100 | to 1086630 | unknown |
| SEQ ID NO. 331 | LM-1361.1 | From 1085806 | to 1086078 | Unknown, similar to B. subtilis |
| | | | | YktA protein |
| SEQ ID NO. 332 | LM-1363.1 | From 1084853 | to 1085806 | Unknown, similar to L- |
| | | | | lactate dehydrogenase |
| SEQ ID NO. 333 | LM-1364.1 | From 1084413 | to 1084724 | unknown |
| SEQ ID NO. 334 | LM-1367.1 | From 1082822 | to 1084225 | Unknown, highly similar to |
| | | | | dihydrolipoamide |
| | | | | dehydrogenase, E3 subunit |
| | | | | of pyruvate dehydrogenase |
| | | | | complex |
| SEQ ID NO. 335 | LM-1368.1 | From 1081183 | to 1082817 | Unknown, highly similar to |
| | | | | pyruvate dehydrogenase |
| | | | | (dihydrolipoamide |
| | | | | acetyltransferase E2 |
| | | | | subunit) |
| SEQ ID NO. 336 | LM-1369.1 | From 1080095 | to 1081072 | Unknown, highly similar to |
| | | | | pyruvate dehydrogenase |
| | | | | (E1 beta subunit) |
| SEQ ID NO. 337 | LM-1370.1 | From 1078977 | to 1080092 | Unknown, highly similar to |
| | | | | pyruvate dehydrogenase |
| | | | | (E1 alpha subunit) |
| SEQ ID NO. 338 | LM-1371.1 | From 1077592 | to 1078143 | Unknown, similar to |
| | | | | formylmethionine |
| | | | | deformylase and to B. subtilis |
| | | | | YkrB protein |
| SEQ ID NO. 339 | LM-1372.1 | From 1076989 | to 1077543 | Unknown, similar to B. subtilis |
| | | | | YdfE protein |
| SEQ ID NO. 340 | LM-1373.1 | From 1075860 | to 1076858 | Unknown, similar to |
| | | | | molybdopterin biosynthesis |
| | | | | protein MoeB |
| SEQ ID NO. 341 | LM-1374.1 | From 1075360 | to 1075848 | Unknown, similar to |
| | | | | molybdenum cofactor |
| | | | | biosynthesis protein B |
| SEQ ID NO. 342 | LM-1376.1 | From 1074324 | to 1075325 | Unknown, similar to |
| | | | | molybdenum cofactor |
| | | | | biosynthesis protein A |
| SEQ ID NO. 343 | LM-1377.1 | From 1073813 | to 1074295 | Unknown, similar to |
| | | | | molybdenum cofactor |
| | | | | biosynthesis protein C |
| SEQ ID NO. 344 | LM-1379.1 | From 1073552 | to 1073800 | Unknown, similar to |
| | | | | molybdopterin converting |
| | | | | factor (subunit 1). |
| SEQ ID NO. 345 | LM-1380.1 | From 1073146 | to 1073568 | Unknown, similar to |
| | | | | molybdopterin converting |
| | | | | factor, subunit 2 |
| SEQ ID NO. 346 | LM-1381.1 | From 1072664 | to 1073149 | Unknown, similar to |
| | | | | molybdopterin-guanine |
| | | | | dinucleotide biosynthesis |
| | | | | MobB |
| SEQ ID NO. 347 | LM-1383.1 | From 1071462 | to 1072685 | Unknown, similar to |
| | | | | molybdopterin biosynthesis |
| | | | | protein moeA |
| SEQ ID NO. 348 | LM-1385.1 | From 1070597 | to 1071367 | Unknown, similar to |
| | | | | molybdate binding protein |
| SEQ ID NO. 349 | LM-1386.1 | From 1069821 | to 1070492 | Unknown, similar to |
| | | | | molybdenum transport |
| | | | | protein |
| SEQ ID NO. 350 | LM-1387.1 | From 1069156 | to 1069818 | unknown, similar to ABC |
| | | | | transporter |
| SEQ ID NO. 351 | LM-1388.1 | From 1068594 | to 1069175 | unknown, weakly similar to |
| | | | | molybdopterin-guanine |
| | | | | dinucleotide biosynthesis |
| | | | | protein A |
| SEQ ID NO. 352 | LM-1389.1 | From 1067723 | to 1068526 | unknown, highly similar to |
| | | | | B. subtilis YoaT protein |
| SEQ ID NO. 353 | LM-1390.1 | From 1066397 | to 1067662 | unknown |
| SEQ ID NO. 354 | LM-1391.1 | From 1064521 | to 1066377 | unknown, similar to |
| | | | | phosphotransferase system |
| | | | | (PTS) beta-glucoside- |
| | | | | specific enzyme IIABC |
| SEQ ID NO. 355 | LM-1392.1 | From 1063061 | to 1064524 | unknown, similar to glycerol |
| | | | | kinase |
| SEQ ID NO. 356 | LM-1394.1 | From 1062108 | to 1063064 | unknown, similar to |
| | | | | transketolase |
| SEQ ID NO. 357 | LM-1396.1 | From 1061291 | to 1062115 | unknown, similar to |
| | | | | transketolase |
| SEQ ID NO. 358 | LM-1398.3 | From 1059872 | to 1061275 | unknown, similar to |
| | | | | hypothetical proteins |
| SEQ ID NO. 359 | LM-1401.1 | From 235524 | to 237020 | lysyl-tRNA synthetase |
| SEQ ID NO. 360 | LM-1402.1 | From 234414 | to 235409 | Unknown, conserved |
| | | | | hypothetical protein |
| SEQ ID NO. 361 | LM-1404.1 | From 233858 | to 234337 | unknown, similar to 7,8- |
| | | | | dihydro-6- |
| | | | | hydroxymethylpterin |
| | | | | pyrophosphokinase |
| SEQ ID NO. 362 | LM-1405.1 | From 233491 | to 233865 | unknown, highly similar to |
| | | | | dihydroneopterin aldolase |
| SEQ ID NO. 363 | LM-1407.1 | From 232662 | to 233477 | unknown, highly similar to |
| | | | | dihydropteroate synthases |
| SEQ ID NO. 364 | LM-1408.1 | From 230840 | to 231766 | Unknown, highly similar to |
| | | | | cysteine synthase |
| SEQ ID NO. 365 | LM-1410.1 | From 229840 | to 230724 | Unknown, conserved |
| | | | | hypothetical protein |
| SEQ ID NO. 366 | LM-1411.1 | From 229045 | to 229824 | Unknown, conserved |
| | | | | hypothetical protein |
| SEQ ID NO. 367 | LM-1413.1 | From 226854 | to 228929 | Unknown, highly similar to |
| | | | | cell division protein ftsH |
| SEQ ID NO. 368 | LM-1415.1 | From 224562 | to 226508 | Unknown, fusion protein, N- |
| | | | | terminal part similar to B. subtilis |
| | | | | YacA protein, C- |
| | | | | terminal part similar to |
| | | | | hypoxanthine-guanine |
| | | | | phosphoribosyltransferase |
| SEQ ID NO. 369 | LM-1418.1 | From 224027 | to 224455 | Unknown, |
| | | | | polyribonucleotide |
| | | | | nucleotidyltransferase |
| | | | | domain present |
| SEQ ID NO. 370 | LM-1420.1 | From 223490 | to 223876 | Unknown, similar to B. subtilis |
| | | | | DivlC protein |
| SEQ ID NO. 371 | LM-1426.1 | From 221370 | to 222959 | Unknown, conserved |
| | | | | membrane-spanning protein |
| SEQ ID NO. 372 | LM-1429.1 | From 217800 | to 221339 | Transcription-repair |
| | | | | coupling factor |
| SEQ ID NO. 373 | LM-1430.1 | From 217125 | to 217685 | Unknown, similar to |
| | | | | peptidyl-tRNA hydrolase |
| SEQ ID NO. 374 | LM-1432.1 | From 216434 | to 217018 | Unknown |
| SEQ ID NO. 375 | LM-1433.2 | From 215721 | to 216344 | Unknown, similar to B. subtilis |
| | | | | general stress |
| | | | | protein |
| SEQ ID NO. 376 | LM-1434.2 | From 214486 | to 215427 | Unknown, similar to L- |
| | | | | lactate dehydrogenase |
| SEQ ID NO. 377 | LM-1435.1 | From 213735 | to 214409 | Unknown |
| SEQ ID NO. 378 | LM-1436.1 | From 213336 | to 213668 | Unknown, conserved |
| | | | | hypothetical protein |
| SEQ ID NO. 379 | LM-1437.1 | From 212824 | to 213285 | Unknown, hypothetical |
| | | | | lipoprotein |
| SEQ ID NO. 380 | LM-1438.1 | From 212366 | to 212689 | Unknown |
| SEQ ID NO. 381 | LM-1439.1 | From 211425 | to 212294 | Phospholipase C |
| SEQ ID NO. 382 | LM-1442.1 | From 209470 | to 211389 | Actin-assembly inducing |
| | | | | protein precursor |
| SEQ ID NO. 383 | LM-1444.1 | From 207739 | to 209271 | Zinc metalloproteinase |
| | | | | precursor |
| SEQ ID NO. 384 | LM-1445.1 | From 205819 | to 207408 | Listeriolysin O precursor |
| SEQ ID NO. 385 | LM-1446.1 | From 204624 | to 205577 | Phosphatidylinositol-specific |
| | | | | phospholipase c |
| SEQ ID NO. 386 | LM-1447.1 | From 203640 | to 204353 | Listeriolysin positive |
| | | | | regulatory protein |
| SEQ ID NO. 387 | LM-1448.1 | From 202641 | to 203597 | phosphoribosyl |
| | | | | pyrophosphate synthetase |
| SEQ ID NO. 388 | LM-1449.3 | From 201217 | to 202590 | Unknown, highly similar to |
| | | | | UDP-N-acetylglucosamine |
| | | | | pyrophosphorylase |
| SEQ ID NO. 389 | LM-1451.1 | From 1891236 | to 1891880 | Unknown, weakly similar to |
| | | | | thiamin pyrophosphokinase |
| SEQ ID NO. 390 | LM-1453.1 | From 1891944 | to 1892600 | Unknown, similar to |
| | | | | ribulose-5-phosphate 3- |
| | | | | epimerase |
| SEQ ID NO. 391 | LM-1454.1 | From 1892603 | to 1893478 | Unknown, similar to |
| | | | | unknown proteins |
| SEQ ID NO. 392 | LM-1457.1 | From 1893497 | to 1895464 | Unknown, similar to putative |
| | | | | serine/threonine-specific |
| | | | | protein kinase |
| SEQ ID NO. 393 | LM-1458.1 | From 1895461 | to 1896219 | Unknown, similar to putative |
| | | | | phosphoprotein |
| | | | | phosphatase |
| SEQ ID NO. 394 | LM-1459.1 | From 1896242 | to 1897576 | Unknown, similar to RNA- |
| | | | | binding Sun protein |
| SEQ ID NO. 395 | LM-1461.1 | From 1897577 | to 1898515 | Unknown, similar to |
| | | | | methionyl-tRNA |
| | | | | formyltransferase |
| SEQ ID NO. 396 | LM-1463.1 | From 1898529 | to 1900922 | Unknown, similar to |
| | | | | primosomal replication |
| | | | | factor Y |
| SEQ ID NO. 397 | LM-1465.1 | From 1900927 | to 1902126 | Unknown, similar to |
| | | | | pantothenate metabolism |
| | | | | flavoprotein homolog |
| SEQ ID NO. 398 | LM-1466.2 | From 1902484 | to 1903101 | Unknown, similar to |
| | | | | guanylate kinases |
| SEQ ID NO. 399 | LM-1467.2 | From 1903120 | to 1903995 | Unknown, simlar to |
| | | | | conserved hypothetical |
| | | | | protein |
| SEQ ID NO. 400 | LM-1468.1 | From 1904152 | to 1905864 | Unknown, similar to |
| | | | | fibronectin binding proteins |
| SEQ ID NO. 401 | LM-1469.1 | From 1905952 | to 1906551 | Unknown, similar to |
| | | | | conserved hypotheticl |
| | | | | proteins |
| SEQ ID NO. 402 | LM-1470.1 | From 1906592 | to 1907221 | Unknown, highly similar to |
| | | | | orotate |
| | | | | phosphoribosyltransferases |
| SEQ ID NO. 403 | LM-1471.1 | From 1907218 | to 1907919 | Unknown, highly similar to |
| | | | | orotidine 5-phosphate |
| | | | | decarboxylases |
| SEQ ID NO. 404 | LM-1472.1 | From 1907916 | to 1908830 | Unknown, highly similar to |
| | | | | dihydroorotase |
| | | | | dehydrogenase |
| SEQ ID NO. 405 | LM-1473.1 | From 1908827 | to 1909591 | Unknown, highly similar to |
| | | | | dihydroorotate |
| | | | | dehydrogenase (electron |
| | | | | transfer subunit) |
| SEQ ID NO. 406 | LM-1474.1 | From 1909614 | to 1912826 | Unknown, highly similar to |
| | | | | carbamoyl-phosphate |
| | | | | synthetase (catalytic |
| | | | | subunit) |
| SEQ ID NO. 407 | LM-1476.1 | From 1912819 | to 1913910 | Unknown, highly similar to |
| | | | | carbamoyl-phosphate |
| | | | | synthetase (glutaminase |
| | | | | subunit) |
| SEQ ID NO. 408 | LM-1477.1 | From 1913907 | to 1915187 | Unknown, highly similar to |
| | | | | dihydroorotase |
| SEQ ID NO. 409 | LM-1478.1 | From 1915175 | to 1916086 | Unknown, highly similar to |
| | | | | aspartate |
| | | | | carbamoyltransferase |
| SEQ ID NO. 410 | LM-1480.1 | From 1916166 | to 1917452 | Unknown, highly similar to |
| | | | | uracil permease |
| SEQ ID NO. 411 | LM-1481.2 | From 1917581 | to 1918132 | Unknown, highly similar to |
| | | | | pyrimidine operon |
| | | | | regulatory protein |
| SEQ ID NO. 412 | LM-1482.2 | From 1918363 | to 1918593 | unknown |
| SEQ ID NO. 413 | LM-1483.2 | From 645397 | to 645858 | Unknown, similar to |
| | | | | transcription regulator MarR |
| | | | | family |
| SEQ ID NO. 414 | LM-1484.1 | From 645878 | to 647590 | Unknown, similar to ABC |
| | | | | transporter, ATP-binding |
| | | | | protein |
| SEQ ID NO. 415 | LM-1486.1 | From 647587 | to 649404 | Unknown, similar to ABC |
| | | | | transporter, ATP-binding |
| | | | | protein |
| SEQ ID NO. 416 | LM-1487.1 | From 649520 | to 649819 | Unknown, similar to E. coli |
| | | | | phage shock protein E |
| SEQ ID NO. 417 | LM-1491.1 | From 651794 | to 652420 | Unknown, similar to acyl- |
| | | | | carrier protein |
| | | | | phosphodiesterase and |
| | | | | NAD(P)H dehydrogenase |
| SEQ ID NO. 418 | LM-1492.1 | From 652589 | to 653044 | Unknown, similar to |
| | | | | transcription regulator MarR |
| | | | | family |
| SEQ ID NO. 419 | LM-1493.1 | From 653041 | to 653982 | Unknown, similar to |
| | | | | oxidoreductase |
| SEQ ID NO. 420 | LM-1494.1 | From 654078 | to 654611 | Unknown, conserved |
| | | | | hypothetical protein |
| SEQ ID NO. 421 | LM-1495.1 | From 654608 | to 654850 | Unknown |
| SEQ ID NO. 422 | LM-1498.1 | From 654957 | to 656708 | Unknown, C-terminal |
| | | | | domain similar to |
| | | | | glycerophosphoryl diester |
| | | | | phosphodiesterase |
| SEQ ID NO. 423 | LM-1499.1 | From 656749 | to 657243 | Unknown |
| SEQ ID NO. 424 | LM-15.1 | From 2720497 | to 2721489 | Unknown, similar to |
| | | | | Phosphotriesterase |
| SEQ ID NO. 425 | LM-1500.1 | From 657323 | to 658465 | Unknown, similar to protein |
| | | | | kinase |
| SEQ ID NO. 426 | LM-1501.1 | From 658572 | to 659027 | Unknown |
| SEQ ID NO. 427 | LM-1502.1 | From 659083 | to 659475 | Unknown |
| SEQ ID NO. 428 | LM-1504.1 | From 659572 | to 660312 | Unknown, conserved |
| | | | | hypothetical protein |
| SEQ ID NO. 429 | LM-1506.1 | From 660398 | to 660676 | Unknown, hypothetical |
| SEQ ID NO. 430 | LM-1507.1 | From 660692 | to 660958 | Unknown |
| SEQ ID NO. 431 | LM-1508.1 | From 660996 | to 661439 | Unknown, similar to |
| | | | | unknown proteins |
| SEQ ID NO. 432 | LM-1510.3 | From 661470 | to 662171 | Unknown |
| SEQ ID NO. 433 | LM-1511.3 | From 662257 | to 663936 | Unknown, similar to |
| | | | | unknown protein |
| SEQ ID NO. 434 | LM-1516.1 | From 669423 | to 669950 | Unknown |
| SEQ ID NO. 435 | LM-1518.1 | From 670309 | to 672339 | Unknown, similar to |
| | | | | transcription antiterminator |
| | | | | BglG family |
| SEQ ID NO. 436 | LM-1519.1 | From 672341 | to 672793 | Unknown, similar to PTS |
| | | | | system, fructose-specific IIA |
| | | | | component |
| SEQ ID NO. 437 | LM-1520.1 | From 672794 | to 673855 | Unknown, similar to PTS |
| | | | | system, fructose-specific IIC |
| | | | | component |
| SEQ ID NO. 438 | LM-1521.1 | From 673870 | to 674178 | Unknown, similar to PTS |
| | | | | system, fructose-specific IIB |
| | | | | component |
| SEQ ID NO. 439 | LM-1523.1 | From 674205 | to 675473 | Unknown, similar to an E. coli |
| | | | | putative tagatose 6- |
| | | | | phosphate kinase |
| SEQ ID NO. 440 | LM-1524.1 | From 675563 | to 676267 | Unknown |
| SEQ ID NO. 441 | LM-1525.1 | From 676372 | to 676788 | Unknown, similar to |
| | | | | unknown proteins |
| SEQ ID NO. 442 | LM-1527.1 | From 676802 | to 677395 | Unknown, weakly similar to |
| | | | | methyltransferase |
| SEQ ID NO. 443 | LM-1528.1 | From 678494 | to 679123 | Unknown |
| SEQ ID NO. 444 | LM-1529.1 | From 679763 | to 680296 | Unknown, similar to a |
| | | | | transcription regulator |
| | | | | (surface protein PAg |
| | | | | negative regulator par) |
| SEQ ID NO. 445 | LM-153.1 | From 1216820 | to 1218178 | Unknown, similar to |
| | | | | cobyrinic acid a,c-diamide |
| | | | | synthase |
| SEQ ID NO. 446 | LM-1530.1 | From 680360 | to 681277 | Unknown, similar to |
| | | | | oxidoreductase |
| SEQ ID NO. 447 | LM-1532.1 | From 681543 | to 683423 | Unknown, similar to heavy |
| | | | | metal-transporting ATPase |
| SEQ ID NO. 448 | LM-1533.1 | From 683519 | to 684247 | Unknown |
| SEQ ID NO. 449 | LM-1535.1 | From 684390 | to 685040 | Unknown, similar to putative |
| | | | | transaldolase |
| SEQ ID NO. 450 | LM-1536.3 | From 685083 | to 686903 | Unknown, similar to |
| | | | | conserved hypothetical |
| | | | | proteins |
| SEQ ID NO. 451 | LM-1537.2 | From 468169 | to 469437 | Unknown, weakly similar to |
| | | | | a module of peptide |
| | | | | synthetase |
| SEQ ID NO. 452 | LM-1538.1 | From 467519 | to 468136 | unknown |
| SEQ ID NO. 453 | LM-154.1 | From 1215993 | to 1216487 | unknown |
| SEQ ID NO. 454 | LM-1540.1 | From 466517 | to 467362 | Unknown, conserved |
| | | | | hypothetical protein |
| SEQ ID NO. 455 | LM-1541.1 | From 465934 | to 466419 | Unknown, similar to |
| | | | | unknown proteins |
| SEQ ID NO. 456 | LM-1545.1 | From 459681 | to 465722 | Unknown, putative |
| | | | | peptidoglycan linked protein |
| | | | | (LPXTG motif) |
| SEQ ID NO. 457 | LM-1547.1 | From 457021 | to 458913 | Internalin B |
| SEQ ID NO. 458 | LM-1549.1 | From 454534 | to 456936 | Internalin A |
| SEQ ID NO. 459 | LM-155.1 | From 1215128 | to 1215679 | Unknown, similar to |
| | | | | transcriptional regulator |
| SEQ ID NO. 460 | LM-1551.1 | From 453107 | to 453853 | Unknown, similar to |
| | | | | oxidoreductase |
| SEQ ID NO. 461 | LM-1553.1 | From 452524 | to 453093 | Unknown, similar to |
| | | | | acetyltransferase |
| SEQ ID NO. 462 | LM-1554.1 | From 451531 | to 452406 | Unknown, similar to |
| | | | | transcriptional regulator |
| | | | | (LysR family) |
| SEQ ID NO. 463 | LM-1557.1 | From 448926 | to 451508 | Unknown, similar to sugar |
| | | | | hydrolase |
| SEQ ID NO. 464 | LM-1558.1 | From 447804 | to 448910 | Unknown, similar to PTS |
| | | | | fructose-specific enzyme IIC |
| | | | | component |
| SEQ ID NO. 465 | LM-156.1 | From 1213636 | to 1215087 | unknown |
| SEQ ID NO. 466 | LM-1560.1 | From 447471 | to 447791 | Unknown, similar to PTS |
| | | | | fructose-specific enzyme IIB |
| | | | | component |
| SEQ ID NO. 467 | LM-1561.1 | From 447010 | to 447474 | Unknown, similar to PTS |
| | | | | fructose-specific enzyme IIA |
| | | | | component |
| SEQ ID NO. 468 | LM-1564.1 | From 445049 | to 447010 | Unknown, similar to |
| | | | | transcription antiterminator |
| | | | | BglG family |
| SEQ ID NO. 469 | LM-1566.1 | From 444031 | to 444888 | Unknown, similar to |
| | | | | Staphylococcus xylosus |
| | | | | glucose uptake protein |
| SEQ ID NO. 470 | LM-1568.1 | From 443303 | to 443851 | Unknown, similar to RNA |
| | | | | polymerase ECF-type |
| | | | | sigma factor |
| SEQ ID NO. 471 | LM-1569.1 | From 442869 | to 443300 | Unknown, similar to |
| | | | | unknown protein |
| SEQ ID NO. 472 | LM-157.1 | From 1212983 | to 1213426 | Similar to ethanolamine |
| | | | | utilization protein EutQ |
| SEQ ID NO. 473 | LM-1570.1 | From 441622 | to 442872 | Unknown, similar to rod |
| | | | | shape-determining protein |
| | | | | RodA |
| SEQ ID NO. 474 | LM-1571.1 | From 440758 | to 441570 | Unknown, conserved |
| | | | | hypothetical protein |
| SEQ ID NO. 475 | LM-1572.1 | From 440056 | to 440610 | Unknown, similar to |
| | | | | unknown protein |
| SEQ ID NO. 476 | LM-1573.1 | From 439677 | to 439976 | Unknown |
| SEQ ID NO. 477 | LM-1574.1 | From 439214 | to 439624 | Unknown |
| SEQ ID NO. 478 | LM-1575.2 | From 437482 | to 438882 | Unknown, similar to endo- |
| | | | | 1,4-beta-xylanase |
| SEQ ID NO. 479 | LM-1577.4 | From 436379 | to 437188 | Unknown, conserved |
| | | | | membrane protein |
| SEQ ID NO. 480 | LM-1578.2 | From 977051 | to 9978856 | Unknown, similar to heat |
| | | | | shock protein HtpG |
| SEQ ID NO. 481 | LM-1579.1 | From 976065 | to 977039 | Unknown |
| SEQ ID NO. 482 | LM-158.1 | From 1211869 | to 1212990 | Unknown, similar to |
| | | | | ethanolamine utilization |
| | | | | protein EutH - Escherichia |
| | | | | coli |
| SEQ ID NO. 483 | LM-1580.1 | From 975071 | to 976033 | Unknown |
| SEQ ID NO. 484 | LM-1581.1 | From 974229 | to 974975 | Unknown |
| SEQ ID NO. 485 | LM-1582.1 | From 973758 | to 974216 | Unknown, similar to protein- |
| | | | | tyrosine-phosphatase |
| SEQ ID NO. 486 | LM-1583.1 | From 972719 | to 973459 | Unknown, similar to |
| | | | | Nitroflavin-reductase |
| SEQ ID NO. 487 | LM-1584.1 | From 972197 | to 972706 | Unknown, similar to B. subtilis |
| | | | | CspR protein, rRNA |
| | | | | methylase homolog |
| SEQ ID NO. 488 | LM-1585.1 | From 971037 | to 972176 | Unknown, similar to B. subtilis |
| | | | | YhbA protein |
| SEQ ID NO. 489 | LM-1586.1 | From 969549 | to 970496 | Unknown, similar to B. subtilis |
| | | | | YkcC protein |
| SEQ ID NO. 490 | LM-1587.1 | From 968819 | to 969424 | Unknown, conserved |
| | | | | hypothetical protein |
| SEQ ID NO. 491 | LM-1589.1 | From 967784 | to 968779 | Unknown, similar to lipoate |
| | | | | protein ligase A |
| SEQ ID NO. 492 | LM-159.1 | From 1211308 | to 1211853 | Unknown, similar to |
| | | | | Salmonella enterica PduT |
| | | | | protein |
| SEQ ID NO. 493 | LM-1590.1 | From 967029 | to 967760 | Unknown, conserved |
| | | | | hypothetical protein, similar |
| | | | | to B. subtilis Yhfl protein |
| SEQ ID NO. 494 | LM-1592.1 | From 966245 | to 966913 | Unknown, similar to sortase |
| SEQ ID NO. 495 | LM-1593.1 | From 965513 | to 966136 | Unknown, similar to 3- |
| | | | | methyladenine DNA |
| | | | | glycosylase |
| SEQ ID NO. 496 | LM-1594.1 | From 963294 | to 965255 | Unknown, hypothetical |
| | | | | transmembrane protein |
| SEQ ID NO. 497 | LM-1596.1 | From 962607 | to 963263 | Unknown, similar to |
| | | | | transcription regulator |
| | | | | (TetR/AcrR family) |
| SEQ ID NO. 498 | LM-1597.1 | From 961469 | to 962584 | Unknown, putative |
| | | | | membrane protein |
| SEQ ID NO. 499 | LM-16.1 | From 2721559 | to 2722857 | Unknown, similar to |
| | | | | membrane proteins |
| SEQ ID NO. 500 | LM-160.1 | From 1211046 | to 1211315 | Unknown, similar to carbondioxide |
| | | | | concentrating |
| | | | | mechanism protein |
| SEQ ID NO. 501 | LM-1600.1 | From 960748 | to 961221 | Unknown, similar to ABC |
| | | | | transporter, ATP-binding |
| | | | | protein (N-terminal part) |
| SEQ ID NO. 502 | LM-1602.1 | From 959711 | to 960631 | Unknown, similar to |
| | | | | pantothenate kinase |
| SEQ ID NO. 503 | LM-1603.1 | From 958783 | to 959622 | Unknown, similar to B. subtilis |
| | | | | YcgQ protein |
| SEQ ID NO. 504 | LM-1605.1 | From 957724 | to 958764 | Unknown, similar to B. subtilis |
| | | | | YcgR protein |
| SEQ ID NO. 505 | LM-1606.1 | From 956031 | to 957602 | Unknown, similar to ABC |
| | | | | transporter ATP-binding |
| | | | | protein (antibiotic |
| | | | | resistance) |
| SEQ ID NO. 506 | LM-1607.1 | From 953842 | to 955740 | Unknown, similar to |
| | | | | transcription antiterminator |
| | | | | BglG family |
| SEQ ID NO. 507 | LM-1608.1 | From 952318 | to 953769 | Unknown, similar to beta- |
| | | | | glucosidase |
| SEQ ID NO. 508 | LM-1609.1 | From 951956 | to 952306 | Unknown, similar to |
| | | | | phosphotransferase system |
| | | | | enzyme IIA |
| SEQ ID NO. 509 | LM-161.1 | From 1210459 | to 1211031 | unknown |
| SEQ ID NO. 510 | LM-1610.1 | From 950673 | to 951941 | Unknown, similar to |
| | | | | phosphotransferase system |
| | | | | enzyme IIC |
| SEQ ID NO. 511 | LM-1611.1 | From 950363 | to 950668 | Unknown, similar to PTS |
| | | | | system, IIB component |
| SEQ ID NO. 512 | LM-1613.1 | From 948754 | to 950220 | Unknown, similar to |
| | | | | succinate semialdehyde |
| | | | | dehydrogenase |
| SEQ ID NO. 513 | LM-1614.1 | From 947718 | to 948530 | Unknown, similar to |
| | | | | transporters (formate) |
| SEQ ID NO. 514 | LM-1615.1 | From 947101 | to 947604 | Unknown |
| SEQ ID NO. 515 | LM-1617.1 | From 945915 | to 947018 | Unknown |
| SEQ ID NO. 516 | LM-1618.1 | From 945520 | to 945912 | Unknown, similar to |
| | | | | transcription regulator, GntR |
| | | | | family |
| SEQ ID NO. 517 | LM-1619.1 | From 944243 | to 945379 | Unknown, similar to |
| | | | | membrane proteins |
| SEQ ID NO. 518 | LM-162.1 | From 1209821 | to 1210462 | Unknown, similar to |
| | | | | Salmonella enterica PduL |
| | | | | protein |
| SEQ ID NO. 519 | LM-1620.1 | From 943526 | to 944200 | Unknown, similar to |
| | | | | phosphoglycerate mutase |
| SEQ ID NO. 520 | LM-1622.1 | From 942148 | to 943497 | Unknown, similar to |
| | | | | glutathione Reductase |
| SEQ ID NO. 521 | LM-1623.1 | From 941615 | to 942112 | unknown |
| SEQ ID NO. 522 | LM-1624.2 | From 940810 | to 941484 | Unknown |
| SEQ ID NO. 523 | LM-1625.2 | From 469575 | to 470078 | Unknown |
| SEQ ID NO. 524 | LM-1626.1 | From 470122 | to 472158 | Unknown, similar to |
| | | | | penicillin-binding protein (D- |
| | | | | alanyl-D-alanine |
| | | | | carboxypeptidase) |
| SEQ ID NO. 525 | LM-1627.1 | From 472330 | to 472644 | Unknown |
| SEQ ID NO. 526 | LM-1629.1 | From 472781 | to 473710 | Unknown, similar to B. subtilis |
| | | | | transcription |
| | | | | regulator LytR |
| SEQ ID NO. 527 | LM-163.1 | From 1209053 | to 1209808 | Unknown, similar to |
| | | | | cobalamin adenosyl |
| | | | | transferase |
| SEQ ID NO. 528 | LM-1631.1 | From 473936 | to 476716 | Unknown, conserved |
| | | | | hypothetical protein |
| SEQ ID NO. 529 | LM-1632.1 | From 476960 | to 478447 | Unknown, similar to |
| | | | | transcription regulator |
| SEQ ID NO. 530 | LM-1634.1 | From 478721 | to 479710 | Unknown, similar to |
| | | | | penicillin acylase and to |
| | | | | conjugated bile acid |
| | | | | hydrolase |
| SEQ ID NO. 531 | LM-1635.1 | From 479765 | to 481153 | Unknown, similar to |
| | | | | glutamate decarboxylase |
| SEQ ID NO. 532 | LM-1636.1 | From 481250 | to 482701 | Unknown, similar to amino |
| | | | | acid antiporter |
| SEQ ID NO. 533 | LM-1637.1 | From 483166 | to 483891 | Unknown |
| SEQ ID NO. 534 | LM-1638.1 | From 483934 | to 484599 | Unknown, similar to |
| | | | | unknown proteins |
| SEQ ID NO. 535 | LM-1639.1 | From 484620 | to 485375 | Unknown |
| SEQ ID NO. 536 | LM-164.1 | From 1208603 | to 1208887 | Unknown, similar to putative |
| | | | | carboxysome structural |
| | | | | protein |
| SEQ ID NO. 537 | LM-1644.1 | From 485493 | to 487652 | Unknown, similar to |
| | | | | unknown proteins |
| SEQ ID NO. 538 | LM-1645.1 | From 487649 | to 488797 | Unknown, conserved |
| | | | | hypothetical proteins |
| SEQ ID NO. 539 | LM-1646.1 | From 488802 | to 489749 | Unknown, conserved |
| | | | | hypothetical protein similar |
| | | | | to B. subtilis YeaC |
| SEQ ID NO. 540 | LM-1647.1 | From 489905 | to 491500 | Unknown, similar to |
| | | | | unknown proteins |
| SEQ ID NO. 541 | LM-1650.1 | From 491634 | to 492917 | Unknown, similar to |
| | | | | permeases |
| SEQ ID NO. 542 | LM-1652.1 | From 492918 | to 494018 | Unknown, similar to |
| | | | | unknown proteins |
| SEQ ID NO. 543 | LM-1653.1 | From 494011 | to 495561 | Unknown, similar to |
| | | | | hydantoinase |
| SEQ ID NO. 544 | LM-1655.1 | From 496381 | to 497919 | Unknown, similar to |
| | | | | transcription regulator (VirR |
| | | | | From Streptococcus |
| | | | | pyogenes) |
| SEQ ID NO. 545 | LM-1656.1 | From 498171 | to 500240 | Unknown, putative |
| | | | | membrane associated |
| | | | | lipoprotein |
| SEQ ID NO. 546 | LM-1658.1 | From 500306 | to 500779 | Unknown |
| SEQ ID NO. 547 | LM-1659.3 | From 500799 | to 501284 | Unknown |
| SEQ ID NO. 548 | LM-166.1 | From 1207111 | to 1208571 | Unknown, similar to |
| | | | | acetaldehyde |
| | | | | dehydrogenase/alcohol |
| | | | | dehydrogenase |
| SEQ ID NO. 549 | LM-1661.4 | From 2632907 | to 2633761 | Unknown, similar to |
| | | | | fructose-1,6-bisphosphate |
| | | | | aldolase |
| SEQ ID NO. 550 | LM-1662.2 | From 2631303 | to 2632586 | Unknown, weakly similar to |
| | | | | human N- |
| | | | | acetylglucosaminyl- |
| | | | | phosphatidylinositol |
| | | | | biosynthetic protein |
| SEQ ID NO. 551 | LM-1663.1 | From 2630285 | to 2631310 | Unknown, similar to |
| | | | | galactosyltransferase |
| SEQ ID NO. 552 | LM-1664.1 | From 2629208 | to 2630278 | Unknown, conserved |
| | | | | hypothetical protein |
| SEQ ID NO. 553 | LM-1665.1 | From 2627816 | to 2629087 | Unknown, highly similar to |
| | | | | UDP-N-acetylglucosamine |
| | | | | 1-carboxyvinyltransferase |
| SEQ ID NO. 554 | LM-1666.2 | From 2626442 | to 2627713 | Unknown, highly similar to |
| | | | | transcription terminator |
| | | | | factor rho |
| SEQ ID NO. 555 | LM-1668.2 | From 2625414 | to 2626361 | Unknown, similar to glycosyl |
| | | | | transferases |
| SEQ ID NO. 556 | LM-1669.1 | From 2624980 | to 2625417 | wall teichoic acid |
| | | | | glycosylation protein GtcA |
| SEQ ID NO. 557 | LM-167.1 | From 1206590 | to 1207111 | Unknown, similar to putative |
| | | | | carboxysome structural |
| | | | | protein |
| SEQ ID NO. 558 | LM-1671.1 | From 2623125 | to 2624411 | Unknown, highly similar to |
| | | | | homoserine dehydrogenase |
| SEQ ID NO. 559 | LM-1674.1 | From 2622067 | to 2623122 | Unknown, highly similar to |
| | | | | threonine synthase |
| SEQ ID NO. 560 | LM-1676.1 | From 2621201 | to 2622067 | Unknown, highly similar to |
| | | | | homoserine kinase |
| SEQ ID NO. 561 | LM-1678.1 | From 2620514 | to 2621089 | Unknown, similar to |
| | | | | thymidine kinase |
| SEQ ID NO. 562 | LM-1679.1 | From 2619415 | to 2620491 | Unknown, highly similar to |
| | | | | peptide chain release |
| | | | | factor 1 |
| SEQ ID NO. 563 | LM-168.1 | From 1205922 | to 1206575 | Unknown, similar to putative |
| | | | | carboxysome structural |
| | | | | protein (eutL) |
| SEQ ID NO. 564 | LM-1681.1 | From 2618577 | to 2619428 | Unknown, similar to |
| | | | | protoporphyrinogen oxidase |
| SEQ ID NO. 565 | LM-1682.1 | From 2617239 | to 2618276 | Unknown, similar to yeast |
| | | | | translation initiation protein |
| SEQ ID NO. 566 | LM-1683.1 | From 2616835 | to 2617242 | Unknown, similar to |
| | | | | phosphatases |
| SEQ ID NO. 567 | LM-1684.1 | From 2615458 | to 2616699 | Unknown, highly similar to |
| | | | | glycine |
| | | | | hydroxymethyltransferase |
| SEQ ID NO. 568 | LM-1685.1 | From 2614692 | to 2615321 | Unknown, highly similar to |
| | | | | uracil |
| | | | | phosphoribosyltransferase |
| SEQ ID NO. 569 | LM-1686.1 | From 2613435 | to 2614574 | Unknown, similar to UDP-N- |
| | | | | acetylglucosamine 2- |
| | | | | epimerase |
| SEQ ID NO. 570 | LM-1691.1 | From 2612205 | to 2612603 | unknown, highly similar to |
| | | | | ATP synthase subunit i |
| SEQ ID NO. 571 | LM-1693.1 | From 2611482 | to 2612198 | unknown, highly similar to |
| | | | | H+-transporting ATP |
| | | | | synthase chain a |
| SEQ ID NO. 572 | LM-1694.1 | From 2610592 | to 2611104 | unknown, highly similar to |
| | | | | H+-transporting ATP |
| | | | | synthase chain b |
| SEQ ID NO. 573 | LM-1695.1 | From 2610056 | to 2610595 | unknown, highly similar to |
| | | | | H+-transporting ATP |
| | | | | synthase chain delta |
| SEQ ID NO. 574 | LM-1697.1 | From 2608515 | to 2610029 | unknown, highly similar to |
| | | | | H+-transporting ATP |
| | | | | synthase chain alpha |
| SEQ ID NO. 575 | LM-1698.1 | From 2607605 | to 2608477 | unknown, highly similar to |
| | | | | H+-transporting ATP |
| | | | | synthase chain gamma |
| SEQ ID NO. 576 | LM-170.1 | From 1205018 | to 1205899 | Unknown, similar to |
| | | | | ethanolamine ammonia- |
| | | | | lyase, light chain |
| SEQ ID NO. 577 | LM-1700.1 | From 2606123 | to 2607544 | unknown, highly similar to |
| | | | | H+-transporting ATP |
| | | | | synthase chain beta |
| SEQ ID NO. 578 | LM-1701.1 | From 2605697 | to 2606101 | unknown, highly similar to |
| | | | | H+-transporting ATP |
| | | | | synthase chain epsilon |
| SEQ ID NO. 579 | LM-1702.1 | From 2605337 | to 2605573 | Unknown, similar to B. subtilis |
| | | | | YwzB protein |
| SEQ ID NO. 580 | LM-1704.1 | From 2603863 | to 2605155 | unknown, UDP-N- |
| | | | | acetylglucosamine 1- |
| | | | | carboxyvinyltransferase |
| SEQ ID NO. 581 | LM-1705.2 | From 2602705 | to 2603700 | unknown, similar to MreB- |
| | | | | like protein |
| SEQ ID NO. 582 | LM-1706.2 | From 521863 | to 522591 | Unknown |
| SEQ ID NO. 583 | LM-1707.1 | From 522628 | to 523521 | Unknown, similar to |
| | | | | transcriptional regulator |
| | | | | (LysR family) |
| SEQ ID NO. 584 | LM-1708.1 | From 523719 | to 525713 | Unknown, similar to |
| | | | | NADH: flavin oxidoreductase |
| SEQ ID NO. 585 | LM-171.1 | From 1203634 | to 1204998 | Unknown, similar to |
| | | | | ethanolamine ammonia- |
| | | | | lyase, heavy chain |
| SEQ ID NO. 586 | LM-1710.1 | From 525813 | to 526688 | Unknown, similar to |
| | | | | shikimate 5-dehydrogenase |
| SEQ ID NO. 587 | LM-1712.1 | From 526754 | to 527512 | Unknown, similar to 3- |
| | | | | dehydroquinate |
| | | | | dehydratase |
| SEQ ID NO. 588 | LM-1713.1 | From 527580 | to 528488 | Unknown, similar to |
| | | | | transcriptional regulator |
| | | | | (LysR family) |
| SEQ ID NO. 589 | LM-1714.1 | From 528563 | to 530323 | Unknown, similar to acylase |
| SEQ ID NO. 590 | LM-1715.1 | From 530515 | to 531108 | Unknown, weakly similar to |
| | | | | esterase |
| SEQ ID NO. 591 | LM-1716.1 | From 531294 | to 532253 | Unknown, similar to |
| | | | | transmembrane protein |
| SEQ ID NO. 592 | LM-1717.1 | From 532328 | to 532552 | Unknown, similar to B. subtilis |
| | | | | YnzC protein |
| SEQ ID NO. 593 | LM-1718.2 | From 532603 | to 534111 | Unknown, similar to sugar |
| | | | | transferase |
| SEQ ID NO. 594 | LM-1719.1 | From 534307 | to 534756 | Unknown, similar to ribose |
| | | | | 5-phosphate isomerase |
| SEQ ID NO. 595 | LM-1721.1 | From 534753 | to 535421 | Unknown, similar to |
| | | | | ribulose-5-phosphate 3 |
| | | | | epimerase |
| SEQ ID NO. 596 | LM-1722.1 | From 535428 | to 536078 | Unknown, similar to |
| | | | | transaldolase |
| SEQ ID NO. 597 | LM-1723.1 | From 536187 | to 538247 | Unknown, similar to |
| | | | | transcription antiterminator |
| | | | | BglG family |
| SEQ ID NO. 598 | LM-1724.1 | From 538251 | to 538853 | Unknown, similar to putative |
| | | | | sugar-phosphate isomerase |
| SEQ ID NO. 599 | LM-1725.1 | From 538881 | to 539348 | Unknown, similar to PTS |
| | | | | fructose-specific enzyme IIA |
| | | | | component |
| SEQ ID NO. 600 | LM-1726.1 | From 539365 | to 539763 | Unknown |
| SEQ ID NO. 601 | LM-1727.1 | From 539774 | to 540424 | Unknown, similar to |
| | | | | ribulose-5-phosphate 3- |
| | | | | epimerase |
| SEQ ID NO. 602 | LM-1728.1 | From 540421 | to 541467 | Unknown, similar to polyol |
| | | | | (sorbitol) dehydrogenase |
| SEQ ID NO. 603 | LM-1729.1 | From 541483 | to 541776 | Unknown, similar to PTS |
| | | | | system, Galactitol-specific |
| | | | | IIB component |
| SEQ ID NO. 604 | LM-173.1 | From 1202171 | to 1203592 | Unknown, similar to |
| | | | | ethanolamine utilization |
| | | | | protein EutA (putative |
| | | | | chaperonin) |
| SEQ ID NO. 605 | LM-1731.1 | From 541791 | to 543062 | Unknown, similar to PTS |
| | | | | system, Galactitol-specific |
| | | | | IIC component |
| SEQ ID NO. 606 | LM-1733.1 | From 543202 | to 544137 | Unknown, similar to |
| | | | | phosphoribosyl |
| | | | | pyrophosphate synthetase |
| SEQ ID NO. 607 | LM-1734.1 | From 544923 | to 545501 | Unknown |
| SEQ ID NO. 608 | LM-1735.1 | From 545609 | to 546289 | Unknown, conserved |
| | | | | hypothetical protein |
| SEQ ID NO. 609 | LM-1736.1 | From 546314 | to 546673 | Unknown |
| SEQ ID NO. 610 | LM-1737.1 | From 546680 | to 547138 | Unknown, weakly similar to |
| | | | | transcription regulator |
| SEQ ID NO. 611 | LM-174.1 | From 1200625 | to 1202082 | unknown, similar to sensory |
| | | | | transduction histidine kinase |
| SEQ ID NO. 612 | LM-1740.1 | From 549438 | to 549869 | Unknown, conserved |
| | | | | hypothetical protein |
| SEQ ID NO. 613 | LM-1741.1 | From 549916 | to 551346 | Unknown, similar to Bacillus |
| | | | | anthracis encapsulation |
| | | | | protein CapA |
| SEQ ID NO. 614 | LM-1742.2 | From 551467 | to 552282 | Unknown, similar to |
| | | | | phosphoglycerate mutase |
| SEQ ID NO. 615 | LM-175.1 | From 1200051 | to 1200632 | unknown, similar to two- |
| | | | | component response |
| | | | | regulator |
| SEQ ID NO. 616 | LM-1755.2 | From 2678302 | to 2679330 | Unknown, similar to ATP |
| | | | | binding proteins |
| SEQ ID NO. 617 | LM-1757.1 | From 2681167 | to 2682018 | Unknown, similar to |
| | | | | oxidoreductase, aldo/keto |
| | | | | reductase family |
| SEQ ID NO. 618 | LM-1758.1 | From 2682040 | to 2682462 | Unknown, similar to |
| | | | | transcription regulators |
| | | | | (MerR family) |
| SEQ ID NO. 619 | LM-1759.1 | From 2682584 | to 2682943 | Unknown |
| SEQ ID NO. 620 | LM-176.1 | From 1198637 | to 1199776 | unknown, similar to |
| | | | | NADPH-dependent butanol |
| | | | | dehydrogenase |
| SEQ ID NO. 621 | LM-1760.1 | From 2683187 | to 2684056 | Unknown, similar to |
| | | | | unknown proteins |
| SEQ ID NO. 622 | LM-1761.1 | From 2684251 | to 2684643 | ribosomal protein S9 |
| SEQ ID NO. 623 | LM-1762.1 | From 2684665 | to 2685102 | ribosomal protein L13 |
| SEQ ID NO. 624 | LM-1763.1 | From 2685297 | to 2686043 | unknown, highly similar to |
| | | | | pseudouridylate synthase I |
| SEQ ID NO. 625 | LM-1766.1 | From 2686049 | to 2686846 | Unknown, highly similar to |
| | | | | B. subtilis YbaF protein |
| SEQ ID NO. 626 | LM-1769.1 | From 2686849 | to 2687715 | Uknown similar to ABC |
| | | | | transporter (ATP-binding |
| | | | | protein) |
| SEQ ID NO. 627 | LM-1771.1 | From 2687691 | to 2688530 | Unknown, similar to ABC |
| | | | | transporter (ATP-binding |
| | | | | protein) |
| SEQ ID NO. 628 | LM-1772.1 | From 2688661 | to 2689323 | Unknown, conserved |
| | | | | hypothetical protein |
| SEQ ID NO. 629 | LM-1773.1 | From 2689442 | to 2690332 | Unknown |
| SEQ ID NO. 630 | LM-1774.1 | From 2690371 | to 2691465 | Unknown |
| SEQ ID NO. 631 | LM-1775.2 | From 2691673 | to 2692080 | ribosomal protein L17 |
| SEQ ID NO. 632 | LM-1776.3 | From 1441313 | to 1441540 | Unknown |
| SEQ ID NO. 633 | LM-1777.3 | From 1441581 | to 1442114 | Unknown, modulates DNA |
| | | | | topology |
| SEQ ID NO. 634 | LM-1779.1 | From 1443873 | to 1445042 | Unknown, similar to Acetyl- |
| | | | | CoA: acetyltransferase |
| SEQ ID NO. 635 | LM-1780.2 | From 1445192 | to 1446358 | Unknown, similar to |
| | | | | hydroxy-3-methylglutaryl |
| | | | | coenzyme A synthase |
| SEQ ID NO. 636 | LM-1781.2 | From 1446383 | to 1446982 | Unknown |
| SEQ ID NO. 637 | LM-1782.1 | From 1447052 | to 1448296 | Unknown, highly similar to |
| | | | | B. subtilis YxiO protein |
| SEQ ID NO. 638 | LM-1783.1 | From 1448315 | to 1449583 | Unknown, weakly similar to |
| | | | | pyrophosphatase |
| SEQ ID NO. 639 | LM-1784.1 | From 1449647 | to 1450684 | Unknown, conserved |
| | | | | hypothetical protein |
| SEQ ID NO. 640 | LM-1785.1 | From 1450701 | to 1451597 | Unknown, weakly similar to |
| | | | | UDP-N-acetylglucosaminyl- |
| | | | | 3-enolpyruvate reductase |
| SEQ ID NO. 641 | LM-1787.1 | From 1451813 | to 1452799 | Unknown, similar to glycine |
| | | | | betaine/carnitine/choline |
| | | | | ABC transporter |
| SEQ ID NO. 642 | LM-1789.1 | From 1452796 | to 1454310 | Unknown, similar to glycine |
| | | | | betaine/carnitine/choline |
| | | | | ABC transporter (membrane |
| | | | | protein) |
| SEQ ID NO. 643 | LM-179.1 | From 1197172 | to 1198047 | unknown, similar to |
| | | | | Salmonella enterica PduX |
| | | | | protein |
| SEQ ID NO. 644 | LM-1790.1 | From 1454347 | to 1455177 | Unknown |
| SEQ ID NO. 645 | LM-1791.2 | From 1455316 | to 1456662 | Unknown, similar to metal |
| | | | | ion transport proteins |
| SEQ ID NO. 646 | LM-1794.2 | From 1456775 | to 1457446 | Unknown, similar to |
| | | | | betaine/carnitine/choline |
| | | | | ABC transporter |
| | | | | (membrane p) |
| SEQ ID NO. 647 | LM-1795.1 | From 1457461 | to 1458387 | Unknown, similar to glycine |
| | | | | betaine/carnitine/choline |
| | | | | ABC transporter |
| | | | | (osmoprotectant-binding |
| | | | | protein) |
| SEQ ID NO. 648 | LM-1796.1 | From 1458389 | to 1459045 | Unknown, similar to glycine |
| | | | | betaine/carnitine/choline |
| | | | | ABC transporter (membrane |
| | | | | protein) |
| SEQ ID NO. 649 | LM-1798.1 | From 1459049 | to 1460242 | Unknown, similar to glycine |
| | | | | betaine/carnitine/choline |
| | | | | ABC transporter (ATP- |
| | | | | binding protein) |
| SEQ ID NO. 650 | LM-1799.1 | From 1460534 | to 1461094 | Unknown, similar to |
| | | | | unknown proteins |
| SEQ ID NO. 651 | LM-18.1 | From 2722884 | to 2723156 | Unknown, similar to |
| | | | | hypothetical PTS enzyme |
| | | | | IIB component |
| SEQ ID NO. 652 | LM-1800.2 | From 1461343 | to 1461726 | Unknown, similar to |
| | | | | unknown proteins |
| SEQ ID NO. 653 | LM-1801.2 | From 1461866 | to 1463467 | Unknown, similar to ABC |
| | | | | transporter (ATP-binding |
| | | | | protein) |
| SEQ ID NO. 654 | LM-1802.1 | From 1463506 | to 1464156 | Unknown |
| SEQ ID NO. 655 | LM-1803.1 | From 1464209 | to 1465549 | Unknown, similar to |
| | | | | glutathione reductase |
| SEQ ID NO. 656 | LM-1805.1 | From 1465638 | to 1467305 | Unknown, similar to |
| | | | | unknown proteins |
| SEQ ID NO. 657 | LM-1806.1 | From 1467325 | to 1468206 | Unknown, similar to |
| | | | | dihydrodipicolinate synthase |
| SEQ ID NO. 658 | LM-1807.1 | From 1468221 | to 1469432 | Unknown, similar to |
| | | | | aspartokinase I (alpha and |
| | | | | beta subunits) |
| SEQ ID NO. 659 | LM-1809.1 | From 1469444 | to 1470487 | Unknown, similar to |
| | | | | aspartate-semialdehyde |
| | | | | dehydrogenase |
| SEQ ID NO. 660 | LM-1810.1 | From 1470685 | to 1472850 | unknown, similar to |
| | | | | penicillin-binding protein |
| SEQ ID NO. 661 | LM-1811.1 | From 1472980 | to 1473588 | superoxide dismutase |
| SEQ ID NO. 662 | LM-1813.1 | From 1473889 | to 1474689 | unknown, similar to |
| | | | | unknown proteins |
| SEQ ID NO. 663 | LM-1814.1 | From 1474802 | to 1475908 | Unknown, similar to putative |
| | | | | peptidoglycan acetylation |
| | | | | protein |
| SEQ ID NO. 664 | LM-1815.1 | From 1475944 | to 1476651 | Unknown, similar to |
| | | | | transport proteins |
| SEQ ID NO. 665 | LM-1816.1 | From 1476642 | to 1476968 | Unknown |
| SEQ ID NO. 666 | LM-1817.1 | From 1477050 | to 1477934 | Unknown, similar to protein |
| | | | | secretion PrsA (post- |
| | | | | translocation molecular |
| | | | | chaperone) |
| SEQ ID NO. 667 | LM-1818.1 | From 1478071 | to 1478496 | transcriptional regulator |
| | | | | ZurR (ferric uptake |
| | | | | regulation) |
| SEQ ID NO. 668 | LM-182.1 | From 1194890 | to 1196083 | Unknown, similar to acetate |
| | | | | kinase |
| SEQ ID NO. 669 | LM-1820.1 | From 1478477 | to 1479355 | metal transport protein |
| SEQ ID NO. 670 | LM-1821.1 | From 1479330 | to 1480103 | ABC transporter |
| SEQ ID NO. 671 | LM-1823.1 | From 1480246 | to 1481172 | Unknown, conserved |
| | | | | hypothetical protein |
| SEQ ID NO. 672 | LM-1825.1 | From 1481188 | to 1482081 | unknown, similar to |
| | | | | endonuclease IV |
| SEQ ID NO. 673 | LM-1826.1 | From 1482096 | to 1483403 | Unknown, similar to ATP- |
| | | | | dependent RNA helicase, |
| | | | | DEAD-box family (deaD) |
| SEQ ID NO. 674 | LM-1827.1 | From 1483549 | to 1484544 | Unknown, similar to E. coli |
| | | | | LytB protein |
| SEQ ID NO. 675 | LM-1829.1 | From 1484590 | to 1485711 | Unknown, conserved |
| | | | | hypothetical protein |
| SEQ ID NO. 676 | LM-183.1 | From 1194120 | to 1194824 | Unknown, similar to glycerol |
| | | | | uptake facilitator protein |
| SEQ ID NO. 677 | LM-1830.1 | From 1485708 | to 1486412 | Unknown, conserved |
| | | | | hypothetical protein |
| SEQ ID NO. 678 | LM-1833.2 | From 1486480 | to 1487604 | RNA polymerase sigma |
| | | | | factor RpoD |
| SEQ ID NO. 679 | LM-1834.2 | From 289562 | to 290521 | Unknown, similar to other |
| | | | | proteins |
| SEQ ID NO. 680 | LM-1835.1 | From 289139 | to 289555 | Unknown, similar to |
| | | | | transcriptional regulators |
| SEQ ID NO. 681 | LM-1836.1 | From 287853 | to 288992 | Unknown, similar to |
| | | | | succinyldiaminopimelate |
| | | | | desuccinylase |
| SEQ ID NO. 682 | LM-1838.1 | From 286219 | to 287718 | internalin E |
| SEQ ID NO. 683 | LM-184.1 | From 1192974 | to 1194092 | unknown, similar to |
| | | | | NADPH-dependent butanol |
| | | | | dehydrogenase |
| SEQ ID NO. 684 | LM-1840.1 | From 284365 | to 286011 | internalin H |
| SEQ ID NO. 685 | LM-1842.1 | From 282755 | to 284227 | internalin G |
| SEQ ID NO. 686 | LM-1843.1 | From 281021 | to 282481 | Unknown, similar to |
| | | | | phospho-beta-glucosidase |
| SEQ ID NO. 687 | LM-1844.1 | From 280410 | to 280955 | Unknown, similar to |
| | | | | unknown proteins |
| SEQ ID NO. 688 | LM-185.1 | From 1191549 | to 1192958 | Unknown, similar to |
| | | | | ethanolamine utilization |
| | | | | protein EutE |
| SEQ ID NO. 689 | LM-1852.1 | From 276728 | to 280333 | RNA polymerase (beta |
| | | | | subunit) |
| SEQ ID NO. 690 | LM-1854.1 | From 273003 | to 276557 | RNA polymerase (beta |
| | | | | subunit) |
| SEQ ID NO. 691 | LM-1856.2 | From 271324 | to 272502 | Unknown, similar to |
| | | | | unknown protein |
| SEQ ID NO. 692 | LM-1858.1 | From 269754 | to 270257 | Unknown, similar to |
| | | | | unknown protein |
| SEQ ID NO. 693 | LM-1859.2 | From 269067 | to 269729 | Unknown |
| SEQ ID NO. 694 | LM-1860.2 | From 1993372 | to 1994637 | Unknown, similar to |
| | | | | unknown proteins |
| SEQ ID NO. 695 | LM-1861.1 | From 1991047 | to 1993326 | Unknown, similar to |
| | | | | pyruvate formate-lyase |
| SEQ ID NO. 696 | LM-1862.1 | From 1989805 | to 1990812 | Unknown, similar to |
| | | | | peptidase |
| SEQ ID NO. 697 | LM-1863.1 | From 1988159 | to 1989802 | Unknown, similar to |
| | | | | malolactic enzyme (malate |
| | | | | dehydrogenase) |
| SEQ ID NO. 698 | LM-1864.1 | From 1987365 | to 1988048 | Unknown, similar to |
| | | | | unknown proteins |
| SEQ ID NO. 699 | LM-1865.1 | From 1986342 | to 1987346 | Unknown, similar to |
| | | | | unknown proteins |
| SEQ ID NO. 700 | LM-1866.1 | From 1985218 | to 1986345 | Unknown, similar to |
| | | | | unknown proteins |
| | | | | (hypothetical sensory |
| | | | | transduction histidine |
| | | | | kinase) |
| SEQ ID NO. 701 | LM-1867.1 | From 1984056 | to 1985192 | Unknown, similar to |
| | | | | unknown proteins |
| | | | | (hypothetical sensory |
| | | | | transduction histidine |
| | | | | kinase) |
| SEQ ID NO. 702 | LM-1869.1 | From 1982630 | to 1983736 | Unknown, similar to |
| | | | | oxidoreductases |
| SEQ ID NO. 703 | LM-1871.1 | From 1981774 | to 1982640 | Unknown, similar to |
| | | | | unknown proteins |
| SEQ ID NO. 704 | LM-1872.1 | From 1981299 | to 1981634 | Unknown, similar to |
| | | | | unknown proteins |
| SEQ ID NO. 705 | LM-1874.1 | From 1980511 | to 1981302 | Unknown, similar to |
| | | | | dihydrodipicolinate |
| | | | | reductase |
| SEQ ID NO. 706 | LM-1875.1 | From 1980091 | to 1980495 | Unknown, similar to |
| | | | | methylglyoxal synthase |
| SEQ ID NO. 707 | LM-1876.1 | From 1978895 | to 1980076 | Unknown, similar to tRNA |
| | | | | CCA-adding enzyme |
| SEQ ID NO. 708 | LM-1877.1 | From 1977928 | to 1978905 | Unknown, similar to |
| | | | | transcriptional regulator and |
| | | | | biotin acetyl-CoA- |
| | | | | carboxylase synthetase |
| SEQ ID NO. 709 | LM-1879.1 | From 1977307 | to 1977780 | unknown; similar to |
| | | | | thioredoxin |
| SEQ ID NO. 710 | LM-188.1 | From 1190547 | to 1191542 | Unknown, hyghly similar to |
| | | | | Salmonella enterica PduO |
| | | | | protein |
| SEQ ID NO. 711 | LM-1880.1 | From 1976307 | to 1977140 | unknown, similar to |
| | | | | ketopantoate |
| | | | | hydroxymethyltransferases |
| SEQ ID NO. 712 | LM-1881.1 | From 1975446 | to 1976303 | unknown, similar to |
| | | | | panthotenate synthetases |
| SEQ ID NO. 713 | LM-1882.1 | From 1975059 | to 1975442 | unknown, similar to |
| | | | | aspartate 1-decarboxylases |
| SEQ ID NO. 714 | LM-1884.1 | From 1972176 | to 1974962 | unknown, similar to ATP- |
| | | | | dependent helicases |
| SEQ ID NO. 715 | LM-1885.1 | From 1971547 | to 1972131 | unknown, similar to |
| | | | | hypothetical proteins |
| SEQ ID NO. 716 | LM-1886.1 | From 1970343 | to 1971524 | unknown, similar to |
| | | | | aspartate |
| | | | | aminotransferases |
| SEQ ID NO. 717 | LM-1888.1 | From 1969037 | to 1970329 | unknown, similar to |
| | | | | sparaginyl-tRNA |
| | | | | synthetases |
| SEQ ID NO. 718 | LM-189.1 | From 1190269 | to 1190532 | Unknown, similar to carbondioxide |
| | | | | concentrating |
| | | | | mechanism protein |
| SEQ ID NO. 719 | LM-1891.1 | From 1968169 | to 1968888 | unknown, similar to |
| | | | | chromosome replication |
| | | | | initiation protein |
| SEQ ID NO. 720 | LM-1893.1 | From 1967499 | to 1968158 | unknown, probable |
| | | | | endonuclease III (DNA |
| | | | | repair) |
| SEQ ID NO. 721 | LM-1894.1 | From 1967015 | to 1967506 | Unknown |
| SEQ ID NO. 722 | LM-1896.1 | From 1964489 | to 1966972 | unknown, similar to |
| | | | | penicillin-binding protein 2A |
| SEQ ID NO. 723 | LM-1897.2 | From 1963852 | to 1964457 | unknown, similar to DNA |
| | | | | repair and homologous |
| | | | | recombination protein |
| SEQ ID NO. 724 | LM-1898.2 | From 2208576 | to 2210351 | Unknown, similar to |
| | | | | maltogenic amylase |
| SEQ ID NO. 725 | LM-19.1 | From 2723159 | to 2723593 | Unknown, similar to |
| | | | | mannitol-specific PTS |
| | | | | enzyme IIA component |
| SEQ ID NO. 726 | LM-190.1 | From 1189785 | to 1190264 | Unknown |
| SEQ ID NO. 727 | LM-1900.1 | From 2207103 | to 2208362 | Unknown, similar to |
| | | | | maltose/maltodextrin- |
| | | | | binding protein |
| SEQ ID NO. 728 | LM-1904.1 | From 2205709 | to 2207016 | Unknown, similar to |
| | | | | maltodextrin transport |
| | | | | system permease |
| SEQ ID NO. 729 | LM-1905.1 | From 2204857 | to 2205708 | Unknown, similar to |
| | | | | maltodextrin transport |
| | | | | system permease |
| SEQ ID NO. 730 | LM-1906.1 | From 2203996 | to 2204829 | Unknown, similar to |
| | | | | maltodextrose utilization |
| | | | | protein MalA |
| SEQ ID NO. 731 | LM-1907.1 | From 2201719 | to 2203980 | Unknown, similar to |
| | | | | maltosephosphorylase |
| SEQ ID NO. 732 | LM-1908.1 | From 2200723 | to 2201544 | Unknown, similar to |
| | | | | unknown proteins |
| SEQ ID NO. 733 | LM-1910.1 | From 2199365 | to 2200723 | Unknown, similar to |
| | | | | unknown proteins |
| SEQ ID NO. 734 | LM-1911.1 | From 2197763 | to 2199115 | Unknown, similar to |
| | | | | phosphoglucomutase |
| SEQ ID NO. 735 | LM-1912.1 | From 2197267 | to 2197728 | Unknown, similar to |
| | | | | unknown proteins |
| SEQ ID NO. 736 | LM-1913.1 | From 2196382 | to 2197206 | Unknown |
| SEQ ID NO. 737 | LM-1915.1 | From 2194399 | to 2196339 | Unknown, similar to ABC |
| | | | | transporter (permease) |
| SEQ ID NO. 738 | LM-1916.1 | From 2193645 | to 2194412 | Unknown, similar to ABC |
| | | | | transporter (ATP-binding |
| | | | | protein) |
| SEQ ID NO. 739 | LM-1918.2 | From 2192693 | to 2193448 | Unknown, similar to |
| | | | | unknown proteins |
| SEQ ID NO. 740 | LM-1919.2 | From 2191525 | to 2192256 | Unknown, similar to FMN- |
| | | | | containing NADPH-linked |
| | | | | nitro/flavin reductase |
| SEQ ID NO. 741 | LM-192.1 | From 1188949 | to 1189788 | Unknown, similar to |
| | | | | ethanolamine utilization |
| | | | | protein EutJ |
| SEQ ID NO. 742 | LM-1920.1 | From 2190489 | to 2191445 | Unknown, similar to |
| | | | | mannnose-6 phospate |
| | | | | isomelase |
| SEQ ID NO. 743 | LM-1921.1 | From 2189605 | to 2190402 | Unknown, similar to |
| | | | | hydrolase |
| SEQ ID NO. 744 | LM-1922.1 | From 2188430 | to 2189563 | Unknown, similar to N- |
| | | | | acetylglucosamine-6- |
| | | | | phosphate deacetylase |
| SEQ ID NO. 745 | LM-1923.1 | From 2187663 | to 2188424 | Unknown, similar to |
| | | | | transcriptional regulator |
| | | | | (DeoR family) |
| SEQ ID NO. 746 | LM-1924.1 | From 2186557 | to 2187411 | Unknown, similar to |
| | | | | unknown proteins |
| SEQ ID NO. 747 | LM-1927.1 | From 2184344 | to 2186338 | Unknown, similar to ferrous |
| | | | | iron transport protein B |
| SEQ ID NO. 748 | LM-1930.1 | From 2182823 | to 2183800 | Unknown, similar to |
| | | | | phosphotransacetylase |
| SEQ ID NO. 749 | LM-1932.1 | From 2182218 | to 2182784 | Unknown |
| SEQ ID NO. 750 | LM-1933.2 | From 2181329 | to 2182216 | Unknown, similar to a |
| | | | | protein required for |
| | | | | pyridoxine synthesis |
| SEQ ID NO. 751 | LM-1935.1 | From 1610206 | to 1611165 | unknown, highly similar to |
| | | | | 6-phosphofructokinase |
| SEQ ID NO. 752 | LM-1936.1 | From 1611449 | to 1612405 | unknown, highly similar to |
| | | | | acetyl CoA carboxylase |
| | | | | (alpha subunit) |
| SEQ ID NO. 753 | LM-1938.1 | From 1612395 | to 1613279 | unknown, highly similar to |
| | | | | acetyl-CoA carboxylase |
| | | | | beta subunit |
| SEQ ID NO. 754 | LM-1942.1 | From 1613497 | to 1616823 | unknown, highly similar to |
| | | | | DNA polymerase III (alpha |
| | | | | subunit) DnaE |
| SEQ ID NO. 755 | LM-1944.1 | From 1616945 | to 1617880 | Unknown, similar to |
| | | | | unknown proteins |
| SEQ ID NO. 756 | LM-1945.1 | From 1617901 | to 1619214 | Unknown, similar to |
| | | | | unknown proteins |
| SEQ ID NO. 757 | LM-1946.1 | From 1619240 | to 1619926 | Unknown, similar to |
| | | | | unknown proteins |
| SEQ ID NO. 758 | LM-1947.1 | From 1620091 | to 1621188 | Unknown, similar to X-Pro |
| | | | | dipeptidase |
| SEQ ID NO. 759 | LM-1948.1 | From 1621230 | to 1622342 | Unknown, similar to alanine |
| | | | | dehydrogenase |
| SEQ ID NO. 760 | LM-1949.1 | From 1622583 | to 1623047 | Unknown, similar to |
| | | | | unknown protein |
| SEQ ID NO. 761 | LM-195.1 | From 1188293 | to 1188928 | Unknown, similar to |
| | | | | Salmonella enterica PduL |
| | | | | protein |
| SEQ ID NO. 762 | LM-1950.1 | From 1623099 | to 1624292 | unknown, highly similar to |
| | | | | acetate kinase |
| SEQ ID NO. 763 | LM-1951.1 | From 1624316 | to 1625314 | Unknown, weakly similar to |
| | | | | site specific DNA- |
| | | | | methyltransferase |
| SEQ ID NO. 764 | LM-1952.1 | From 1625505 | to 1626002 | Unknown, similar to thiol |
| | | | | peroxidases |
| SEQ ID NO. 765 | LM-1953.1 | From 1626064 | to 1626600 | Unknown, similar to |
| | | | | unknown proteins |
| SEQ ID NO. 766 | LM-1954.1 | From 1626620 | to 1627633 | Unknown, similar to |
| | | | | proteases |
| SEQ ID NO. 767 | LM-1955.1 | From 1627812 | to 1628615 | Unknown, similar to |
| | | | | unknown proteins |
| SEQ ID NO. 768 | LM-1957.1 | From 1628647 | to 1629591 | unknown, highly similar to |
| | | | | ornithine |
| | | | | carbamoyltransferase |
| SEQ ID NO. 769 | LM-1958.1 | From 1629594 | to 1630754 | unknown, highly similar to |
| | | | | N-acetylornithine |
| | | | | aminotransferase |
| SEQ ID NO. 770 | LM-1959.1 | From 1630751 | to 1631503 | unknown, highly similar to |
| | | | | N-acetylglutamate 5- |
| | | | | phosphotransferase |
| SEQ ID NO. 771 | LM-1960.1 | From 1631516 | to 1632712 | unknown, highly similar to |
| | | | | ornithine acetyltransferase |
| | | | | and amino-acid |
| | | | | acetyltransferases |
| SEQ ID NO. 772 | LM-1961.2 | From 1632728 | to 1633759 | unknown, similar to N- |
| | | | | acetylglutamate gamma- |
| | | | | semialdehyde |
| | | | | dehydrogenases |
| SEQ ID NO. 773 | LM-1962.2 | From 1633933 | to 1635144 | Unknown, similar to thiamin |
| | | | | biosynthesis protein Thil |
| SEQ ID NO. 774 | LM-1964.1 | From 1635146 | to 1636285 | Unknown, similar to iron- |
| | | | | sulfur cofactor synthesis |
| | | | | protein nifS |
| SEQ ID NO. 775 | LM-1966.2 | From 1636412 | to 1638127 | Unknown, similar to B. subtilis |
| | | | | negative regulator of |
| | | | | FtsZ ring formation (EzrA) |
| SEQ ID NO. 776 | LM-1968.2 | From 1308351 | to 1310318 | unknown, highly similar to |
| | | | | DNA gyrase-like protein |
| | | | | (subunit B) |
| SEQ ID NO. 777 | LM-197.1 | From 1187552 | to 1187989 | Unknown, similar to |
| | | | | Salmonella enterica PduK |
| | | | | protein |
| SEQ ID NO. 778 | LM-1970.1 | From 1310315 | to 1312774 | unknown, highly similar to |
| | | | | DNA gyrase-like protein |
| | | | | (subunit A) |
| SEQ ID NO. 779 | LM-1971.1 | From 1312857 | to 1313324 | Unknown, conserved |
| | | | | hypothetical protein |
| SEQ ID NO. 780 | LM-1976.1 | From 1317596 | to 1319464 | Unknown; similar to |
| | | | | acyltransferase (to B. subtilis |
| | | | | YrhL protein) |
| SEQ ID NO. 781 | LM-1978.1 | From 1319677 | to 1320375 | Unknown similar to |
| | | | | glycerophosphodiester |
| | | | | phosphodiesterase |
| SEQ ID NO. 782 | LM-1979.1 | From 1320608 | to 1322284 | Unknown, similar to glycerol |
| | | | | 3 phosphate |
| | | | | dehydrogenase |
| SEQ ID NO. 783 | LM-198.1 | From 1187192 | to 1187539 | unknown, similar to diol |
| | | | | dehydratase-reactivating |
| | | | | factor small chain |
| SEQ ID NO. 784 | LM-1980.1 | From 1322410 | to 1323327 | Unknown, similar to tRNA |
| | | | | isopentenylpyrophosphate |
| | | | | transferase |
| SEQ ID NO. 785 | LM-1981.1 | From 1323450 | to 1323683 | Unknown, similar to host |
| | | | | factor-1 protein |
| SEQ ID NO. 786 | LM-1983.1 | From 1323794 | to 1325017 | Unknown, conserved |
| | | | | hypothetical protein similar |
| | | | | to B. subtilis YnbA protein |
| SEQ ID NO. 787 | LM-1984.1 | From 1325010 | to 1326236 | Unknown, similar to |
| | | | | aluminum resistance protein |
| | | | | and to B. subtilis YnbB |
| | | | | protein |
| SEQ ID NO. 788 | LM-1985.1 | From 1326440 | to 1326808 | Unknown, similar to |
| | | | | glutamine synthetase |
| | | | | repressor |
| SEQ ID NO. 789 | LM-1986.1 | From 1326879 | to 1328213 | unknown, highly similar to |
| | | | | glutamine synthetases |
| SEQ ID NO. 790 | LM-1988.1 | From 1328357 | to 1329652 | Unknown, similar to arsenic |
| | | | | efflux pump protein |
| SEQ ID NO. 791 | LM-1989.1 | From 1329696 | to 1330217 | Unknown, conserved |
| | | | | hypothetical protein |
| SEQ ID NO. 792 | LM-1991.2 | From 1330247 | to 1330861 | unknown, highly similar to |
| | | | | SOS response regulator |
| | | | | lexA, transcription repressor |
| | | | | protein |
| SEQ ID NO. 793 | LM-1992.2 | From 1331018 | to 1331347 | unknown, similar to B. subtilis |
| | | | | YneA protein |
| SEQ ID NO. 794 | LM-1995.1 | From 1331813 | to 1333807 | unknown, highly similar to |
| | | | | transketolase |
| SEQ ID NO. 795 | LM-1996.1 | From 1334028 | to 1334267 | unknown, highly similar to |
| | | | | B. subtilis YneF protein |
| SEQ ID NO. 796 | LM-1997.1 | From 1334318 | to 1335160 | Unknown |
| SEQ ID NO. 797 | LM-1998.1 | From 1335179 | to 1335910 | unknown, weakly similar to |
| | | | | arginine N- |
| | | | | methyltransferases |
| SEQ ID NO. 798 | LM-1999.1 | From 1335932 | to 1336438 | unknown, similar to E. coli |
| | | | | YbdM protein |
| SEQ ID NO. 799 | LM-200.1 | From 1185375 | to 1187195 | diol dehydratase- |
| | | | | reactivating factor large |
| | | | | subunit |
| SEQ ID NO. 800 | LM-2000.1 | From 1336448 | to 1337752 | unknown, similar to E. coli |
| | | | | YbdN protein |
| SEQ ID NO. 801 | LM-2001.1 | From 1337742 | to 1338947 | Unknown |
| SEQ ID NO. 802 | LM-2002.1 | From 1338925 | to 1339299 | Unknown |
| SEQ ID NO. 803 | LM-2003.1 | From 1339592 | to 1340320 | unknown, highly similar to |
| | | | | uridylate kinases |
| SEQ ID NO. 804 | LM-2004.1 | From 1340320 | to 1340877 | unknown, highly similar to |
| | | | | ribosome recycling factors |
| SEQ ID NO. 805 | LM-2005.2 | From 1341107 | to 1341865 | Unknown, similar to |
| | | | | undecaprenyl diphosphate |
| | | | | synthase |
| SEQ ID NO. 806 | LM-2006.2 | From 290593 | to 291228 | Unknown, similar to |
| | | | | phosphoglycerate mutase |
| SEQ ID NO. 807 | LM-2008.1 | From 291312 | to 293198 | Unknown, similar to |
| | | | | transporter |
| SEQ ID NO. 808 | LM-2009.1 | From 293212 | to 293841 | Unknown |
| SEQ ID NO. 809 | LM-201.1 | From 1184818 | to 1185330 | Unknown, similar to diol |
| | | | | dehydrase (diol |
| | | | | dehydratase) gamma |
| | | | | subunit (pddC) |
| SEQ ID NO. 810 | LM-2010.1 | From 293845 | to 295281 | unknown, highly similar to |
| | | | | phospho-beta-glucosidase |
| SEQ ID NO. 811 | LM-2011.1 | From 295401 | to 296213 | Unknown, conserved |
| | | | | hypothetical protein similar |
| | | | | to B. subtilis YxeH protein |
| SEQ ID NO. 812 | LM-2012.1 | From 296349 | to 296852 | Unknown |
| SEQ ID NO. 813 | LM-2013.1 | From 296889 | to 297551 | Unknown |
| SEQ ID NO. 814 | LM-2014.1 | From 297810 | to 298652 | Unknown, C-terminal part |
| | | | | similar to B. subtilis ComEC |
| | | | | protein |
| SEQ ID NO. 815 | LM-2015.1 | From 298669 | to 299490 | Unknown, conserved |
| | | | | hypothetical protein |
| SEQ ID NO. 816 | LM-2017.1 | From 299607 | to 300578 | Unknown, similar to |
| | | | | oxidoreductase |
| SEQ ID NO. 817 | LM-2018.1 | From 300617 | to 301717 | Unknown, similar to sugar |
| | | | | ABC transporter, ATP- |
| | | | | binding protein |
| SEQ ID NO. 818 | LM-2019.1 | From 302008 | to 304140 | Unknown, highly similar to |
| | | | | anaerobic ribonucleoside- |
| | | | | triphosphate reductase |
| SEQ ID NO. 819 | LM-202.1 | From 1184142 | to 1184801 | Unknown, similar to diol |
| | | | | dehydrase (diol |
| | | | | dehydratase) gamma |
| | | | | subunit |
| SEQ ID NO. 820 | LM-2020.1 | From 304133 | to 304684 | Unknown, highly similar to |
| | | | | anaerobic ribonucleotide |
| | | | | reductase activator protein |
| SEQ ID NO. 821 | LM-2022.1 | From 304942 | to 305613 | Unknown |
| SEQ ID NO. 822 | LM-2023.1 | From 305775 | to 306554 | Unknown, conserved |
| | | | | hypothetical protein |
| SEQ ID NO. 823 | LM-2024.1 | From 306644 | to 307306 | Unknown, similar to ABC |
| | | | | transporter permease |
| | | | | protein |
| SEQ ID NO. 824 | LM-2026.1 | From 307303 | to 308319 | Unknown, similar to ABC |
| | | | | transporter (ATP-binding |
| | | | | protein) |
| SEQ ID NO. 825 | LM-2027.2 | From 308334 | to 309155 | Unknown, putative |
| | | | | lipoprotein |
| SEQ ID NO. 826 | LM-203.1 | From 1182440 | to 1184104 | unknown, highly similar to |
| | | | | propanediol dehydratase, |
| | | | | alpha subunit |
| SEQ ID NO. 827 | LM-2035.1 | From 2671624 | to 2672511 | Unknown, similar to |
| | | | | transcription regulator |
| | | | | TetR/AcrR family |
| SEQ ID NO. 828 | LM-2037.1 | From 2670042 | to 2671523 | Unknown, similar to drug- |
| | | | | export proteins |
| SEQ ID NO. 829 | LM-2038.1 | From 2669152 | to 2669994 | Unknown, conserved |
| | | | | hypothetical proteins |
| SEQ ID NO. 830 | LM-2039.1 | From 2665666 | to 2668653 | Unknown, similar to formate |
| | | | | dehydrogenase alpha chain |
| SEQ ID NO. 831 | LM-204.1 | From 1181720 | to 1182421 | Unknown, similar to |
| | | | | Salmonella typhimurium |
| | | | | PduB protein |
| SEQ ID NO. 832 | LM-2040.1 | From 2665190 | to 2665666 | Unknown, similar to B. subtilis |
| | | | | YrhD protein |
| SEQ ID NO. 833 | LM-2042.1 | From 2664343 | to 2665128 | Unknown, similar to formate |
| | | | | dehydrogenase associated |
| | | | | protein |
| SEQ ID NO. 834 | LM-2043.1 | From 2663619 | to 2664296 | unknown, similar to two- |
| | | | | component response |
| | | | | regulator |
| SEQ ID NO. 835 | LM-2044.1 | From 2662243 | to 2663622 | Unknown, similar to |
| | | | | response regulator histidine |
| | | | | kinase |
| SEQ ID NO. 836 | LM-2045.1 | From 2661076 | to 2662164 | Unknown, conserved |
| | | | | hypothetical protein |
| SEQ ID NO. 837 | LM-2046.1 | From 2660408 | to 2661076 | Unknown, similar to ABC |
| | | | | transporter, ATP-binding |
| | | | | protein |
| SEQ ID NO. 838 | LM-2047.1 | From 2659852 | to 2660145 | Unknown, conserved |
| | | | | hypothetical protein |
| SEQ ID NO. 839 | LM-2048.2 | From 2658966 | to 2659841 | Unknown |
| SEQ ID NO. 840 | LM-2049.3 | From 403743 | to 404198 | Unknown |
| SEQ ID NO. 841 | LM-205.1 | From 1181338 | to 1181625 | Unknown, similar to |
| | | | | Salmonella typhimurium |
| | | | | PduA protein |
| SEQ ID NO. 842 | LM-2050.1 | From 403279 | to 403725 | Unknown |
| SEQ ID NO. 843 | LM-2051.1 | From 402641 | to 403060 | Unknown |
| SEQ ID NO. 844 | LM-2053.1 | From 401619 | to 402539 | Unknown, similar to putative |
| | | | | transcription regulator |
| SEQ ID NO. 845 | LM-2054.1 | From 400997 | to 401299 | Unknown, similar to PTS |
| | | | | betaglucoside-specific |
| | | | | enzyme IIB component |
| SEQ ID NO. 846 | LM-2055.1 | From 399645 | to 400979 | Unknown, similar to PTS |
| | | | | betaglucoside-specific |
| | | | | enzyme IIC component |
| SEQ ID NO. 847 | LM-2056.1 | From 398210 | to 399652 | Unknown, similar to beta- |
| | | | | glucosidase |
| SEQ ID NO. 848 | LM-2058.1 | From 397340 | to 398053 | Unknown, similar to |
| | | | | transcription regulator |
| | | | | (GntR family) |
| SEQ ID NO. 849 | LM-2059.1 | From 396964 | to 397299 | Unknown, conserved |
| | | | | hypothetical protein |
| SEQ ID NO. 850 | LM-2060.1 | From 396208 | to 396927 | Unknown, conserved |
| | | | | hypothetical protein, highly |
| | | | | similar to B. subtilis Yeel |
| | | | | protein |
| SEQ ID NO. 851 | LM-2061.1 | From 395602 | to 396111 | Unknown, similar to |
| | | | | different proteins |
| SEQ ID NO. 852 | LM-2063.1 | From 394264 | to 395529 | Unknown, conserved |
| | | | | hypothetical protein similar |
| | | | | to B. subtilis YwbN protein |
| SEQ ID NO. 853 | LM-2064.1 | From 393086 | to 394246 | Unknown, conserved |
| | | | | hypothetical protein, |
| | | | | putative lippoprotein |
| SEQ ID NO. 854 | LM-2065.1 | From 391604 | to 393052 | Unknown, similar to |
| | | | | conserved hypothetical |
| | | | | protein |
| SEQ ID NO. 855 | LM-2066.1 | From 390529 | to 391473 | Unknown, similar to |
| | | | | transcription regulator |
| SEQ ID NO. 856 | LM-2067.1 | From 389813 | to 390430 | Unknown, similar to |
| | | | | Salmonella typhimurium |
| | | | | peptidase E |
| SEQ ID NO. 857 | LM-2068.1 | From 388807 | to 389541 | Unknown, similar to |
| | | | | conserved hypothetical |
| | | | | integral membrane protein |
| SEQ ID NO. 858 | LM-2069.1 | From 387697 | to 388467 | Unknown, similar to |
| | | | | transcriptional regulator |
| | | | | (DeoR family) |
| SEQ ID NO. 859 | LM-2070.1 | From 386780 | to 387640 | Unknown, similar to D- |
| | | | | fructose-1,6-biphosphate |
| | | | | aldolase |
| SEQ ID NO. 860 | LM-2072.1 | From 385386 | to 386780 | Unknown, similar to PTS |
| | | | | system, fructose-specific |
| | | | | enzyme IIBC component |
| SEQ ID NO. 861 | LM-2073.1 | From 384926 | to 385372 | Unknown, similar to PTS |
| | | | | system, enzyme IIA |
| | | | | component |
| SEQ ID NO. 862 | LM-2074.1 | From 383710 | to 384726 | Unknown, similar to |
| | | | | oxidoreductase |
| SEQ ID NO. 863 | LM-2076.1 | From 382016 | to 383536 | Unknown, similar to |
| | | | | Flavocytochrome C |
| | | | | Fumarate Reductase |
| | | | | chain A |
| SEQ ID NO. 864 | LM2077.1 | From 380253 | to 381779 | Unknown, similar to fatty- |
| | | | | acid-CoA ligase |
| SEQ ID NO. 865 | LM-2078.1 | From 379794 | to 380207 | Unknown, similar to |
| | | | | unknown proteins |
| SEQ ID NO. 866 | LM-2080.2 | From 378964 | to 379728 | unknown, highly similar to |
| | | | | regulatory proteins (DeoR |
| | | | | family) |
| SEQ ID NO. 867 | LM-2082.3 | From 2052233 | to 2052664 | Unknown, similar to |
| | | | | unknown proteins |
| SEQ ID NO. 868 | LM-2083.1 | From 2052816 | to 2053301 | Unknown, similar to |
| | | | | unknown proteins |
| SEQ ID NO. 869 | LM-2084.1 | From 2053274 | to 2053801 | Unknown, similar to |
| | | | | unknown proteins |
| SEQ ID NO. 870 | LM-2088.1 | From 2054493 | to 2056187 | Unknown, similar to |
| | | | | dihydroxy-acid dehydratase |
| SEQ ID NO. 871 | LM-2089.1 | From 2056205 | to 2057926 | Unknown, similar to |
| | | | | acetolactate synthase |
| | | | | (acetohydroxy-acid |
| | | | | synthase) (large subunit) |
| SEQ ID NO. 872 | LM-2091.1 | From 2057927 | to 2058418 | Unknown, similar to |
| | | | | acetolactate synthase |
| | | | | (acetohydroxy-acid |
| | | | | synthase) (small subunit) |
| SEQ ID NO. 873 | LM-2092.1 | From 2058516 | to 2059511 | Unknown, similar to ketol- |
| | | | | acid reductoisomerase |
| | | | | (acetohydroxy-acid |
| | | | | isomeroreductase) |
| SEQ ID NO. 874 | LM-2097.1 | From 2059669 | to 2061207 | Unknown, similar to 2- |
| | | | | isopropylmalate synthase |
| SEQ ID NO. 875 | LM-2098.1 | From 2061209 | to 2062261 | Unknown, similar to 3- |
| | | | | isopropylmalate |
| | | | | dehydrogenase |
| SEQ ID NO. 876 | LM-21.1 | From 2723786 | to 2725852 | Unknown, similar to |
| | | | | transcriptional |
| | | | | antiterminator |
| SEQ ID NO. 877 | LM-2100.1 | From 2062263 | to 2063651 | Unknown, similar to 3- |
| | | | | isopropylmalate |
| | | | | dehydratase (large subunit) |
| SEQ ID NO. 878 | LM-2101.1 | From 2063638 | to 2064219 | Unknown, similar to 3- |
| | | | | isopropylmalate |
| | | | | dehydratase (small subunit) |
| SEQ ID NO. 879 | LM-2102.1 | From 2064238 | to 2065506 | Unknown, similar to |
| | | | | threonine dehydratase |
| SEQ ID NO. 880 | LM-2104.1 | From 2065698 | to 2066417 | Unknown, similar to alpha- |
| | | | | acetolactate decarboxylase |
| SEQ ID NO. 881 | LM-2106.1 | From 2066455 | to 2067756 | Unknown, similar to |
| | | | | pyrimidine-nucleoside |
| | | | | phosphorylase |
| SEQ ID NO. 882 | LM-2107.3 | From 2067887 | to 2068897 | Unknown, similar to |
| | | | | transcription regulators |
| | | | | (Lacl family) |
| SEQ ID NO. 883 | LM-2109.2 | From 1677409 | to 1680009 | Unknown, similar to |
| | | | | Alcohol-acetaldehyde |
| | | | | dehydrogenase |
| SEQ ID NO. 884 | LM-2110.1 | From 1680132 | to 1680560 | Unknown, similar to |
| | | | | unknown proteins |
| SEQ ID NO. 885 | LM-2111.1 | From 1680644 | to 1681564 | Unknown, similar to similar |
| | | | | to ABC transporter (ATP- |
| | | | | binding protein) |
| SEQ ID NO. 886 | LM-2113.1 | From 1681561 | to 1682628 | Unknown, similar to |
| | | | | membrane proteins |
| SEQ ID NO. 887 | LM-2114.1 | From 1682664 | to 1683638 | Unknown, similar to |
| | | | | unknown proteins |
| SEQ ID NO. 888 | LM-2115.1 | From 1683635 | to 1684216 | Unknown, similar to dna-3- |
| | | | | methyladenine glycosidase |
| SEQ ID NO. 889 | LM-2116.1 | From 1684229 | to 1684462 | Unknown |
| SEQ ID NO. 890 | LM-2119.1 | From 1684563 | to 1687265 | unknown, highly similar to |
| | | | | aconitate hydratases |
| SEQ ID NO. 891 | LM-2120.1 | From 1687420 | to 1688223 | Unknown, similar to putative |
| | | | | sigma factor regulator |
| SEQ ID NO. 892 | LM-2121.1 | From 1688248 | to 1688670 | Unknown |
| SEQ ID NO. 893 | LM-2123.1 | From 1688670 | to 1691888 | Unknown, similar to SNF2- |
| | | | | type helicase |
| SEQ ID NO. 894 | LM-2127.1 | From 1691972 | to 1695043 | Unknown, similar to ATP- |
| | | | | dependent dsDNA |
| | | | | exonuclease SbcC |
| SEQ ID NO. 895 | LM-2128.1 | From 1695040 | to 1696164 | unknown, similar to putative |
| | | | | exonucleases SbcD |
| SEQ ID NO. 896 | LM-2129.1 | From 1696280 | to 1696888 | unknown, similar to 1- |
| | | | | acylglycerol-3-phosphate O- |
| | | | | acyltransferases |
| SEQ ID NO. 897 | LM-2130.1 | From 1696940 | to 1697302 | Unknown |
| SEQ ID NO. 898 | LM-2131.1 | From 1697476 | to 1697991 | Unknown |
| SEQ ID NO. 899 | LM-2133.1 | From 1698165 | to 1698653 | unknown, similar to |
| | | | | hypothetical proteins |
| SEQ ID NO. 900 | LM-2134.1 | From 1698734 | to 1700539 | unknown, similar to ABC |
| | | | | transporter (ATP-binding |
| | | | | protein) |
| SEQ ID NO. 901 | LM-2135.2 | From 1700523 | to 1702292 | unknown, similar to ABC |
| | | | | transporter (ATP-binding |
| | | | | protein) |
| SEQ ID NO. 902 | LM-2138.1 | From 366508 | to 367032 | Unknown |
| SEQ ID NO. 903 | LM-214.1 | From 1177116 | to 1177865 | Unknown |
| SEQ ID NO. 904 | LM-2140.1 | From 367533 | to 367901 | Unknown |
| SEQ ID NO. 905 | LM-2141.1 | From 367898 | to 369373 | Unknown |
| SEQ ID NO. 906 | LM-2142.1 | From 369377 | to 369757 | Unknown |
| SEQ ID NO. 907 | LM-2143.1 | From 370032 | to 370403 | Unknown, weakly similar to |
| | | | | inorganic pyrophosphatase |
| SEQ ID NO. 908 | LM-2145.1 | From 370732 | to 371064 | Unknown |
| SEQ ID NO. 909 | LM-2147.1 | From 371130 | to 373127 | Unknown, similar to |
| | | | | transketolase |
| SEQ ID NO. 910 | LM-2148.1 | From 373129 | to 373785 | Unknown, similar to |
| | | | | transaldolase |
| SEQ ID NO. 911 | LM-215.1 | From 1176660 | to 1177091 | unknown, similar to |
| | | | | Salmonella enterica PduV |
| | | | | protein |
| SEQ ID NO. 912 | LM-2150.1 | From 373832 | to 374596 | Unknown, similar to |
| | | | | dehydrogenase/reductase |
| SEQ ID NO. 913 | LM.-2151.1 | From 374621 | to 375067 | Unknown, similar to sugar- |
| | | | | phosphate isomerase |
| SEQ ID NO. 914 | LM-2152.1 | From 375074 | to 375838 | Unknown, similar to |
| | | | | triosephosphate isomerase |
| SEQ ID NO. 915 | LM-2154.1 | From 375842 | to 376492 | Unknown, similar to |
| | | | | dihydroxyacetone kinase |
| SEQ ID NO. 916 | LM-2155.1 | From 376514 | to 377509 | Unknown, similar to |
| | | | | dihydroxyacetone kinase |
| SEQ ID NO. 917 | LM-2156.1 | From 377531 | to 377872 | Unknown |
| SEQ ID NO. 918 | LM-2157.2 | From 377888 | to 378319 | Unknown |
| SEQ ID NO. 919 | LM-2158.2 | From 378404 | to 378781 | Unknown, similar to |
| | | | | unknown proteins |
| SEQ ID NO. 920 | LM-216.1 | From 1176304 | to 1176654 | Unknown, similar to |
| | | | | Salmonelle enterica PduU |
| | | | | protein |
| SEQ ID NO. 921 | LM-2161.2 | From 72408 | to 74222 | Unknown, similar to toxin |
| | | | | components |
| SEQ ID NO. 922 | LM-2162.1 | From 72062 | to 72394 | Unknown |
| SEQ ID NO. 923 | LM-2163.1 | From 71360 | to 72061 | Unknown |
| SEQ ID NO. 924 | LM-2164.1 | From 71064 | to 71363 | Unknown |
| SEQ ID NO. 925 | LM-2166.1 | From 70676 | to 71071 | Unknown |
| SEQ ID NO. 926 | LM-217.1 | From 1175602 | to 1176156 | Unknown, similar to |
| | | | | Salmonella enterica PduT |
| | | | | protein |
| SEQ ID NO. 927 | LM-2170.1 | From 66160 | to 70656 | Unknown, highly similar to |
| | | | | B. subtilis YukA protein |
| SEQ ID NO. 928 | LM-2171.1 | From 64950 | to 66146 | Unknown, similar to B. subtilis |
| | | | | YukC protein |
| SEQ ID NO. 929 | LM-2172.1 | From 64677 | to 64928 | Unknown, similar to B. subtilis |
| | | | | YukD protein |
| SEQ ID NO. 930 | LM-2174.1 | From 64144 | to 64659 | Unknown |
| SEQ ID NO. 931 | LM-2178.1 | From 60948 | to 64154 | Unknown, similar to B. subtilis |
| | | | | YueB protein |
| SEQ ID NO. 932 | LM-2179.1 | From 60506 | to 60799 | Unknown, similar to a small |
| | | | | heat shock protein of |
| | | | | Clostridium acetobutylicum |
| SEQ ID NO. 933 | LM-218.1 | From 1174241 | to 1175605 | unknown, similar to |
| | | | | Salmonella enterica PduS |
| | | | | protein |
| SEQ ID NO. 934 | LM-2180.2 | From 58897 | to 60189 | unknown, highly similar to |
| | | | | adenylosuccinate |
| | | | | synthetase |
| SEQ ID NO. 935 | LM-2182.2 | From 1814403 | to 1815080 | Unknown, similar to two- |
| | | | | component response |
| | | | | regulator |
| SEQ ID NO. 936 | LM-2184.1 | From 1813457 | to 1814332 | Unknown, similar to |
| | | | | unknown proteins |
| SEQ ID NO. 937 | LM-2185.1 | From 1812978 | to 1813436 | Unknown |
| SEQ ID NO. 938 | LM-2186.1 | From 1811219 | to 1812961 | unknown, highly similar to |
| | | | | adenine deaminases |
| SEQ ID NO. 939 | LM-2188.1 | From 1810157 | to 1811197 | Unknown, similar to two- |
| | | | | component sensor histidine |
| | | | | kinase |
| SEQ ID NO. 940 | LM-2189.1 | From 1809118 | to 1809759 | Unknown, similar to amino |
| | | | | acid (glutamine) ABC |
| | | | | transporter, permease |
| | | | | protein |
| SEQ ID NO. 941 | LM-2191.1 | From 1808460 | to 1809107 | Unknown, similar to amino |
| | | | | acid (glutamine) ABC |
| | | | | transporter (ATP-binding |
| | | | | protein) |
| SEQ ID NO. 942 | LM-2193.1 | From 1807643 | to 1808458 | Unknown, similar to amino |
| | | | | acid ABC transporter |
| | | | | (binding protein) |
| SEQ ID NO. 943 | LM-2194.1 | From 1806446 | to 1807552 | Unknown, similar to glycerol |
| | | | | dehydrogenase |
| SEQ ID NO. 944 | LM-2195.1 | From 1805925 | to 1806443 | Unknown, similar to |
| | | | | unknown proteins |
| SEQ ID NO. 945 | LM-2196.1 | From 1805041 | to 1805928 | transcription activator of |
| | | | | glutamate synthase operon |
| | | | | GltC |
| SEQ ID NO. 946 | LM-2199.1 | From 1800256 | to 1804848 | Unknown, similar to |
| | | | | glutamate synthase (large |
| | | | | subunit) |
| SEQ ID NO. 947 | LM-22.1 | From 2726008 | to 2727195 | unknown, highly similar to |
| | | | | translation elongation factor |
| | | | | EF-Tu |
| SEQ ID NO. 948 | LM-220.1 | From 1173643 | to 1174122 | unknown, similar to |
| | | | | uroporphyrin-III C- |
| | | | | methyltransferase |
| SEQ ID NO. 949 | LM-2201.1 | From 1798772 | to 1800241 | Unknown, similar to |
| | | | | glutamate synthase (small |
| | | | | subunit) |
| SEQ ID NO. 950 | LM-2203.1 | From 1797897 | to 1798727 | Unknown, similar to sugar |
| | | | | transport protein |
| SEQ ID NO. 951 | LM-2204.1 | From 1796987 | to 1797880 | Unknown, similar to sugar |
| | | | | transport protein |
| SEQ ID NO. 952 | LM-2205.2 | From 1795681 | to 1796925 | Unknown, similar to sugar |
| | | | | binding protein |
| SEQ ID NO. 953 | LM-2207.2 | From 2505425 | to 2505835 | Unknown |
| SEQ ID NO. 954 | LM-2209.1 | From 2505858 | to 2506370 | Unknown |
| SEQ ID NO. 955 | LM-221.1 | From 1173144 | to 1173551 | Unknown |
| SEQ ID NO. 956 | LM-2210.1 | From 2506494 | to 2506835 | Unknown |
| SEQ ID NO. 957 | LM-2212.1 | From 2506867 | to 2507250 | Unknown |
| SEQ ID NO. 958 | LM-2213.1 | From 2507306 | to 2508202 | Unknown, similar to |
| | | | | transcription regulator |
| SEQ ID NO. 959 | LM-2214.1 | From 2508247 | to 2508816 | Unknown |
| SEQ ID NO. 960 | LM-2215.1 | From 2508992 | to 2509411 | Unknown |
| SEQ ID NO. 961 | LM-2216.1 | From 2510361 | to 2514293 | Unknown, similar to |
| | | | | glycosidase |
| SEQ ID NO. 962 | LM-2217.1 | From 2514308 | to 2515210 | Unknown, similar to |
| | | | | internalin |
| SEQ ID NO. 963 | LM-2219.1 | From 2515312 | to 2518587 | Unknown, similar to |
| | | | | glycosidase |
| SEQ ID NO. 964 | LM-2220.1 | From 2518971 | to 2519879 | Unknown, similar to |
| | | | | transcription regulator |
| SEQ ID NO. 965 | LM-2221.1 | From 2519935 | to 2520399 | Unknown, conserved |
| | | | | hypothetical protein |
| SEQ ID NO. 966 | LM-2222.1 | From 2520415 | to 2522796 | Unknown, similar to |
| | | | | exoribonuclease RNase-R |
| SEQ ID NO. 967 | LM-2224.2 | From 2522830 | to 2523576 | Unknown, similar to |
| | | | | carboxylesterase |
| SEQ ID NO. 968 | LM-2225.2 | From 2343056 | to 2343685 | Unknown, similar to |
| | | | | phosphoglucomutase |
| SEQ ID NO. 969 | LM-2226.1 | From 2341865 | to 2343013 | Unknown, similar to |
| | | | | aspartate aminotransferase |
| SEQ ID NO. 970 | LM-2227.1 | From 2341064 | to 2341789 | Unknown, similar to amino |
| | | | | acid ABC transporter (ATP- |
| | | | | binding protein) |
| SEQ ID NO. 971 | LM-2228.1 | From 2339611 | to 2341071 | Unknown, similar to amino |
| | | | | acid ABC transporter, |
| | | | | permease protein |
| SEQ ID NO. 972 | LM-2229.1 | From 2338420 | to 2339418 | Unknown, similar to low- |
| | | | | affinity inorganic phosphate |
| | | | | transporter |
| SEQ ID NO. 973 | LM-223.1 | From 1172797 | to 1173036 | Unknown |
| SEQ ID NO. 974 | LM-2230.1 | From 2337786 | to 2338406 | Unknown, similar to |
| | | | | unknown proteins |
| SEQ ID NO. 975 | LM-2232.1 | From 2336515 | to 2337399 | Unknown, similar to |
| | | | | oxidoreductase |
| SEQ ID NO. 976 | LM-2233.1 | From 2335862 | to 2336518 | Unknown, similar to |
| | | | | unknown proteins |
| SEQ ID NO. 977 | LM-2234.1 | From 2335468 | to 2335860 | Unknown, similar to |
| | | | | unknown proteins |
| SEQ ID NO. 978 | LM-2235.1 | From 2334586 | to 2335455 | Unknown, similar to putative |
| | | | | ribosomal large subunit |
| | | | | pseudouridine synthase |
| SEQ ID NO. 979 | LM-2236.1 | From 2334003 | to 2334569 | Unknown, similar to |
| | | | | methylphosphotriester-DNA |
| | | | | alkyltransferase and |
| | | | | transcriptional regulator |
| SEQ ID NO. 980 | LM-2237.1 | From 2333375 | to 2333857 | Unknown, similar to O6- |
| | | | | methylguanine-DNA |
| | | | | methyltransferase |
| SEQ ID NO. 981 | LM-2238.1 | From 2332888 | to 2333283 | Unknown, similar to |
| | | | | transcriptional regulators |
| | | | | (GntR family) |
| SEQ ID NO. 982 | LM-2239.1 | From 2332020 | to 2332898 | Unknown, similar to ABC |
| | | | | transporter (ATP-binding |
| | | | | protein) |
| SEQ ID NO. 983 | LM-224.1 | From 1172076 | to 1172648 | Unknown, similar to ATP- |
| | | | | dependent Clp protease |
| | | | | proteolytic component |
| SEQ ID NO. 984 | LM-2241.1 | From 2330959 | to 2332023 | Unknown |
| SEQ ID NO. 985 | LM-2242.1 | From 2329731 | to 2330921 | Unknown, similar to |
| | | | | transport system permease |
| | | | | protein |
| SEQ ID NO. 986 | LM-2244.1 | From 2328363 | to 2329571 | Unknown, similar to |
| | | | | transport system permease |
| | | | | protein |
| SEQ ID NO. 987 | LM-2246.1 | From 2327461 | to 2328330 | Unknown, similar to |
| | | | | oxidoreductase |
| SEQ ID NO. 988 | LM-2248.1 | From 2325477 | to 2327402 | Unknown, similar to NADH |
| | | | | oxidase |
| SEQ ID NO. 989 | LM-225.1 | From 1171662 | to 1172009 | Unknown |
| SEQ ID NO. 990 | LM-2250.1 | From 2324554 | to 2325408 | Unknown, similar to |
| | | | | unknown proteins |
| SEQ ID NO. 991 | LM-2251.1 | From 2323375 | to 2324253 | Unknown, similar to |
| | | | | transcriptional regulators |
| | | | | (LysR family) |
| SEQ ID NO. 992 | LM-2253.1 | From 2322031 | to 2323335 | Unknown, similar to |
| | | | | unknown proteins |
| SEQ ID NO. 993 | LM-2254.2 | From 2320906 | to 2321775 | Unknown, similar to |
| | | | | unknown proteins |
| SEQ ID NO. 994 | LM-2255.2 | From 2320416 | to 2320841 | Unknown, similar to |
| | | | | arsenate reductase |
| SEQ ID NO. 995 | LM-2256.3 | From 1405578 | to 1405859 | Unknown |
| SEQ ID NO. 996 | LM-2257.3 | From 1404714 | to 1405541 | Unknown, similar to B. subtilis |
| | | | | SpoIIIJ protein |
| SEQ ID NO. 997 | LM-2258.3 | From 1403227 | to 1404678 | two-component sensor |
| | | | | histidine kinase |
| SEQ ID NO. 998 | LM-2260.1 | From 1402550 | to 1403230 | two-component response |
| | | | | regulator |
| SEQ ID NO. 999 | LM-2261.2 | From 1400941 | to 1402359 | Unknown, similar to 6- |
| | | | | phosphogluconate |
| | | | | dehydrogenase |
| SEQ ID NO. 1000 | LM-2262.2 | From 1399699 | to 1400796 | Unknown, similar to |
| | | | | aminotripeptidase |
| SEQ ID NO. 1001 | LM-2263.1 | From 1398077 | to 1399327 | Unknown, similar to |
| | | | | branched-chain alpha-keto |
| | | | | acid dehydrogenase E2 |
| | | | | subunit (lipoamide |
| | | | | acyltransferase) |
| SEQ ID NO. 1002 | LM-2266.1 | From 1397063 | to 1398046 | Unknown, similar to |
| | | | | branched-chain alpha-keto |
| | | | | acid dehydrogenase E1 |
| | | | | subunit (2-oxoisovalerate |
| | | | | dehydrogenase beta |
| | | | | subunit) |
| SEQ ID NO. 1003 | LM-2267.1 | From 1396051 | to 1397046 | Unknown, similar to |
| | | | | branched-chain alpha-keto |
| | | | | acid dehydrogenase E1 |
| | | | | subunit (2-oxoisovalerate |
| | | | | dehydrogenase alpha |
| | | | | subunit) |
| SEQ ID NO. 1004 | LM-2269.1 | From 1394599 | to 1396026 | Unknown, similar to |
| | | | | branched-chain alpha-keto |
| | | | | acid dehydrogenase E3 |
| | | | | subunit |
| SEQ ID NO. 1005 | LM-2270.1 | From 1393517 | to 1394584 | Unknown, similar to |
| | | | | branched-chain fatty-acid |
| | | | | kinase |
| SEQ ID NO. 1006 | LM-2271.1 | From 1392514 | to 1393380 | Unknown, similar to |
| | | | | phosphotransbutyrylase |
| SEQ ID NO. 1007 | LM-2273.1 | From 1390694 | to 1392385 | DNA repair and genetic |
| | | | | recombination |
| SEQ ID NO. 1008 | LM-2274.1 | From 1390222 | to 1390671 | Unknown, similar to arginine |
| | | | | repressor |
| SEQ ID NO. 1009 | LM-2275.1 | From 1389207 | to 1390031 | Unknown, conserved |
| | | | | hypothetical protein |
| SEQ ID NO. 1010 | LM-2276.1 | From 1387396 | to 1389210 | Unknown, similar to D-1- |
| | | | | deoxyxylulose 5-phosphate |
| | | | | synthase |
| SEQ ID NO. 1011 | LM-2278.1 | From 1385934 | to 1386815 | Unknown, similar to |
| | | | | geranyltranstransferase |
| SEQ ID NO. 1012 | LM-2280.1 | From 1384362 | to 1385714 | Unknown, similar to |
| | | | | exodeoxyribonuclease VII |
| | | | | (large subunit) |
| SEQ ID NO. 1013 | LM-2283.1 | From 1383489 | to 1384343 | unknown, highly similar to |
| | | | | methylenetetrahydrofolate |
| | | | | dehydrogenase and |
| | | | | methenyltetrahydrofolate |
| | | | | cyclohydrolase |
| SEQ ID NO. 1014 | LM-2284.1 | From 1383007 | to 1383393 | Unknown, similar to |
| | | | | transcription termination |
| | | | | protein (NusB) |
| SEQ ID NO. 1015 | LM-2285.1 | From 1382569 | to 1382976 | unknown, similar to B. subtilis |
| | | | | YqhY protein |
| SEQ ID NO. 1016 | LM-2287.3 | From 1381201 | to 1382565 | acetyl-CoA carboxylase |
| | | | | subunit (biotin carboxylase |
| | | | | subunit) |
| SEQ ID NO. 1017 | LM-2289.3 | From 1380720 | to 1381187 | Unknown, similar to acetyl- |
| | | | | CoA carboxylase subunit |
| | | | | (biotin carboxyl carrier |
| | | | | subunit) |
| SEQ ID NO. 1018 | LM-2290.3 | From 1380004 | to 1380561 | unknown, highly similar to |
| | | | | elongation factor P (EF-P) |
| SEQ ID NO. 1019 | LM-2291.2 | From 1275845 | to 1276693 | unknown, similar to B. subtilis |
| | | | | YxkD protein |
| SEQ ID NO. 1020 | LM-2292.1 | From 1274883 | to 1275542 | unknown, similar to |
| | | | | regulator of the Fnr CRP |
| | | | | family (including PrfA) |
| SEQ ID NO. 1021 | LM-2293.1 | From 1273473 | to 1274678 | unknown; similar to |
| | | | | antibiotic resistance protein |
| SEQ ID NO. 1022 | LM-2294.1 | From 1273231 | to 1273476 | Unknown |
| SEQ ID NO. 1023 | LM-2295.1 | From 1272717 | to 1273193 | unknown, weakly similar to |
| | | | | 8-oxo-dGTPase (mutT) |
| SEQ ID NO. 1024 | LM-2296.1 | From 1272286 | to 1272549 | Unknown |
| SEQ ID NO. 1025 | LM-2298.1 | From 1270849 | to 1272261 | Unknown, similar to ATP- |
| | | | | dependent RNA helicase |
| | | | | (DEAD motif) |
| SEQ ID NO. 1026 | LM-2299.1 | From 1269834 | to 1270433 | unknown weakly similar to |
| | | | | phosphoglycerate mutase 1 |
| SEQ ID NO. 1027 | LM-230.1 | From 1168390 | to 1169541 | Unknown |
| SEQ ID NO. 1028 | LM-2300.1 | From 1269262 | to 1269669 | Unknown |
| SEQ ID NO. 1029 | LM-2301.1 | From 1268515 | to 1269108 | unknown, similar to B. subtilis |
| | | | | Ydel protein |
| SEQ ID NO. 1030 | LM-2302.1 | From 1267115 | to 1268473 | Unknown |
| SEQ ID NO. 1031 | LM-2303.1 | From 1266040 | to 1266564 | unknown, conserved |
| | | | | hypothetical protein, similar |
| | | | | to B. subtilis YsnB protein |
| SEQ ID NO. 1032 | LM-2304.1 | From 1265392 | to 1266003 | unknown, conserved |
| | | | | hypothetical protein, similar |
| | | | | to B. subtilis YsnA protein |
| SEQ ID NO. 1033 | LM-2305.1 | From 1264642 | to 1265388 | Unknown, similar to |
| | | | | ribonuclease PH |
| SEQ ID NO. 1034 | LM-2306.1 | From 1263829 | to 1264629 | Unknown, similar to |
| | | | | glutamate racemase |
| SEQ ID NO. 1035 | LM-2307.1 | From 1263187 | to 1263681 | unknown, similar to B. subtilis |
| | | | | YslB protein |
| SEQ ID NO. 1036 | LM-2308.1 | From 1261917 | to 1263131 | Unknown, similar to |
| | | | | aspartokinase II alpha |
| | | | | subunit |
| SEQ ID NO. 1037 | LM-231.1 | From 1168021 | to 1168368 | unknown, similar to |
| | | | | regulatory proteins |
| SEQ ID NO. 1038 | LM-2310.1 | From 1259917 | to 1261728 | unknown, highly similar to |
| | | | | excinuclease ABC |
| | | | | subunit C |
| SEQ ID NO. 1039 | LM-2312.1 | From 1259530 | to 1259841 | Thioredoxin |
| SEQ ID NO. 1040 | LM-2315.1 | From 1257092 | to 1259449 | unknown, similar to MutS |
| | | | | protein (MutS2) |
| SEQ ID NO. 1041 | LM-2317.1 | From 1255357 | to 1257069 | unknown, similar to DNA |
| | | | | polymerase beta, to B. subtilis |
| | | | | YshC protein |
| SEQ ID NO. 1042 | LM-2318.3 | From 1254722 | to 1255264 | unknown, similar to B. subtilis |
| | | | | YshB protein |
| SEQ ID NO. 1043 | LM-2319.2 | From 347658 | to 348428 | Unknown, similar to |
| | | | | unknown proteins |
| SEQ ID NO. 1044 | LM-232.1 | From 1167645 | to 1167953 | unknown, similar to B. subtilis |
| | | | | YjcS protein |
| SEQ ID NO. 1045 | LM-2320.1 | From 347219 | to 347611 | Unknown, similar to |
| | | | | unknown proteins |
| SEQ ID NO. 1046 | LM-2322.1 | From 346377 | to 347036 | Unknown, similar to |
| | | | | unknown proteins |
| SEQ ID NO. 1047 | LM-2324.1 | From 343221 | to 344636 | Unknown, similar to |
| | | | | phospho-beta-glucosidase |
| SEQ ID NO. 1048 | LM-2325.1 | From 342551 | to 343195 | Unknown, similar to thiamin- |
| | | | | phosphate |
| | | | | pyrophosphorylase (ThiE) |
| SEQ ID NO. 1049 | LM-2327.1 | From 341751 | to 342554 | Unknown, similar to |
| | | | | phosphomethylpyrimidine |
| | | | | kinase (ThiD) |
| SEQ ID NO. 1050 | LM-2329.1 | From 340945 | to 341754 | Unknown, similar to |
| | | | | hydroxyethylthiazole kinase |
| | | | | (ThiM) |
| SEQ ID NO. 1051 | LM-2332.1 | From 340278 | to 340952 | Unknown, similar to thiamin |
| | | | | biosynthesis protein |
| SEQ ID NO. 1052 | LM-2333.1 | From 339270 | to 340055 | Unknown, similar to |
| | | | | unknown protein |
| SEQ ID NO. 1053 | LM-2334.1 | From 338341 | to 339087 | Unknown, conserved |
| | | | | hypothetical protein |
| SEQ ID NO. 1054 | LM-2335.2 | From 337161 | to 338363 | Unknown, similar to |
| | | | | unknown proteins |
| SEQ ID NO. 1055 | LM-2336.3 | From 336541 | to 337161 | Unknown |
| SEQ ID NO. 1056 | LM-2338.2 | From 1026871 | to 1029045 | ATP-dependent protease |
| SEQ ID NO. 1057 | LM-2339.1 | From 1026376 | to 1026855 | unknown, similar to |
| | | | | methylated-DNA-protein- |
| | | | | cystein methyltransferase |
| SEQ ID NO. 1058 | LM-234.1 | From 1165962 | to 1167608 | unknown, similar to ABC |
| | | | | transporters, ATP-binding |
| | | | | proteins (HI0664) |
| SEQ ID NO. 1059 | LM-2340.1 | From 1025159 | to 1026190 | unknown, similar to B. subtilis |
| | | | | YkrP protein |
| SEQ ID NO. 1060 | LM-2341.1 | From 1024761 | to 1025129 | Unknown |
| SEQ ID NO. 1061 | LM-2342.1 | From 1023346 | to 1024680 | unknown, similar to Na+- |
| | | | | transporting ATP synthase |
| | | | | subunit J |
| SEQ ID NO. 1062 | LM-2344.1 | From 1022535 | to 1023302 | unknown, conserved |
| | | | | hypothetical protein |
| SEQ ID NO. 1063 | LM-2345.1 | From 1021665 | to 1022432 | unknown, conserved |
| | | | | hypothetical protein |
| SEQ ID NO. 1064 | LM-2346.1 | From 1020022 | to 1021383 | unknown, conserved |
| | | | | hypothetical protein |
| SEQ ID NO. 1065 | LM-2347.1 | From 1019545 | to 1019970 | unknown, similar to |
| | | | | regulatory proteins (MarR |
| | | | | family) |
| SEQ ID NO. 1066 | LM-2348.1 | From 1017870 | to 1019438 | unknown, similar to peptide |
| | | | | chain release factor 3 |
| | | | | (RF-3) |
| SEQ ID NO. 1067 | LM-2349.1 | From 1016934 | to 1017788 | unknown, similar to |
| | | | | Streptococcus agalactiae |
| | | | | CylB protein |
| SEQ ID NO. 1068 | LM-235.2 | From 1164245 | to 1165960 | ATP-binding transport |
| | | | | protein |
| SEQ ID NO. 1069 | LM-2350.1 | From 1016030 | to 1016941 | unknown, similar to |
| | | | | antibiotic ABC transporter, |
| | | | | ATP-binding protein, |
| SEQ ID NO. 1070 | LM-2351.1 | From 1015605 | to 1016033 | Unknown |
| SEQ ID NO. 1071 | LM-2352.1 | From 1015153 | to 1015608 | unknown, weakly similar to |
| | | | | two-component response |
| | | | | regulator |
| SEQ ID NO. 1072 | LM-2353.1 | From 1014540 | to 1015019 | unknown, similar to |
| | | | | glutathione peroxidase |
| SEQ ID NO. 1073 | LM-2355.1 | From 1013475 | to 1014527 | unknown, similar to |
| | | | | glucanase and peptidase |
| SEQ ID NO. 1074 | LM-2358.1 | From 1011860 | to 1013335 | unknown, similar to efflux |
| | | | | transporter |
| SEQ ID NO. 1075 | LM-2359.1 | From 1011074 | to 1011826 | unknown, similar to ABC |
| | | | | transporter transmembrane |
| | | | | component |
| SEQ ID NO. 1076 | LM-236.2 | From 1163277 | to 1163996 | unknown, similar to |
| | | | | transcription regulators |
| SEQ ID NO. 1077 | LM-2361.1 | From 1010313 | to 1011077 | unknown, similar to |
| | | | | daunorubicin resistance |
| | | | | ATP-binding proteins |
| SEQ ID NO. 1078 | LM-2362.1 | From 1009098 | to 1010117 | unknown; similar to |
| | | | | branched-chain amino acid |
| | | | | aminotransferase |
| SEQ ID NO. 1079 | LM-2363.1 | From 1008130 | to 1008879 | unknown, similar to B. subtilis |
| | | | | YjcH protein |
| SEQ ID NO. 1080 | LM-2364.1 | From 1007669 | to 1008112 | unknown, similar to B. subtilis |
| | | | | YjcF protein |
| SEQ ID NO. 1081 | LM-2365.2 | From 1006959 | to 1007633 | unknown, similar to ribose |
| | | | | 5-phosphate isomerase |
| SEQ ID NO. 1082 | LM-2366.2 | From 1937783 | to 1938415 | unknown, similar to |
| | | | | hemolysinIII proteins, |
| | | | | putative integral membrane |
| | | | | protein |
| SEQ ID NO. 1083 | LM-2368.1 | From 1938538 | to 1939155 | unknown, similar to |
| | | | | conserved hypothetical |
| | | | | proteins |
| SEQ ID NO. 1084 | LM-2369.1 | From 1939168 | to 1939980 | unknown, similar to |
| | | | | conserved hypothetical |
| | | | | proteins |
| SEQ ID NO. 1085 | LM-237.1 | From 1162666 | to 1163280 | Unknown, similar to |
| | | | | unknown proteins |
| SEQ ID NO. 1086 | LM-2371.1 | From 1939999 | to 1942638 | unknown, similar to |
| | | | | pyruvate phosphate |
| | | | | dikinase |
| SEQ ID NO. 1087 | LM-2372.1 | From 1942687 | to 1943064 | unknown, similar to |
| | | | | conserved hypothetical |
| | | | | proteins |
| SEQ ID NO. 1088 | LM-2373.1 | From 1943068 | to 1944051 | unknown, similar to |
| | | | | conserved hypothetical |
| | | | | proteins, putative integral |
| | | | | membrane protein |
| SEQ ID NO. 1089 | LM-2375.1 | From 1944145 | to 1944768 | unknown; similar to alkaline |
| | | | | phosphatase |
| SEQ ID NO. 1090 | LM-2378.1 | From 1944908 | to 1946419 | unknown, similar to |
| | | | | phosphoglucomutases |
| SEQ ID NO. 1091 | LM-2379.1 | From 1946488 | to 1947363 | unknown, similar to |
| | | | | methyltransferases |
| SEQ ID NO. 1092 | LM-238.1 | From 1161689 | to 1162696 | Unknown |
| SEQ ID NO. 1093 | LM-2380.1 | From 1947416 | to 1947898 | unknown, similar to |
| | | | | dihydrofolate reductases |
| SEQ ID NO. 1094 | LM-2381.1 | From 1947914 | to 1948858 | unknown, similar to |
| | | | | thymidylate synthase |
| SEQ ID NO. 1095 | LM-2385.1 | From 1948871 | to 1950763 | unknown, similar to putative |
| | | | | ABC transporters (ATP- |
| | | | | binding protein) |
| SEQ ID NO. 1096 | LM-2386.1 | From 1950883 | to 1951305 | Unknown, similar to formyltetrahydrofolate |
| | | | | synthetase |
| | | | | C-terminal part |
| SEQ ID NO. 1097 | LM-2387.2 | From 1951284 | to 1952564 | Unknown, similar to formyltetrahydrofolate |
| | | | | synthetase |
| | | | | N-terminal part |
| SEQ ID NO. 1098 | LM-2388.2 | From 1952721 | to 1953149 | unknown, similar to |
| | | | | transcriptional regulators |
| SEQ ID NO. 1099 | LM-239.1 | From 1161220 | to 1161675 | Unknown |
| SEQ ID NO. 1100 | LM-2390.2 | From 148354 | to 150009 | Unknown, similar to ABC |
| | | | | transporter oligopeptide- |
| | | | | binding protein |
| SEQ ID NO. 1101 | LM-2391.1 | From 147884 | to 148291 | Unknown |
| SEQ ID NO. 1102 | LM-2392.1 | From 147399 | to 147764 | Unknown |
| SEQ ID NO. 1103 | LM-2393.1 | From 146764 | to 147381 | |
| SEQ ID NO. 1104 | LM-2394.1 | From 146306 | to 146656 | Unknown |
| SEQ ID NO. 1105 | LM-2395.1 | From 145869 | to 146306 | Unknown |
| SEQ ID NO. 1106 | LM-2397.1 | From 144397 | to 144840 | Unknown |
| SEQ ID NO. 1107 | LM-2398.1 | From 144097 | to 144273 | Unknown |
| SEQ ID NO. 1108 | LM-24.1 | From 2727304 | to 2729391 | unknown, highly similar to |
| | | | | translation elongation |
| | | | | factor G |
| SEQ ID NO. 1109 | LM-240.1 | From 1160778 | to 1161230 | unknown, similar to E. coli |
| | | | | YjaB protein |
| SEQ ID NO. 1110 | LM-2400.1 | From 143366 | to 143722 | Unknown |
| SEQ ID NO. 1111 | LM-2401.1 | From 141618 | to 143006 | Unknown |
| SEQ ID NO. 1112 | LM-2402.1 | From 141337 | to 141705 | Unknown |
| SEQ ID NO. 1113 | LM-2404.1 | From 141052 | to 141336 | Unknown |
| SEQ ID NO. 1114 | LM-2406.1 | From 139960 | to 140853 | Unknown, similar to |
| | | | | oligopeptide ABC |
| | | | | transporter, permease |
| | | | | protein |
| SEQ ID NO. 1115 | LM-2409.1 | From 138999 | to 139949 | Unknown, similar to |
| | | | | oligopeptide ABC |
| | | | | transporter, permease |
| | | | | protein |
| SEQ ID NO. 1116 | LM-241.2 | From 1160220 | to 1160753 | Unknown |
| SEQ ID NO. 1117 | LM-2411.2 | From 137323 | to 138897 | Unknown, similar to |
| | | | | oligopeptide ABC transport |
| | | | | system substrate-binding |
| | | | | proteins |
| SEQ ID NO. 1118 | LM-2412.2 | From 2164106 | to 2165377 | Unknown, weakly similar to |
| | | | | transcription regulators |
| SEQ ID NO. 1119 | LM-2414.1 | From 2166012 | to 2167343 | Unknown, similar to |
| | | | | unknown proteins |
| SEQ ID NO. 1120 | LM-2415.1 | From 2167353 | to 2167943 | Unknown, similar to |
| | | | | transcription regulators |
| SEQ ID NO. 1121 | LM-2416.1 | From 2168054 | to 2169097 | Unknown, similar to lipases |
| SEQ ID NO. 1122 | LM-2417.1 | From 2169312 | to 2170526 | Unknown, similar to |
| | | | | argininosuccinate synthase |
| SEQ ID NO. 1123 | LM-2418.1 | From 2170530 | to 2171900 | Unknown, similar to |
| | | | | argininosuccinate lyase |
| SEQ ID NO. 1124 | LM-242.2 | From 1159393 | to 1159830 | Unknown |
| SEQ ID NO. 1125 | LM-2421.1 | From 2172068 | to 2173591 | glycine betaine transporter |
| | | | | BetL |
| SEQ ID NO. 1126 | LM-2423.1 | From 2173836 | to 2174486 | Unknown, similar to L- |
| | | | | fuculose-phosphate |
| | | | | aldolase |
| SEQ ID NO. 1127 | LM-2424.1 | From 2174488 | to 2175420 | Unknown, similar to 1- |
| | | | | phosphofructokinase |
| SEQ ID NO. 1128 | LM-2426.1 | From 2175438 | to 2176787 | Unknown, similar to PTS |
| | | | | system galactitol-specific |
| | | | | enzyme IIC component |
| SEQ ID NO. 1129 | LM-2428.1 | From 2176815 | to 2177090 | Unknown, similar to PTS |
| | | | | system galactitol-specific |
| | | | | enzyme IIB component |
| SEQ ID NO. 1130 | LM-2429.1 | From 2177096 | to 2177563 | Unknown, similar to PTS |
| | | | | system galactitol-specific |
| | | | | enzyme IIA component |
| SEQ ID NO. 1131 | LM-243.1 | From 1158930 | to 1159403 | Unknown |
| SEQ ID NO. 1132 | LM-2431.1 | From 2177571 | to 2179577 | Unknown, similar to |
| | | | | transcription antiterminator |
| SEQ ID NO. 1133 | LM-2432.2 | From 2179758 | to 2181203 | Unknown, similar to |
| | | | | transcriptional regulator |
| | | | | (GntR family) and to |
| | | | | aminotransferase |
| | | | | (MocR-like) |
| SEQ ID NO. 1134 | LM-2433.2 | From 161420 | to 162250 | Unknown |
| SEQ ID NO. 1135 | LM-2439.1 | From 155993 | to 156805 | Unknwown, conserved |
| | | | | hypothetical protein |
| SEQ ID NO. 1136 | LM-244.1 | From 1158425 | to 1158844 | Unknown |
| SEQ ID NO. 1137 | LM-2441.1 | From 153608 | to 155947 | Unknown, similar to ATP |
| | | | | dependent helicase |
| SEQ ID NO. 1138 | LM-2442.1 | From 152648 | to 153328 | Unknown |
| SEQ ID NO. 1139 | LM-2444.1 | From 151839 | to 152645 | Unknown, similar to high- |
| | | | | affinity zinc ABC transporter |
| | | | | (membrane protein) |
| SEQ ID NO. 1140 | LM-2446.1 | From 151186 | to 151890 | Unknown, similar to high- |
| | | | | affinity zinc ABC transporter |
| | | | | (ATP-binding protein) |
| SEQ ID NO. 1141 | LM-2447.2 | From 150232 | to 151173 | Unknown, similar to a |
| | | | | probable high-affinity zinc |
| | | | | ABC transporter (Zn(II)- |
| | | | | binding lipoprotein) |
| SEQ ID NO. 1142 | LM-2448.2 | From 2635167 | to 2637920 | autolysin, amidase |
| SEQ ID NO. 1143 | LM-245.1 | From 1158020 | to 1158388 | Unknown |
| SEQ ID NO. 1144 | LM-2450.1 | From 2637964 | to 2639562 | unknown, highly similar to |
| | | | | CTP synthases |
| SEQ ID NO. 1145 | LM-2452.1 | From 2639930 | to 2640466 | Unknown, similar to B. subtilis |
| | | | | RNA polymerase |
| | | | | delta subunit |
| SEQ ID NO. 1146 | LM-2453.1 | From 2640832 | to 2642502 | arginyl tRNA synthetase |
| SEQ ID NO. 1147 | LM-2455.1 | From 2642499 | to 2642948 | Unknown |
| SEQ ID NO. 1148 | LM-2456.1 | From 2643026 | to 2643679 | Unknown, conserved |
| | | | | hypothetical protein |
| SEQ ID NO. 1149 | LM-2457.1 | From 2643973 | to 2645295 | Unknown, conserved |
| | | | | hypothetical protein |
| SEQ ID NO. 1150 | LM-2458.1 | From 2645310 | to 2646146 | Unknown |
| SEQ ID NO. 1151 | LM-2459.1 | From 2646686 | to 2647009 | Unknown |
| SEQ ID NO. 1152 | LM-246.1 | From 1157592 | to 1158008 | Unknown |
| SEQ ID NO. 1153 | LM-2460.1 | From 2647164 | to 2648825 | Unknown, similar to |
| | | | | dipeptide ABC transporter |
| | | | | (dipeptide-binding protein) |
| SEQ ID NO. 1154 | LM-2461.1 | From 2648960 | to 2649574 | Unknown |
| SEQ ID NO. 1155 | LM-2462.1 | From 2649598 | to 2650230 | Unknown, similar to |
| | | | | nicotinamidase |
| SEQ ID NO. 1156 | LM-2463.1 | From 2650231 | to 2650755 | Unknown, similar to Chain |
| | | | | A, Dihydrofolate Reductase |
| SEQ ID NO. 1157 | LM-2464.1 | From 2650758 | to 2651756 | Unknown, similar to zinc- |
| | | | | binding dehydrogenase |
| SEQ ID NO. 1158 | LM-2465.1 | From 2651853 | to 2652125 | Unknown |
| SEQ ID NO. 1159 | LM-2467.2 | From 2652174 | to 2653085 | Unknown, similar to cation |
| | | | | transport protein |
| SEQ ID NO. 1160 | LM-2469.1 | From 331543 | to 332688 | Unknown |
| SEQ ID NO. 1161 | LM-247.3 | From 1156384 | to 1157241 | unknown, similar to |
| | | | | methylases |
| SEQ ID NO. 1162 | LM-2470.1 | From 331063 | to 331446 | Unknown |
| SEQ ID NO. 1163 | LM-2473.1 | From 329923 | to 330999 | Unknown, similar to low |
| | | | | specificity L-allo-threonine |
| | | | | aldolase |
| SEQ ID NO. 1164 | LM-2474.1 | From 328701 | to 329966 | Unknown |
| SEQ ID NO. 1165 | LM-2475.1 | From 327941 | to 328495 | Unknown, putaive secreted, |
| | | | | lysin rich protein |
| SEQ ID NO. 1166 | LM-2476.1 | From 327621 | to 327905 | Unknown |
| SEQ ID NO. 1167 | LM-2477.1 | From 327113 | to 327454 | Unknown, similar to PTS |
| | | | | beta-glucoside-specific |
| | | | | enzyme IIA component |
| SEQ ID NO. 1168 | LM-2478.1 | From 325729 | to 327120 | Unknown, similar to |
| | | | | phospho-beta-glucosidase |
| | | | | and phospho-beta- |
| | | | | galactosidase |
| SEQ ID NO. 1169 | LM-2480.1 | From 325422 | to 325712 | Unknown, similar to PTS |
| | | | | beta-glucoside-specific |
| | | | | enzyme IIB component |
| SEQ ID NO. 1170 | LM-2482.1 | From 324104 | to 325417 | Unknown, similar to PTS |
| | | | | beta-glucoside-specific |
| | | | | enzyme IIC component |
| SEQ ID NO. 1171 | LM-2483.1 | From 322134 | to 324005 | Unknown, similar to |
| | | | | transcriptional |
| | | | | antiterminator (BglG family) |
| SEQ ID NO. 1172 | LM-2485.1 | From 321490 | to 322128 | Unknown |
| SEQ ID NO. 1173 | LM-2486.1 | From 320542 | to 321279 | Unknown, similar to FMN- |
| | | | | containing NADPH-linked |
| | | | | nitro/flavin reductase |
| SEQ ID NO. 1174 | LM-2487.1 | From 319560 | to 320423 | Unknown, similar to |
| | | | | transcription regulator LysR- |
| | | | | gltR family |
| SEQ ID NO. 1175 | LM-2488.2 | From 319049 | to 319528 | Unknown, conserved |
| | | | | hypothetical protein, highly |
| | | | | similar to B. subtilis YydA |
| | | | | proteinYyd |
| SEQ ID NO. 1176 | LM-2489.2 | From 2118812 | to 2119786 | Unknown, similar to |
| | | | | phospho-N-acetylmuramoyl- |
| | | | | pentapeptide transferase |
| SEQ ID NO. 1177 | LM-249.2 | From 2788504 | to 2790243 | unknown, highly similar to |
| | | | | ABC transporter (ATP- |
| | | | | binding protein) required for |
| | | | | expression of |
| | | | | cytochrome BD |
| SEQ ID NO. 1178 | LM-2490.1 | From 2117304 | to 2118671 | Unknown, similar to UDP-N- |
| | | | | acetylmuramoylalanine |
| | | | | D-glutamate ligase |
| SEQ ID NO. 1179 | LM-2491.1 | From 2116216 | to 2117307 | Unknown, similar to |
| | | | | peptidoglycan synthesis |
| | | | | enzymes, putative |
| | | | | phospho-N-acetylmuramoyl- |
| | | | | pentapeptide-transferase |
| SEQ ID NO. 1180 | LM-2493.1 | From 2115381 | to 2116193 | Unknown, similar to cell- |
| | | | | division initiation protein |
| | | | | divIB |
| SEQ ID NO. 1181 | LM-2495.1 | From 2113734 | to 2115014 | Unknown, highly similar to |
| | | | | cell-division protein FtsA |
| SEQ ID NO. 1182 | LM-2496.1 | From 2112492 | to 2113667 | Unknown, highly similar to |
| | | | | cell-division initiation protein |
| | | | | FtsZ |
| SEQ ID NO. 1183 | LM-2497.1 | From 2111684 | to 2112373 | Unknown, similar to |
| | | | | unknown proteins |
| SEQ ID NO. 1184 | LM-2499.1 | From 2111222 | to 2111680 | Unknown, similar to |
| | | | | unknown proteins |
| SEQ ID NO. 1185 | LM-25.1 | From 2729457 | to 2729927 | Ribosomal protein S7 |
| SEQ ID NO. 1186 | LM-2500.1 | From 2110909 | to 2111199 | Unknown, similar to |
| | | | | unknown proteins |
| SEQ ID NO. 1187 | LM-2501.1 | From 2110009 | to 2110785 | Unknown, similar to |
| | | | | unknown proteins |
| SEQ ID NO. 1188 | LM-2506.1 | From 2104901 | to 2106001 | Unknown, similar to |
| | | | | quinolinate synthetase |
| SEQ ID NO. 1189 | LM-2507.1 | From 2104059 | to 2104904 | Unknown, similar to |
| | | | | nicotinate-nucleotide |
| | | | | pyrophosphorylase |
| SEQ ID NO. 1190 | LM-2508.2 | From 2102608 | to 2104062 | Unknown, similar to L- |
| | | | | aspartate oxidase |
| SEQ ID NO. 1191 | LM-2509.2 | From 2101371 | to 2102477 | Unknown, similar to a NifS- |
| | | | | like protein required for |
| | | | | NAD biosynthesis |
| SEQ ID NO. 1192 | LM-251.1 | From 2790243 | to 2791967 | Unknown, highly similar to |
| | | | | ABC transporter required for |
| | | | | expression of |
| | | | | cytochrome BD |
| SEQ ID NO. 1193 | LM-2510.2 | From 2034693 | to 2035517 | Unknown, similar to |
| | | | | ferrichrome ABC transporter |
| | | | | (ATP-binding protein) |
| SEQ ID NO. 1194 | LM-2511.1 | From 2035507 | to 2036505 | Unknown, similar to |
| | | | | oxidoreductases |
| SEQ ID NO. 1195 | LM-2512.1 | From 2036521 | to 2037141 | Unknown, similar to |
| | | | | transcription regulators |
| | | | | (TetR family) |
| SEQ ID NO. 1196 | LM-2514.2 | From 2037141 | to 2037956 | Unknown, similar to |
| | | | | unknown proteins |
| SEQ ID NO. 1197 | LM-2515.2 | From 2037953 | to 2038840 | Unknown, similar to ABC |
| | | | | transporter, ATP-binding |
| | | | | protein |
| SEQ ID NO. 1198 | LM-2517.1 | From 2039441 | to 2039998 | Unknown, similar to |
| | | | | unknown proteins |
| SEQ ID NO. 1199 | LM-2518.1 | From 2040274 | to 2040933 | Unknown, similar to |
| | | | | unknown proteins |
| SEQ ID NO. 1200 | LM-252.1 | From 2791967 | to 2792980 | unknown, highly similar to |
| | | | | cytochrome D ubiquinol |
| | | | | oxidase subunit II |
| SEQ ID NO. 1201 | LM-2520.1 | From 2040911 | to 2042110 | Unknown, similar to toxic |
| | | | | ion resistance proteins |
| SEQ ID NO. 1202 | LM-2521.1 | From 2042157 | to 2042900 | unknown, similar to |
| | | | | creatinine amidohydrolases |
| SEQ ID NO. 1203 | LM-2522.1 | From 2042913 | to 2043521 | Unknown, similar to 2-keto- |
| | | | | 3-deoxygluconate-6- |
| | | | | phosphate aldolase |
| SEQ ID NO. 1204 | LM-2523.1 | From 2043540 | to 2044457 | Unknown, similar to putative |
| | | | | phosphotriesterase related |
| | | | | proteins |
| SEQ ID NO. 1205 | LM-2524.1 | From 2044481 | to 2045749 | Unknown, similar to |
| | | | | unknown proteins |
| SEQ ID NO. 1206 | LM-2527.1 | From 2046046 | to 2046489 | Unknown, similar to PTS |
| | | | | system enzyme II A |
| | | | | component |
| SEQ ID NO. 1207 | LM-2528.1 | From 2046507 | to 2047256 | Unknown, similar to |
| | | | | transcription regulators, |
| | | | | (GntR family) |
| SEQ ID NO. 1208 | LM-2529.1 | From 2047444 | to 2048514 | Unknown, similar to E. coli |
| | | | | DNA-damage-inducibile |
| | | | | protein dinP |
| SEQ ID NO. 1209 | LM-2530.1 | From 2048623 | to 2049414 | Unknown, similar to |
| | | | | oxidoreductase |
| SEQ ID NO. 1210 | LM-2531.1 | From 2049419 | to 2050339 | Unknown, similar to |
| | | | | unknown proteins |
| SEQ ID NO. 1211 | LM-2532.4 | From 2050639 | to 2052114 | Unknown, similar to |
| | | | | glucose-6-phosphate |
| | | | | 1-dehydrogenase |
| SEQ ID NO. 1212 | LM-2533.2 | From 1731955 | to 1732893 | Unknown, similar to |
| | | | | menaquinone biosynthesis |
| | | | | proteins |
| SEQ ID NO. 1213 | LM-2536.1 | From 1732926 | to 1734779 | unknown, similar to |
| | | | | 5-methyltetrahydrofolate |
| | | | | homocysteine |
| | | | | methyltransferase (metH) |
| SEQ ID NO. 1214 | LM-2538.1 | From 1734776 | to 1735948 | unknown; similar to |
| | | | | cystathionine beta-lyase |
| SEQ ID NO. 1215 | LM-2539.1 | From 1735941 | to 1737065 | unknown, similar to |
| | | | | cystathionine gamma- |
| | | | | synthase |
| SEQ ID NO. 1216 | LM-254.1 | From 2792967 | to 2794373 | Unknown, highly similar to |
| | | | | cytochrome D ubiquinol |
| | | | | oxidase subunit I |
| SEQ ID NO. 1217 | LM-2540.2 | From 1737087 | to 1739384 | unknown, similar to |
| | | | | cobalamin-independent |
| | | | | methionine synthase |
| SEQ ID NO. 1218 | LM-2542.2 | From 2088797 | to 2091454 | Unknown, similar to putative |
| | | | | sugar hydrolases |
| SEQ ID NO. 1219 | LM-2543.1 | From 2087489 | to 2088793 | Unknown, similar to |
| | | | | unknown proteins |
| SEQ ID NO. 1220 | LM-2544.1 | From 2086817 | to 2087428 | unknown, similar to |
| | | | | unknown proteins |
| SEQ ID NO. 1221 | LM-2545.1 | From 2085021 | to 2086760 | Unknown, similar to two- |
| | | | | component sensor histidine |
| | | | | kinase |
| SEQ ID NO. 1222 | LM-2546.1 | From 2083537 | to 2085021 | Unknown, similar to two- |
| | | | | component response |
| | | | | regulator |
| SEQ ID NO. 1223 | LM-2547.1 | From 2082495 | to 2083424 | Unknown, similar to putative |
| | | | | transport system integral |
| | | | | membrane protein |
| SEQ ID NO. 1224 | LM-2548.1 | From 2081505 | to 2082476 | Unknown, similar to ABC |
| | | | | transporter, permease |
| | | | | protein |
| SEQ ID NO. 1225 | LM-2549.1 | From 2080027 | to 2081484 | Unknown, similar to putative |
| | | | | sugar-binding lipoproteins |
| SEQ ID NO. 1226 | LM-255.1 | From 2794755 | to 2795225 | Unknown, conserved |
| | | | | hypothetical proteins |
| SEQ ID NO. 1227 | LM-2553.1 | From 2078132 | to 2079829 | Unknown, similar to alpha- |
| | | | | acetolactate synthase |
| | | | | protein, AIsS |
| SEQ ID NO. 1228 | LM-2554.3 | From 2077019 | to 2078014 | Unknown, similar to |
| | | | | oxidoreductase |
| SEQ ID NO. 1229 | LM-2557.2 | From 634479 | to 635279 | Unknown, similar to |
| | | | | transport proteins |
| | | | | (formate?) |
| SEQ ID NO. 1230 | LM-2559.2 | From 633709 | to 634251 | Unknown |
| SEQ ID NO. 1231 | LM-2560.1 | From 632811 | to 633581 | Unknown, similar to |
| | | | | unknown membrane |
| | | | | proteins |
| SEQ ID NO. 1232 | LM-2561.1 | From 631045 | to 632811 | Unknown, similar to a fusion |
| | | | | of two types of conserved |
| | | | | hypothetical |
| | | | | proteinconserved |
| | | | | hypothetical |
| SEQ ID NO. 1233 | LM-2562.1 | From 630629 | to 631042 | Unknown |
| SEQ ID NO. 1234 | LM-2563.1 | From 629088 | to 630491 | Unknown, similar to DNA |
| | | | | photolyase |
| SEQ ID NO. 1235 | LM-2565.1 | From 626506 | to 628971 | Unknown, putative secreted |
| | | | | protein |
| SEQ ID NO. 1236 | LM-2566.1 | From 625463 | to 626488 | Unknown |
| SEQ ID NO. 1237 | LM-2567.2 | From 624682 | to 625395 | Unknown, putative secreted |
| | | | | protein |
| SEQ ID NO. 1238 | LM-2568.3 | From 702088 | to 702954 | Unknown, conserved |
| | | | | hypothetical proteins |
| SEQ ID NO. 1239 | LM-2570.1 | From 701247 | to 702062 | Unknown, highly similar to |
| | | | | phosphomethylpyrimidine |
| | | | | kinase thiD |
| SEQ ID NO. 1240 | LM-2571.1 | From 700876 | to 701184 | Unknown, similar to |
| | | | | unknown proteins |
| SEQ ID NO. 1241 | LM-2573.1 | From 700537 | to 700821 | unknown, similar to |
| | | | | transposases |
| SEQ ID NO. 1242 | LM-2574.1 | From 699410 | to 700306 | Unknown, similar to |
| | | | | transcription regulator |
| | | | | (Rgg type) |
| SEQ ID NO. 1243 | LM-2575.1 | From 698785 | to 699420 | Unknown, conserved |
| | | | | hypothetical protein |
| SEQ ID NO. 1244 | LM-2577.1 | From 698317 | to 698733 | Unknown |
| SEQ ID NO. 1245 | LM-2578.1 | From 697634 | to 698197 | Unknown, conserved |
| | | | | hypothetical protein |
| SEQ ID NO. 1246 | LM-2579.1 | From 696883 | to 697590 | Unknown, similar to |
| | | | | phosphoprotein |
| | | | | phosphatases |
| SEQ ID NO. 1247 | LM-258.1 | From 2795420 | to 2796997 | Unknown, similar to |
| | | | | acetate-CoA ligase |
| SEQ ID NO. 1248 | LM-2581.1 | From 695496 | to 696416 | Unknown |
| SEQ ID NO. 1249 | LM-2583.1 | From 694973 | to 695509 | Unknown, similar to |
| | | | | unknown proteins |
| SEQ ID NO. 1250 | LM-2584.1 | From 694317 | to 694964 | Unknown, similar to |
| | | | | transcription regulator |
| SEQ ID NO. 1251 | LM-2587.1 | From 691581 | to 694271 | Unknown, conserved |
| | | | | membrane protein |
| SEQ ID NO. 1252 | LM-2589.1 | From 690897 | to 691538 | Unknown, similar to |
| | | | | transcription regulators |
| SEQ ID NO. 1253 | LM-259.1 | From 2797039 | to 2797758 | Unknown, similar to |
| | | | | glucosamine-6-phosphate |
| | | | | isomerase |
| SEQ ID NO. 1254 | LM-2590.1 | From 689923 | to 690873 | Unknown, similar to |
| | | | | membrane proteins |
| SEQ ID NO. 1255 | LM-2592.1 | From 689541 | to 689825 | Unknown |
| SEQ ID NO. 1256 | LM-2593.1 | From 688619 | to 689458 | Unknown, similar to |
| | | | | unknown proteins |
| SEQ ID NO. 1257 | LM-2594.2 | From 687138 | to 688529 | Unknown, similar to amino |
| | | | | acid transporter |
| SEQ ID NO. 1258 | LM-2597.3 | From 1113759 | to 1115630 | unknown, similar to B. subtilis |
| | | | | minor teichoic acids |
| | | | | biosynthesis protein GgaB |
| SEQ ID NO. 1259 | LM-2598.3 | From 1115647 | to 1116513 | Unknown, similar to |
| | | | | glucose-1-phosphate |
| | | | | thymidyl transferase |
| SEQ ID NO. 1260 | LM-2599.1 | From 1116532 | to 1117092 | Unknown, similar to dTDP- |
| | | | | sugar epimerase |
| SEQ ID NO. 1261 | LM-26.1 | From 2729958 | to 2730371 | ribosomal protein S12 |
| SEQ ID NO. 1262 | LM-260.1 | From 2797838 | to 2798572 | Unknown, similar to merR- |
| | | | | family transcriptional |
| | | | | regulator |
| SEQ ID NO. 1263 | LM-2600.1 | From 1117093 | to 1118079 | Unknown, similar to dTDP- |
| | | | | D-glucose 4,6-dehydratase |
| SEQ ID NO. 1264 | LM-2601.1 | From 1118082 | to 1118912 | unknown, similar to DTDP- |
| | | | | L-rhamnose synthetase |
| SEQ ID NO. 1265 | LM-2602.1 | From 1118992 | to 1121022 | unknown, similar to teichoic |
| | | | | acid biosynthesis protein B |
| SEQ ID NO. 1266 | LM-2603.1 | From 1121098 | to 1121808 | unknown, similar to CDP- |
| | | | | ribitol pyrophosphorylase |
| SEQ ID NO. 1267 | LM-2604.1 | From 1121805 | to 1122830 | unknown, similar to glucitol |
| | | | | dehydrogenase |
| SEQ ID NO. 1268 | LM-2606.1 | From 1122918 | to 1124078 | unknown, similar to teichoic |
| | | | | acid biosynthesis protein B |
| | | | | precursor |
| SEQ ID NO. 1269 | LM-2607.1 | From 1124079 | to 1124462 | unknown, highly similar to |
| | | | | glycerol-3-phosphate |
| | | | | cytidylyltransferase (gct), |
| | | | | CDP-glycerol |
| | | | | pyrophosphorylase (teichoic |
| | | | | acid biosynthesis protein D) |
| SEQ ID NO. 1270 | LM-2608.1 | From 1124484 | to 1125467 | unknown, similar to |
| | | | | glycosyltransferases |
| SEQ ID NO. 1271 | LM-2609.1 | From 1125482 | to 1126495 | unknown, siumilar to |
| | | | | glysosyltransferases |
| SEQ ID NO. 1272 | LM-261.1 | From 2798626 | to 2799087 | Unknown, similar to |
| | | | | unknown proteins |
| SEQ ID NO. 1273 | LM-2611.1 | From 1126704 | to 1128194 | unknown, conserved |
| | | | | hypothetical protein, similar |
| | | | | to B. subtilis YueK protein |
| SEQ ID NO. 1274 | LM-2613.1 | From 1128206 | to 1129030 | unknown, similar to NH(3)- |
| | | | | dependent NAD(+) |
| | | | | synthetases, nitrogen |
| | | | | regulatory protein |
| SEQ ID NO. 1275 | LM-2614.1 | From 1129043 | to 1129351 | Unknown |
| SEQ ID NO. 1276 | LM-2615.1 | From 1129412 | to 1129816 | unknown, similar to PTS |
| | | | | system, cellobiose-specific |
| | | | | IIB component (cel A) |
| SEQ ID NO. 1277 | LM-2616.1 | From 1129945 | to 1131501 | unknown, highly similar to |
| | | | | GMP synthetase |
| SEQ ID NO. 1278 | LM-2618.1 | From 1131558 | to 1132760 | unknown, similar to |
| | | | | integrases |
| SEQ ID NO. 1279 | LM-2619.1 | From 1133698 | to 1134117 | unknown, similar to a |
| | | | | protein encoded by Tn916 |
| SEQ ID NO. 1280 | LM-262.1 | From 2799108 | to 2799551 | Unknown, similar to |
| | | | | unknown proteins |
| SEQ ID NO. 1281 | LM-2621.4 | From 1134472 | to 1136595 | cadmium resistance protein |
| SEQ ID NO. 1282 | LM-2623.2 | From 2119833 | to 2121308 | Unknown, similar to UDP-N- |
| | | | | acetylmuramoylalanyl-D- |
| | | | | glutamate-2,6- |
| | | | | diaminopimelate ligase |
| SEQ ID NO. 1283 | LM-2625.2 | From 2121483 | to 2123738 | Unknown, similar to |
| | | | | penicillin-binding protein 2B |
| SEQ ID NO. 1284 | LM-2626.2 | From 2123735 | to 2124097 | Unknown, similar to cell- |
| | | | | division protein FtsL |
| SEQ ID NO. 1285 | LM-2628.1 | From 2124114 | to 2125052 | Unknown, similar to |
| | | | | unknown proteins |
| SEQ ID NO. 1286 | LM-2629.1 | From 2125065 | to 2125496 | Unknown, similar to |
| | | | | unknown proteins |
| SEQ ID NO. 1287 | LM-263.1 | From 2799609 | to 2800961 | Unknown, conserved |
| | | | | hypothetical proteins |
| SEQ ID NO. 1288 | LM-2631.2 | From 2125699 | to 2126946 | Unknown, similar to integral |
| | | | | membrane proteins |
| SEQ ID NO. 1289 | LM-2632.1 | From 2127062 | to 2128876 | Unknown, similar to |
| | | | | transporter binding proteins |
| SEQ ID NO. 1290 | LM-2633.1 | From 2128806 | to 2129192 | Unknown |
| SEQ ID NO. 1291 | LM-2635.1 | From 2129185 | to 2130078 | Unknown, weakly similar to |
| | | | | ketopantoate reductase |
| | | | | involved in thiamin |
| | | | | biosynthesis |
| SEQ ID NO. 1292 | LM-2637.1 | From 2130492 | to 2131028 | Unknown, similar to |
| | | | | unknown proteins |
| SEQ ID NO. 1293 | LM-2638.1 | From 2131158 | to 2132330 | Unknown, similar to |
| | | | | unknown proteins |
| SEQ ID NO. 1294 | LM-2639.1 | From 2132412 | to 2134652 | Unknown, similar to |
| | | | | excinuclease ABC |
| | | | | (subunit A) |
| SEQ ID NO. 1295 | LM-264.1 | From 2800958 | to 2801407 | Unknown, similar to |
| | | | | transcription regulators |
| SEQ ID NO. 1296 | LM-2641.1 | From 2134695 | to 2135735 | Unknown, weakly similar to |
| | | | | proteases |
| SEQ ID NO. 1297 | LM-2642.1 | From 2135754 | to 2136236 | Unknown, similar to |
| | | | | phosphopantetheine |
| | | | | adenylyltransferase |
| SEQ ID NO. 1298 | LM-2643.1 | From 2136239 | to 2136796 | Unknown, similar to |
| | | | | unknown proteins |
| SEQ ID NO. 1299 | LM-2645.1 | From 2136893 | to 2137174 | Unknown, similar to |
| | | | | unknown proteins |
| SEQ ID NO. 1300 | LM-2647.1 | From 2137205 | to 2137654 | Unknown, similar to |
| | | | | unknown proteins |
| SEQ ID NO. 1301 | LM-2648.1 | From 2137702 | to 2138757 | Unknown, similar to |
| | | | | unknown proteins |
| SEQ ID NO. 1302 | LM-265.1 | From 2801577 | to 2802458 | Unknown, similar to |
| | | | | unknown proteins |
| SEQ ID NO. 1303 | LM-2650.2 | From 2138904 | to 2139809 | unknown, highly similar to |
| | | | | heme A farnesyltransferase |
| SEQ ID NO. 1304 | LM-2651.2 | From 1994751 | to 1995434 | Unknown, similar to |
| | | | | unknown proteins |
| SEQ ID NO. 1305 | LM-2652.1 | From 1995520 | to 1996116 | Unknown, similar to |
| | | | | unknown proteins |
| SEQ ID NO. 1306 | LM-2653.1 | From 1996204 | to 1996749 | Unknown, similar to |
| | | | | unknown proteins |
| SEQ ID NO. 1307 | LM-2654.1 | From 1996784 | to 1998037 | Unknown, similar to |
| | | | | unknown proteins |
| SEQ ID NO. 1308 | LM-2657.1 | From 1998168 | to 1999454 | Unknown, similar to 5- |
| | | | | enolpyruvylshikimate-3- |
| | | | | phosphate synthase |
| SEQ ID NO. 1309 | LM-2659.1 | From 1999468 | to 2000571 | Unknown, similar to |
| | | | | prephenate dehydrogenase |
| SEQ ID NO. 1310 | LM-266.1 | From 2802543 | to 2802998 | Unknown, similar to |
| | | | | transcription regulator MerR |
| | | | | family |
| SEQ ID NO. 1311 | LM-2660.1 | From 2000592 | to 2001674 | Unknown, similar to |
| | | | | histidinol-phosphate |
| | | | | aminotransferase and |
| | | | | tyrosine/phenylalanine |
| | | | | aminotransferase |
| SEQ ID NO. 1312 | LM-2661.1 | From 2001674 | to 2002048 | Unknown, similar to |
| | | | | chorismate mutase |
| SEQ ID NO. 1313 | LM-2662.1 | From 2002045 | to 2003142 | Unknown, similar to |
| | | | | 3-dehydroquinate synthase |
| SEQ ID NO. 1314 | LM-2664.2 | From 2003145 | to 2004311 | Unknown, similar to |
| | | | | chorismate synthase |
| SEQ ID NO. 1315 | LM-267.1 | From 2802998 | to 2803402 | Unknown, similar to |
| | | | | unknown proteins |
| SEQ ID NO. 1316 | LM-2675.4 | From 1748263 | to 1748709 | unknown, similar to |
| | | | | transcription regulators (Fur |
| | | | | family), PerR in B. subtilis |
| SEQ ID NO. 1317 | LM-2676.2 | From 1356769 | to 1357683 | Unknown, highly similar to |
| | | | | tRNA pseudouridine 55 |
| | | | | synthase |
| SEQ ID NO. 1318 | LM-2677.1 | From 1356327 | to 1356671 | Unknown, highly similar to |
| | | | | ribosome-binding factor A |
| SEQ ID NO. 1319 | LM-2679.1 | From 1356032 | to 1356310 | unknown, conserved |
| | | | | hypothetical protein similar |
| | | | | to B. subtilis YlxP protein |
| SEQ ID NO. 1320 | LM-268.1 | From 2803445 | to 2804056 | unknown, similar to |
| | | | | phosphatase |
| SEQ ID NO. 1321 | LM-2683.1 | From 1353696 | to 1356035 | Unknown, highly similar to |
| | | | | translation initiation factor |
| | | | | IF-2 |
| SEQ ID NO. 1322 | LM-2684.1 | From 1353374 | to 1353673 | unknown, conserved |
| | | | | hypothetical protein, similar |
| | | | | to B. subtilis YlxQ protein |
| SEQ ID NO. 1323 | LM-2685.1 | From 1353097 | to 1353381 | unknown, similar to B. subtilis |
| | | | | YlxR protein |
| SEQ ID NO. 1324 | LM-2687.1 | From 1351964 | to 1353082 | unknown, highly similar to N |
| | | | | utilization substance protein |
| | | | | A (NusA protein) |
| SEQ ID NO. 1325 | LM-2689.1 | From 1351467 | to 1351934 | unknown, conserved |
| | | | | hypothetical protein, similar |
| | | | | to B. subtilis YlxS protein |
| SEQ ID NO. 1326 | LM-269.1 | From 2804070 | to 2804438 | unknown, similar to |
| | | | | transcription regulator (RpiR |
| | | | | family) |
| SEQ ID NO. 1327 | LM-2692.1 | From 1346951 | to 1351285 | unknown, highly similar to |
| | | | | DNA polymerase III (alpha |
| | | | | subunit) |
| SEQ ID NO. 1328 | LM-2693.1 | From 1345140 | to 1346846 | prolyl-tRNA synthetase |
| SEQ ID NO. 1329 | LM-2696.2 | From 1343838 | to 1345100 | unknown, conserved |
| | | | | hypothetical protein similar |
| | | | | to B. subtilis YluC protein |
| SEQ ID NO. 1330 | LM-2698.2 | From 1342682 | to 1343824 | Unknown, similar to |
| | | | | deoxyxylulose 5-phosphate |
| | | | | reductoisomerase |
| SEQ ID NO. 1331 | LM-2699.2 | From 1341879 | to 1342667 | unknown, similar to |
| | | | | phosphatidate |
| | | | | cytidylyltransferase (CDP- |
| | | | | diglyceride synthase) |
| SEQ ID NO. 1332 | LM-27.1 | From 2730564 | to 2731961 | Unknown, similar to dGTP |
| | | | | triphosphohydrolase |
| SEQ ID NO. 1333 | LM-270.1 | From 2804522 | to 2805274 | unknown |
| SEQ ID NO. 1334 | LM-2701.3 | From 1779714 | to 1780802 | unknown, similar to putative |
| | | | | outer surface protein |
| SEQ ID NO. 1335 | LM-2702.1 | From 1780824 | to 1781126 | unknown, similar to |
| | | | | phosphotransferase system |
| | | | | (PTS) lichenan-specific |
| | | | | enzyme IIA component |
| SEQ ID NO. 1336 | LM-2703.1 | From 1781196 | to 1781504 | unknown, similar to |
| | | | | phosphotransferase system |
| | | | | (PTS) lichenan-specific |
| | | | | enzyme IIB component |
| SEQ ID NO. 1337 | LM-2705.2 | From 1781661 | to 1784339 | unknown, similar to |
| | | | | transcriptional regulator |
| | | | | (NifA/NtrC family) |
| SEQ ID NO. 1338 | LM-2707.2 | From 1784473 | to 1785798 | unknown, similar to ATP- |
| | | | | dependent RNA helicases |
| SEQ ID NO. 1339 | LM-2708.1 | From 1785841 | to 1786614 | unknown |
| SEQ ID NO. 1340 | LM-2710.1 | From 1786607 | to 1787287 | unknown, similar to ABC |
| | | | | transporter, ATP-binding |
| | | | | protein |
| SEQ ID NO. 1341 | LM-2711.1 | From 1787280 | to 1787648 | unknown, similar to |
| | | | | transcriptional regulator |
| | | | | (GntR family) |
| SEQ ID NO. 1342 | LM-2712.1 | From 1787769 | to 1788752 | unknown, similar to |
| | | | | hypothetical proteins |
| SEQ ID NO. 1343 | LM-2713.1 | From 1788812 | to 1789777 | unknown, similar to |
| | | | | transcription regulators |
| | | | | (Lacl family) |
| SEQ ID NO. 1344 | LM-2714.1 | From 1789825 | to 1793085 | unknown, some similarities |
| | | | | to cellobiose-phosphorylase |
| SEQ ID NO. 1345 | LM-2717.2 | From 1793082 | to 1795253 | unknown, similar to beta- |
| | | | | glucosidases |
| SEQ ID NO. 1346 | LM-2718.2 | From 1561978 | to 1564242 | Unknown, similar to protein- |
| | | | | export membrane protein |
| | | | | SecDF |
| SEQ ID NO. 1347 | LM-272.1 | From 2805440 | to 2807398 | Unknown, similar to PTS |
| | | | | system, fructose-specific |
| | | | | IIABC component |
| SEQ ID NO. 1348 | LM-2720.1 | From 1564343 | to 1564633 | Unknown, similar to |
| | | | | unknown proteins |
| SEQ ID NO. 1349 | LM-2721.1 | From 1564776 | to 1565105 | Unknown, similar to |
| | | | | unknown proteins |
| SEQ ID NO. 1350 | LM-2722.1 | From 1565139 | to 1566278 | Unknown, similar to tRNA- |
| | | | | guanine transglycosylase |
| | | | | Tgt |
| SEQ ID NO. 1351 | LM-2723.1 | From 1566365 | to 1567393 | Unknown, similar to S- |
| | | | | adenosylmethionine: tRNA |
| | | | | ribosyltransferase- |
| | | | | isomerase |
| SEQ ID NO. 1352 | LM-2725.1 | From 1567397 | to 1568404 | unknown, highly similar to |
| | | | | Holliday junction DNA |
| | | | | helicase RuvB |
| SEQ ID NO. 1353 | LM-2726.1 | From 1568420 | to 1569025 | unknown, highly similar to |
| | | | | Holliday junction DNA |
| | | | | helicase (ruvA) |
| SEQ ID NO. 1354 | LM-2728.1 | From 1569149 | to 1570084 | Unknown, similar to L- |
| | | | | lactate dehydrogenase |
| SEQ ID NO. 1355 | LM-2729.1 | From 1570154 | to 1570879 | Unknown, similar to |
| | | | | unknown proteins |
| SEQ ID NO. 1356 | LM-273.1 | From 2807459 | to 2810107 | Unknown, weakly similar to |
| | | | | sugar hydrolase |
| SEQ ID NO. 1357 | LM-2730.1 | From 1570990 | to 1571838 | Unknown, similar to |
| | | | | prephenate dehydratase |
| | | | | PheA |
| SEQ ID NO. 1358 | LM-2732.1 | From 1571905 | to 1573194 | Unknown, conserved GTP |
| | | | | binding protein |
| SEQ ID NO. 1359 | LM-2735.1 | From 1573354 | to 1574847 | Unknown, similar to glycerol |
| | | | | kinase |
| SEQ ID NO. 1360 | LM-2736.2 | From 1574922 | to 1575740 | Unknown, similar to glycerol |
| | | | | uptake facilitator |
| SEQ ID NO. 1361 | LM-2737.2 | From 623251 | to 624423 | Unknown, conserved |
| | | | | hypothetical membrane |
| | | | | protein |
| SEQ ID NO. 1362 | LM-274.1 | From 2810109 | to 2811791 | Unknown, similar to |
| | | | | Sucrose phosphorylase |
| SEQ ID NO. 1363 | LM-2741.1 | From 620805 | to 623135 | Unknown, similar to |
| | | | | preprotein translocase SecA |
| | | | | subunit |
| SEQ ID NO. 1364 | LM-2743.3 | From 618932 | to 620380 | P60 extracellular protein, |
| | | | | invasion associated protein |
| | | | | lap |
| SEQ ID NO. 1365 | LM-2746.1 | From 617660 | to 618844 | Unknown, conserved |
| | | | | hypothetical protein |
| SEQ ID NO. 1366 | LM-2747.1 | From 616973 | to 617629 | Unknown, weakly similar to |
| | | | | carboxylesterase |
| SEQ ID NO. 1367 | LM-275.1 | From 2811788 | to 2812921 | Unknown, conserved |
| | | | | hypothetical protein |
| SEQ ID NO. 1368 | LM-2752.1 | From 615825 | to 616577 | Unknown, putative |
| | | | | conserved membrane |
| | | | | protein |
| SEQ ID NO. 1369 | LM-2753.1 | From 615353 | to 615811 | Unknown |
| SEQ ID NO. 1370 | LM-2754.1 | From 613836 | to 615299 | Unknown |
| SEQ ID NO. 1371 | LM-2755.2 | From 612678 | to 613406 | Unknown, similar to |
| | | | | transcription regulator GntR |
| | | | | family |
| SEQ ID NO. 1372 | LM-2758.2 | From 2343858 | to 2345150 | Unknown, similar to |
| | | | | unknown proteins |
| SEQ ID NO. 1373 | LM-2759.1 | From 2345260 | to 2345526 | Unknown |
| SEQ ID NO. 1374 | LM-276.1 | From 2812925 | to 2813881 | Unknown, similar to |
| | | | | transcriptional regulator |
| | | | | (Lacl family) |
| SEQ ID NO. 1375 | LM-2760.1 | From 2345567 | to 2346088 | Unknown, similar to |
| | | | | unknown proteins |
| SEQ ID NO. 1376 | LM-2761.1 | From 2346252 | to 2346596 | Unknown, hypothetical CDS |
| SEQ ID NO. 1377 | LM-2762.1 | From 2346935 | to 2347285 | unknown, similar to |
| | | | | phosphotransferase system |
| | | | | (PTS) beta-glucoside- |
| | | | | specific enzyme IIA |
| SEQ ID NO. 1378 | LM-2763.1 | From 2347276 | to 2348151 | Unknown, similar to |
| | | | | unknown proteins |
| SEQ ID NO. 1379 | LM-2764.1 | From 2348231 | to 2348539 | Unknown, similar to |
| | | | | unknown proteins |
| SEQ ID NO. 1380 | LM-2765.1 | From 2348604 | to 2349356 | Unknown, similar to |
| | | | | unknown proteins |
| SEQ ID NO. 1381 | LM-2766.1 | From 2349475 | to 2350320 | unknown, similar to unknown |
| | | | | proteins |
| SEQ ID NO. 1382 | LM-2768.1 | From 2350398 | to 2351300 | Unknown, similar to |
| | | | | unknown proteins |
| SEQ ID NO. 1383 | LM-2769.1 | From 2351384 | to 2351746 | Unknown, similar to |
| | | | | unknown proteins |
| SEQ ID NO. 1384 | LM-2770.1 | From 2351767 | to 2352615 | Unknown, similar to |
| | | | | unknown proteins |
| SEQ ID NO. 1385 | LM-2771.1 | From 2352616 | to 2356323 | Unknown, similar to ATP- |
| | | | | dependent |
| | | | | deoxyribonuclease |
| | | | | (subunit A) |
| SEQ ID NO. 1386 | LM-2772.3 | From 2356325 | to 2359798 | Unknown, similar to ATP- |
| | | | | dependent |
| | | | | deoxyribonuclease |
| | | | | (subunit B) |
| SEQ ID NO. 1387 | LM-2774.2 | From 2097176 | to 2099941 | isoleucyl-tRNA synthetase |
| SEQ ID NO. 1388 | LM-2776.1 | From 2096075 | to 2097064 | Unknown, similar to |
| | | | | diaminopimelate epimerase |
| SEQ ID NO. 1389 | LM-2778.1 | From 2095366 | to 2096061 | Unknown, similar to |
| | | | | unknown proteins |
| SEQ ID NO. 1390 | LM-278.1 | From 2813949 | to 2815283 | Unknown, conserved |
| | | | | hypothetical protein similar |
| | | | | to hypothetical hemolysin |
| SEQ ID NO. 1391 | LM-2780.2 | From 2091698 | to 2094808 | Unknown, similar to alpha- |
| | | | | mannosidase |
| SEQ ID NO. 1392 | LM-2781.3 | From 2504300 | to 2505157 | Unknown, similar to |
| | | | | transcription antiterminator |
| SEQ ID NO. 1393 | LM-2782.1 | From 2503931 | to 2504236 | Unknown, similar to B. subtilis |
| | | | | YfhL protein |
| SEQ ID NO. 1394 | LM-2784.1 | From 2502401 | to 2503804 | unknown, highly similar to |
| | | | | glutamate decarboxylases |
| SEQ ID NO. 1395 | LM-2785.1 | From 2501603 | to 2502361 | Unknown, similar to |
| | | | | acetylesterase |
| SEQ ID NO. 1396 | LM-2788.1 | From 2500179 | to 2501150 | Unknown, similar to B. subtilis |
| | | | | ferrichrome ABC |
| | | | | transporter fhuD precursor |
| | | | | (ferrichrome-binding |
| | | | | protein) |
| SEQ ID NO. 1397 | LM-2790.1 | From 2499169 | to 2500179 | Unknown, similarto B. subtilis |
| | | | | ferrichrome ABC |
| | | | | transporter (permease) |
| | | | | FhuG |
| SEQ ID NO. 1398 | LM-2792.1 | From 2498381 | to 2499172 | Unknown, similar to B. subtilis |
| | | | | ferrichrome ABC |
| | | | | transporter (ATP-binding |
| | | | | protein) FhuC |
| SEQ ID NO. 1399 | LM-2793.1 | From 2497069 | to 2498238 | Unknown, similar to cell |
| | | | | division proteins RodA, |
| | | | | FtsW |
| SEQ ID NO. 1400 | LM-2794.1 | From 2495818 | to 2496993 | Unknown, similar to cell |
| | | | | division proteins RodA, |
| | | | | FtsW |
| SEQ ID NO. 1401 | LM-2795.2 | From 2495309 | to 2495662 | Unknown, conserved |
| | | | | hypothetical proteins |
| SEQ ID NO. 1402 | LM-2796.1 | From 1674961 | to 1676325 | unknown, highly similar to |
| | | | | anthranilate synthase alpha |
| | | | | subunit |
| SEQ ID NO. 1403 | LM-2797.1 | From 1674359 | to 1674964 | unknown, highly similar to |
| | | | | anthranilate synthase beta |
| | | | | subunit |
| SEQ ID NO. 1404 | LM-2798.1 | From 1673368 | to 1674387 | unknown, highly similar to |
| | | | | anthranilate |
| | | | | phosphoribosyltransferase |
| SEQ ID NO. 1405 | LM-2799.1 | From 1672613 | to 1673371 | unknown, highly similar to |
| | | | | indol-3-glycerol phosphate |
| | | | | synthases |
| SEQ ID NO. 1406 | LM-28.1 | From 2731976 | to 2732455 | Unknown, similar to |
| | | | | spermidine/spermine N1- |
| | | | | acetyl transferase |
| SEQ ID NO. 1407 | LM-280.1 | From 2815401 | to 2816090 | unknown, similar to |
| | | | | regulatory proteins of the |
| | | | | SIR2 family |
| SEQ ID NO. 1408 | LM-2801.1 | From 1672008 | to 1672616 | phosphoribosyl anthranilate |
| | | | | isomerase |
| SEQ ID NO. 1409 | LM-2804.1 | From 1670803 | to 1672005 | unknown, highly similar to |
| | | | | tryptophan synthase (beta |
| | | | | subunit) |
| SEQ ID NO. 1410 | LM-2805.1 | From 1670037 | to 1670810 | unknown, highly similar to |
| | | | | tryptophan synthase (alpha |
| | | | | subunit) |
| SEQ ID NO. 1411 | LM-2806.1 | From 1669529 | to 1669966 | Unknown |
| SEQ ID NO. 1412 | LM-2808.1 | From 1667831 | to 1669444 | Unknown, similar to putative |
| | | | | transporters |
| SEQ ID NO. 1413 | LM-2809.1 | From 1666107 | to 1667720 | Unknown, similar to putative |
| | | | | transporters |
| SEQ ID NO. 1414 | LM-.2810.1 | From 1665444 | to 1666097 | Unknown, similar to |
| | | | | unknown proteins |
| SEQ ID NO. 1415 | LM-2811.2 | From 1664575 | to 1665405 | Unknown, similar to |
| | | | | unknown proteins |
| SEQ ID NO. 1416 | LM-2812.2 | From 49585 | to 49878 | 30S ribosomal protein S6 |
| SEQ ID NO. 1417 | LM-2813.2 | From 49934 | to 50470 | unknown, highly similar to |
| | | | | single-strand binding protein |
| | | | | (SSB) |
| SEQ ID NO. 1418 | LM-2816.1 | From 50907 | to 51518 | Unknown |
| SEQ ID NO. 1419 | LM-2817.1 | From 51775 | to 52389 | Unknown, similar to |
| | | | | Staphylococcus AgrB |
| | | | | protein |
| SEQ ID NO. 1420 | LM-2818.1 | From 52630 | to 53925 | Unknown, similar to sensor |
| | | | | histidine kinase (AgrC from |
| | | | | Staphylococcus) |
| SEQ ID NO. 1421 | LM-2819.1 | From 53944 | to 54672 | Unknown, similar to 2- |
| | | | | components response |
| | | | | regulator protein (AgrA from |
| | | | | Staphylococcus) |
| SEQ ID NO. 1422 | LM-282.1 | From 2816083 | to 2816373 | Unknown |
| SEQ ID NO. 1423 | LM-2820.1 | From 54839 | to 56812 | Unknown, highly similar to |
| | | | | B. subtilis YybT protein |
| SEQ ID NO. 1424 | LM-2822.1 | From 56815 | to 57261 | 50S ribosomal protein L9 |
| SEQ ID NO. 1425 | LM-2823.2 | From 57286 | to 58638 | unknown, highly similar to |
| | | | | replicative DNA helicases |
| SEQ ID NO. 1426 | LM-2826.1 | From 2580470 | to 2581780 | Unknown, similar to cell wall |
| | | | | binding proteins |
| SEQ ID NO. 1427 | LM-2827.1 | From 2581900 | to 2583105 | peptidoglycan lytic protein |
| | | | | P45 |
| SEQ ID NO. 1428 | LM-2829.1 | From 2583181 | to 2584065 | unknown, highly similar to |
| | | | | cell-division protein FtsX |
| SEQ ID NO. 1429 | LM-283.1 | From 2816363 | to 2817571 | Unknown, similar to drug- |
| | | | | efflux transporters |
| SEQ ID NO. 1430 | LM-2831.1 | From 2584055 | to 2584741 | unknown, highly similar to |
| | | | | the cell-division ATP- |
| | | | | binding protein FtsE |
| SEQ ID NO. 1431 | LM-2832.1 | From 2585245 | to 2586108 | Unknown, similar to |
| | | | | conserved hypothetical |
| | | | | proteins |
| SEQ ID NO. 1432 | LM-2833.1 | From 2586114 | to 2587097 | unknown, highly similar to |
| | | | | peptide chain release |
| | | | | factor 2 |
| SEQ ID NO. 1433 | LM-2834.2 | From 2587300 | to 2589813 | translocase binding subunit |
| | | | | (ATPase) |
| SEQ ID NO. 1434 | LM-2835.2 | From 1052509 | to 1053429 | Unknown |
| SEQ ID NO. 1435 | LM-2836.1 | From 1051695 | to 1052354 | unknown, similar to a |
| | | | | bacterial K(+)-uptake |
| | | | | system |
| SEQ ID NO. 1436 | LM-2838.1 | From 1051038 | to 1051676 | unknown, similar to two- |
| | | | | component response |
| | | | | regulator, in particular B. subtilis |
| | | | | YvqC protein |
| SEQ ID NO. 1437 | LM-2839.1 | From 1049983 | to 1051041 | unknown, similar to two- |
| | | | | component sensor histidine |
| | | | | kinase in particular B. subtilis |
| | | | | YvqE protein |
| SEQ ID NO. 1438 | LM-284.1 | From 2817688 | to 2818035 | Unknown |
| SEQ ID NO. 1439 | LM-2840.1 | From 1049273 | to 1049986 | unknown, similar to B. subtilis |
| | | | | YvqF protein |
| SEQ ID NO. 1440 | LM-2842.1 | From 1048266 | to 1049138 | unknown, similar to B. subtilis |
| | | | | YitL protein |
| SEQ ID NO. 1441 | LM-2843.1 | From 1047490 | to 1048185 | unknown, similar to E. coli |
| | | | | copper homeostasis protein |
| | | | | CutC |
| SEQ ID NO. 1442 | LM-2845.1 | From 1046886 | to 1047377 | unknown, similar to |
| | | | | phosphotransferase system |
| | | | | glucose-specific enzyme IIA |
| SEQ ID NO. 1443 | LM-2846.1 | From 1045869 | to 1046771 | unknown, highly similar to |
| | | | | glycine betaine ABC |
| | | | | transporters (glycine |
| | | | | betaine-binding protein) |
| SEQ ID NO. 1444 | LM-2847.1 | From 1045007 | to 1045855 | unknown, highly similar to |
| | | | | glycine betaine ABC |
| | | | | transporters (permease) |
| SEQ ID NO. 1445 | LM-285.1 | From 2818054 | to 2818698 | Unknown, similar to |
| | | | | transaldolase |
| SEQ ID NO. 1446 | LM-2851.1 | From 1043821 | to 1045014 | unknown, highly similar to |
| | | | | glycine betaine ABC |
| | | | | transporter (ATP-binding |
| | | | | protein) |
| SEQ ID NO. 1447 | LM-2853.1 | From 1042605 | to 1043450 | unknown, similar to |
| | | | | conserved hypothetical |
| | | | | proteins like to B. subtilis |
| | | | | YkuT protein |
| SEQ ID NO. 1448 | LM-2854.1 | From 1041446 | to 1042561 | unknown, similar to N-acyl |
| | | | | L-amino acid |
| | | | | amidohydrolases |
| SEQ ID NO. 1449 | LM-2856.1 | From 1040672 | to 1041382 | unknown, similar to |
| | | | | tetrahydrodipicolinate |
| | | | | succinylase |
| SEQ ID NO. 1450 | LM-2857.1 | From 1039746 | to 1040624 | unknown, similar to |
| | | | | transcription regulator (LysR |
| | | | | family). |
| SEQ ID NO. 1451 | LM-2858.1 | From 1039297 | to 1039749 | unknown, similar to B. subtilis |
| | | | | YkuL protein |
| SEQ ID NO. 1452 | LM-2859.2 | From 1038863 | to 1039096 | unknown, similar to B. subtilis |
| | | | | YkuJ protein |
| SEQ ID NO. 1453 | LM-286.1 | From 2818841 | to 2819530 | Unknown, weakly similar to |
| | | | | transcription regulators |
| | | | | CRP/FNR family |
| SEQ ID NO. 1454 | LM-2860.1 | From 600837 | to 601148 | Unknown, similar to |
| | | | | phosphorybosil-AMP- |
| | | | | cyclohydrolase (HisI2 |
| | | | | protein) |
| SEQ ID NO. 1455 | LM-2861.1 | From 601149 | to 601466 | Unknown, similar to |
| | | | | phosphoribosyl-AMP |
| | | | | cyclohydrolase (HisI1 |
| | | | | protein) |
| SEQ ID NO. 1456 | LM-2863.1 | From 601463 | to 602218 | unknown, highly similar to |
| | | | | cyclase HisF |
| SEQ ID NO. 1457 | LM-2864.1 | From 602208 | to 602930 | unknown, highly similar to |
| | | | | phosphoribosylformimino-5- |
| | | | | aminoimidazole |
| | | | | carboxamide ribotide |
| | | | | isomerase |
| SEQ ID NO. 1458 | LM-2866.1 | From 602909 | to 603535 | unknown, similar to |
| | | | | amidotransferases |
| SEQ ID NO. 1459 | LM-2867.1 | From 603536 | to 604120 | Imidazoleglycerol- |
| | | | | phosphate dehydratase |
| SEQ ID NO. 1460 | LM-2868.1 | From 604121 | to 605404 | unknown, highly similar to |
| | | | | histidinol dehydrogenases |
| SEQ ID NO. 1461 | LM-287.1 | From 2819860 | to 2821587 | Unknown, similar to ABC |
| | | | | transporter (ATP-binding |
| | | | | protein) |
| SEQ ID NO. 1462 | LM-2870.1 | From 605401 | to 606042 | Unknown, similar to ATP |
| | | | | phosphoribosyltransferase |
| SEQ ID NO. 1463 | LM-2871.2 | From 606039 | to 607220 | histidyl-tRNA synthetase |
| SEQ ID NO. 1464 | LM-2872.2 | From 1645958 | to 1646413 | Unknown, similar to |
| | | | | unknown proteins |
| SEQ ID NO. 1465 | LM-2873.3 | From 1646669 | to 1647781 | Unknown, similar to |
| | | | | aminopeptidase |
| SEQ ID NO. 1466 | LM-2875.1 | From 1647821 | to 1648366 | Unknown, similar to 2-cys |
| | | | | peroxiredoxin |
| SEQ ID NO. 1467 | LM-2877.1 | From 1648511 | to 1649854 | unknown, similar to UDP-N- |
| | | | | acetyl muramate-alanine |
| | | | | ligases |
| SEQ ID NO. 1468 | LM-2878.2 | From 1650149 | to 1652500 | Unknown, similar to DNA |
| | | | | translocase |
| SEQ ID NO. 1469 | LM-288.1 | From 2821641 | to 2821892 | Unknown |
| SEQ ID NO. 1470 | LM-2880.1 | From 1652810 | to 1653427 | Unknown, similar |
| | | | | phenylalanyl-tRNA |
| | | | | synthetase (beta subunit) |
| SEQ ID NO. 1471 | LM-2881.1 | From 1653433 | to 1654233 | Unknown, similar to |
| | | | | unknown proteins |
| SEQ ID NO. 1472 | LM-2882.1 | From 1654268 | to 1654579 | Unknown, similar to |
| | | | | thioredoxin |
| SEQ ID NO. 1473 | LM-2883.1 | From 1654902 | to 1655975 | Unknown, similar to |
| | | | | aminopeptidase |
| SEQ ID NO. 1474 | LM-2884.1 | From 1656173 | to 1656484 | Unknown, similar to |
| | | | | unknown proteins |
| SEQ ID NO. 1475 | LM-2885.1 | From 1656521 | to 1656769 | Unknown, similar to |
| | | | | unknown proteins |
| SEQ ID NO. 1476 | LM-2886.1 | From 1656903 | to 1657754 | Unknown, similar to |
| | | | | unknown proteins |
| SEQ ID NO. 1477 | LM-2887.2 | From 1657815 | to 1658459 | Unknown, similar to |
| | | | | unknown proteins |
| SEQ ID NO. 1478 | LM-2889.2 | From 1658466 | to 1659248 | Unknown, similar to |
| | | | | unknown proteins |
| SEQ ID NO. 1479 | LM-289.1 | From 2821912 | to 2823195 | seryl-trna synthetase |
| SEQ ID NO. 1480 | LM-2890.2 | From 1307009 | to 1307605 | unknown, conserved |
| | | | | hypothetical protein, similar |
| | | | | to B. subtilis YneS protein |
| SEQ ID NO. 1481 | LM-2892.1 | From 1306095 | to 1306967 | Unknown, similar to |
| | | | | Lactococcus lactis LacX |
| | | | | protein |
| SEQ ID NO. 1482 | LM-2894.1 | From 1305781 | to 1306092 | Unknown, similar to B. subtilis |
| | | | | YneQ protein |
| SEQ ID NO. 1483 | LM-2895.1 | From 1305390 | to 1305758 | Unknown, similar to B. subtilis |
| | | | | YneP protein |
| SEQ ID NO. 1484 | LM-2898.1 | From 1304457 | to 1305236 | unknown, highly similar to |
| | | | | B. subtilis CodY protein |
| SEQ ID NO. 1485 | LM-29.1 | From 2732589 | to 2733233 | Unknown, similar to |
| | | | | ribulose-phosphate 3- |
| | | | | epimerase |
| SEQ ID NO. 1486 | LM-290.1 | From 2823530 | to 2823949 | Unknown, similar to B. subtilis |
| | | | | stress protein YdaG |
| SEQ ID NO. 1487 | LM-2900.1 | From 1303027 | to 1304436 | unknown, highly similar to |
| | | | | ATP-dependent Clp |
| | | | | protease-like proteins |
| SEQ ID NO. 1488 | LM-2901.1 | From 1302474 | to 1303013 | unknown, highly similar to |
| | | | | beta-type subunit of the 20S |
| | | | | proteasome |
| SEQ ID NO. 1489 | LM-2902.2 | From 1301551 | to 1302453 | unknown, similar to |
| | | | | integrase/recombinase |
| SEQ ID NO. 1490 | LM-2903.2 | From 1299964 | to 1301268 | unknown, similar glucose |
| | | | | inhibited division protein A |
| SEQ ID NO. 1491 | LM-2907.2 | From 1297823 | to 1299901 | unknown, highly similar to |
| | | | | DNA topoisomerase I TopA |
| SEQ ID NO. 1492 | LM-2908.2 | From 1296691 | to 1297551 | Unknown, similar to |
| | | | | polypeptide deformylase, |
| | | | | similar to B. subtilis Smf |
| | | | | protein |
| SEQ ID NO. 1493 | LM-2909.1 | From 1295772 | to 1296557 | unknown, similar to |
| | | | | ribonuclease H rnh |
| SEQ ID NO. 1494 | LM-291.1 | From 2824079 | to 2824651 | Unknown, similar to |
| | | | | glutamine amidotransferase |
| SEQ ID NO. 1495 | LM-2910.1 | From 1294912 | to 1295775 | unknown, conserved |
| | | | | hypothetical protein similar |
| | | | | to B. subtilis YlqF protein |
| SEQ ID NO. 1496 | LM-2911.1 | From 1294360 | to 1294902 | Unknown, similar to signal |
| | | | | peptidase I |
| SEQ ID NO. 1497 | LM-2912.2 | From 1293689 | to 1294258 | Unknown, similar to signal |
| | | | | peptidase I |
| SEQ ID NO. 1498 | LM-2914.2 | From 1498096 | to 1500252 | Unknown, similar to |
| | | | | unknown proteins |
| SEQ ID NO. 1499 | LM-2915.1 | From 1500268 | to 1501227 | Unknown, similar to |
| | | | | phosphate starvation |
| | | | | induced protein PhoH |
| SEQ ID NO. 1500 | LM-2916.1 | From 1501415 | to 1501861 | Unknown, similar to |
| | | | | unknown proteins |
| SEQ ID NO. 1501 | LM-2917.1 | From 1502252 | to 1503019 | Unknown, similar to |
| | | | | unknown proteins |
| SEQ ID NO. 1502 | LM-2918.1 | From 1503020 | to 1503964 | Unknown, similar to |
| | | | | ribosomal protein L11 |
| | | | | methyltransferase |
| SEQ ID NO. 1503 | LM-292.1 | From 2824648 | to 2826354 | Unknown, similar to para- |
| | | | | aminobenzoate synthase |
| | | | | component I |
| SEQ ID NO. 1504 | LM-2921.1 | From 1504037 | to 1505170 | heat shock protein DnaJ |
| SEQ ID NO. 1505 | LM-2922.1 | From 1505312 | to 1507153 | class I heat-shock protein |
| | | | | (molecular chaperone) |
| | | | | DnaK |
| SEQ ID NO. 1506 | LM-2924.1 | From 1507187 | to 1507762 | heat shock protein GrpE |
| SEQ ID NO. 1507 | LM-2925.1 | From 1507804 | to 1508841 | transcription repressor of |
| | | | | class I heat-shock gene |
| | | | | HrcA |
| SEQ ID NO. 1508 | LM-2926.1 | From 1508988 | to 1510145 | unknown, highly similar to |
| | | | | coproporphyrinogen III |
| | | | | oxidase |
| SEQ ID NO. 1509 | LM-2928.1 | From 1510234 | to 1511262 | Unknown, similar to |
| | | | | oxidoreductase |
| SEQ ID NO. 1510 | LM-2929.1 | From 1511375 | to 1511812 | Unknown, similar to |
| | | | | transcriptional regulator |
| | | | | (MerR family) |
| SEQ ID NO. 1511 | LM-2931.3 | From 1511858 | to 1513684 | unknown, highly similar to |
| | | | | GTP-binding protein LepA |
| SEQ ID NO. 1512 | LM-2932.2 | From 2535342 | to 2536268 | Unknown, similar to |
| | | | | dipeptidases |
| SEQ ID NO. 1513 | LM-2933.1 | From 2533840 | to 2535183 | RNA polymerase sigma-54 |
| | | | | factor (sigma-L) |
| SEQ ID NO. 1514 | LM-2935.1 | From 2532352 | to 2533398 | Unknown, similar to B. subtilis |
| | | | | CggR hypothetical |
| | | | | transcriptional regulator |
| SEQ ID NO. 1515 | LM-2936.1 | From 2531310 | to 2532320 | unknown, highly similar to |
| | | | | glyceraldehyde 3-phosphate |
| | | | | dehydrogenase |
| SEQ ID NO. 1516 | LM-2937.1 | From 2529985 | to 2531175 | unknown, highly similar to |
| | | | | phosphoglycerate kinase |
| SEQ ID NO. 1517 | LM-2939.1 | From 2529184 | to 2529939 | unknown, highly similar to |
| | | | | triose phosphate isomerase |
| SEQ ID NO. 1518 | LM-294.1 | From 2826515 | to 2828236 | Unknown, similar to ABC |
| | | | | transporter, ATP-binding |
| | | | | protein |
| SEQ ID NO. 1519 | LM-2942.1 | From 2527650 | to 2529182 | unknown, highly similar to |
| | | | | phosphoglycerate mutase |
| SEQ ID NO. 1520 | LM-2944.2 | From 2526222 | to 2527514 | unknown, highly similar to |
| | | | | enolase |
| SEQ ID NO. 1521 | LM-2945.1 | From 2525946 | to 2526119 | Unknown |
| SEQ ID NO. 1522 | LM-2946.2 | From 2525094 | to 2525813 | Unknown, similar to lipolytic |
| | | | | enzyme |
| SEQ ID NO. 1523 | LM-2950.2 | From 316873 | to 318375 | Unknown, similar to heat- |
| | | | | shock protein htrA serine |
| | | | | protease |
| SEQ ID NO. 1524 | LM-2952.1 | From 315945 | to 316775 | Unknown, conserved |
| | | | | hypothetical protein similar |
| | | | | to B. subtilis YycJ protein |
| SEQ ID NO. 1525 | LM-2953.1 | From 314984 | to 315823 | Unknown, similar to B. subtilis |
| | | | | Yycl protein |
| SEQ ID NO. 1526 | LM-2954.1 | From 313659 | to 314981 | Unknown, similar to B. subtilis |
| | | | | YycH protein |
| SEQ ID NO. 1527 | LM-2957.1 | From 311830 | to 313662 | Unknown, similar to two- |
| | | | | component sensor histidine |
| | | | | kinase |
| SEQ ID NO. 1528 | LM-2958.1 | From 310932 | to 311645 | Unknown, similar to two- |
| | | | | component response |
| | | | | regulator |
| SEQ ID NO. 1529 | LM-2959.2 | From 309538 | to 310719 | Unknown, similar to |
| | | | | aminotransferase |
| SEQ ID NO. 1530 | LM-296.1 | From 2828236 | to 2830008 | Unknown, similar to ABC |
| | | | | transporter, ATP-binding |
| | | | | protein |
| SEQ ID NO. 1531 | LM-2960.3 | From 2692097 | to 2693041 | unknown, highly similar to |
| | | | | RNA polymerase (alpha |
| | | | | subunit) |
| SEQ ID NO. 1532 | LM-2962.2 | From 2693151 | to 2693540 | ribosomal protein S11 |
| SEQ ID NO. 1533 | LM-2964.2 | From 2693563 | to 2693928 | ribosomal protein S13 |
| SEQ ID NO. 1534 | LM-2965.1 | From 2694164 | to 2694382 | unknown, highly similar to |
| | | | | initiation factor IF-I |
| SEQ ID NO. 1535 | LM-2967.1 | From 2694769 | to 2695416 | unknown, highly similar to |
| | | | | adenylate kinases |
| SEQ ID NO. 1536 | LM-2968.1 | From 2695476 | to 2696771 | unknown, highly similar to |
| | | | | preprotein translocase |
| | | | | subunit |
| SEQ ID NO. 1537 | LM-297.1 | From 2830284 | to 2830523 | Unknown |
| SEQ ID NO. 1538 | LM-2970.1 | From 2696771 | to 2697211 | ribosomal protein L15 |
| SEQ ID NO. 1539 | LM-2972.1 | From 2697463 | to 2697966 | ribosomal protein S5 |
| SEQ ID NO. 1540 | LM-2973.1 | From 2697988 | to 2698347 | ribosomal protein L18 |
| SEQ ID NO. 1541 | LM-2974.1 | From 2698387 | to 2698923 | ribosomal protein L6 |
| SEQ ID NO. 1542 | LM-2975.1 | From 2698954 | to 2699352 | ribosomal protein S8 |
| SEQ ID NO. 1543 | LM-2977.1 | From 2699600 | to 2700139 | ribosomal protein L5 |
| SEQ ID NO. 1544 | LM-2979.1 | From 2700166 | to 2700477 | ribosomal protein L24 |
| SEQ ID NO. 1545 | LM-298.1 | From 2830542 | to 2831879 | Unknown, similar to D- |
| | | | | alanyl-D-alanine |
| | | | | carboxypeptidase |
| | | | | (penicillin-binding protein 5) |
| SEQ ID NO. 1546 | LM-2981.1 | From 2700515 | to 2700883 | ribosomal protein L14 |
| SEQ ID NO. 1547 | LM-2983.1 | From 2701435 | to 2701869 | ribosomal protein L16 |
| SEQ ID NO. 1548 | LM-2984.1 | From 2701872 | to 2702528 | ribosomal protein S3 |
| SEQ ID NO. 1549 | LM-2985.2 | From 2702532 | to 2702888 | ribosomal protein L22 |
| SEQ ID NO. 1550 | LM-2986.2 | From 2702909 | to 2703187 | ribosomal protein S19 |
| SEQ ID NO. 1551 | LM-2990.2 | From 1593100 | to 1594407 | unknown, highly similar to |
| | | | | glutamyl-tRNA reductase |
| SEQ ID NO. 1552 | LM-2991.1 | From 1592167 | to 1593096 | unknown, highly similar to |
| | | | | porphobilinogen |
| | | | | deaminases |
| | | | | (hydroxymethylbilane |
| | | | | synthase) |
| SEQ ID NO. 1553 | LM-2992.1 | From 1591448 | to 1592170 | Unknown, similar to |
| | | | | uroporphyrinogen III |
| | | | | cosynthase (HemD) |
| SEQ ID NO. 1554 | LM-2993.1 | From 1590477 | to 1591451 | unknown, highly similar to |
| | | | | delta-aminolevulinic acid |
| | | | | dehydratases |
| | | | | (porphobilinogen synthase) |
| SEQ ID NO. 1555 | LM-2995.1 | From 1589175 | to 1590464 | unknown, highly similar to |
| | | | | glutamate-1-semialdehyde |
| | | | | 2,1-aminotransferases |
| SEQ ID NO. 1556 | LM-2997.1 | From 1586201 | to 1588852 | valyl-tRNA synthetase |
| SEQ ID NO. 1557 | LM-2999.2 | From 1584850 | to 1586139 | unknown, similar to Folyl- |
| | | | | polyglutamate synthetase |
| SEQ ID NO. 1558 | LM-3.1 | From 2712931 | to 2713365 | Unknown |
| SEQ ID NO. 1559 | LM-3000.1 | From 1583928 | to 1584638 | Unknown, similar to B. subtilis |
| | | | | late competence |
| | | | | protein ComC (type IV |
| | | | | prepilin peptidase) |
| SEQ ID NO. 1560 | LM-3001.1 | From 1583242 | to 1583916 | Unknown, similar to DNA |
| | | | | repair protein RadC |
| SEQ ID NO. 1561 | LM-3004.1 | From 1581777 | to 1582790 | unknown, similar to cell- |
| | | | | shape determining protein |
| | | | | MreB |
| SEQ ID NO. 1562 | LM-3005.1 | From 1580804 | to 1581691 | unknown, similar to cell- |
| | | | | shape determining protein |
| | | | | MreC |
| SEQ ID NO. 1563 | LM-3006.1 | From 1580283 | to 1580801 | unknown, similar to cell- |
| | | | | shape determining protein |
| | | | | MreD |
| SEQ ID NO. 1564 | LM-3007.2 | From 1579428 | to 1580105 | unknown, similar to cell- |
| | | | | division inhibition (septum |
| | | | | placement) protein MinC |
| SEQ ID NO. 1565 | LM-301.1 | From 2832058 | to 2833725 | Unknown, similar to acylase |
| | | | | and diesterase |
| SEQ ID NO. 1566 | LM-3010.1 | From 1149406 | to 1149720 | unknown, highly similar to |
| | | | | TN916 ORF23 |
| SEQ ID NO. 1567 | LM-3011.1 | From 1149011 | to 1149385 | unknown, highly similar to |
| | | | | TN916 ORF22 |
| SEQ ID NO. 1568 | LM-3012.1 | From 1147603 | to 1149003 | unknown, highly similar to |
| | | | | TN916 ORF21 |
| SEQ ID NO. 1569 | LM-3013.1 | From 1146228 | to 1147412 | unknown, highly similar to |
| | | | | TN916 ORF20 |
| SEQ ID NO. 1570 | LM-3014.1 | From 1145941 | to 1146231 | unknown, similar to |
| | | | | unknown proteins |
| SEQ ID NO. 1571 | LM-3016.1 | From 1145211 | to 1145711 | unknown, highly similar to |
| | | | | TN916 ORF18 |
| SEQ ID NO. 1572 | LM-3017.1 | From 1144533 | to 1144928 | unknown, highly similar to |
| | | | | TN916 ORF17 |
| SEQ ID NO. 1573 | LM-3018.1 | From 1142099 | to 1144549 | unknown, highly similar to |
| | | | | TN916 ORF16 |
| SEQ ID NO. 1574 | LM-3020.1 | From 1139940 | to 1142099 | unknown, highly similar to |
| | | | | TN916 ORF15 |
| SEQ ID NO. 1575 | LM-3022.1 | From 1138930 | to 1139940 | unknown, highly similar to |
| | | | | TN916 ORF14 and to L. monocytogenes |
| | | | | P60 protein |
| SEQ ID NO. 1576 | LM-3023.1 | From 1137997 | to 1138914 | unknown, highly similar to |
| | | | | TN916 ORF13 |
| SEQ ID NO. 1577 | LM-3024.1 | From 1137533 | to 1137868 | unknown, similar to |
| | | | | cadmium efflux system |
| | | | | accessory proteins |
| SEQ ID NO. 1578 | LM-3025.3 | From 1254459 | to 1254722 | unknown, similar to B. subtilis |
| | | | | YshA protein |
| SEQ ID NO. 1579 | LM-3027.3 | From 1253379 | to 1254311 | unknown, similar to B. subtilis |
| | | | | ribonuclease HIII |
| SEQ ID NO. 1580 | LM-3028.1 | From 1252667 | to 1253341 | Unknown, similar to uracil- |
| | | | | DNA glycosylase |
| SEQ ID NO. 1581 | LM-3029.1 | From 1249360 | to 1252560 | unknown; similar to |
| | | | | transporter, (to B. subtilis |
| | | | | YdgH protein) |
| SEQ ID NO. 1582 | LM-3030.1 | From 1248892 | to 1249344 | unknown; similar to |
| | | | | transcriptional regulator |
| | | | | (MarR family). |
| SEQ ID NO. 1583 | LM-3031.2 | From 1245384 | to 1248794 | unknown, similar to different |
| | | | | proteins |
| SEQ ID NO. 1584 | LM-3032.2 | From 1244669 | to 1245370 | unknown, similar to ABC |
| | | | | transporter, ATP-binding |
| | | | | proteins |
| SEQ ID NO. 1585 | LM-3035.2 | From 1608166 | to 1609923 | unknown, highly similar to |
| | | | | pyruvate kinases |
| SEQ ID NO. 1586 | LM-3036.1 | From 1607671 | to 1608048 | Unknown, similar to |
| | | | | unknown proteins |
| SEQ ID NO. 1587 | LM-3037.1 | From 1607053 | to 1607514 | Unknown, similar to |
| | | | | unknown proteins |
| SEQ ID NO. 1588 | LM-3038.1 | From 1605895 | to 1607016 | unknown, highly similar to |
| | | | | citrate synthase subunit II |
| SEQ ID NO. 1589 | LM-3039.1 | From 1604613 | to 1605875 | unknown, highly similar to |
| | | | | isocitrate dehyrogenases |
| SEQ ID NO. 1590 | LM-304.1 | From 2833834 | to 2835987 | Unknown, similar to DNA |
| | | | | topoisomerase III |
| SEQ ID NO. 1591 | LM-3041.1 | From 1601820 | to 1604447 | DNA polymerase I |
| SEQ ID NO. 1592 | LM-3042.1 | From 1600975 | to 1601796 | unknown, highly similar to |
| | | | | formamidopyrimidine-DNA |
| | | | | glycosylases |
| SEQ ID NO. 1593 | LM-3043.2 | From 1600356 | to 1600958 | Unknown, similar to |
| | | | | unknown proteins |
| SEQ ID NO. 1594 | LM-3044.2 | From 1875574 | to 1876560 | Unknown, similar to FtsY of |
| | | | | E. coli and SRP receptor |
| | | | | alpha-subunit |
| SEQ ID NO. 1595 | LM-3049.1 | From 1876575 | to 1880135 | Unknown, similar to Smc |
| | | | | protein essential for |
| | | | | chromosome condensation |
| | | | | and partition |
| SEQ ID NO. 1596 | LM-3050.1 | From 1880158 | to 1880847 | Unknown, similar to |
| | | | | ribonuclease III |
| SEQ ID NO. 1597 | LM-3053.1 | From 1881381 | to 1882124 | Unknown, similar to 3- |
| | | | | ketoacyl-acyl carrier protein |
| | | | | reductase |
| SEQ ID NO. 1598 | LM-3054.1 | From 1882128 | to 1883069 | Unknown, similar to malonyl |
| | | | | CoA-acyl carrier protein |
| | | | | transacylase |
| SEQ ID NO. 1599 | LM-3056.1 | From 1883062 | to 1884075 | Unknown, similar to plsX |
| | | | | protein involved in fatty |
| | | | | acid/phospholipid synthesis |
| SEQ ID NO. 1600 | LM-3058.2 | From 1884096 | to 1884665 | Unknown, similar to |
| | | | | unknown proteins |
| SEQ ID NO. 1601 | LM-3059.2 | From 817914 | to 818762 | Unknown, similar to |
| | | | | conserved hypothetical |
| | | | | proteins |
| SEQ ID NO. 1602 | LM-3062.1 | From 813451 | to 817878 | Unknown |
| SEQ ID NO. 1603 | LM-3065.3 | From 811813 | to 813189 | Unknown, similar to amino |
| | | | | acid transporter |
| SEQ ID NO. 1604 | LM-3066.2 | From 2159224 | to 2159604 | Unknown |
| SEQ ID NO. 1605 | LM-3067.1 | From 2157949 | to 2159094 | Unknown |
| SEQ ID NO. 1606 | LM-3068.1 | From 2157383 | to 2157844 | Unknown, similar to |
| | | | | unknown proteins |
| SEQ ID NO. 1607 | LM-3069.1 | From 2156694 | to 2157386 | Unknown, similar to |
| | | | | glycoprotease |
| SEQ ID NO. 1608 | LM-307.1 | From 2836011 | to 2837783 | Unknown, similar to ATP- |
| | | | | dependent DNA helicases |
| SEQ ID NO. 1609 | LM-3070.1 | From 2156242 | to 2156697 | Unknown, similar to |
| | | | | ribosomal protein alanine |
| | | | | acetyltransferase |
| SEQ ID NO. 1610 | LM-3072.1 | From 2155223 | to 2156245 | Unknown, similar to |
| | | | | glycoprotein endopeptidase |
| SEQ ID NO. 1611 | LM-3073.1 | From 2153720 | to 2154679 | Unknown, similar to |
| | | | | unknown proteins |
| SEQ ID NO. 1612 | LM-3074.1 | From 2151744 | to 2153696 | Unknown, similar to ABC |
| | | | | transporter (ATP-binding |
| | | | | protein) |
| SEQ ID NO. 1613 | LM-3075.1 | From 2150786 | to 2151433 | Unknown, similar to a |
| | | | | putative DNA binding |
| | | | | proteins |
| SEQ ID NO. 1614 | LM-3076.2 | From 2149868 | to 2150554 | Unknown, similar to |
| | | | | unknown proteins |
| SEQ ID NO. 1615 | LM-3077.2 | From 992978 | to 993886 | unknown, similar to |
| | | | | proteases |
| SEQ ID NO. 1616 | LM-3080.1 | From 993900 | to 995126 | unknown, similar to |
| | | | | proteases |
| SEQ ID NO. 1617 | LM-3081.1 | From 995210 | to 995767 | unknown, Listeria epitope |
| | | | | LemA |
| SEQ ID NO. 1618 | LM-3083.1 | From 995790 | to 996704 | unknown, similar to putative |
| | | | | heat shock protein HtpX, |
| | | | | Listeria epitope LemB |
| SEQ ID NO. 1619 | LM-3084.1 | From 996739 | to 997557 | unknown, similar to B. subtilis |
| | | | | YjbH protein |
| SEQ ID NO. 1620 | LM-3085.1 | From 997795 | to 998379 | unknown, similar to B. subtilis |
| | | | | YjbK protein |
| SEQ ID NO. 1621 | LM-3086.2 | From 998396 | to 998863 | Unknown |
| SEQ ID NO. 1622 | LM-3087.2 | From 999022 | to 999690 | unknown, similar to B. subtilis |
| | | | | YjbM protein |
| SEQ ID NO. 1623 | LM-3088.1 | From 999722 | to 1000516 | unknown, similar to |
| | | | | conserved hypothetical |
| | | | | proteins like to B. subtilis |
| | | | | YjbN protein |
| SEQ ID NO. 1624 | LM-3089.1 | From 1000535 | to 1001425 | unknown, similar to |
| | | | | ribosomal large subunit |
| | | | | pseudouridine synthetase |
| SEQ ID NO. 1625 | LM-309.2 | From 2837944 | to 2839410 | Unknown, similar to inosine- |
| | | | | monophosphate |
| | | | | dehydrogenase |
| SEQ ID NO. 1626 | LM-3090.1 | From 1001503 | to 1002291 | unknown, similar to enoyl- |
| | | | | acyl-carrier protein |
| | | | | reductase |
| SEQ ID NO. 1627 | LM-3091.1 | From 1002319 | to 1003593 | DltD protein for D-alanine |
| | | | | esterification of lipoteichoic |
| | | | | acid and wall teichoic acid |
| SEQ ID NO. 1628 | LM-3092.1 | From 1003855 | to 1005039 | DltB protein for D-alanine |
| | | | | esterification of lipoteichoic |
| | | | | acid and wall teichoic acid |
| SEQ ID NO. 1629 | LM-3093.2 | From 1005036 | to 1006568 | D-alanine-activating |
| | | | | enzyme (dae), D-alanine-D- |
| | | | | alanyl carrier protein ligase |
| | | | | (dcl) |
| SEQ ID NO. 1630 | LM-3095.2 | From 1364031 | to 1364570 | unknown, similar to 5- |
| | | | | formyltetrahydrofolate cycloligase |
| SEQ ID NO. 1631 | LM-3097.1 | From 1364669 | to 1366207 | unknown similar to B. subtilis |
| | | | | yqgP |
| SEQ ID NO. 1632 | LM-3098.1 | From 1366457 | to 1367425 | Unknown, similar to glucose |
| | | | | kinase |
| SEQ ID NO. 1633 | LM-3099.1 | From 1367559 | to 1368650 | unknown, similar to B. subtilis |
| | | | | YqgU protein |
| SEQ ID NO. 1634 | LM-3100.1 | From 1368668 | to 1368985 | Unknown, weakly similar to |
| | | | | B. subtilis comG operon |
| | | | | protein 7 (comGG) |
| SEQ ID NO. 1635 | LM-3101.1 | From 1368982 | to 1369449 | Unknown, similar to B. subtilis |
| | | | | comG operon |
| | | | | protein 6 |
| SEQ ID NO. 1636 | LM-3102.1 | From 1369412 | to 1369696 | Unknown, similar to comG |
| | | | | operon protein 5 (comGE) |
| SEQ ID NO. 1637 | LM-3103.1 | From 1369683 | to 1370111 | Unknown, similar to comG |
| | | | | operon protein 4 (comGD) |
| SEQ ID NO. 1638 | LM-3104.1 | From 1370108 | to 1370431 | Unknown, similar to B. subtilis |
| | | | | comG operon |
| | | | | protein 3 |
| SEQ ID NO. 1639 | LM-3105.1 | From 1370445 | to 1371476 | Unknown, similar to B. subtilis |
| | | | | comG operon |
| | | | | protein 2 |
| SEQ ID NO. 1640 | LM-3106.1 | From 1371454 | to 1372476 | Unknown, similar to B. subtilis |
| | | | | comG operon |
| | | | | protein 1 |
| SEQ ID NO. 1641 | LM-3107.1 | From 1373015 | to 1374103 | Unknown, similar to |
| | | | | aminomethyltransferase |
| SEQ ID NO. 1642 | LM-3110.3 | From 1374119 | to 1375465 | Unknown, similar to glycine |
| | | | | dehydrogenase |
| | | | | (decarboxylating) subunit 1 |
| SEQ ID NO. 1643 | LM-3111.1 | From 1375462 | to 1376928 | Unknown, similar to glycine |
| | | | | dehydrogenase |
| | | | | (decarboxylating) subunit 2 |
| SEQ ID NO. 1644 | LM-3112.1 | From 1376968 | to 1377348 | Unknown |
| SEQ ID NO. 1645 | LM-3113.1 | From 1377410 | to 1377682 | Unknown |
| SEQ ID NO. 1646 | LM-3114.2 | From 1377695 | to 1378660 | Unknown, similar to B. subtilis |
| | | | | YqhQ protein |
| SEQ ID NO. 1647 | LM-3115.3 | From 1962698 | to 1963243 | unknown, similar to |
| | | | | conserved hypothetical |
| | | | | proteins |
| SEQ ID NO. 1648 | LM-3116.2 | From 1962225 | to 1962566 | unknown, similar to |
| | | | | hypothetical proteins |
| SEQ ID NO. 1649 | LM-3119.1 | From 1960496 | to 1961644 | unknown, similar to |
| | | | | conserved hypothetical |
| | | | | proteins |
| SEQ ID NO. 1650 | LM-312.1 | From 130652 | to 131380 | Unknown, similar to |
| | | | | autolysin: N- |
| | | | | acetylmuramoyl-L-alanine |
| | | | | amidase |
| SEQ ID NO. 1651 | LM-3120.1 | From 1958968 | to 1960476 | unknown, similar to |
| | | | | probable thermostable |
| | | | | carboxypeptidases |
| SEQ ID NO. 1652 | LM-3121.1 | From 1958162 | to 1958740 | unknown, similar to |
| | | | | xanthine |
| | | | | phosphoribosyltransferase |
| SEQ ID NO. 1653 | LM-3124.1 | From 1956848 | to 1958155 | unknown, similar to |
| | | | | probable permeases |
| SEQ ID NO. 1654 | LM-3126.1 | From 1955598 | to 1956656 | unknown, similar to |
| | | | | chitinases |
| SEQ ID NO. 1655 | LM-3127.2 | From 1955205 | to 1955474 | unknown, similar to |
| | | | | ribosomal protein S14 |
| SEQ ID NO. 1656 | LM-3128.3 | From 1954255 | to 1955127 | unknown, similar to 5′-3′- |
| | | | | exonuclease |
| SEQ ID NO. 1657 | LM-3129.1 | From 248555 | to 249013 | Unknown, highly similar to |
| | | | | transcription repressor of |
| | | | | class III stress genes (CtsR) |
| SEQ ID NO. 1658 | LM-313.1 | From 130249 | to 130671 | Unknown, similar to a |
| | | | | protein from Bacteriophage |
| | | | | phi-105 (ORF 45) |
| SEQ ID NO. 1659 | LM-3130.1 | From 249026 | to 249544 | Unknown, similar to B. subtilis |
| | | | | YacH protein |
| SEQ ID NO. 1660 | LM-3131.1 | From 249541 | to 250563 | Unknown, similar to arginine |
| | | | | kinase |
| SEQ ID NO. 1661 | LM-3135.1 | From 250592 | to 253054 | endopeptidase Clp ATP- |
| | | | | binding chain C |
| SEQ ID NO. 1662 | LM-3136.2 | From 253200 | to 254573 | unknown, similar to DNA |
| | | | | repair protein Sms |
| SEQ ID NO. 1663 | LM-3139.2 | From 254707 | to 255780 | Unknown, highly similar to |
| | | | | B. subtilis YacL protein |
| SEQ ID NO. 1664 | LM-314.1 | From 129694 | to 130230 | Unknown, weakly similar to |
| | | | | protein gp20 from |
| | | | | Bacteriophage A118 |
| SEQ ID NO. 1665 | LM-3140.2 | From 255800 | to 256498 | Unknown, similar to |
| | | | | nucleotidylyl transferase; |
| | | | | pyrophosphorylase |
| SEQ ID NO. 1666 | LM-3141.2 | From 348586 | to 349065 | Unknown |
| SEQ ID NO. 1667 | LM-3142.2 | From 349326 | to 350228 | Unknown, similar to |
| | | | | transcriptional regulators |
| SEQ ID NO. 1668 | LM-3143.1 | From 350392 | to 351264 | Unknown, similar to |
| | | | | transcriptional regulators |
| SEQ ID NO. 1669 | LM-3147.1 | From 355643 | to 356323 | Unknown |
| SEQ ID NO. 1670 | LM-3149.2 | From 2602249 | to 2602683 | Unknown, similar to |
| | | | | hydroxymyristoyl-(acyl |
| | | | | carrier protein) dehydratase |
| SEQ ID NO. 1671 | LM-315.1 | From 129218 | to 129697 | Unknown |
| SEQ ID NO. 1672 | LM-3150.1 | From 2601632 | to 2601997 | Unknown, similar to single- |
| | | | | strand DNA-binding protein |
| SEQ ID NO. 1673 | LM-3151.1 | From 2600408 | to 2601241 | Unknown, similar to |
| | | | | hypothetical cell wall |
| | | | | binding protein from B. subtilis |
| SEQ ID NO. 1674 | LM-3152.1 | From 2599548 | to 2600282 | Unknown, similar to B. subtilis |
| | | | | TagA protein |
| | | | | involved in polyglycerol |
| | | | | phosphate biosynthesis |
| SEQ ID NO. 1675 | LM-3153.1 | From 2598423 | to 2599547 | Unknown, similar to B. subtilis |
| | | | | O-succinylbenzoate- |
| | | | | CoA synthase (MenC) |
| SEQ ID NO. 1676 | LM-3154.1 | From 2597363 | to 2598415 | Unknown, similar to B. subtilis |
| | | | | TagO teichoic acid |
| | | | | linkage unit synthesis |
| | | | | protein |
| SEQ ID NO. 1677 | LM-3155.1 | From 2596266 | to 2597324 | Unknown, similar to B. subtilis |
| | | | | putative |
| | | | | transcriptional regulator |
| | | | | LytR |
| SEQ ID NO. 1678 | LM-3156.1 | From 2595485 | to 2596090 | Unknown |
| SEQ ID NO. 1679 | LM-3157.1 | From 2594908 | to 2595543 | Unknown, similar to |
| | | | | conserved hypothetical |
| | | | | proteins |
| SEQ ID NO. 1680 | LM-3158.1 | From 2593837 | to 2594523 | Unknown, similar to B. subtilis |
| | | | | two-component |
| | | | | response regulator DegU |
| SEQ ID NO. 1681 | LM-3159.1 | From 2592965 | to 2593816 | Unknown, similar to B. subtilis |
| | | | | YviA (DegV) protein |
| SEQ ID NO. 1682 | LM-316.1 | From 128628 | to 129203 | Unknown |
| SEQ ID NO. 1683 | LM-3162.1 | From 2591451 | to 2592770 | unknown, similar to late |
| | | | | competence protein comFA |
| SEQ ID NO. 1684 | LM-3163.2 | From 2590802 | to 2591458 | unknown, similar to late |
| | | | | competence protein comFC |
| SEQ ID NO. 1685 | LM-3164.2 | From 2590044 | to 2590607 | Unknown, similar to |
| | | | | conserved hypothetical |
| | | | | proteins like to B. subtilis |
| | | | | YvyD protein |
| SEQ ID NO. 1686 | LM-3167.1 | From 2020738 | to 2021256 | Unknown, similar to similar |
| | | | | to acyl-CoA hydrolase |
| SEQ ID NO. 1687 | LM-3168.1 | From 2021272 | to 2023062 | Unknown, similar to two- |
| | | | | component sensor histidine |
| | | | | kinase (ResE) |
| SEQ ID NO. 1688 | LM-3169.1 | From 2023163 | to 2023879 | Unknown, similar to two- |
| | | | | component response |
| | | | | regulator (ResD) |
| SEQ ID NO. 1689 | LM-317.1 | From 128314 | to 128613 | Unknown |
| SEQ ID NO. 1690 | LM-3171.1 | From 2024062 | to 2024796 | Unknown, similar to |
| | | | | unknown proteins |
| SEQ ID NO. 1691 | LM-3172.1 | From 2024799 | to 2025395 | Unknown, similar to |
| | | | | unknown proteins |
| SEQ ID NO. 1692 | LM-3174.1 | From 2025392 | to 2026141 | Unknown, similar to |
| | | | | unknown proteins |
| SEQ ID NO. 1693 | LM-3177.1 | From 2026157 | to 2027467 | Unknown, similar to |
| | | | | diaminopimelate |
| | | | | decarboxylase |
| SEQ ID NO. 1694 | LM-3178.1 | From 2027648 | to 2028466 | Unknown, similar to purine- |
| | | | | nucleoside phosphorylase |
| SEQ ID NO. 1695 | LM-3179.2 | From 2028485 | to 2029669 | Unknown, similar to |
| | | | | phosphopentomutase |
| SEQ ID NO. 1696 | LM-3181.2 | From 2029698 | to 2030591 | Unknown, similar to |
| | | | | integrase/recombinase |
| SEQ ID NO. 1697 | LM-3184.2 | From 635317 | to 636423 | Unknown, similar to |
| | | | | homoserine O- |
| | | | | acetyltransferase |
| SEQ ID NO. 1698 | LM-3186.1 | From 636440 | to 637717 | Unknown, similar to O- |
| | | | | acetylhomoserine |
| | | | | sulfhydrylase |
| SEQ ID NO. 1699 | LM-3188.1 | From 638165 | to 638692 | Unknown, similar to |
| | | | | unknown proteins |
| SEQ ID NO. 1700 | LM-3189.1 | From 638781 | to 639482 | Unknown, similar to |
| | | | | transcription regulator |
| | | | | CRP/FNR family |
| SEQ ID NO. 1701 | LM-319.1 | From 127188 | to 128324 | Unknown, similar to protein |
| | | | | gp18 from Bacteriophage |
| | | | | A118 |
| SEQ ID NO. 1702 | LM-3190.2 | From 639558 | to 640106 | Unknown, similar to proteins |
| | | | | involved in biotin |
| | | | | metabolism (BioY) |
| SEQ ID NO. 1703 | LM-3191.2 | From 1537815 | to 1538501 | Unknown, similar to two- |
| | | | | component response |
| | | | | regulators |
| SEQ ID NO. 1704 | LM-3192.1 | From 1538498 | to 1539937 | Unknown, similar to two- |
| | | | | component sensor histidine |
| | | | | kinase |
| SEQ ID NO. 1705 | LM-3194.1 | From 1539979 | to 1542375 | Unknown, similar to |
| | | | | exodeoxyribonuclease V |
| SEQ ID NO. 1706 | LM-3195.1 | From 1542403 | to 1543041 | Unknown, similar to |
| | | | | unknown proteins |
| SEQ ID NO. 1707 | LM-3196.1 | From 1543082 | to 1543822 | Unknown, similar to |
| | | | | unknown proteins |
| SEQ ID NO. 1708 | LM-3198.1 | From 1543940 | to 1545055 | Unknown, similar to putative |
| | | | | tRNA (5- |
| | | | | methylaminomethyl-2- |
| | | | | thiouridylate)- |
| | | | | methyltransferase |
| SEQ ID NO. 1709 | LM-32.1 | From 2733230 | to 2735245 | Unknown, similar to |
| | | | | transketolase |
| SEQ ID NO. 1710 | LM-3200.2 | From 1545074 | to 1546222 | Unknown, similar to iron- |
| | | | | sulfur cofactor synthesis |
| | | | | protein |
| SEQ ID NO. 1711 | LM-3201.2 | From 1527422 | to 1527904 | Unknown, similar to |
| | | | | transcription elongation |
| | | | | factor GreA |
| SEQ ID NO. 1712 | LM-3203.2 | From 1526748 | to 1527371 | Unknown, similar to |
| | | | | unknown proteins |
| SEQ ID NO. 1713 | LM-3204.1 | From 1525997 | to 1526698 | Unknown, similar to 5- |
| | | | | methylthioadenosine/S- |
| | | | | adenosylhomocysteine |
| | | | | nucleosidase |
| SEQ ID NO. 1714 | LM-3205.1 | From 1524154 | to 1525962 | Unknown, similar to |
| | | | | oligopeptidase |
| SEQ ID NO. 1715 | LM-3206.2 | From 1523471 | to 1523992 | Unknown, similar to |
| | | | | unknown proteins |
| SEQ ID NO. 1716 | LM-3208.2 | From 1522374 | to 1523474 | Unknown, similar to |
| | | | | unknown proteins |
| SEQ ID NO. 1717 | LM-3209.1 | From 1521533 | to 1522354 | Unknown, similar to |
| | | | | shikimate 5-dehydrogenase |
| | | | | (AroD) |
| SEQ ID NO. 1718 | LM-321.1 | From 126360 | to 127178 | Unknown, similar to phage |
| | | | | proteins |
| SEQ ID NO. 1719 | LM-3210.1 | From 1521243 | to 1521533 | Unknown, similar to |
| | | | | unknown proteins |
| SEQ ID NO. 1720 | LM-3211.1 | From 1520659 | to 1521225 | Unknown, similar to |
| | | | | unknown proteins |
| SEQ ID NO. 1721 | LM-3212.1 | From 1520097 | to 1520672 | Unknown, similar to |
| | | | | unknown proteins |
| SEQ ID NO. 1722 | LM-3213.1 | From 1519736 | to 1520092 | Unknown |
| SEQ ID NO. 1723 | LM-3214.1 | From 1518950 | to 1519681 | Unknown, similar to |
| | | | | unknown proteins |
| SEQ ID NO. 1724 | LM-3215.1 | From 1518245 | to 1518847 | Unknown, similar to integral |
| | | | | membrane protein ComEA |
| SEQ ID NO. 1725 | LM-3216.1 | From 1517613 | to 1518173 | Unknown, similar to B. subtilis |
| | | | | ComEB protein |
| SEQ ID NO. 1726 | LM-3217.2 | From 1515356 | to 1517578 | Unknown, similar to putative |
| | | | | integral membrane protein |
| | | | | ComEC specifically required |
| | | | | for DNA uptake but not for |
| | | | | binding |
| SEQ ID NO. 1727 | LM-3218.2 | From 1418803 | to 1420095 | Unknown, similar to putative |
| | | | | proteases |
| SEQ ID NO. 1728 | LM-3220.1 | From 1417735 | to 1418685 | Unknown, similar to sugar |
| | | | | ABC transporter, permease |
| | | | | protein |
| SEQ ID NO. 1729 | LM-3221.1 | From 1416686 | to 1417738 | Unknown, similar to |
| | | | | permease proteins |
| SEQ ID NO. 1730 | LM-3223.1 | From 1415152 | to 1416693 | Unknown, similar to sugar |
| | | | | ABC transporter, ATP- |
| | | | | binding protein |
| SEQ ID NO. 1731 | LM-3226.3 | From 1413646 | to 1414719 | Unknown, CD4+ T cell- |
| | | | | stimulating antigen, |
| | | | | lipoprotein |
| SEQ ID NO. 1732 | LM-3228.3 | From 1412455 | to 1413294 | Unknown, similar to |
| | | | | pyrroline-5-carboxylate |
| | | | | reductase |
| SEQ ID NO. 1733 | LM-3231.1 | From 1410148 | to 1412421 | Unknown, similar to DNA |
| | | | | translocase |
| SEQ ID NO. 1734 | LM-3234.2 | From 843311 | to 843724 | Unknown, similar to E. coli |
| | | | | PhnB protein |
| SEQ ID NO. 1735 | LM-3235.1 | From 842781 | to 843287 | Unknown, similar to B. subtilis |
| | | | | regulatory protein |
| | | | | PaiA |
| SEQ ID NO. 1736 | LM-3237.1 | From 842313 | to 842765 | unknown, similar to |
| | | | | transcription regulators |
| SEQ ID NO. 1737 | LM-3239.1 | From 841157 | to 842086 | unknown, similar to |
| | | | | oxidoreductases |
| SEQ ID NO. 1738 | LM-324.1 | From 124495 | to 126363 | unknown, similar to |
| | | | | bacteriophage minor tail |
| | | | | proteins |
| SEQ ID NO. 1739 | LM-3241.1 | From 840200 | to 841072 | unknown, similar to |
| | | | | fructokinases |
| SEQ ID NO. 1740 | LM-3243.3 | From 839520 | to 840062 | unknown |
| SEQ ID NO. 1741 | LM-3244.3 | From 838751 | to 839452 | unknown, similar to |
| | | | | carbonic anhydrase |
| SEQ ID NO. 1742 | LM-3246.1 | From 837548 | to 838621 | Unknown, similar to |
| | | | | spermidine/putrescine- |
| | | | | binding protein |
| SEQ ID NO. 1743 | LM-3248.1 | From 836745 | to 837551 | unknown, similar to |
| | | | | spermidine/putrescine ABC |
| | | | | transporter, permease |
| | | | | protein |
| SEQ ID NO. 1744 | LM-3249.1 | From 835939 | to 836748 | unknown, similar to |
| | | | | spermidine/putrescine ABC |
| | | | | transporter, permease |
| | | | | protein |
| SEQ ID NO. 1745 | LM-3250.1 | From 834845 | to 835939 | unknown, similar to |
| | | | | spermidine/putrescine ABC |
| | | | | transporter, ATP-binding |
| | | | | protein |
| SEQ ID NO. 1746 | LM-3251.2 | From 834287 | to 834829 | unknown, similar to |
| | | | | transcription regulator |
| SEQ ID NO. 1747 | LM-3252.2 | From 1724612 | to 1725565 | unknown, similar to ABC |
| | | | | transporter and adhesion |
| | | | | proteins |
| SEQ ID NO. 1748 | LM-3254.1 | From 1725584 | to 1726993 | similar to O-succinylbenzoic |
| | | | | acid-CoA ligase |
| SEQ ID NO. 1749 | LM-3255.1 | From 1727009 | to 1727827 | unknown, similar to |
| | | | | dihydroxynapthoic acid |
| | | | | synthetase |
| SEQ ID NO. 1750 | LM-3256.1 | From 1727824 | to 1728651 | unknown, similar to prolyl |
| | | | | aminopetidases |
| SEQ ID NO. 1751 | LM-3258.1 | From 1728653 | to 1730395 | unknown, similar to 2- |
| | | | | succinyl-6-hydroxy-2,4- |
| | | | | cyclohexadiene-1- |
| | | | | carboxylate synthase/2- |
| | | | | oxoglutarate decarboxylase |
| SEQ ID NO. 1752 | LM-326.1 | From 124104 | to 124508 | unknown |
| SEQ ID NO. 1753 | LM-3260.2 | From 1730392 | to 1731780 | unknown, similar to |
| | | | | menaquinone-specific |
| | | | | isochorismate synthase |
| SEQ ID NO. 1754 | LM-3261.2 | From 2308741 | to 2311464 | Unknown, similar to |
| | | | | unknown proteins |
| SEQ ID NO. 1755 | LM-3262.1 | From 2311461 | to 2312696 | Unknown, similar to |
| | | | | unknown proteins |
| SEQ ID NO. 1756 | LM-3263.1 | From 2312839 | to 2313192 | Unknown, similar to |
| | | | | unknown proteins |
| SEQ ID NO. 1757 | LM-3265.1 | From 2313274 | to 2314407 | Unknown, similar to |
| | | | | unknown proteins |
| SEQ ID NO. 1758 | LM-3266.1 | From 2314511 | to 2315878 | Unknown, similar to |
| | | | | fumarate hydratase |
| SEQ ID NO. 1759 | LM-3267.1 | From 2315948 | to 2316742 | Unknown, similar to |
| | | | | unknown proteins |
| SEQ ID NO. 1760 | LM-3268.1 | From 2316739 | to 2317644 | Unknown, similar to ABC |
| | | | | transporter (ATP-binding |
| | | | | protein) |
| SEQ ID NO. 1761 | LM-3269.2 | From 2317950 | to 2320094 | Unknown, similar to |
| | | | | penicillin-binding protein |
| SEQ ID NO. 1762 | LM-3274.2 | From 558526 | to 560187 | Unknown, similar to putative |
| | | | | sulfate transporter |
| SEQ ID NO. 1763 | LM-3275.1 | From 558037 | to 558480 | Unknown, similar to B. subtilis |
| | | | | YybC protein |
| SEQ ID NO. 1764 | LM-3276.1 | From 557178 | to 557930 | Unknown, similar to |
| | | | | transcription regulator |
| SEQ ID NO. 1765 | LM-3278.1 | From 555660 | to 556976 | Unknown, similar to 6- |
| | | | | phospho-beta-glucosidase |
| SEQ ID NO. 1766 | LM-328.1 | From 123760 | to 124062 | Unknown |
| SEQ ID NO. 1767 | LM-3281.1 | From 554624 | to 555628 | Unknown, similar to |
| | | | | transcription regulator |
| SEQ ID NO. 1768 | LM-3283.2 | From 553078 | to 554493 | Unknown, similar to |
| | | | | multidrug resistance protein |
| SEQ ID NO. 1769 | LM-3284.1 | From 514004 | to 514381 | Unknown, putative secreted |
| | | | | protein |
| SEQ ID NO. 1770 | LM-3285.1 | From 514681 | to 515058 | Unknown, putative secreted |
| | | | | protein |
| SEQ ID NO. 1771 | LM-3286.1 | From 515391 | to 515750 | Unknown, putative secreted |
| | | | | protein |
| SEQ ID NO. 1772 | LM-3288.1 | From 516076 | to 516636 | Unknown |
| SEQ ID NO. 1773 | LM-329.1 | From 123200 | to 123712 | antigen A |
| SEQ ID NO. 1774 | LM-3290.1 | From 516746 | to 518446 | Unknown, similar to |
| | | | | unknown proteins |
| SEQ ID NO. 1775 | LM-3292.1 | From 518597 | to 519700 | Unknown, similar to |
| | | | | conserved hypothetical |
| | | | | proteins, highly similar to B. subtilis |
| | | | | YloN protein |
| SEQ ID NO. 1776 | LM-3293.1 | From 519789 | to 520268 | Unknown |
| SEQ ID NO. 1777 | LM-3294.2 | From 520426 | to 520791 | Unknown |
| SEQ ID NO. 1778 | LM-3296.2 | From 1561538 | to 1561882 | Unknown, similar to |
| | | | | unknown proteins |
| SEQ ID NO. 1779 | LM-3298.1 | From 1559081 | to 1561432 | Unknown, similar to single- |
| | | | | stranded-DNA-specific |
| | | | | exonuclease (RecJ) |
| SEQ ID NO. 1780 | LM-3299.1 | From 1558570 | to 1559091 | unknown, similar to adenine |
| | | | | phosphoribosyltransferase |
| SEQ ID NO. 1781 | LM-330.1 | From 122798 | to 123187 | antigen B |
| SEQ ID NO. 1782 | LM-3301.1 | From 1556148 | to 1558364 | unknown, similar to |
| | | | | (p)ppGpp synthetase |
| SEQ ID NO. 1783 | LM-3302.1 | From 1555680 | to 1556132 | Unknown, similar to |
| | | | | unknown proteins |
| SEQ ID NO. 1784 | LM-3303.2 | From 1554360 | to 1555643 | Unknown, similar to N- |
| | | | | acetylmuramoyl-L-alanine |
| | | | | amidase |
| SEQ ID NO. 1785 | LM-3305.2 | From 2279524 | to 2279919 | Unknown, similar to |
| | | | | unknown proteins |
| SEQ ID NO. 1786 | LM-3306.1 | From 2280238 | to 2281206 | Unknown, similar to |
| | | | | oligopeptide ABC |
| | | | | transporter (ATP-binding |
| | | | | protein) |
| SEQ ID NO. 1787 | LM-3307.1 | From 2281203 | to 2282279 | Unknown, similar to |
| | | | | oligopeptide ABC |
| | | | | transporter (ATP-binding |
| | | | | protein) |
| SEQ ID NO. 1788 | LM-3309.1 | From 2282297 | to 2283331 | Unknown, similar to |
| | | | | oligopeptide ABC |
| | | | | transporter (permease) |
| SEQ ID NO. 1789 | LM-331.1 | From 122104 | to 122520 | unknown, similar to |
| | | | | Antigen C |
| SEQ ID NO. 1790 | LM-3310.1 | From 2283331 | to 2284260 | Unknown, similar to |
| | | | | oligopeptide ABC |
| | | | | transporter (permease) |
| SEQ ID NO. 1791 | LM-3312.2 | From 2284539 | to 2286215 | Unknown, similar to |
| | | | | pheromone binding protein |
| SEQ ID NO. 1792 | LM-3315.1 | From 1528098 | to 1528727 | unknown, similar to Uridine |
| | | | | kinase |
| SEQ ID NO. 1793 | LM-3316.1 | From 1528724 | to 1529377 | Unknown, similar to O- |
| | | | | methyltransferase |
| SEQ ID NO. 1794 | LM-3319.1 | From 1529454 | to 1530524 | Unknown, similar to |
| | | | | unknown proteins |
| SEQ ID NO. 1795 | LM-332.1 | From 121664 | to 122092 | unknown, similar to |
| | | | | Antigen D |
| SEQ ID NO. 1796 | LM-3320.1 | From 1530727 | to 1531353 | Unknown, similar to |
| | | | | unknown proteins |
| SEQ ID NO. 1797 | LM-3321.1 | From 1531398 | to 1531700 | Unknown, similar to |
| | | | | unknown proteins |
| SEQ ID NO. 1798 | LM-3322.1 | From 1531716 | to 1532132 | Unknown, similar to |
| | | | | unknown proteins |
| SEQ ID NO. 1799 | LM-3323.1 | From 1532129 | to 1532401 | Unknown |
| SEQ ID NO. 1800 | LM-3326.1 | From 1532493 | to 1535132 | alanyl-tRNA synthetase |
| SEQ ID NO. 1801 | LM-3327.1 | From 1535439 | to 1536134 | Unknown, similar to ABC |
| | | | | transporter, ATP-binding |
| | | | | protein |
| SEQ ID NO. 1802 | LM-3329.2 | From 1536143 | to 1537648 | Unknown, similar to |
| | | | | transporter |
| SEQ ID NO. 1803 | LM-333.1 | From 121112 | to 121447 | Unknown, similar to putative |
| | | | | repressor C1 From |
| | | | | lactococcal bacteriophage |
| | | | | Tuc2009 |
| SEQ ID NO. 1804 | LM-3330.2 | From 512291 | to 513268 | Unknown, similar to |
| | | | | oxetanocin A resistance |
| | | | | protein oxrB |
| SEQ ID NO. 1805 | LM-3331.1 | From 511966 | to 512208 | Unknown |
| SEQ ID NO. 1806 | LM-3332.1 | From 511215 | to 511562 | Unknown |
| SEQ ID NO. 1807 | LM-3333.1 | From 510163 | to 511212 | Unknown |
| SEQ ID NO. 1808 | LM-3334.1 | From 509400 | to 510287 | Unknown |
| SEQ ID NO. 1809 | LM-3335.2 | From 507723 | to 508733 | Unknown |
| SEQ ID NO. 1810 | LM-3336.3 | From 506368 | to 506997 | Unknown, weakly similar to |
| | | | | site-specific DNA- |
| | | | | methyltransferase |
| SEQ ID NO. 1811 | LM-3337.3 | From 505351 | to 506223 | Unknown |
| SEQ ID NO. 1812 | LM-3338.2 | From 1487699 | to 1489579 | DNA primase |
| SEQ ID NO. 1813 | LM-3339.1 | From 1489599 | to 1490039 | Unknown, similar to |
| | | | | unknown proteins |
| SEQ ID NO. 1814 | LM-334.1 | From 120654 | to 121106 | Unknown, similar to protein |
| | | | | gp35 From Bacteriophage |
| | | | | A118 |
| SEQ ID NO. 1815 | LM-3340.1 | From 1490229 | to 1491053 | Unknown, similar to |
| | | | | unknown protein |
| SEQ ID NO. 1816 | LM-3341.1 | From 1491087 | to 1493153 | Unknown, similar to glycyl- |
| | | | | tRNA synthetase beta chain |
| SEQ ID NO. 1817 | LM-3342.1 | From 1493146 | to 1494036 | Unknown, similar to glycyl- |
| | | | | tRNA synthetase alpha |
| | | | | chain |
| SEQ ID NO. 1818 | LM-3343.1 | From 1494317 | to 1495084 | Unknown, similar to B. subtilis |
| | | | | RecO protein |
| | | | | involved in DNA repair and |
| | | | | homologous recombination |
| SEQ ID NO. 1819 | LM-3344.1 | From 1495221 | to 1495850 | Unknown |
| SEQ ID NO. 1820 | LM-3345.1 | From 1495899 | to 1496804 | Unknown, similar to GTP |
| | | | | binding proteins |
| SEQ ID NO. 1821 | LM-3347.1 | From 1496801 | to 1497196 | Unknown, similar to cytidine |
| | | | | deaminase |
| SEQ ID NO. 1822 | LM-3348.1 | From 1497221 | to 1497616 | Unknown, similar to |
| | | | | diacylglycerol kinase |
| SEQ ID NO. 1823 | LM-3349.1 | From 1497594 | to 1498079 | Unknown, similar to |
| | | | | unknown proteins |
| SEQ ID NO. 1824 | LM-335.1 | From 119740 | to 120435 | Unknown, weakly similar to |
| | | | | transcription regulators, |
| | | | | Fnr/Crp family |
| SEQ ID NO. 1825 | LM-3355.1 | From 2012582 | to 2013727 | Unknown, similar to similar |
| | | | | to ribosomal protein S1 like |
| | | | | protein |
| SEQ ID NO. 1826 | LM-3356.1 | From 2014086 | to 2014760 | Unknown, similar to |
| | | | | cytidylate kinase |
| SEQ ID NO. 1827 | LM-3357.1 | From 2014776 | to 2015738 | Unknown, similar to |
| | | | | asparaginase |
| SEQ ID NO. 1828 | LM-3359.1 | From 2015819 | to 2016538 | Unknown, similar to |
| | | | | unknown proteins |
| SEQ ID NO. 1829 | LM-336.1 | From 119063 | to 119770 | Unknown |
| SEQ ID NO. 1830 | LM-3360.1 | From 2016858 | to 2018261 | Unknown, similar to ATP- |
| | | | | dependent DNA helicase |
| SEQ ID NO. 1831 | LM-3361.2 | From 2018258 | to 2019265 | Unknown, similar to |
| | | | | unknown proteins |
| SEQ ID NO. 1832 | LM-3362.2 | From 2019420 | to 2019644 | Unknown, similar to |
| | | | | ferredoxin |
| SEQ ID NO. 1833 | LM-3363.3 | From 2019686 | to 2020297 | Unknown, similar to |
| | | | | unknown protein |
| SEQ ID NO. 1834 | LM-3364.2 | From 1867802 | to 1869460 | Unknown, similar to |
| | | | | unknown protein |
| SEQ ID NO. 1835 | LM-3371.2 | From 1872724 | to 1873620 | Unknown, similar to protein- |
| | | | | tyrosine phosphatase |
| SEQ ID NO. 1836 | LM-3373.2 | From 1873764 | to 1875116 | Unknown, similar to signal |
| | | | | recognition particle protein |
| | | | | Ffh |
| SEQ ID NO. 1837 | LM-3374.2 | From 1875129 | to 1875461 | Unknown, similar to |
| | | | | unknown proteins |
| SEQ ID NO. 1838 | LM-3375.2 | From 1358834 | to 1359103 | ribosomal protein S15 |
| SEQ ID NO. 1839 | LM-3376.1 | From 1359352 | to 1361523 | Polynucleotide |
| | | | | phosphorylase (PNPase) |
| SEQ ID NO. 1840 | LM-3377.1 | From 1361564 | to 1362604 | unknown, similar to |
| | | | | conserved hypothetical |
| | | | | proteins |
| SEQ ID NO. 1841 | LM-3378.2 | From 1362765 | to 1363244 | unknown, similar to B. subtilis |
| | | | | YqzC protein |
| SEQ ID NO. 1842 | LM-3379.2 | From 1363272 | to 1363628 | unknown, similar to B. subtilis |
| | | | | YqzD protein |
| SEQ ID NO. 1843 | LM-338.1 | From 117841 | to 118956 | Unknown, similar to lipase |
| SEQ ID NO. 1844 | LM-3380.2 | From 1918681 | to 1919505 | unknown, similar to |
| | | | | unknown proteins |
| SEQ ID NO. 1845 | LM-3381.1 | From 1919525 | to 1920436 | unknown, similar to |
| | | | | conserved hypothetical |
| | | | | proteins |
| SEQ ID NO. 1846 | LM-3382.1 | From 1920436 | to 1920900 | unknown, highly similar to |
| | | | | signal peptidase II |
| SEQ ID NO. 1847 | LM-3384.1 | From 1920982 | to 1922265 | unknown, similar to |
| | | | | conserved hypothetical |
| | | | | proteins |
| SEQ ID NO. 1848 | LM-3385.1 | From 1922439 | to 1923809 | unknown, similar to |
| | | | | conserved hypothetical |
| | | | | proteins |
| SEQ ID NO. 1849 | LM-3387.1 | From 1923825 | to 1924757 | unknown, similar to |
| | | | | adhesion binding proteins |
| | | | | and lipoproteins with |
| | | | | multiple specificity for metal |
| | | | | cations |
| SEQ ID NO. 1850 | LM-3389.1 | From 1924754 | to 1925596 | unknown, similar to integral |
| | | | | membrane proteins, ABC |
| | | | | transporter |
| SEQ ID NO. 1851 | LM-3390.3 | From 1925600 | to 1926322 | unknown, similar to |
| | | | | probable ABC transporter, |
| | | | | ATP-binding proteins |
| SEQ ID NO. 1852 | LM-3391.3 | From 262657 | to 263262 | RNA polymerase sigma-30 |
| | | | | factor (sigma-H) |
| SEQ ID NO. 1853 | LM-3392.1 | From 262064 | to 262576 | Unknown, similar to B. subtilis |
| | | | | Yacp protein |
| SEQ ID NO. 1854 | LM-3393.1 | From 261306 | to 262061 | Unknown, similar to |
| | | | | conserved hypothetical |
| | | | | proteins like to B. subtilis |
| | | | | YacO protein |
| SEQ ID NO. 1855 | LM-3394.1 | From 260896 | to 261306 | Unknown, highly similar to |
| | | | | B. subtilis YazC protein |
| SEQ ID NO. 1856 | LM-3398.1 | From 259477 | to 260892 | cysteinyl-tRNA synthetase |
| SEQ ID NO. 1857 | LM-340.1 | From 116972 | to 117805 | Unknown, similar to |
| | | | | transcriptional regulatory |
| | | | | proteins, AraC family |
| SEQ ID NO. 1858 | LM-3400.1 | From 258856 | to 259470 | unknown, similar to serine |
| | | | | O-acetyltransferase |
| SEQ ID NO. 1859 | LM-3403.3 | From 256983 | to 258458 | unknown, highly similar to |
| | | | | glutamyl-tRNA synthetase |
| SEQ ID NO. 1860 | LM-3404.3 | From 256491 | to 256964 | Unknown, similar to B. subtilis |
| | | | | YacN protein |
| SEQ ID NO. 1861 | LM-3405.2 | From 1862459 | to 1862893 | Unknown, similar to |
| | | | | transcription regulator |
| SEQ ID NO. 1862 | LM-3406.2 | From 1862893 | to 1863480 | Unknown, weakly similar to |
| | | | | Nad(P)h Oxidoreductase |
| | | | | chain B |
| SEQ ID NO. 1863 | LM-3407.1 | From 1863501 | to 1864217 | Unknown, similar to |
| | | | | unknown proteins |
| SEQ ID NO. 1864 | LM-3408.1 | From 1864248 | to 1864637 | Unknown |
| SEQ ID NO. 1865 | LM-3409.1 | From 1864657 | to 1865394 | Unknown, similar to E. coli |
| | | | | tRNA (guanine-N1) |
| | | | | methyltransferase |
| SEQ ID NO. 1866 | LM-3410.1 | From 1865394 | to 1865912 | Unknown, similar to putative |
| | | | | 16S rRNA processing |
| | | | | protein RimM |
| SEQ ID NO. 1867 | LM-3411.1 | From 1865922 | to 1866308 | Unknown, similar to |
| | | | | unknown proteins |
| SEQ ID NO. 1868 | LM-3412.1 | From 1866336 | to 1867070 | Unknown, similar to |
| | | | | unknown proteins |
| SEQ ID NO. 1869 | LM-3414.2 | From 1867427 | to 1867699 | ribosomal protein S16 |
| SEQ ID NO. 1870 | LM-3415.4 | From 1723649 | to 1724122 | unknown, some similarity to |
| | | | | hypothetical proteins |
| SEQ ID NO. 1871 | LM-3416.3 | From 1723356 | to 1723598 | unknown, some similarity to |
| | | | | hypothetical proteins |
| SEQ ID NO. 1872 | LM-3417.3 | From 1722431 | to 1723339 | unknown, similar to L- |
| | | | | lactate dehydrogenases |
| SEQ ID NO. 1873 | LM-342.1 | From 115087 | to 116829 | Unknown, ABC transporter, |
| | | | | ATP-binding protein |
| SEQ ID NO. 1874 | LM-3420.4 | From 1716858 | to 1717160 | Unknown |
| SEQ ID NO. 1875 | LM-3421.2 | From 1748792 | to 1749733 | unknown; similar to |
| | | | | glycerate dehydrogenases |
| SEQ ID NO. 1876 | LM-3422.1 | From 1749878 | to 1751176 | glutamate-1-semialdehyde |
| | | | | aminotransferase |
| SEQ ID NO. 1877 | LM-3424.1 | From 1751283 | to 1752371 | unknown, similar to |
| | | | | hypothetical proteins |
| SEQ ID NO. 1878 | LM-3426.1 | From 1752413 | to 1752940 | unknown, similar to |
| | | | | hypothetical proteins |
| SEQ ID NO. 1879 | LM-3427.1 | From 1753095 | to 1753841 | unknown, similar to glucose |
| | | | | 1-dehydrogenase |
| SEQ ID NO. 1880 | LM-3428.1 | From 1753845 | to 1754942 | unknown, similar to A/G- |
| | | | | specific adenine |
| | | | | glycosylase |
| SEQ ID NO. 1881 | LM-3429.1 | From 1755143 | to 1756123 | unknown, similar to |
| | | | | hypothetical proteins |
| SEQ ID NO. 1882 | LM-343.1 | From 113313 | to 115094 | Unknown, similar to ABC |
| | | | | transporter, ATP-binding |
| | | | | protein |
| SEQ ID NO. 1883 | LM-3430.2 | From 1756156 | to 1756617 | unknown, similar to |
| | | | | deoxyuridine triphosphate |
| | | | | nucleotidohydrolases |
| SEQ ID NO. 1884 | LM-3431.2 | From 2891269 | to 2891652 | Unknown, hypothetical |
| | | | | secreted protein |
| SEQ ID NO. 1885 | LM-3432.2 | From 2890779 | to 2891171 | Unknown, hypothetical |
| | | | | secreted protein |
| SEQ ID NO. 1886 | LM-3433.1 | From 2890274 | to 2890666 | Unknown, hypothetical |
| | | | | secreted protein |
| SEQ ID NO. 1887 | LM-3434.1 | From 2888994 | to 2890277 | Unknown |
| SEQ ID NO. 1888 | LM-3436.1 | From 2888639 | to 2889001 | Unknown |
| SEQ ID NO. 1889 | LM-3439.2 | From 2887499 | to 2888215 | GidB protein |
| SEQ ID NO. 1890 | LM-3442.2 | From 885440 | to 886882 | unknown, similar to |
| | | | | Glutamine binding and |
| | | | | transport protein |
| SEQ ID NO. 1891 | LM-3444.1 | From 886875 | to 887603 | unknown, similar to amino |
| | | | | acid ABC transporter, ATP- |
| | | | | binding protein |
| SEQ ID NO. 1892 | LM-3445.2 | From 887652 | to 889502 | Unknown, similar to |
| | | | | amidases |
| SEQ ID NO. 1893 | LM-3447.3 | From 2068977 | to 2069648 | Unknown, similar to |
| | | | | deoxyribose-phosphate |
| | | | | aldolase |
| SEQ ID NO. 1894 | LM-3448.1 | From 2069710 | to 2070657 | Unknown, similar to |
| | | | | transcription repressor of |
| | | | | dra/nupC/pdp operon DeoR |
| SEQ ID NO. 1895 | LM-3449.1 | From 2070754 | to 2071170 | Unknown, similar to PTS |
| | | | | mannose-specific enzyme |
| | | | | IIA component |
| SEQ ID NO. 1896 | LM-345.1 | From 112386 | to 113288 | Unknown, similar to |
| | | | | transcription regulator |
| SEQ ID NO. 1897 | LM-3450.1 | From 2071187 | to 2072182 | Unknown, similar to opine |
| | | | | catabolism protein |
| SEQ ID NO. 1898 | LM-3451.1 | From 2072205 | to 2073269 | Unknown, weakly similar to |
| | | | | glucosamine-fructose-6- |
| | | | | phosphate |
| | | | | aminotransferase |
| SEQ ID NO. 1899 | LM-3452.1 | From 2073282 | to 2074103 | Unknown, similar to |
| | | | | mannose-specific enzyme |
| | | | | IID component |
| SEQ ID NO. 1900 | LM-3453.1 | From 2074084 | to 2074902 | Unknown, similar to PTS |
| | | | | mannose-specific enzyme |
| | | | | IIC component |
| SEQ ID NO. 1901 | LM-3455.1 | From 2074925 | to 2075392 | Unknown, similar to PTS |
| | | | | mannose-specific enzyme |
| | | | | IIB component |
| SEQ ID NO. 1902 | LM-3456.2 | From 2075412 | to 2076113 | Unknown, similar to |
| | | | | transcription regulator GntR |
| | | | | family |
| SEQ ID NO. 1903 | LM-3458.2 | From 2076115 | to 2076846 | Unknown, similar to |
| | | | | transcription regulator GntR |
| | | | | family |
| SEQ ID NO. 1904 | LM-3459.2 | From 607372 | to 608199 | Unknown, similar histidinol |
| | | | | phosphate phosphatase |
| SEQ ID NO. 1905 | LM-3460.1 | From 608184 | to 608480 | Unknown, similar to |
| | | | | methyltransferase |
| SEQ ID NO. 1906 | LM-3461.1 | From 608557 | to 609588 | Unknown |
| SEQ ID NO. 1907 | LM-3462.2 | From 609629 | to 610924 | Unknown, conserved |
| | | | | hypothetical protein |
| SEQ ID NO. 1908 | LM-3463.2 | From 1776257 | to 1776829 | Unknown |
| SEQ ID NO. 1909 | LM-3465.1 | From 1774991 | to 1775983 | unknown, similar to cell- |
| | | | | shape determining proteins |
| SEQ ID NO. 1910 | LM-3466.1 | From 1773514 | to 1774773 | unknown, similar to |
| | | | | multidrug resistance protein, |
| | | | | integral membrane protein |
| SEQ ID NO. 1911 | LM-3468.3 | From 1772178 | to 1773410 | unknown, highly similar to |
| | | | | aminopeptidases |
| SEQ ID NO. 1912 | LM-3469.2 | From 833117 | to 833587 | Unknown |
| SEQ ID NO. 1913 | LM-3473.1 | From 831025 | to 833073 | Unknown, similar to putative |
| | | | | Na+/H+ antiporter |
| SEQ ID NO. 1914 | LM-3475.1 | From 830259 | to 830897 | Unknown, weakly similar to |
| | | | | GTP-pyrophosphokinase |
| SEQ ID NO. 1915 | LM-3479.2 | From 827666 | to 828016 | Unknown, similar to B. subtilis |
| | | | | YqkB protein |
| SEQ ID NO. 1916 | LM-3480.3 | From 826752 | to 827513 | Unknown |
| SEQ ID NO. 1917 | LM-3481.2 | From 2011128 | to 2012438 | Unknown, similar to |
| | | | | unknown protein |
| SEQ ID NO. 1918 | LM-3482.1 | From 2010089 | to 2011105 | Unknown, similar to |
| | | | | NAD(P)H-dependent |
| | | | | glycerol-3-phosphate |
| | | | | dehydrogenase |
| SEQ ID NO. 1919 | LM-3483.2 | From 2009006 | to 2009986 | Unknown, similar to protein- |
| | | | | tyrosine/serine phosphatase |
| SEQ ID NO. 1920 | LM-3484.1 | From 2008319 | to 2008594 | Unknown, similar to non- |
| | | | | specific DNA-binding |
| | | | | protein HU |
| SEQ ID NO. 1921 | LM-3485.1 | From 2007515 | to 2008084 | Unknown, similar to GTP |
| | | | | cyclohydrolase I |
| SEQ ID NO. 1922 | LM-3486.1 | From 2006689 | to 2007456 | Unknown, similar to |
| | | | | heptaprenyl diphosphate |
| | | | | synthase component I |
| SEQ ID NO. 1923 | LM-3487.1 | From 2005953 | to 2006666 | Unknown, similar to 2- |
| | | | | heptaprenyl-1,4- |
| | | | | naphthoquinone |
| | | | | methyltransferase |
| SEQ ID NO. 1924 | LM-3488.1 | From 2004977 | to 2005942 | Unknown, similar to |
| | | | | heptaprenyl diphosphate |
| | | | | synthase component II |
| | | | | (menaquinone biosynthesis) |
| SEQ ID NO. 1925 | LM-3489.2 | From 2004515 | to 2004958 | Unknown, similar to |
| | | | | nucleoside diphosphate |
| | | | | kinase |
| SEQ ID NO. 1926 | LM-349.1 | From 110013 | to 112283 | Unknown, highly similar to |
| | | | | chitinase B |
| SEQ ID NO. 1927 | LM-3490.2 | From 1029208 | to 1029819 | unknown, similar to |
| | | | | hypothetical protein |
| SEQ ID NO. 1928 | LM-3491.1 | From 1029965 | to 1030756 | Unknown |
| SEQ ID NO. 1929 | LM-3493.3 | From 1030757 | to 1032229 | unknown, similar to |
| | | | | PHYTOENE |
| | | | | DEHYDROGENASE |
| | | | | (EC 1.3.—.—) (PHYTOENE |
| | | | | DESATURASE) |
| SEQ ID NO. 1930 | LM-3494.3 | From 1032333 | to 1032617 | Unknown, similar to B. subtilis |
| | | | | protein YkvS |
| SEQ ID NO. 1931 | LM-3495.3 | From 1032924 | to 1033190 | PHOSPHOCARRIER |
| | | | | PROTEIN HPR |
| | | | | (HISTIDINE-CONTAINING |
| | | | | PROTEIN). |
| SEQ ID NO. 1932 | LM-3497.4 | From 1033190 | to 1034908 | Phosphotransferase system |
| | | | | enzyme I |
| SEQ ID NO. 1933 | LM-3498.1 | From 1035021 | to 1036055 | Unknown, conserved |
| | | | | hypothetical protein |
| SEQ ID NO. 1934 | LM-35.1 | From 2735279 | to 2735953 | Unknown, similar to |
| | | | | ribulose-5-phosphate 3- |
| | | | | epimerase |
| SEQ ID NO. 1935 | LM-3500.1 | From 1036068 | to 1036928 | Unknown, similar to 3- |
| | | | | hydroxyisobutyrate |
| | | | | dehydrogenase (B. subtilis |
| | | | | YkwC protein) |
| SEQ ID NO. 1936 | LM-3501.2 | From 1036971 | to 1038116 | Unknown, similar to |
| | | | | aminotransferases (to B. subtilis |
| | | | | PatA protein) |
| SEQ ID NO. 1937 | LM-3504.2 | From 641208 | to 642308 | Unknown, similar to cell |
| | | | | surface protein |
| SEQ ID NO. 1938 | LM-3505.1 | From 642414 | to 642914 | Unknown, weakly similar to |
| | | | | transcription regulator |
| SEQ ID NO. 1939 | LM-3506.1 | From 642962 | to 643348 | Unknown |
| SEQ ID NO. 1940 | LM-3507.1 | From 643400 | to 643744 | Unknown, similar to B. subtilis |
| | | | | YvlA protein |
| SEQ ID NO. 1941 | LM-3508.2 | From 643891 | to 645231 | Unknown, conserved |
| | | | | hypothetical membrane |
| | | | | protein |
| SEQ ID NO. 1942 | LM-351.2 | From 109349 | to 109720 | Unknown |
| SEQ ID NO. 1943 | LM-3510.2 | From 2145652 | to 2146044 | Unknown |
| SEQ ID NO. 1944 | LM-3511.1 | From 2146089 | to 2146298 | Unknown |
| SEQ ID NO. 1945 | LM-3512.1 | From 2146449 | to 2147426 | Unknown, similar to |
| | | | | conjugated bile acid |
| | | | | hydrolase |
| SEQ ID NO. 1946 | LM-3514.2 | From 2147684 | to 2149312 | Class I heat-shock protein |
| | | | | (chaperonin) GroEL |
| SEQ ID NO. 1947 | LM-3517.2 | From 1888615 | to 1890273 | Unknown, similar to |
| | | | | unknown proteins |
| SEQ ID NO. 1948 | LM-3518.1 | From 1887913 | to 1888575 | Unknown, similar to |
| | | | | phosphoglycerate |
| | | | | dehydrogenase |
| SEQ ID NO. 1949 | LM-3519.1 | From 1886887 | to 1887777 | Unknown, similar to L- |
| | | | | serine dehydratase |
| SEQ ID NO. 1950 | LM-352.2 | From 108624 | to 109256 | Unknown, similar to NADH |
| | | | | oxidase |
| SEQ ID NO. 1951 | LM-3520.2 | From 1884846 | to 1886894 | Unknown, similar to ATP- |
| | | | | dependent DNA helicase |
| | | | | recG |
| SEQ ID NO. 1952 | LM-3523.1 | From 2143109 | to 2144734 | Unknown, similar to copper |
| | | | | export proteins |
| SEQ ID NO. 1953 | LM-3524.1 | From 2142480 | to 2143097 | Unknown, similar to |
| | | | | unknown protein |
| SEQ ID NO. 1954 | LM-3525.1 | From 2141820 | to 2142461 | Unknown, similar to |
| | | | | unknown protein |
| SEQ ID NO. 1955 | LM-3527.1 | From 2141071 | to 2141814 | Unknown, similar to |
| | | | | potassium channel subunit |
| SEQ ID NO. 1956 | LM-3528.2 | From 2140046 | to 2140963 | Unknown, similar to heme O |
| | | | | oxygenase |
| SEQ ID NO. 1957 | LM-353.1 | From 108320 | to 108610 | Unknown |
| SEQ ID NO. 1958 | LM-3533.1 | From 1053426 | to 1054277 | unknown |
| SEQ ID NO. 1959 | LM-3534.1 | From 1054319 | to 1055284 | Unknown, similar to B. subtilis |
| | | | | LytR protein |
| SEQ ID NO. 1960 | LM-3535.1 | From 1055393 | to 1057060 | Unknown, similar to |
| | | | | conserved hypothetical |
| | | | | proteins (in particular B. subtilis |
| | | | | YkqC) |
| SEQ ID NO. 1961 | LM-3537.1 | From 1057761 | to 1058531 | Unknown, similar to |
| | | | | conserved hypothetical |
| | | | | proteins |
| SEQ ID NO. 1962 | LM-3538.3 | From 1058580 | to 1059608 | Unknown, similar to |
| | | | | transcriptional regulator, |
| | | | | LacI family |
| SEQ ID NO. 1963 | LM-3539.1 | From 2286839 | to 2287183 | Unknown |
| SEQ ID NO. 1964 | LM-354.1 | From 107915 | to 108208 | Unknown, similar to |
| | | | | transcription regulator |
| SEQ ID NO. 1965 | LM-3541.1 | From 2287518 | to 2288513 | Tryptophanyl-tRNA |
| | | | | synthetase |
| SEQ ID NO. 1966 | LM-3543.1 | From 2288571 | to 2288987 | Unknown, similar to |
| | | | | unknown protein |
| SEQ ID NO. 1967 | LM-3544.1 | From 2288971 | to 2289423 | Unknown, similar to |
| | | | | transcription regulator |
| SEQ ID NO. 1968 | LM-3547.1 | From 2289545 | to 2290786 | Unknown, similar to 3- |
| | | | | oxoacyl-acyl-carrier protein |
| | | | | synthase |
| SEQ ID NO. 1969 | LM-3549.2 | From 2290989 | to 2291927 | Unknown, similar to 3- |
| | | | | oxoacyl-acyl-carrier protein |
| | | | | synthase |
| SEQ ID NO. 1970 | LM-355.1 | From 107499 | to 107849 | Unknown |
| SEQ ID NO. 1971 | LM-3551.1 | From 1242100 | to 1244508 | Phenylalanyl-tRNA |
| | | | | synthetase beta subunit |
| SEQ ID NO. 1972 | LM-3553.2 | From 1241048 | to 1242100 | Phenylalanyl-tRNA |
| | | | | synthetase beta subunit |
| SEQ ID NO. 1973 | LM-3555.2 | From 2305632 | to 2305994 | Unknown, similar to |
| | | | | unknown protein |
| SEQ ID NO. 1974 | LM-3556.1 | From 2305180 | to 2305602 | Unknown, similar to |
| | | | | histidine triad (HIT) protein |
| SEQ ID NO. 1975 | LM-3557.1 | From 2304267 | to 2305019 | Unknown, similar to ABC |
| | | | | transporter (ATP-binding |
| | | | | protein) |
| SEQ ID NO. 1976 | LM-3558.1 | From 2303060 | to 2304283 | Unknown, similar to ABC |
| | | | | transporter (membrane |
| | | | | protein) |
| SEQ ID NO. 1977 | LM-3559.1 | From 2302518 | to 2303021 | Unknown, similar to |
| | | | | unknown protein |
| SEQ ID NO. 1978 | LM-356.1 | From 106988 | to 107377 | unknown |
| SEQ ID NO. 1979 | LM-3560.1 | From 2301260 | to 2302321 | Unknown, similar to |
| | | | | uroporphyrinogen III |
| | | | | decarboxylase |
| SEQ ID NO. 1980 | LM-3561.1 | From 2300334 | to 2301263 | Unknown, similar to |
| | | | | ferrochelatase |
| SEQ ID NO. 1981 | LM-3562.2 | From 2299833 | to 2300177 | Unknown |
| SEQ ID NO. 1982 | LM-3563.2 | From 1548034 | to 1548456 | Unknown, similar to |
| | | | | unknown protein |
| SEQ ID NO. 1983 | LM-3565.2 | From 1548701 | to 1549906 | Unknown, similar to |
| | | | | ammonium transporter |
| | | | | NrgA |
| SEQ ID NO. 1984 | LM-3566.1 | From 1549920 | to 1550285 | Unknown, similar to |
| | | | | nitrogen regulatory PII |
| | | | | protein |
| SEQ ID NO. 1985 | LM-3567.1 | From 1550435 | to 1550815 | Unknown |
| SEQ ID NO. 1986 | LM-3569.1 | From 1550854 | to 1552629 | Aspartyl-tRNA synthetase |
| SEQ ID NO. 1987 | LM-357.1 | From 105951 | to 106862 | Unknown, similar to PTS |
| | | | | system mannose-specific |
| | | | | factor IID |
| SEQ ID NO. 1988 | LM-3570.2 | From 1552632 | to 1553909 | Histidyl-tRNA synthetase |
| SEQ ID NO. 1989 | LM-3571.4 | From 1713234 | to 1715099 | Unknown, similar to |
| | | | | asparagine synthetase |
| SEQ ID NO. 1990 | LM-3572.1 | From 1712634 | to 1713209 | Unknown, similar to |
| | | | | conserved hypothetical |
| | | | | protein |
| SEQ ID NO. 1991 | LM-3573.2 | From 1711672 | to 1712637 | Unknown, similar to |
| | | | | conserved hypothetical |
| | | | | proteins |
| SEQ ID NO. 1992 | LM-3574.3 | From 896003 | to 897028 | Unknown, similar to |
| | | | | transcription regulator lacl |
| | | | | family |
| SEQ ID NO. 1993 | LM-3576.1 | From 895278 | to 895991 | Unknown, similar to |
| | | | | carboxylesterase |
| SEQ ID NO. 1994 | LM-3577.1 | From 893838 | to 895211 | UDP-N- |
| | | | | acetylmuramoylalanyl-D- |
| | | | | glutamyl-2,6- |
| | | | | diaminopimelate-D-alanyl- |
| | | | | D-alanyl ligase |
| SEQ ID NO. 1995 | LM-3578.1 | From 892662 | to 893774 | D-alanine-D-alanine ligase |
| SEQ ID NO. 1996 | LM-3579.1 | From 892192 | to 892512 | Unknown, similar to E. coli |
| | | | | SugE protein |
| | | | | (transmembrane |
| | | | | chaperone) |
| SEQ ID NO. 1997 | LM-358.1 | From 105123 | to 105929 | Unknown, similar to PTS |
| | | | | system mannose-specific, |
| | | | | factor IIC |
| SEQ ID NO. 1998 | LM-3581.1 | From 891848 | to 892189 | Unknown, similar to E. coli |
| | | | | SugE protein |
| | | | | (transmembrane |
| | | | | chaperone) |
| SEQ ID NO. 1999 | LM-3582.1 | From 891277 | to 891831 | Unknown, similar to |
| | | | | transcription regulator |
| | | | | TetR/AcrR family |
| SEQ ID NO. 2000 | LM-3583.3 | From 890439 | to 891197 | Unknown |
| SEQ ID NO. 2001 | LM-3585.2 | From 1926935 | to 1928425 | Unknown, similar to |
| | | | | carboxy-terminal processing |
| | | | | proteinase |
| SEQ ID NO. 2002 | LM-3588.1 | From 1928798 | to 1931011 | Unknown, similar to heavy |
| | | | | metal-transporting ATPases |
| SEQ ID NO. 2003 | LM-3589.1 | From 1931026 | to 1931319 | Unknown, similar to |
| | | | | conserved hypothetical |
| | | | | proteins |
| SEQ ID NO. 2004 | LM-359.1 | From 104134 | to 105099 | Unknown, similar to PTS |
| | | | | system mannose-specific, |
| | | | | factor IIAB |
| SEQ ID NO. 2005 | LM-3590.2 | From 1931442 | to 1932266 | Unknown, similar to similar |
| | | | | to D-alanyl-D-alanine |
| | | | | carboxypeptidases |
| SEQ ID NO. 2006 | LM-3591.3 | From 1932324 | to 1933025 | Purine nucleoside |
| | | | | phosphorylase |
| SEQ ID NO. 2007 | LM-3592.2 | From 2487261 | to 2488337 | Unknown |
| SEQ ID NO. 2008 | LM-3593.1 | From 2488379 | to 2489209 | Unknown, conserved |
| | | | | lipoprotein |
| SEQ ID NO. 2009 | LM-3596.1 | From 2489274 | to 2489948 | Unknown, similar to ABC |
| | | | | transporter, permease |
| | | | | protein |
| SEQ ID NO. 2010 | LM-3597.1 | From 2489945 | to 2490967 | Unknown, similar to ABC |
| | | | | transporter (ATP-binding |
| | | | | protein) |
| SEQ ID NO. 2011 | LM-3599.1 | From 2491552 | to 2492694 | Unknown, similar to two- |
| | | | | component sensor histidine |
| | | | | kinase |
| SEQ ID NO. 2012 | LM-36.1 | From 2735962 | to 2736402 | Unknown, similar to ribose |
| | | | | 5-phosphate epimerase |
| SEQ ID NO. 2013 | LM-360.1 | From 103204 | to 103836 | unknown |
| SEQ ID NO. 2014 | LM-3600.1 | From 2492684 | to 2493379 | Unknown, similar to two- |
| | | | | component response |
| | | | | regulator |
| SEQ ID NO. 2015 | LM-3601.3 | From 2493454 | to 2494329 | Unknown, conserved |
| | | | | hypothetical protein |
| SEQ ID NO. 2016 | LM-3602.2 | From 1435635 | to 1437866 | Pyruvate-formate lyase |
| SEQ ID NO. 2017 | LM-3603.1 | From 1437943 | to 1438689 | Pyruvate-formate lyase |
| | | | | activating enzyme |
| SEQ ID NO. 2018 | LM-3604.1 | From 1438724 | to 1439254 | Unknown, similar to |
| | | | | unknown proteins |
| SEQ ID NO. 2019 | LM-3605.2 | From 1439421 | to 1440614 | Unknown, similar to |
| | | | | multidrug-efflux transporter |
| SEQ ID NO. 2020 | LM-3608.2 | From 1104863 | to 1105864 | Unknown, similar to |
| | | | | TEICHOIC ACID |
| | | | | TRANSLOCATION ATP- |
| | | | | BINDING PROTEIN TAGH |
| | | | | (ABC transporter) |
| SEQ ID NO. 2021 | LM-361.1 | From 102468 | to 103028 | unknown |
| SEQ ID NO. 2022 | LM-3612.1 | From 1107975 | to 1109663 | Unknown, similar to |
| | | | | TEICHOIC ACID |
| | | | | BIOSYNTHESIS PROTEIN |
| | | | | B PRECURSOR |
| SEQ ID NO. 2023 | LM-3613.1 | From 1109705 | to 1110577 | Unknown, similar to putative |
| | | | | UDP-glucose |
| | | | | pyrophosphorylases |
| SEQ ID NO. 2024 | LM-3614.3 | From 1110769 | to 1113627 | Unknown, similar to B. subtilis |
| | | | | YfhO protein |
| SEQ ID NO. 2025 | LM-3615.2 | From 1277312 | to 1278025 | Unknown, similar to |
| | | | | transcription regulator GntR |
| | | | | family |
| SEQ ID NO. 2026 | LM-3616.1 | From 1278056 | to 1279702 | Unknown, similar to |
| | | | | alpha, alpha- |
| | | | | phosphotrehalase |
| SEQ ID NO. 2027 | LM-3617.3 | From 1279721 | to 1281205 | Unknown, similar to PTS |
| | | | | system trehalose specific |
| | | | | enzyme IIBC |
| SEQ ID NO. 2028 | LM-3618.3 | From 1434661 | to 1435209 | Unknown, similar to putative |
| | | | | anti-terminator regulatory |
| | | | | protein |
| SEQ ID NO. 2029 | LM-362.1 | From 102155 | to 102478 | Unknown, similar to ATP |
| | | | | synthase epsilon chain |
| SEQ ID NO. 2030 | LM-3620.2 | From 1432839 | to 1434644 | DNA mismatch repair |
| | | | | protein |
| SEQ ID NO. 2031 | LM-3623.1 | From 1430237 | to 1432819 | DNA mismatch repair |
| | | | | (recognition) |
| SEQ ID NO. 2032 | LM-3624.2 | From 1429765 | to 1430127 | Unknown, similar to B. subtilis |
| | | | | YmcA protein |
| SEQ ID NO. 2033 | LM-3625.2 | From 758656 | to 759396 | Unknown, similar to |
| | | | | riboflavin kinase/FAD |
| | | | | synthase |
| SEQ ID NO. 2034 | LM-3626.2 | From 756738 | to 758543 | Unknown, similar to L- |
| | | | | glutamine-D-fructose-6- |
| | | | | phosphate |
| | | | | amidotransferase |
| SEQ ID NO. 2035 | LM-3627.2 | From 598020 | to 598925 | Unknown, putative |
| | | | | membrane protein |
| SEQ ID NO. 2036 | LM-3628.2 | From 598968 | to 600344 | Unknown, similar to NADP- |
| | | | | specific glutamate |
| | | | | dehydrogenase |
| SEQ ID NO. 2037 | LM-363.1 | From 100772 | to 102142 | Unknown, similar to ATP |
| | | | | synthase beta chain |
| SEQ ID NO. 2038 | LM-3630.4 | From 134782 | to 136290 | Unknown, similar to inosine |
| | | | | monophosphate |
| | | | | dehydrogenase |
| SEQ ID NO. 2039 | LM-3631.2 | From 133961 | to 134710 | Unknown, conserved |
| | | | | hypothetical protein |
| SEQ ID NO. 2040 | LM-3632.2 | From 2292047 | to 2293174 | Unknown, similar to N- |
| | | | | acetylmuramoyl-L-alanine |
| | | | | amidase and to internalin B |
| SEQ ID NO. 2041 | LM-3633.1 | From 2293775 | to 2294464 | Unknown, similar to |
| | | | | phosphoglyceromutase 1 |
| SEQ ID NO. 2042 | LM-3635.2 | From 2294555 | to 2297155 | Unknown, similar to |
| | | | | endopeptidase Clp ATP- |
| | | | | binding chain B (ClpB) |
| SEQ ID NO. 2043 | LM-3636.2 | From 1281321 | to 1281773 | unknown |
| SEQ ID NO. 2044 | LM-3637.1 | From 1281820 | to 1282284 | unknown |
| SEQ ID NO. 2045 | LM-3638.1 | From 1282473 | to 1283393 | unknown |
| SEQ ID NO. 2046 | LM-3639.1 | From 1283413 | to 1284660 | Gamma-glutamyl phosphate |
| | | | | reductase |
| SEQ ID NO. 2047 | LM-364.1 | From 99902 | to 100771 | Unknown, similar to ATP |
| | | | | synthase gamma chain |
| SEQ ID NO. 2048 | LM-3640.1 | From 1284644 | to 1285474 | Gamma-glutamyl kinase |
| SEQ ID NO. 2049 | LM-3643.3 | From 1285610 | to 1286749 | unknown |
| SEQ ID NO. 2050 | LM-3644.4 | From 1286830 | to 1287225 | Unknown, similar to |
| | | | | transcriptional regulator |
| | | | | (phage-related) |
| SEQ ID NO. 2051 | LM-3645.2 | From 1765915 | to 1767294 | unknown, similar to similar |
| | | | | to RNA methyltransferases |
| SEQ ID NO. 2052 | LM-3647.1 | From 1765499 | to 1765900 | unknown, similar to |
| | | | | glutathione transferase - |
| | | | | fosfomycin resistance |
| | | | | protein |
| SEQ ID NO. 2053 | LM-3648.1 | From 1765117 | to 1765470 | unknown |
| SEQ ID NO. 2054 | LM-3649.1 | From 1763941 | to 1764843 | unknown, some similarities |
| | | | | to methyl-accepting |
| | | | | chemotaxis proteins |
| SEQ ID NO. 2055 | LM-3650.1 | From 1763209 | to 1763751 | unknown, similar to |
| | | | | ribosomal-protein-alanine |
| | | | | N-acetyltransferase |
| SEQ ID NO. 2056 | LM-3652.2 | From 1762200 | to 1763165 | unknown, similar to putative |
| | | | | transmembrane proteins |
| SEQ ID NO. 2057 | LM-3656.2 | From 2840286 | to 2841896 | Unknown, similar to ABC |
| | | | | transporter (ATP-binding |
| | | | | protein) |
| SEQ ID NO. 2058 | LM-3657.2 | From 2839698 | to 2840228 | Unknown, similar to |
| | | | | unknown protein |
| SEQ ID NO. 2059 | LM-3658.1 | From 1595227 | to 1597149 | threonyl-tRNA synthetase |
| SEQ ID NO. 2060 | LM-3659.1 | From 1597499 | to 1598422 | primosome component |
| | | | | (helicase loader) Dnal |
| SEQ ID NO. 2061 | LM-366.1 | From 98409 | to 99905 | Unknown, similar to ATP |
| | | | | synthase alpha chain |
| SEQ ID NO. 2062 | LM-3660.2 | From 1598432 | to 1599808 | chromosome replication |
| | | | | initiation/membrane |
| | | | | attachment protein DnaB |
| SEQ ID NO. 2063 | LM-3662.3 | From 2708276 | to 2709358 | Unknown, conserved |
| | | | | hypothetical lipoprotein |
| SEQ ID NO. 2064 | LM-3663.2 | From 2707235 | to 2708194 | Unknown, weakly similar to |
| | | | | E. coli MenA protein |
| SEQ ID NO. 2065 | LM-3664.1 | From 2706415 | to 2707215 | Unknown, similar to B. subtilis |
| | | | | YbaF protein |
| SEQ ID NO. 2066 | LM-3666.1 | From 2705746 | to 2706054 | ribosomal protein S10 |
| SEQ ID NO. 2067 | LM-3667.1 | From 2705082 | to 2705711 | ribosomal protein L3 |
| SEQ ID NO. 2068 | LM-3668.1 | From 2704433 | to 2705056 | ribosomal protein L4 |
| SEQ ID NO. 2069 | LM-3669.1 | From 2704149 | to 2704433 | ribosomal protein L23 |
| SEQ ID NO. 2070 | LM-367.1 | From 97381 | to 98412 | Unknown, weakly similar to |
| | | | | ATP synthase delta chain |
| SEQ ID NO. 2071 | LM-3671.2 | From 2703275 | to 2704108 | ribosomal protein L2 |
| SEQ ID NO. 2072 | LM-3673.2 | From 2033587 | to 2034528 | Unknown, similar to |
| | | | | ferrichrome binding protein |
| SEQ ID NO. 2073 | LM-3674.1 | From 2032460 | to 2033485 | Unknown, similar to |
| | | | | ferrichrome ABC transporter |
| | | | | (permease) |
| SEQ ID NO. 2074 | LM-3676.2 | From 2031438 | to 2032460 | Unknown, similar to |
| | | | | ferrichrome ABC transporter |
| | | | | (permease) |
| SEQ ID NO. 2075 | LM-3677.2 | From 1756864 | to 1757673 | unknown, similar to |
| | | | | hypothetical proteins |
| SEQ ID NO. 2076 | LM-3678.1 | From 1757771 | to 1758673 | unknown, similar to CDP- |
| | | | | abequose synthase |
| SEQ ID NO. 2077 | LM-3679.2 | From 1758694 | to 1761291 | unknown, similar to putative |
| | | | | membrane proteins |
| SEQ ID NO. 2078 | LM-368.1 | From 97127 | to 97369 | Unknown, similar to ATP |
| | | | | synthase C chain |
| SEQ ID NO. 2079 | LM-3680.2 | From 1761318 | to 1761992 | unknown, similar to |
| | | | | unknown proteins |
| SEQ ID NO. 2080 | LM-3681.2 | From 1708312 | to 1708518 | unknown |
| SEQ ID NO. 2081 | LM-3684.3 | From 1708857 | to 1711268 | leucyl-tRNA synthetase |
| SEQ ID NO. 2082 | LM-3692.1 | From 2161161 | to 2162054 | Unknown |
| SEQ ID NO. 2083 | LM-3693.1 | From 2160538 | to 2161164 | unknown |
| SEQ ID NO. 2084 | LM-3694.1 | From 2160043 | to 2160432 | Unknown, similar to |
| | | | | unknown protein |
| SEQ ID NO. 2085 | LM-3695.2 | From 980177 | to 981307 | Unknown, similar to B. subtilis |
| | | | | ComEC protein |
| SEQ ID NO. 2086 | LM-3696.1 | From 981743 | to 982960 | unknown, hypothetical |
| | | | | transport protein |
| SEQ ID NO. 2087 | LM-3697.2 | From 982957 | to 983646 | unknown, similar to |
| | | | | transcription regulator |
| SEQ ID NO. 2088 | LM-3698.2 | From 983768 | to 984814 | unknown, conserved |
| | | | | hypothetical membrane |
| | | | | protein |
| SEQ ID NO. 2089 | LM-3699.2 | From 2658024 | to 2658851 | Unknown, conserved |
| | | | | hypothetical protein |
| SEQ ID NO. 2090 | LM-370.1 | From 94764 | to 97070 | unknown |
| SEQ ID NO. 2091 | LM-3701.3 | From 1936764 | to 1937603 | unknown, similar to |
| | | | | hypothetical proteins |
| SEQ ID NO. 2092 | LM-3703.3 | From 1935964 | to 1936749 | unknown, similar to |
| | | | | hypothetical proteins |
| SEQ ID NO. 2093 | LM-3704.3 | From 1935334 | to 1935948 | unknown, similar to |
| | | | | hypothetical proteins |
| SEQ ID NO. 2094 | LM-3705.1 | From 1934765 | to 1935298 | unknown, similar to peptidyl |
| | | | | methionine sulfoxide |
| | | | | reductases |
| SEQ ID NO. 2095 | LM-3706.1 | From 1934321 | to 1934758 | unknown, similar to |
| | | | | transcriptional regulator |
| | | | | (PilB family) |
| SEQ ID NO. 2096 | LM-3707.3 | From 1933333 | to 1934238 | unknown, similar to |
| | | | | dehydogenases and |
| | | | | hypothetical proteins |
| SEQ ID NO. 2097 | LM-3708.2 | From 822611 | to 823531 | Unknown, conserved |
| | | | | hypothetical protein |
| SEQ ID NO. 2098 | LM-3709.1 | From 821886 | to 822527 | Unknown, similar to B. subtilis |
| | | | | YwnB protein |
| SEQ ID NO. 2099 | LM-3710.1 | From 821070 | to 821771 | Unknown, similar to |
| | | | | conserved hypothetical |
| | | | | protein |
| SEQ ID NO. 2100 | LM-3711.3 | From 820145 | to 821035 | Unknown, similar to |
| | | | | conserved hypothetical |
| | | | | protein |
| SEQ ID NO. 2101 | LM-3712.2 | From 1767359 | to 1767739 | unknown, similar to |
| | | | | conserved hypothetical |
| | | | | proteins |
| SEQ ID NO. 2102 | LM-3713.1 | From 1767846 | to 1768490 | unknown, similar to |
| | | | | deoxyguanosine |
| | | | | kinase/deoxyadenosine |
| | | | | kinase(I) subunit |
| SEQ ID NO. 2103 | LM-3714.1 | From 1768520 | to 1769389 | unknown, similar to |
| | | | | transport proteins |
| SEQ ID NO. 2104 | LM-3715.1 | From 1769720 | to 1770526 | unknown, similar to |
| | | | | aminoglycoside N3- |
| | | | | acetyltransferases |
| SEQ ID NO. 2105 | LM-3716.2 | From 1770648 | to 1771406 | unknown, similar to |
| | | | | methionine |
| | | | | aminopeptidases |
| SEQ ID NO. 2106 | LM-3717.2 | From 1818007 | to 1818516 | Unknown |
| SEQ ID NO. 2107 | LM-3718.1 | From 1817112 | to 1817879 | Unknown, similar to ABC |
| | | | | transporter (ATP-binding |
| | | | | protein) |
| SEQ ID NO. 2108 | LM-3719.3 | From 1815143 | to 1817122 | Unknown, similar to ABC |
| | | | | transporter (permease) |
| SEQ ID NO. 2109 | LM-3720.2 | From 1293088 | to 1293654 | unknown, similar to type-I |
| | | | | signal peptidase |
| SEQ ID NO. 2110 | LM-3722.1 | From 1291710 | to 1292969 | ATP-dependent Clp |
| | | | | protease ATP-binding |
| | | | | subunit ClpX |
| SEQ ID NO. 2111 | LM-3724.1 | From 1290241 | to 1291524 | trigger factor (prolyl |
| | | | | isomerase) |
| SEQ ID NO. 2112 | LM-3725.2 | From 1289188 | to 1290126 | unknown |
| SEQ ID NO. 2113 | LM-3726.3 | From 1703330 | to 1703824 | unknown, putative cell |
| | | | | surface protein |
| SEQ ID NO. 2114 | LM-3727.1 | From 1704618 | to 1705181 | unknown, similar to |
| | | | | unknown proteins |
| SEQ ID NO. 2115 | LM-3728.1 | From 1705199 | to 1705630 | unknown |
| SEQ ID NO. 2116 | LM-373.1 | From 88888 | to 94767 | Unknown |
| SEQ ID NO. 2117 | LM-3732.2 | From 1706204 | to 1707088 | similar to translation |
| | | | | elongation factor |
| SEQ ID NO. 2118 | LM-3733.3 | From 1707168 | to 1707917 | 30S ribosomal protein S2 |
| SEQ ID NO. 2119 | LM-3735.3 | From 1645412 | to 1645936 | Unknown, similar to general |
| | | | | stress protein |
| SEQ ID NO. 2120 | LM-3736.3 | From 1644106 | to 1645191 | 3-deoxy-D-arabino- |
| | | | | heptulosonate 7-phosphate |
| | | | | synthase |
| SEQ ID NO. 2121 | LM-3737.1 | From 1642865 | to 1643872 | catabolite control protein A |
| SEQ ID NO. 2122 | LM-3739.2 | From 1641284 | to 1642543 | tyrosyl-tRNA synthetase |
| SEQ ID NO. 2123 | LM-374.1 | From 88121 | to 88810 | unknown |
| SEQ ID NO. 2124 | LM-3746.2 | From 1428442 | to 1428939 | Unknown, similar to N- |
| | | | | acetyltransferase |
| SEQ ID NO. 2125 | LM-3747.1 | From 1426766 | to 1428328 | Unknown, similar to |
| | | | | unknown protein |
| SEQ ID NO. 2126 | LM-3749.2 | From 1425419 | to 1426465 | Recombination protein recA |
| SEQ ID NO. 2127 | LM-375.1 | From 86747 | to 87730 | Unknown, similar to |
| | | | | oxidoreductases |
| SEQ ID NO. 2128 | LM-3750.2 | From 356890 | to 357705 | Unknown, similar to |
| | | | | transposase |
| SEQ ID NO. 2129 | LM-3754.2 | From 360172 | to 360507 | Unknown |
| SEQ ID NO. 2130 | LM-3756.2 | From 1407859 | to 1408818 | Unknown, similar to |
| | | | | unknown protein |
| SEQ ID NO. 2131 | LM-3757.2 | From 1406742 | to 1407818 | Unknown, similar to |
| | | | | unknown protein |
| SEQ ID NO. 2132 | LM-3758.2 | From 824936 | to 826396 | Unknown, similar to lysine- |
| | | | | specific permease |
| SEQ ID NO. 2133 | LM-3759.2 | From 824423 | to 824884 | Unknown |
| SEQ ID NO. 2134 | LM-376.1 | From 86323 | to 86691 | Unknown, similar to |
| | | | | transcription regulator |
| | | | | (merR family) |
| SEQ ID NO. 2135 | LM-3763.1 | From 1659457 | to 1660926 | Unknown, similar to |
| | | | | multidrug-efflux transporter |
| SEQ ID NO. 2136 | LM-3764.1 | From 1660923 | to 1661363 | Unknown, similar to |
| | | | | transcription regulator MarR |
| | | | | family |
| SEQ ID NO. 2137 | LM-3766.3 | From 1661588 | to 1662457 | D-Amino Acid |
| | | | | Aminotransferase |
| SEQ ID NO. 2138 | LM-3767.2 | From 2212384 | to 2212620 | unknown |
| SEQ ID NO. 2139 | LM-3768.2 | From 2213218 | to 2215041 | Unknown, similar to |
| | | | | unknown protein |
| SEQ ID NO. 2140 | LM-3769.2 | From 2306177 | to 2306716 | Unknown |
| SEQ ID NO. 2141 | LM-377.1 | From 85902 | to 86228 | Unknown |
| SEQ ID NO. 2142 | LM-3770.2 | From 2306833 | to 2307714 | Unknown, similar to post- |
| | | | | translocation molecular |
| | | | | chaperone |
| SEQ ID NO. 2143 | LM-3772.2 | From 2307755 | to 2308696 | Unknown, similar to S. aureus |
| | | | | Cbf1 protein |
| SEQ ID NO. 2144 | LM-3774.3 | From 991810 | to 992865 | unknown, similar to |
| | | | | UNDECAPRENYL- |
| | | | | PHOSPHATE N- |
| | | | | ACETYLGLUCOSAMINYL |
| | | | | TRANSFERASE |
| SEQ ID NO. 2145 | LM-3775.1 | From 990968 | to 991690 | unknown, similar to |
| | | | | transcription regulator |
| | | | | (GntR family) |
| SEQ ID NO. 2146 | LM-3776.2 | From 990248 | to 990952 | GLUCOSAMINE-6- |
| | | | | PHOSPHATE ISOMERASE |
| | | | | (EC 5.3.1.10) |
| | | | | (GLUCOSAMINE-6- |
| | | | | PHOSPHATE |
| | | | | DEAMINASE) (GNPDA) |
| | | | | (GLCN6P DEAMINASE). |
| SEQ ID NO. 2147 | LM-3778.2 | From 1861907 | to 1862251 | ribosomal protein L19 |
| SEQ ID NO. 2148 | LM-3779.3 | From 1860200 | to 1861090 | internalin C |
| SEQ ID NO. 2149 | LM-378.1 | From 85025 | to 85627 | Unknown |
| SEQ ID NO. 2150 | LM-3783.2 | From 989099 | to 990232 | N- |
| | | | | ACETYLGLUCOSAMINE-6- |
| | | | | PHOSPHATE |
| | | | | DEACETYLASE (EC |
| | | | | 3.5.1.25) (GLCNAC 6-P |
| | | | | DEACETYLASE). |
| SEQ ID NO. 2151 | LM-3785.2 | From 988065 | to 988910 | unknown |
| SEQ ID NO. 2152 | LM-379.1 | From 84451 | to 84849 | Unknown |
| SEQ ID NO. 2153 | LM-3790.3 | From 1420076 | to 1421362 | Unknown, similar to putative |
| | | | | protease |
| SEQ ID NO. 2154 | LM-3791.2 | From 1778415 | to 1779482 | unknown, similar to |
| | | | | hypothetical proteins |
| SEQ ID NO. 2155 | LM-3792.1 | From 1777763 | to 1778338 | unknown, similar to putative |
| | | | | transcription regulators |
| SEQ ID NO. 2156 | LM-3793.2 | From 1776910 | to 1777578 | unknown, similar to |
| | | | | hypothetical proteins |
| SEQ ID NO. 2157 | LM-3795.3 | From 1421454 | to 1422185 | Unknown, similar to 3- |
| | | | | ketoacyl-acyl carrier protein |
| | | | | reductase |
| SEQ ID NO. 2158 | LM-3797.2 | From 1422236 | to 1423165 | Unknown, similar to |
| | | | | unknown protein |
| SEQ ID NO. 2159 | LM-3798.1 | From 1423255 | to 1423833 | Unknown, similar to |
| | | | | phosphatidylglycerophosphate |
| | | | | synthase |
| SEQ ID NO. 2160 | LM-380.1 | From 82959 | to 84437 | Unknown |
| SEQ ID NO. 2161 | LM-3800.3 | From 1423902 | to 1425146 | Unknown, similar to |
| | | | | competence-damage |
| | | | | inducible protein CinA |
| SEQ ID NO. 2162 | LM-3801.3 | From 984823 | to 985722 | unknown |
| SEQ ID NO. 2163 | LM-3802.4 | From 985743 | to 986564 | unknown |
| SEQ ID NO. 2164 | LM-3806.3 | From 1578625 | to 1579425 | unknown, highly similar to |
| | | | | cell division inhibitor |
| | | | | (septum placement) protein |
| | | | | MinD |
| SEQ ID NO. 2165 | LM-3807.3 | From 1577212 | to 1578573 | Unknown, similar to |
| | | | | ribonuclease G |
| SEQ ID NO. 2166 | LM-3808.2 | From 2298927 | to 2299712 | Unknown |
| SEQ ID NO. 2167 | LM-3809.2 | From 2298093 | to 2298863 | Unknown, similar to |
| | | | | unknown protein |
| SEQ ID NO. 2168 | LM-3810.3 | From 333534 | to 335195 | Unknown, similar to |
| | | | | unknown protein |
| SEQ ID NO. 2169 | LM-3811.3 | From 335350 | to 336426 | Unknown |
| SEQ ID NO. 2170 | LM-3812.3 | From 1662570 | to 1663982 | Unknown, similar to Xaa-His |
| | | | | dipeptidase |
| SEQ ID NO. 2171 | LM-3818.1 | From 2211280 | to 2212245 | Unknown, similar to |
| | | | | transcription regulator, Lacl |
| | | | | family |
| SEQ ID NO. 2172 | LM-3819.1 | From 2210609 | to 2211244 | Unknown |
| SEQ ID NO. 2173 | LM-382.1 | From 81661 | to 82617 | Unknown, similar to |
| | | | | phosphoglycerate |
| | | | | dehydrogenase |
| SEQ ID NO. 2174 | LM-3821.1 | From 2159697 | to 2160053 | Unknown, similar to |
| | | | | unknown protein |
| SEQ ID NO. 2175 | LM-3822.1 | From 2149348 | to 2149632 | class I heat-shock protein |
| | | | | (chaperonin) GroES |
| SEQ ID NO. 2176 | LM-3823.3 | From 2943120 | to 2943479 | ribonuclease P protein |
| | | | | component |
| SEQ ID NO. 2177 | LM-3825.4 | From 2943871 | to 2944341 | hypothetical protein |
| SEQ ID NO. 2178 | LM-3826.1 | From 1240358 | to 1240681 | unknown, similar to |
| | | | | unknown protein |
| SEQ ID NO. 2179 | LM-383.1 | From 80892 | to 81530 | Unknown, conserved |
| | | | | hypothetical protein |
| SEQ ID NO. 2180 | LM-3845.2 | From 979762 | to 980064 | unknown, similar to B. subtilis |
| | | | | YneR protein |
| SEQ ID NO. 2181 | LM-3846.1 | From 979059 | to 979529 | non-heme iron-binding |
| | | | | ferritin |
| SEQ ID NO. 2182 | LM-3847.1 | From 940316 | to 940696 | unknown, conserved |
| | | | | hypothetical protein |
| SEQ ID NO. 2183 | LM-3848.1 | From 1357753 | to 1358697 | unknown, highly similar to |
| | | | | riboflavin kinase and FAD |
| | | | | synthase |
| SEQ ID NO. 2184 | LM-385.1 | From 79817 | to 80869 | Unknown, similar to E. coli |
| | | | | Ada protein (O6- |
| | | | | methylguanine-DNA |
| | | | | methyltransferase) |
| SEQ ID NO. 2185 | LM-3851.1 | From 1378840 | to 1379901 | Unknown, similar to |
| | | | | aminopeptidase P |
| SEQ ID NO. 2186 | LM-3853.2 | From 501693 | to 501959 | Unknown, weakly similar to |
| | | | | transposase |
| SEQ ID NO. 2187 | LM-3856.2 | From 1953836 | to 1954237 | unknown, similar to similar |
| | | | | to RNase HI |
| SEQ ID NO. 2188 | LM-3857.1 | From 1963240 | to 1963605 | unknown, similar to |
| | | | | conserved hypothetical |
| | | | | proteins |
| SEQ ID NO. 2189 | LM-3858.2 | From 2030744 | to 2031196 | Unknown, similar to |
| | | | | transcriptional regulator (Fur |
| | | | | family) |
| SEQ ID NO. 2190 | LM-386.1 | From 79047 | to 79820 | Unknown, similar to |
| | | | | carboxyphosphonoenol- |
| | | | | pyruvate phosphonomutase |
| SEQ ID NO. 2191 | LM-3863.2 | From 2494891 | to 2495268 | Unknown, similar to glycine |
| | | | | cleavage system protein H |
| SEQ ID NO. 2192 | LM-3865.2 | From 200094 | to 200402 | Unknown, similar to B. subtilis |
| | | | | SpoVG protein |
| SEQ ID NO. 2193 | LM-3867.1 | From 1890288 | to 1890653 | Unknown, similar to |
| | | | | unknown protein |
| SEQ ID NO. 2194 | LM-3868.2 | From 1428945 | to 1429748 | Unknown, conserved |
| | | | | hypothetical protein |
| SEQ ID NO. 2195 | LM-3869.2 | From 1440748 | to 1441296 | Unknown |
| SEQ ID NO. 2196 | LM-387.1 | From 78362 | to 78811 | Unknown |
| SEQ ID NO. 2197 | LM-3871.1 | From 2100845 | to 2101366 | Unknown, similar to |
| | | | | unknown protein |
| SEQ ID NO. 2198 | LM-3872.1 | From 2100224 | to 2100751 | Unknown, similar to cell- |
| | | | | division initiation protein |
| | | | | (septum placement) |
| SEQ ID NO. 2199 | LM-3873.1 | From 2891649 | to 2892032 | Unknown, hypothetical |
| | | | | secreted protein |
| SEQ ID NO. 2200 | LM-3876.1 | From 520961 | to 521536 | Unknown |
| SEQ ID NO. 2201 | LM-3878.2 | From 640625 | to 641215 | Unknown |
| SEQ ID NO. 2202 | LM-3879.2 | From 640300 | to 640632 | Unknown, conserved |
| | | | | hypothetical protein |
| SEQ ID NO. 2203 | LM-388.1 | From 78090 | to 78374 | unknown |
| SEQ ID NO. 2204 | LM-3883.1 | From 1664057 | to 1664470 | Unknown, weakly similar to |
| | | | | E. coli MutT protein (dGTP |
| | | | | pyrophosphohydrolase) |
| SEQ ID NO. 2205 | LM-3886.1 | From 1702695 | to 1703315 | unknown, putative |
| | | | | cellsurface protein |
| SEQ ID NO. 2206 | LM-3887.1 | From 1038336 | to 1038557 | unknown |
| SEQ ID NO. 2207 | LM-3889.1 | From 2524168 | to 2524911 | Unknown, similar to |
| | | | | carboxylesterase |
| SEQ ID NO. 2208 | LM-389.1 | From 77149 | to 77406 | unknown |
| SEQ ID NO. 2209 | LM-3890.1 | From 2523835 | to 2524068 | Unknown |
| SEQ ID NO. 2210 | LM-3891.1 | From 1724196 | to 1724462 | unknown, similar to |
| | | | | conserved hypothetical |
| | | | | proteins |
| SEQ ID NO. 2211 | LM-3893.1 | From 611240 | to 612634 | Unknown, similar to beta- |
| | | | | glucosidase |
| SEQ ID NO. 2212 | LM-3896.1 | From 1546608 | to 1547885 | Unknown, similar to |
| | | | | unknown protein |
| SEQ ID NO. 2213 | LM-3897.3 | From 1514257 | to 1515288 | Unknown, similar to |
| | | | | unknown protein |
| SEQ ID NO. 2214 | LM-3898.1 | From 1594535 | to 1595119 | Unknown, similar to |
| | | | | hypothetical GTP binding |
| | | | | protein |
| SEQ ID NO. 2215 | LM-39.1 | From 2736597 | to 2737628 | Unknown, similar to polyol |
| | | | | dehydrogenase |
| SEQ ID NO. 2216 | LM-390.3 | From 76262 | to 76984 | unknown |
| SEQ ID NO. 2217 | LM-3902.4 | From 1715236 | to 1716435 | Unknown, similar to S- |
| | | | | methionine |
| | | | | adenosyltransferase |
| SEQ ID NO. 2218 | LM-3904.3 | From 819389 | to 820039 | Unknown |
| SEQ ID NO. 2219 | LM-3905.2 | From 502899 | to 504629 | Unknown |
| SEQ ID NO. 2220 | LM-3907.1 | From 1234779 | to 1235330 | unknown |
| SEQ ID NO. 2221 | LM-3908.1 | From 1234465 | to 1234776 | unknown, similar to |
| | | | | unknown protein |
| SEQ ID NO. 2222 | LM-3910.2 | From 1409070 | to 1409996 | Unknown, similar to |
| | | | | unknown protein |
| SEQ ID NO. 2223 | LM-3912.1 | From 2709522 | to 2710421 | Unknown, conserved |
| | | | | lipoprotein |
| SEQ ID NO. 2224 | LM-3914.1 | From 2297304 | to 2297981 | Unknown, similar to |
| | | | | unknown protein |
| SEQ ID NO. 2225 | LM-3915.1 | From 2145127 | to 2145513 | Unknown, similar to large |
| | | | | conductance |
| | | | | mechanosensitive channel |
| | | | | protein |
| SEQ ID NO. 2226 | LM-392.3 | From 75832 | to 76125 | unknown |
| SEQ ID NO. 2227 | LM-3922.2 | From 987735 | to 988049 | Unknown |
| SEQ ID NO. 2228 | LM-3924.2 | From 986722 | to 987180 | Unknown |
| SEQ ID NO. 2229 | LM-3929.1 | From 833615 | to 834076 | unknown |
| SEQ ID NO. 2230 | LM-393.3 | From 75342 | to 75665 | Unknown |
| SEQ ID NO. 2231 | LM-3934.1 | From 356567 | to 356872 | Unknown, similar to |
| | | | | transposase |
| SEQ ID NO. 2232 | LM-3942.3 | From 2633930 | to 2634850 | Unknown, conserved |
| | | | | hypothetical protein |
| SEQ ID NO. 2233 | LM-3943.4 | From 1576107 | to 1576397 | ribosomal protein L27 |
| SEQ ID NO. 2234 | LM-3944.4 | From 1576411 | to 1576728 | Unknown, similar to |
| | | | | unknown protein |
| SEQ ID NO. 2235 | LM-3947.1 | From 1599814 | to 1600278 | Unknown, similar to |
| | | | | unknown protein |
| SEQ ID NO. 2236 | LM-3949.2 | From 1638420 | to 1638908 | Unknown, similar to |
| | | | | unknown protein |
| SEQ ID NO. 2237 | LM-395.2 | From 74239 | to 75228 | Unknown, similar to |
| | | | | dinitrogenase reductase |
| | | | | ADP-ribosylation system |
| SEQ ID NO. 2238 | LM-3950.2 | From 1639120 | to 1639722 | ribosomal protein S4 |
| SEQ ID NO. 2239 | LM-3951.1 | From 1640225 | to 1641004 | Unknown |
| SEQ ID NO. 2240 | LM-3953.1 | From 502424 | to 502660 | Hypothetical orf |
| SEQ ID NO. 2241 | LM-3954.2 | From 504674 | to 504997 | Unknown |
| SEQ ID NO. 2242 | LM-3955.1 | From 1307763 | to 1308170 | unknown, conserved |
| | | | | hypothetical protein, similar |
| | | | | to B. subtilis YneT protein |
| SEQ ID NO. 2243 | LM-3957.1 | From 2360908 | to 2361141 | Unknown |
| SEQ ID NO. 2244 | LM-3958.1 | From 2360435 | to 2360713 | Unknown, similar to |
| | | | | competence transcription |
| | | | | factor ComK, N terminal |
| | | | | part |
| SEQ ID NO. 2245 | LM-3959.2 | From 2359948 | to 2360184 | Unknown |
| SEQ ID NO. 2246 | LM-396.2 | From 1859272 | to 1859787 | translation initiation factor |
| | | | | IF-3 |
| SEQ ID NO. 2247 | LM-3961.1 | From 2494522 | to 2494806 | Unknown, similar to |
| | | | | thioredoxin |
| SEQ ID NO. 2248 | LM-3966.4 | From 1739674 | to 1740849 | unknown, similar to |
| | | | | transmembrane transport |
| | | | | proteins |
| SEQ ID NO. 2249 | LM-397.3 | From 1858651 | to 1859010 | ribosomal protein L20 |
| SEQ ID NO. 2250 | LM-3970.1 | From 1933037 | to 1933270 | unknown, similar to |
| | | | | hypothetical protein |
| SEQ ID NO. 2251 | LM-3972.2 | From 818789 | to 819265 | Unknown, similar to |
| | | | | transcription regulator |
| | | | | (EbsC from Enterococcus |
| | | | | faecalis) |
| SEQ ID NO. 2252 | LM-3973.2 | From 1136900 | to 1137400 | Unknown, similar to |
| | | | | lipoprotein signal peptidase |
| SEQ ID NO. 2253 | LM-3976.4 | From 267530 | to 267916 | Unknown, similar to |
| | | | | repressor (penicilinase |
| | | | | repressor) |
| SEQ ID NO. 2254 | LM-3978.3 | From 267010 | to 267372 | ribosomal protein L12 |
| SEQ ID NO. 2255 | LM-3979.3 | From 266431 | to 266931 | ribosomal protein L10 |
| SEQ ID NO. 2256 | LM-398.1 | From 1857856 | to 1858611 | Unknown, similar to 3-exodeoxyribonuclease |
| | | | | exoA |
| SEQ ID NO. 2257 | LM-3981.1 | From 2361897 | to 2362274 | protein gp30 [Bacteriophage |
| | | | | A118] |
| SEQ ID NO. 2258 | LM-3982.1 | From 2362306 | to 2362656 | protein gp29 [Bacteriophage |
| | | | | A118] |
| SEQ ID NO. 2259 | LM-3984.1 | From 270372 | to 270977 | Unknown, conserved |
| | | | | hypothetical protein |
| SEQ ID NO. 2260 | LM-399.1 | From 1857242 | to 1857772 | Unknown |
| SEQ ID NO. 2261 | LM-3990.1 | From 222983 | to 223261 | Unknown, highly similar to |
| | | | | B. subtilis YabO protein |
| SEQ ID NO. 2262 | LM-3993.1 | From 200522 | to 200830 | Unknown, similar to B. subtilis |
| | | | | SpoVG protein |
| SEQ ID NO. 2263 | LM-3995.1 | From 143016 | to 143258 | unknown |
| SEQ ID NO. 2264 | LM-3998.1 | From 823638 | to 824168 | Unknown, conserved |
| | | | | hypothetical protein |
| SEQ ID NO. 2265 | LM-3999.3 | From 810985 | to 811620 | Unknown, similar to acyl- |
| | | | | carrier protein |
| | | | | phosphodiesterase and to |
| | | | | NAD(P)H dehydrogenase |
| SEQ ID NO. 2266 | LM-4.1 | From 2713398 | to 2713940 | Unknown |
| SEQ ID NO. 2267 | LM-40.1 | From 2737630 | to 2738682 | Unknown, similar to sorbitol |
| | | | | dehydrogenase |
| SEQ ID NO. 2268 | LM-4003.2 | From 1513917 | to 1514171 | ribosomal protein S20 |
| SEQ ID NO. 2269 | LM-401.1 | From 1855952 | to 1857184 | Unknown, similar to |
| | | | | aminotripeptidase |
| | | | | (peptidase T) |
| SEQ ID NO. 2270 | LM-4013.2 | From 1926482 | to 1926898 | unknown, similar to |
| | | | | transcriptional regulator |
| | | | | (MarR family) |
| SEQ ID NO. 2271 | LM-4015.1 | From 1771594 | to 1772040 | unknown, similar to putative |
| | | | | flavodoxin |
| SEQ ID NO. 2272 | LM-402.1 | From 1855571 | to 1855933 | Unknown |
| SEQ ID NO. 2273 | LM-4027.2 | From 263535 | to 263714 | unknown, highly similar to |
| | | | | preprotein translocase |
| | | | | subunit |
| SEQ ID NO. 2274 | LM-4029.2 | From 263844 | to 264377 | transcription antitermination |
| | | | | factor |
| SEQ ID NO. 2275 | LM-403.1 | From 1854748 | to 1855521 | Unknown, similar to ABC |
| | | | | transporter (ATP-binding |
| | | | | protein) |
| SEQ ID NO. 2276 | LM-4031.1 | From 264432 | to 264902 | Unknown |
| SEQ ID NO. 2277 | LM-4032.1 | From 265029 | to 265454 | ribosomal protein L11 |
| SEQ ID NO. 2278 | LM-4033.1 | From 265494 | to 266183 | ribosomal protein L1 |
| SEQ ID NO. 2279 | LM-404.1 | From 1854077 | to 1854682 | Unknown, similar to |
| | | | | unknown protein |
| SEQ ID NO. 2280 | LM-4040.1 | From 504972 | to 505277 | Unknown |
| SEQ ID NO. 2281 | LM-4041.1 | From 552563 | to 552931 | Unknown, similar to |
| | | | | unknown protein |
| SEQ ID NO. 2282 | LM-4053.1 | From 1270465 | to 1270725 | unknown |
| SEQ ID NO. 2283 | LM-4055.2 | From 1288261 | to 1288926 | unknown, weakly similar to |
| | | | | oligopeptide ABC |
| | | | | transporter AppA |
| SEQ ID NO. 2284 | LM-4058.1 | From 1405945 | to 1406226 | Unknown |
| SEQ ID NO. 2285 | LM-4059.1 | From 1406366 | to 1406713 | Unknown |
| SEQ ID NO. 2286 | LM-4065.1 | From 2045765 | to 2046046 | Unknown, similar to pentitol |
| | | | | PTS system enzyme II B |
| | | | | component |
| SEQ ID NO. 2287 | LM-4082.1 | From 2782739 | to 2782894 | Unknown |
| SEQ ID NO. 2288 | LM-4083.1 | From 2780577 | to 2780798 | Unknown |
| SEQ ID NO. 2289 | LM-4084.1 | From 2780392 | to 2780574 | Unknown |
| SEQ ID NO. 2290 | LM-4088.1 | From 2701254 | to 2701445 | ribosomal protein L29 |
| SEQ ID NO. 2291 | LM-4089.1 | From 2700963 | to 2701226 | ribosomal protein S17 |
| SEQ ID NO. 2292 | LM-4090.1 | From 2699383 | to 2699568 | ribosomal protein S14 |
| SEQ ID NO. 2293 | LM-4091.1 | From 2697267 | to 2697446 | ribosomal protein L30 |
| SEQ ID NO. 2294 | LM-4096.1 | From 367248 | to 367532 | Unknown |
| SEQ ID NO. 2295 | LM-4097.1 | From 370416 | to 370634 | Unknown |
| SEQ ID NO. 2296 | LM-4098.1 | From 389538 | to 389717 | Unknown, similar to |
| | | | | conserved hypothetical |
| | | | | protein |
| SEQ ID NO. 2297 | LM-4099.1 | From 401386 | to 401580 | unknown |
| SEQ ID NO. 2298 | LM-41.1 | From 2738719 | to 2739990 | Unknown, similar to PTS |
| | | | | system galactitol-specific |
| | | | | enzyme IIC component |
| SEQ ID NO. 2299 | LM-4106.1 | From 439009 | to 439212 | Unknown, similar to putative |
| | | | | transcription regulator |
| SEQ ID NO. 2300 | LM-4113.1 | From 521601 | to 521771 | ribosomal protein L32 |
| SEQ ID NO. 2301 | LM-4115.1 | From 616742 | to 616951 | Unknown, similar to |
| | | | | unknown protein |
| SEQ ID NO. 2302 | LM-4118.1 | From 669086 | to 669361 | Unknown |
| SEQ ID NO. 2303 | LM-4119.1 | From 696606 | to 696815 | unknown |
| SEQ ID NO. 2304 | LM-4120.1 | From 707310 | to 707486 | unknown |
| SEQ ID NO. 2305 | LM-4121.1 | From 709601 | to 709810 | unknown |
| SEQ ID NO. 2306 | LM-4122.1 | From 731765 | to 732001 | Unknown, weakly similar to |
| | | | | flagellar switch protein |
| SEQ ID NO. 2307 | LM-4123.1 | From 756079 | to 756291 | unknown, LPXTG motif |
| | | | | protein |
| SEQ ID NO. 2308 | LM-4124.1 | From 756494 | to 756637 | Hypothetical CDS |
| SEQ ID NO. 2309 | LM-4130.1 | From 890069 | to 890215 | Unknown, hypothetical |
| | | | | protein |
| SEQ ID NO. 2310 | LM-4134.1 | From 961203 | to 961469 | Unknown, similar to ABC |
| | | | | transporter, ATP-binding |
| | | | | protein (C-terminal part) |
| SEQ ID NO. 2311 | LM-4135.1 | From 973509 | to 973670 | unknown |
| SEQ ID NO. 2312 | LM-4137.1 | From 981317 | to 981544 | unknown |
| SEQ ID NO. 2313 | LM-4139.1 | From 987274 | to 987501 | Unknown |
| SEQ ID NO. 2314 | LM-4142.1 | From 1003593 | to 1003829 | D-alanyl carrier protein |
| SEQ ID NO. 2315 | LM-4144.1 | From 1057067 | to 1057276 | unknown, similar to B. subtilis |
| | | | | YkzG protein |
| SEQ ID NO. 2316 | LM-4147.1 | From 1133466 | to 1133696 | unknown, highly similar to |
| | | | | TN916 ORF8 |
| SEQ ID NO. 2317 | LM-4148.1 | From 1145723 | to 1145944 | unknown, highly similar to |
| | | | | TN916 ORF19 |
| SEQ ID NO. 2318 | LM-4149.2 | From 1154864 | to 1156381 | unknown |
| SEQ ID NO. 2319 | LM-415.1 | From 1848166 | to 1848690 | Unknown, similar to |
| | | | | unknown protein |
| SEQ ID NO. 2320 | LM-4150.1 | From 1188014 | to 1188289 | Unknown, similar to |
| | | | | carboxysome structural |
| | | | | protein |
| SEQ ID NO. 2321 | LM-4152.2 | From 1287376 | to 1287591 | unknown, similar to |
| | | | | transcriptional regulator |
| SEQ ID NO. 2322 | LM-4153.1 | From 1331439 | to 1331666 | unknown, similar to B. subtilis |
| | | | | YnzC protein |
| SEQ ID NO. 2323 | LM-4154.1 | From 1363826 | to 1363975 | ribosomal protein L33 |
| SEQ ID NO. 2324 | LM-4155.1 | From 1366220 | to 1366432 | unknown similar to B. subtilis |
| | | | | yqgQ |
| SEQ ID NO. 2325 | LM-4157.1 | From 1385704 | to 1385931 | Unknown, similar to |
| | | | | exodeoxyribonuclease small |
| | | | | subunit |
| SEQ ID NO. 2326 | LM-4158.1 | From 1387014 | to 1387214 | similar to cold shock protein |
| SEQ ID NO. 2327 | LM-4161.1 | From 1501881 | to 1502054 | 30S ribosomal protein S21 |
| SEQ ID NO. 2328 | LM-4162.2 | From 1576747 | to 1577055 | ribosomal protein L21 |
| SEQ ID NO. 2329 | LM-4166.1 | From 1654691 | to 1654909 | Unknown, hypothetical gene |
| SEQ ID NO. 2330 | LM-417.1 | From 1847472 | to 1847960 | hosphoribosylaminoimidazole |
| | | | | carboxylase I |
| SEQ ID NO. 2331 | LM-4172.1 | From 1756629 | to 1756862 | unknown |
| SEQ ID NO. 2332 | LM-4174.1 | From 1764871 | to 1765077 | unknown |
| SEQ ID NO. 2333 | LM-4175.1 | From 1769479 | to 1769700 | unknown |
| SEQ ID NO. 2334 | LM-4179.1 | From 1859050 | to 1859250 | ribosomal protein L35 |
| SEQ ID NO. 2335 | LM-418.1 | From 1846355 | to 1847479 | Phosphoribosylaminoimidazole |
| | | | | carboxylase II |
| SEQ ID NO. 2336 | LM-4180.1 | From 1867180 | to 1867410 | Unknown, similar to |
| | | | | unknown protein |
| SEQ ID NO. 2337 | LM-4181.1 | From 1881041 | to 1881274 | Unknown, highly similar to |
| | | | | acyl carrier proteins |
| SEQ ID NO. 2338 | LM-4182.1 | From 1890951 | to 1891139 | ribosomal protein L28 |
| SEQ ID NO. 2339 | LM-4183.1 | From 1902281 | to 1902484 | unknown |
| SEQ ID NO. 2340 | LM-4184.1 | From 1928579 | to 1928785 | unknown, similar to putative |
| | | | | mercuric ion binding |
| | | | | proteins |
| SEQ ID NO. 2341 | LM-4186.1 | From 1953513 | to 1953713 | similar to cold shock protein |
| SEQ ID NO. 2342 | LM-4189.1 | From 2094877 | to 2095077 | similar to major cold-shock |
| | | | | protein |
| SEQ ID NO. 2343 | LM-419.1 | From 1845044 | to 1846336 | adenylosuccinate lyase |
| SEQ ID NO. 2344 | LM-4192.1 | From 2144874 | to 2145065 | unknown |
| SEQ ID NO. 2345 | LM-4193.1 | From 2150556 | to 2150759 | unknown |
| SEQ ID NO. 2346 | LM-4195.1 | From 2184120 | to 2184347 | Unknown |
| SEQ ID NO. 2347 | LM-4197.1 | From 2192379 | to 2192609 | Unknown |
| SEQ ID NO. 2348 | LM-420.1 | From 1844250 | to 1844963 | Phosphoribosylaminoimidazole |
| | | | | succinocarboxamide |
| | | | | synthetase |
| SEQ ID NO. 2349 | LM-4200.1 | From 2234357 | to 2234533 | unknown |
| SEQ ID NO. 2350 | LM-4201.1 | From 2242097 | to 2242282 | unknown, similar to B. subtilis |
| | | | | YwmG protein |
| SEQ ID NO. 2351 | LM-4203.1 | From 2293497 | to 2293685 | Unknown, similar to |
| | | | | unknown protein |
| SEQ ID NO. 2352 | LM-4206.1 | From 2346593 | to 2346784 | unknown, |
| SEQ ID NO. 2353 | LM-4207.1 | From 2361582 | to 2361791 | unknown |
| SEQ ID NO. 2354 | LM-4208.1 | From 2366288 | to 2366446 | protein gp22 [Bacteriophage |
| | | | | A118] |
| SEQ ID NO. 2355 | LM-4209.1 | From 2387499 | to 2387663 | Bacteriophage A118 gp65 |
| | | | | protein |
| SEQ ID NO. 2356 | LM-421.1 | From 1843993 to | 1844238 | Unknown, similar to |
| | | | | unknown protein |
| SEQ ID NO. 2357 | LM-4210.1 | From 2388528 | to 2388710 | Hypothetical protein |
| SEQ ID NO. 2358 | LM-4211.1 | From 2389208 | to 2389387 | Unknown |
| SEQ ID NO. 2359 | LM-4212.1 | From 2389491 | to 2389658 | unknown |
| SEQ ID NO. 2360 | LM-4213.1 | From 2391020 | to 2391217 | Unknown |
| SEQ ID NO. 2361 | LM-4214.1 | From 2394790 | to 2394984 | Unknown |
| SEQ ID NO. 2362 | LM-4215.1 | From 2395586 | to 2395801 | gp44 [Bacteriophage A118] |
| SEQ ID NO. 2363 | LM-4216.1 | From 2398287 | to 2398529 | Unknown, similar to |
| | | | | transcription regulator |
| SEQ ID NO. 2364 | LM-422.1 | From 1843306 | to 1843989 | Unknown, similar to |
| | | | | phosphoribosylformylglycinamidine |
| | | | | synthetase II |
| SEQ ID NO. 2365 | LM-4225.1 | From 2467388 | to 2467621 | Unknown, similar to B. subtilis |
| | | | | YuzB protein |
| SEQ ID NO. 2366 | LM-4226.1 | From 2468545 | to 2468703 | Unknown |
| SEQ ID NO. 2367 | LM-4227.1 | From 2480325 | to 2480528 | Unknown, similar to |
| | | | | repressor protein |
| SEQ ID NO. 2368 | LM-4228.1 | From 2501306 | to 2501470 | Unknown |
| SEQ ID NO. 2369 | LM-423.1 | From 1841094 | to 1843313 | Phosphoribosylformylglycinamidine |
| | | | | synthetase I |
| SEQ ID NO. 2370 | LM-4230.1 | From 2559617 | to 2559817 | Unknown, similar to B. subtilis |
| | | | | yvlC protein |
| SEQ ID NO. 2371 | LM-4231.1 | From 2567696 | to 2567935 | Unknown, similar to B. subtilis |
| | | | | CsbA protein |
| SEQ ID NO. 2372 | LM-4233.1 | From 2611192 | to 2611410 | unknown, highly similar to |
| | | | | H+-transporting ATP |
| | | | | synthase chain c |
| SEQ ID NO. 2373 | LM-4234.1 | From 2624633 | to 2624878 | ribosomal protein L31 |
| SEQ ID NO. 2374 | LM-4235.1 | From 2643753 | to 2643938 | Unknown, similar to 4- |
| | | | | oxalocrotonate isomerase |
| SEQ ID NO. 2375 | LM-4236.1 | From 2646464 | to 2646664 | unknown |
| SEQ ID NO. 2376 | LM-4246.1 | From 145354 | to 145560 | Unknown, hypothetical |
| | | | | protein |
| SEQ ID NO. 2377 | LM-4247.1 | From 145085 | to 145171 | Unknown, hypothetical |
| | | | | protein |
| SEQ ID NO. 2378 | LM-4248.1 | From 136714 | to 136992 | Unknown, similar to E. coli |
| | | | | YjdJ protein |
| SEQ ID NO. 2379 | LM-4249.1 | From 136469 | to 136702 | Unknown, similar to E. coli |
| | | | | YjdI protein |
| SEQ ID NO. 2380 | LM-425.1 | From 1839682 | to 1841109 | glutamine |
| | | | | phosphoribosylpyrophosphate |
| | | | | amidotransferase |
| SEQ ID NO. 2381 | LM-4251.1 | From 77863 | to 78066 | Unknown, Hypothetical |
| SEQ ID NO. 2382 | LM-4253.1 | From 52373 | to 52534 | Unknown |
| SEQ ID NO. 2383 | LM-4254.1 | From 50514 | to 50753 | 30S ribosomal protein S18 |
| SEQ ID NO. 2384 | LM-426.1 | From 1838614 | to 1839663 | Phosphoribosylaminoimidazole |
| | | | | synthetase |
| SEQ ID NO. 2385 | LM-4262.2 | From 267909 | to 268949 | Unknown, similar to |
| | | | | penicillinase antirepressor |
| SEQ ID NO. 2386 | LM-4267.1 | From 1153010 | to 1153783 | unknown, similar to |
| | | | | regulatory proteins |
| SEQ ID NO. 2387 | LM-4268.1 | From 1153848 | to 1154204 | unknown |
| SEQ ID NO. 2388 | LM-427.1 | From 1838051 | to 1838617 | unknown, highly similar to |
| | | | | phosphoribosylglycinamide |
| | | | | formyltransferases |
| SEQ ID NO. 2389 | LM-4276.2 | From 2943569 | to 2943703 | ribosomal protein L34 |
| SEQ ID NO. 2390 | LM-4277.1 | From 2693947 | to 2694060 | ribosomal protein L36 |
| SEQ ID NO. 2391 | LM-428.1 | From 1836516 | to 1838045 | Bifunctional |
| | | | | phosphoribosylaminoimidazole |
| | | | | carboxyformyl |
| | | | | formyltransferase and |
| | | | | inosine-monophosphate |
| | | | | cyclohydrolase |
| SEQ ID NO. 2392 | LM-429.1 | From 1835229 | to 1836491 | phosphoribosylglycinamide |
| | | | | synthetase |
| SEQ ID NO. 2393 | LM-4293.1 | From 2130228 | to 2130401 | ribosomal protein L32 |
| SEQ ID NO. 2394 | LM-4295.1 | From 2052696 | to 2052833 | Unknown |
| SEQ ID NO. 2395 | LM-43.1 | From 2740052 | to 2740333 | Unknown, similar to PTS |
| | | | | system galactitol-specific |
| | | | | enzyme IIB component |
| SEQ ID NO. 2396 | LM-430.1 | From 1834795 | to 1835091 | Unknown, similar to |
| | | | | unknown protein |
| SEQ ID NO. 2397 | LM-432.1 | From 1834523 | to 1834777 | Unknown |
| SEQ ID NO. 2398 | LM-433.1 | From 1833125 | to 1834480 | Unknown, similar to putative |
| | | | | sodium-dependent |
| | | | | transporter |
| SEQ ID NO. 2399 | LM-4342.1 | From 2317644 | to 2317841 | Unknown, similar to |
| | | | | unknown protein |
| SEQ ID NO. 2400 | LM-4349.1 | From 1287710 | to 1288243 | Unknown |
| SEQ ID NO. 2401 | LM-435.1 | From 1832242 | to 1832919 | Unknown, similar to |
| | | | | unknown protein |
| SEQ ID NO. 2402 | LM-4352.1 | From 436161 | to 436352 | Unknown |
| SEQ ID NO. 2403 | LM-4353.1 | From 2250846 | to 2251004 | Unknown |
| SEQ ID NO. 2404 | LM-4358.1 | From 2491285 | to 2491473 | unknown |
| SEQ ID NO. 2405 | LM-4359.1 | From 2173635 | to 2173832 | |
| SEQ ID NO. 2406 | LM-436.2 | From 1829982 | to 1832177 | ATP-dependent DNA |
| | | | | helicase |
| SEQ ID NO. 2407 | LM-4360.1 | From 263366 | to 263515 | |
| SEQ ID NO. 2408 | LM-438.1 | From 1827941 | to 1829956 | Unknown, similar to DNA |
| | | | | ligase |
| SEQ ID NO. 2409 | LM-439.1 | From 1826829 | to 1827944 | Unknown, similar to |
| | | | | unknown protein |
| SEQ ID NO. 2410 | LM-44.1 | From 2740390 | to 2740854 | Unknown, similar to PTS |
| | | | | system galactitol-specific |
| | | | | enzyme IIA component |
| SEQ ID NO. 2411 | LM-440.1 | From 1826411 | to 1826704 | glutamyl-tRNA(Gln) |
| | | | | amidotransferase (subunit |
| | | | | C) |
| SEQ ID NO. 2412 | LM-441.1 | From 1824936 | to 1826387 | glutamyl-tRNA(Gln) |
| | | | | amidotransferase (subunit |
| | | | | A) |
| SEQ ID NO. 2413 | LM-442.1 | From 1823494 | to 1824924 | glutamyl-tRNA(Gln) |
| | | | | amidotransferase (subunit |
| | | | | B) |
| SEQ ID NO. 2414 | LM-443.2 | From 1822421 | to 1823353 | Unknown, similar to |
| | | | | unknown protein |
| SEQ ID NO. 2415 | LM-444.2 | From 1821518 | to 1822273 | Unknown |
| SEQ ID NO. 2416 | LM-445.1 | From 1820133 | to 1821494 | Unknown, similar to |
| | | | | hypothetical RNA |
| | | | | methyltransferase |
| SEQ ID NO. 2417 | LM-446.1 | From 1819244 | to 1819798 | Unknown, similar to |
| | | | | unknown protein |
| SEQ ID NO. 2418 | LM-447.2 | From 1818740 | to 1819213 | Unknown, similar to |
| | | | | shikimate kinase |
| SEQ ID NO. 2419 | LM-45.2 | From 2740888 | to 2742957 | unknown, similar to |
| | | | | transcriptional |
| | | | | antiterminator (BglG family) |
| SEQ ID NO. 2420 | LM-451.1 | From 2435023 | to 2436141 | Unknown, similar to S. pyogenes |
| | | | | RofA regulatory |
| | | | | protein |
| SEQ ID NO. 2421 | LM-452.1 | From 2434627 | to 2434866 | Hypothetical protein |
| SEQ ID NO. 2422 | LM-453.1 | From 2433224 | to 2434618 | Unknown, similar to |
| | | | | glutamate decarboxylase |
| SEQ ID NO. 2423 | LM-455.1 | From 2431688 | to 2433211 | Unknown, similar to amino |
| | | | | acid antiporter (acid |
| | | | | resistance) |
| SEQ ID NO. 2424 | LM-456.1 | From 2430826 | to 2431296 | Unknown, conserved |
| | | | | hypothetical protein |
| SEQ ID NO. 2425 | LM-458.1 | From 2428010 | to 2430793 | Unknown, transmembrane |
| | | | | protein |
| SEQ ID NO. 2426 | LM-460.1 | From 2427045 | to 2427890 | Unknown, conserved |
| | | | | hypothetical protein |
| SEQ ID NO. 2427 | LM-463.1 | From 2426270 | to 2427001 | Unknown, similar to N- |
| | | | | acetylglucosamine-6- |
| | | | | phosphate isomerase |
| SEQ ID NO. 2428 | LM-464.1 | From 2425723 | to 2426253 | Unknown, similar to |
| | | | | unknown protein |
| SEQ ID NO. 2429 | LM-466.1 | From 2424922 | to 2425533 | Unknown |
| SEQ ID NO. 2430 | LM-468.1 | From 2423480 | to 2424721 | Unknown; Similar to |
| | | | | multidrug resistance protein |
| SEQ ID NO. 2431 | LM-469.1 | From 2421878 | to 2423476 | Unknown, conserved |
| | | | | hypothetical protein |
| SEQ ID NO. 2432 | LM-47.2 | From 2743326 | to 2744138 | Unknown |
| SEQ ID NO. 2433 | LM-471.1 | From 2419797 | to 2421749 | Unknown, similar to putative |
| | | | | Na+/H+ antiporter |
| SEQ ID NO. 2434 | LM-472.1 | From 2418871 | to 2419770 | Unknown, similar to LysR |
| | | | | family transcription regulator |
| SEQ ID NO. 2435 | LM-473.1 | From 2418083 | to 2418628 | Unknown, similar to NADH- |
| | | | | dependent FMN reductase |
| SEQ ID NO. 2436 | LM-475.1 | From 2417531 | to 2418067 | Unknown, similar to B. subtilis |
| | | | | YtmI protein |
| SEQ ID NO. 2437 | LM-476.1 | From 2416705 | to 2417514 | Unknown, similar to amino |
| | | | | acid ABC transporter |
| | | | | (binding protein) |
| SEQ ID NO. 2438 | LM-477.1 | From 2415967 | to 2416683 | Unknown, similar to amino |
| | | | | acid ABC-transporter |
| | | | | (permease) |
| SEQ ID NO. 2439 | LM-478.1 | From 2415246 | to 2415953 | Unknown, similar to amino |
| | | | | acid ABC transporter |
| | | | | (permease) |
| SEQ ID NO. 2440 | LM-48.1 | From 2744212 | to 2744574 | Unknown, conserved |
| | | | | hypothetical protein |
| SEQ ID NO. 2441 | LM-480.1 | From 2414470 | to 2415249 | Unknown, similar to amino |
| | | | | acid ABC-transporter, ATP- |
| | | | | binding protein |
| SEQ ID NO. 2442 | LM-481.1 | From 2413478 | to 2414473 | Unknown, conserved |
| | | | | hypothetical protein |
| SEQ ID NO. 2443 | LM-482.1 | From 2413219 | to 2413485 | Unknown, similar to B. subtilis |
| | | | | YtnI protein |
| SEQ ID NO. 2444 | LM-483.1 | From 2411900 | to 2413222 | Unknown, similar to |
| | | | | nitrilotriacetate |
| | | | | monooxygenase |
| SEQ ID NO. 2445 | LM-484.1 | From 2411126 | to 2411827 | Unknown, similar to 16S |
| | | | | pseudouridylate synthase |
| SEQ ID NO. 2446 | LM-485.1 | From 2409875 | to 2410993 | similar to kinases |
| SEQ ID NO. 2447 | LM-486.1 | From 2408967 | to 2409878 | Unknown, similar to Erwinia |
| | | | | chrysanthemi IndA protein |
| SEQ ID NO. 2448 | LM-487.1 | From 2408450 | to 2408893 | Unknown, conserved |
| | | | | hypothetical protein |
| SEQ ID NO. 2449 | LM-488.1 | From 2407128 | to 2408453 | aminopeptidase C |
| SEQ ID NO. 2450 | LM-49.1 | From 2744571 | to 2744939 | Unknown |
| SEQ ID NO. 2451 | LM-490.1 | From 2406161 | to 2406913 | Unknown, similar to |
| | | | | regulatory protein DeoR |
| | | | | family |
| SEQ ID NO. 2452 | LM-491.1 | From 2405241 | to 2406164 | fructose-1-phosphate |
| | | | | kinase |
| SEQ ID NO. 2453 | LM-492.1 | From 2403341 | to 2405239 | unknown, highly similar to |
| | | | | phosphotransferase system |
| | | | | (PTS) fructose-specific |
| | | | | enzyme IIABC component |
| SEQ ID NO. 2454 | LM-494.1 | From 2402888 | to 2403235 | Unknown, similar to |
| | | | | transcriptional regulator |
| SEQ ID NO. 2455 | LM-495.1 | From 2402357 | to 2402833 | competence transcription |
| | | | | factor |
| SEQ ID NO. 2456 | LM-496.1 | From 2401008 | to 2402366 | putative integrase |
| | | | | [Bacteriophage A118] |
| SEQ ID NO. 2457 | LM-497.1 | From 2400264 | to 2400944 | Unknown, weakly similar to |
| | | | | gp32_Bacteriophage A118 |
| | | | | protein |
| SEQ ID NO. 2458 | LM-498.1 | From 2399542 | to 2400243 | Unknown, similar to protein |
| | | | | gp33 [Bacteriophage A118] |
| SEQ ID NO. 2459 | LM-5.1 | From 2713962 | to 2714942 | Unknown, similar to |
| | | | | heptaprenyl diphosphate |
| | | | | synthase component II |
| SEQ ID NO. 2460 | LM-500.1 | From 2398685 | to 2399161 | Unknown, similar to a |
| | | | | putative repressor protein |
| | | | | [Bacteriophage A118] |
| SEQ ID NO. 2461 | LM-502.1 | From 2397800 | to 2398084 | Unknown |
| SEQ ID NO. 2462 | LM-503.1 | From 2397493 | to 2397774 | Unknown, similar to protein |
| | | | | gp41 [Bacteriophage A118] |
| SEQ ID NO. 2463 | LM-506.1 | From 2396454 | to 2397230 | Unknown, similar to antirepressor |
| | | | | [Bacteriophage |
| | | | | A118] |
| SEQ ID NO. 2464 | LM-507.1 | From 2395798 | to 2396331 | gp43 [Bacteriophage A118] |
| SEQ ID NO. 2465 | LM-509.1 | From 2394317 | to 2394793 | Unknown, similar to |
| | | | | bacteriophage proteins |
| SEQ ID NO. 2466 | LM-51.1 | From 2744985 | to 2745791 | Unknown, weakly similar to |
| | | | | AraC-like transcription |
| | | | | regulator |
| SEQ ID NO. 2467 | LM-510.1 | From 2393613 | to 2394311 | Unknown |
| SEQ ID NO. 2468 | LM-512.1 | From 2392622 | to 2393596 | Unknown, similar to protein |
| | | | | gp49 [Bacteriophage A118] |
| SEQ ID NO. 2469 | LM-513.1 | From 2391813 | to 2392625 | Unknown, similar to site- |
| | | | | specific DNA- |
| | | | | methyltransferase |
| SEQ ID NO. 2470 | LM-514.1 | From 2391220 | to 2391816 | Unknown, similar to protein |
| | | | | gp51 [Bacteriophage A118] |
| SEQ ID NO. 2471 | LM-517.1 | From 2390580 | to 2391023 | Unknown, similar to a |
| | | | | bacteriophage protein |
| SEQ ID NO. 2472 | LM-518.1 | From 2390113 | to 2390583 | Unknown |
| SEQ ID NO. 2473 | LM-52.1 | From 2745907 | to 2746377 | Unknown, conserved |
| | | | | hypothetical protein |
| SEQ ID NO. 2474 | LM-520.1 | From 2389655 | to 2390116 | Unknown |
| SEQ ID NO. 2475 | LM-522.1 | From 2388729 | to 2389211 | unknown, similar to single- |
| | | | | stranded DNA-binding |
| | | | | protein |
| SEQ ID NO. 2476 | LM-523.1 | From 2388179 | to 2388583 | Unknown |
| SEQ ID NO. 2477 | LM-524.1 | From 2387792 | to 2388175 | unknown |
| SEQ ID NO. 2478 | LM-525.1 | From 2387046 | to 2387480 | Protein gp66 |
| | | | | [Bacteriophage A118] |
| SEQ ID NO. 2479 | LM-527.1 | From 2386141 | to 2386680 | Unknown |
| SEQ ID NO. 2480 | LM-529.1 | From 2385301 | to 2386095 | Unknown, similar to putative |
| | | | | terminase small subunit |
| | | | | from Bacteriophage A118 |
| SEQ ID NO. 2481 | LM-53.1 | From 2746423 | to 2746878 | Unknown, similar to ribose |
| | | | | 5-phosphate epimerase |
| SEQ ID NO. 2482 | LM-530.1 | From 2384001 | to 2385332 | putative terminase large |
| | | | | subunit from Bacteriophage |
| | | | | A118 |
| SEQ ID NO. 2483 | LM-531.1 | From 2382228 | to 2383988 | putative portal protein |
| | | | | [Bacteriophage A118] |
| SEQ ID NO. 2484 | LM-532.1 | From 2381088 | to 2382227 | Protein gp4 [Bacteriophage |
| | | | | A118] |
| SEQ ID NO. 2485 | LM-533.1 | From 2380419 | to 2381009 | Unknown, putative |
| | | | | scaffolding protein |
| | | | | [Bacteriophage A118] |
| SEQ ID NO. 2486 | LM-535.1 | From 2379418 | to 2380419 | Unknown, similar to coat |
| | | | | protein [Bacteriophage |
| | | | | SPP1] |
| SEQ ID NO. 2487 | LM-536.1 | From 2379004 | to 2379399 | Protein gp8 [Bacteriophage |
| | | | | A118] |
| SEQ ID NO. 2488 | LM-537.1 | From 2378642 | to 2379004 | Protein gp9 [Bacteriophage |
| | | | | A118] |
| SEQ ID NO. 2489 | LM-538.1 | From 2378304 | to 2378642 | Protein gp10 |
| | | | | [Bacteriophage A118] |
| SEQ ID NO. 2490 | LM-539.1 | From 2377897 | to 2378304 | Portein gp11 |
| | | | | [Bacteriophage A118] |
| SEQ ID NO. 2491 | LM-54.1 | From 2747073 | to 2747414 | Unknown |
| SEQ ID NO. 2492 | LM-540.1 | From 2377460 | to 2377894 | major tail shaft protein |
| | | | | [Bacteriophage A118] |
| SEQ ID NO. 2493 | LM-541.1 | From 2377198 | to 2377530 | Protein gp13 |
| | | | | [Bacteriophage A118] |
| SEQ ID NO. 2494 | LM-542.1 | From 2376721 | to 2377143 | Protein gp14 |
| | | | | [Bacteriophage A118] |
| SEQ ID NO. 2495 | LM-543.1 | From 2376110 | to 2376715 | Protein gp15 |
| | | | | [Bacteriophage A118] |
| SEQ ID NO. 2496 | LM-548.1 | From 2370736 | to 2376099 | Unknown, putative tape- |
| | | | | measure [Bacteriophage |
| | | | | A118] |
| SEQ ID NO. 2497 | LM-549.1 | From 2369916 | to 2370734 | Protein gp17 |
| | | | | [Bacteriophage A118] |
| SEQ ID NO. 2498 | LM-55.1 | From 2747427 | to 2748683 | Unknown, similar to UV- |
| | | | | damage repair protein |
| SEQ ID NO. 2499 | LM-550.1 | From 2368882 | to 2369907 | Protein gp18 |
| | | | | [Bacteriophage A118] |
| SEQ ID NO. 2500 | LM-552.1 | From 2367853 | to 2368881 | Protein gp19 |
| | | | | [Bacteriophage A118] |
| SEQ ID NO. 2501 | LM-553.1 | From 2366780 | to 2367853 | protein gp20 [Bacteriophage |
| | | | | A118] |
| SEQ ID NO. 2502 | LM-554.1 | From 2366451 | to 2366768 | protein gp21 [Bacteriophage |
| | | | | A118] |
| SEQ ID NO. 2503 | LM-555.1 | From 2365893 | to 2366258 | protein gp23 [Bacteriophage |
| | | | | A118] |
| SEQ ID NO. 2504 | LM-557.1 | From 2365599 | to 2365880 | holin [Bacteriophage A118] |
| SEQ ID NO. 2505 | LM-558.1 | From 2364754 | to 2365599 | L-alanoyl-D-glutamate |
| | | | | peptidase |
| SEQ ID NO. 2506 | LM-559.1 | From 2363709 | to 2364260 | Unknown |
| SEQ ID NO. 2507 | LM-56.1 | From 2748685 | to 2749497 | Unknown, similar to |
| | | | | hydrolase (esterase) |
| SEQ ID NO. 2508 | LM-560.1 | From 2363135 | to 2363632 | Unknown, similar to an |
| | | | | unknown bacteriophage |
| | | | | protein |
| SEQ ID NO. 2509 | LM-562.3 | From 2362661 | to 2363110 | Protein gp28 |
| | | | | [Bacteriophage A118] |
| SEQ ID NO. 2510 | LM-563.3 | From 318 | to 1673 | Chromosomal replication |
| | | | | initiation protein DnaA |
| SEQ ID NO. 2511 | LM-564.1 | From 1867 | to 3012 | DNA polymerase III, beta |
| | | | | chain |
| SEQ ID NO. 2512 | LM-565.1 | From 3121 | to 4464 | Unknown, conserved |
| | | | | hypothetical protein |
| SEQ ID NO. 2513 | LM-566.1 | From 4644 | to 4865 | Unknown, similar to B. subtilis |
| | | | | YaaA protein |
| SEQ ID NO. 2514 | LM-567.1 | From 4869 | to 5981 | RecF protein |
| SEQ ID NO. 2515 | LM-57.1 | From 2749538 | to 2750233 | Unknown, similar to two |
| | | | | components response |
| | | | | regulator |
| SEQ ID NO. 2516 | LM-570.1 | From 6030 | to 7970 | DNA gyrase subunit B |
| SEQ ID NO. 2517 | LM-573.1 | From 8065 | to 10593 | DNA gyrase subunit A |
| SEQ ID NO. 2518 | LM-575.1 | From 10728 | to 12242 | Unknown, similar to |
| | | | | cardiolipin synthase |
| SEQ ID NO. 2519 | LM-576.1 | From 12258 | to 12776 | diamine N-acetyltransferase |
| SEQ ID NO. 2520 | LM-578.1 | From 12918 | to 13886 | Unknown, similar to |
| | | | | mevalonate kinase |
| SEQ ID NO. 2521 | LM-580.1 | From 13843 | to 14814 | unknown, similar to |
| | | | | mevalonate diphosphate |
| | | | | decarboxylase |
| SEQ ID NO. 2522 | LM-581.1 | From 14795 | to 15874 | Unknown, similar to |
| | | | | mevalonate kinases |
| SEQ ID NO. 2523 | LM-582.1 | From 16219 | to 17325 | AA3-600 quinol oxidase |
| | | | | subunit II |
| SEQ ID NO. 2524 | LM-584.1 | From 17344 | to 19323 | AA3-600 quinol oxidase |
| | | | | subunit I |
| SEQ ID NO. 2525 | LM-585.1 | From 19311 | to 19922 | AA3-600 quinol oxidase |
| | | | | subunit III |
| SEQ ID NO. 2526 | LM-586.1 | From 19924 | to 20256 | unknown, highly similar to |
| | | | | quinol oxidase aa3-600 |
| | | | | chain IV |
| SEQ ID NO. 2527 | LM-587.1 | From 20308 | to 21426 | Unknown, similar to Bacillus |
| | | | | anthracis CapA protein |
| | | | | (polyglutamate capsule |
| | | | | biosynthesis) |
| SEQ ID NO. 2528 | LM-589.1 | From 21652 | to 23085 | beta-glucosidase |
| SEQ ID NO. 2529 | LM-591.1 | From 23132 | to 23953 | Unknown |
| SEQ ID NO. 2530 | LM-592.1 | From 24194 | to 24934 | Unknown, similar to |
| | | | | transcriptional regulator |
| | | | | (GntR family) |
| SEQ ID NO. 2531 | LM-593.1 | From 24950 | to 25351 | Unknown, similar to PTS |
| | | | | system, fructose-specific IIA |
| | | | | component |
| SEQ ID NO. 2532 | LM-594.1 | From 25351 | to 25839 | Unknown, similar to PTS |
| | | | | system, fructose-specific IIB |
| | | | | component |
| SEQ ID NO. 2533 | LM-596.1 | From 25862 | to 26665 | Unknown, similar to PTS |
| | | | | system, fructose-specific IIC |
| | | | | component |
| SEQ ID NO. 2534 | LM-597.1 | From 26640 | to 27467 | Unknown, similar to PTS |
| | | | | system, mannose-specific |
| | | | | IID component |
| SEQ ID NO. 2535 | LM-598.1 | From 27495 | to 28199 | Unknown, similar to |
| | | | | phosphoheptose isomerase |
| SEQ ID NO. 2536 | LM-599.1 | From 28254 | to 28895 | Unknown, similar to E. coli |
| | | | | copper homeostasis protein |
| | | | | CutC |
| SEQ ID NO. 2537 | LM-6.1 | From 2715023 | to 2716363 | Unknown |
| SEQ ID NO. 2538 | LM-600.1 | From 29100 | to 31004 | Unknown, similar to PTS |
| | | | | system, beta-glucosides |
| | | | | specific IIABC component |
| SEQ ID NO. 2539 | LM-601.1 | From 31088 | to 31963 | Unknown, similar to E. coli |
| | | | | microcin C7 self-immunity |
| | | | | protein (MccF) |
| SEQ ID NO. 2540 | LM-602.1 | From 32197 | to 32535 | Unknown |
| SEQ ID NO. 2541 | LM-604.1 | From 32571 | to 33380 | Unknown, conserved |
| | | | | hypothetical protein |
| SEQ ID NO. 2542 | LM-605.1 | From 33397 | to 34452 | Unknown, transcriptional |
| | | | | regulator Lacl family |
| SEQ ID NO. 2543 | LM-606.1 | From 34654 | to 35619 | Unknown, similar to xylose |
| | | | | repressor |
| SEQ ID NO. 2544 | LM-608.1 | From 35616 | to 38018 | Unknown, similar to |
| | | | | endoglucanase |
| SEQ ID NO. 2545 | LM-609.1 | From 38031 | to 39383 | Unknown, similar to PTS |
| | | | | system, cellobiose-specific |
| | | | | IIC component |
| SEQ ID NO. 2546 | LM-610.1 | From 39385 | to 40470 | Unknown, similar to |
| | | | | Glucosamine-fructose-6- |
| | | | | phosphate |
| | | | | aminotransferase (C- |
| | | | | terminal domain) |
| SEQ ID NO. 2547 | LM-611.1 | From 40705 | to 41730 | Unknown, similar to |
| | | | | ornithine |
| | | | | carbamoyltransferase |
| SEQ ID NO. 2548 | LM-613.1 | From 41803 | to 43188 | Unknown, similar to amino |
| | | | | acid transporter |
| SEQ ID NO. 2549 | LM-614.1 | From 43175 | to 44266 | Unknown, conserved |
| | | | | hypothetical protein |
| SEQ ID NO. 2550 | LM-615.1 | From 44282 | to 45223 | carbamate kinase |
| SEQ ID NO. 2551 | LM-616.1 | From 45325 | to 46434 | Unknown, conserved |
| | | | | hypothetical protein |
| SEQ ID NO. 2552 | LM-617.1 | From 46451 | to 47230 | Unknown, conserved |
| | | | | hypothetical protein, |
| | | | | hypothetical regulator |
| SEQ ID NO. 2553 | LM-618.1 | From 47335 | to 47994 | Unknown, similar to E. coli |
| | | | | DedA protein |
| SEQ ID NO. 2554 | LM-619.2 | From 48074 | to 49306 | Unknown, similar to arginine |
| | | | | deiminase |
| SEQ ID NO. 2555 | LM-62.1 | From 2750230 | to 2752920 | Unknown, similar to the two |
| | | | | components sensor protein |
| | | | | kdpD |
| SEQ ID NO. 2556 | LM-620.2 | From 703688 | to 703951 | Unknown |
| SEQ ID NO. 2557 | LM-621.3 | From 703128 | to 703691 | Unknown, similar to acetyl |
| | | | | transferase |
| SEQ ID NO. 2558 | LM-622.1 | From 703961 | to 704338 | Unknown, similar to |
| | | | | unknown protein |
| SEQ ID NO. 2559 | LM-623.1 | From 704452 | to 705387 | Unknown, similar to ABC |
| | | | | transporter (ATP-binding |
| | | | | protein) |
| SEQ ID NO. 2560 | LM-624.1 | From 705380 | to 706150 | Unknown, conserved |
| | | | | membrane protein |
| SEQ ID NO. 2561 | LM-625.1 | From 706408 | to 707292 | Unknown, similar to |
| | | | | oxidoreductase |
| SEQ ID NO. 2562 | LM-626.1 | From 707612 | to 708220 | Unknown |
| SEQ ID NO. 2563 | LM-627.1 | From 709134 | to 709562 | Unknown, similar to |
| | | | | unknown protein |
| SEQ ID NO. 2564 | LM-628.1 | From 709829 | to 710749 | Unknown |
| SEQ ID NO. 2565 | LM-629.1 | From 711121 | to 711435 | Unknown |
| SEQ ID NO. 2566 | LM-631.1 | From 711428 | to 712195 | Unknown, similar to flagellar |
| | | | | biosynthesis protein FliP |
| SEQ ID NO. 2567 | LM-632.1 | From 712208 | to 712480 | Unknown, similar to flagellar |
| | | | | biosynthesis protein FliQ |
| SEQ ID NO. 2568 | LM-633.1 | From 712483 | to 713244 | Unknown, similar to flagellar |
| | | | | biosynthetic protein FliR |
| SEQ ID NO. 2569 | LM-635.1 | From 713260 | to 714306 | Unknown, similar to flagellar |
| | | | | biosynthetic protein flhB |
| SEQ ID NO. 2570 | LM-636.1 | From 714353 | to 716428 | Unknown, similar to flagellar- |
| | | | | associated protein flhA |
| SEQ ID NO. 2571 | LM-637.1 | From 716450 | to 717673 | Unknown, similar to |
| | | | | flagellar biosynthesis |
| | | | | protein FlhF |
| SEQ ID NO. 2572 | LM-639.1 | From 717670 | to 718449 | Unknown, similar to flagellar |
| | | | | hook-basal body protein |
| | | | | FlgG |
| SEQ ID NO. 2573 | LM-641.1 | From 718478 | to 719266 | Unknown, similar to |
| | | | | chemotactic |
| | | | | methyltransferase CheR |
| SEQ ID NO. 2574 | LM-642.1 | From 719291 | to 719626 | Unknown |
| SEQ ID NO. 2575 | LM-643.1 | From 719653 | to 720504 | Unknown, similar to motility |
| | | | | protein (flagellar motor |
| | | | | rotation) MotA |
| SEQ ID NO. 2576 | LM-645.1 | From 720464 | to 721291 | Unknown, similar to motility |
| | | | | protein (flagellar motor |
| | | | | rotation) MotB |
| SEQ ID NO. 2577 | LM-647.1 | From 721301 | to 721801 | Unknown |
| SEQ ID NO. 2578 | LM-648.1 | From 721824 | to 723737 | Unknown, similar to |
| | | | | unknown protein |
| SEQ ID NO. 2579 | LM-649.1 | From 723750 | to 724658 | Unknown, similar to CheA |
| | | | | activity-modulating |
| | | | | chemotaxis protein CheV |
| SEQ ID NO. 2580 | LM-65.1 | From 2752936 | to 2753508 | potassium-transporting |
| | | | | ATPase c chain |
| SEQ ID NO. 2581 | LM-650.3 | From 724896 | to 725759 | flagellin protein |
| SEQ ID NO. 2582 | LM-651.1 | From 726034 | to 726393 | Chemotaxis response |
| | | | | regulator CheY |
| SEQ ID NO. 2583 | LM-652.1 | From 726413 | to 728269 | two-component sensor |
| | | | | histidine kinase CheA |
| SEQ ID NO. 2584 | LM-653.1 | From 728282 | to 728581 | Unknown, similar to flagellar |
| | | | | motor switch protein fliY C- |
| | | | | terminal part |
| SEQ ID NO. 2585 | LM-654.1 | From 728600 | to 729010 | Unknown |
| SEQ ID NO. 2586 | LM-657.1 | From 729026 | to 730072 | Unknown |
| SEQ ID NO. 2587 | LM-658.1 | From 730074 | to 730496 | unknown, similar to flagellar |
| | | | | hook assembly protein |
| SEQ ID NO. 2588 | LM-659.1 | From 730516 | to 731751 | Unknown, similar to flagellar |
| | | | | hook protein FlgE |
| SEQ ID NO. 2589 | LM-660.1 | From 732022 | to 733014 | Unknown, similar to flagellar |
| | | | | switch protein FliM |
| SEQ ID NO. 2590 | LM-664.1 | From 733017 | to 734564 | Unknown, similar to flagellar |
| | | | | motor switch protein fliY |
| SEQ ID NO. 2591 | LM-665.1 | From 734570 | to 735973 | Unknown |
| SEQ ID NO. 2592 | LM-668.1 | From 735996 | to 737162 | Unknown |
| SEQ ID NO. 2593 | LM-669.1 | From 737269 | to 737733 | Unknown |
| SEQ ID NO. 2594 | LM-670.1 | From 737743 | to 738171 | Unknown |
| SEQ ID NO. 2595 | LM-671.1 | From 738191 | to 739711 | Unknown, similar to flagellar |
| | | | | hook-associated protein |
| | | | | FlgK |
| SEQ ID NO. 2596 | LM-672.1 | From 739723 | to 740598 | Unknown, similar to flagellar |
| | | | | hook-associated protein 3 |
| | | | | FlgL |
| SEQ ID NO. 2597 | LM-674.1 | From 740610 | to 741899 | Unknown, similar to flagellar |
| | | | | hook-associated protein 2 |
| | | | | FliD |
| SEQ ID NO. 2598 | LM-675.1 | From 741918 | to 742304 | Unknown, similar to |
| | | | | hypothetical flagellar protein |
| SEQ ID NO. 2599 | LM-676.1 | From 742276 | to 742557 | Unknown |
| SEQ ID NO. 2600 | LM-677.1 | From 742578 | to 742979 | Unknown, similar to flagellar |
| | | | | basal-body rod protein flgB |
| SEQ ID NO. 2601 | LM-679.1 | From 742991 | to 743401 | Unknown, similar to flagellar |
| | | | | basal-body rod protein flgC |
| SEQ ID NO. 2602 | LM-680.1 | From 743418 | to 743714 | Unknown, similar to flagellar |
| | | | | hook-basal body complex |
| | | | | protein FliE |
| SEQ ID NO. 2603 | LM-682.1 | From 743782 | to 745434 | Unknown, similar to flagellar |
| | | | | basal-body M-ring protein |
| | | | | fliF |
| SEQ ID NO. 2604 | LM-685.1 | From 745437 | to 746543 | Unknown, similar to flagellar |
| | | | | motor switch protein fliG |
| SEQ ID NO. 2605 | LM-686.1 | From 746530 | to 747222 | Unknown |
| SEQ ID NO. 2606 | LM-688.1 | From 747219 | to 748520 | Unknown, similar to H+- |
| | | | | transporting ATP synthase |
| | | | | alpha chain FliI, flagellar- |
| | | | | specific, |
| SEQ ID NO. 2607 | LM-689.1 | From 748537 | to 749205 | Unknown, similar to |
| | | | | transglycosylase |
| SEQ ID NO. 2608 | LM-69.1 | From 2753523 | to 2755568 | potassium-transporting |
| | | | | atpase b chain |
| SEQ ID NO. 2609 | LM-690.1 | From 749219 | to 749863 | Unknown |
| SEQ ID NO. 2610 | LM-691.1 | From 749985 | to 750311 | Unknown, similar to |
| | | | | unknown protein |
| SEQ ID NO. 2611 | LM-692.1 | From 750311 | to 750634 | Unknown |
| SEQ ID NO. 2612 | LM-693.1 | From 750680 | to 751327 | putative fibronectin-binding |
| | | | | protein |
| SEQ ID NO. 2613 | LM-695.1 | From 751598 | to 753328 | Unknown, similar to |
| | | | | pyruvate oxidase |
| SEQ ID NO. 2614 | LM-697.1 | From 753490 | to 755295 | Unknown, similar to methyl- |
| | | | | accepting chemotaxis |
| | | | | protein |
| SEQ ID NO. 2615 | LM-698.1 | From 755308 | to 756036 | Unknown, similar to B. subtilis |
| | | | | YvpB protein |
| SEQ ID NO. 2616 | LM-699.2 | From 2842413 | to 2843867 | Unknown, similar to beta- |
| | | | | glucosidase |
| SEQ ID NO. 2617 | LM-7.1 | From 2716408 | to 2717394 | Unknown |
| SEQ ID NO. 2618 | LM-700.1 | From 2843914 | to 2844216 | Unknown, similar to PTS |
| | | | | cellobiose-specific enzyme |
| | | | | IIB |
| SEQ ID NO. 2619 | LM-702.1 | From 2844249 | to 2845601 | Unknown, similar to PTS |
| | | | | cellobiose-specific enzyme |
| | | | | IIC component |
| SEQ ID NO. 2620 | LM-703.1 | From 2845613 | to 2846497 | Unknown, similar to xylose |
| | | | | operon regulatory protein |
| | | | | and to glucose kinase |
| SEQ ID NO. 2621 | LM-704.1 | From 2846490 | to 2846798 | Unknown, similar to PTS |
| | | | | cellobiose-specific enzyme |
| | | | | IIA |
| SEQ ID NO. 2622 | LM-705.1 | From 2846839 | to 2847573 | Unknown, similar to |
| | | | | hypothetical transcriptional |
| | | | | regulator |
| SEQ ID NO. 2623 | LM-706.1 | From 2847673 | to 2848335 | Unknown |
| SEQ ID NO. 2624 | LM-707.2 | From 2848406 | to 2849536 | Unknown |
| SEQ ID NO. 2625 | LM-708.3 | From 2849563 | to 2850450 | Unknown, similar to ABC |
| | | | | transporter, ATP-binding |
| | | | | protein |
| SEQ ID NO. 2626 | LM-709.2 | From 2850620 | to 2852950 | Unknown, similar to |
| | | | | gamma-glutamylcysteine |
| | | | | synthetase (for the |
| | | | | N_terminal part) and to |
| | | | | cyanophycin synthetase (C- |
| | | | | terminal part) |
| SEQ ID NO. 2627 | LM-710.1 | From 2852988 | to 2854436 | Unknown, similar to beta- |
| | | | | glucosidase |
| SEQ ID NO. 2628 | LM-711.2 | From 2854429 | to 2856282 | Unknown, similar to beta- |
| | | | | glucoside-specific enzyme |
| | | | | IIABC |
| SEQ ID NO. 2629 | LM-712.2 | From 2856379 | to 2857218 | Unknown, similar to |
| | | | | transcription antiterminator |
| SEQ ID NO. 2630 | LM-714.1 | From 2857580 | to 2858203 | Unknown, similar to ABC |
| | | | | transporter, ATP-binding |
| | | | | protein |
| SEQ ID NO. 2631 | LM-715.1 | From 2858196 | to 2860364 | Unknown |
| SEQ ID NO. 2632 | LM-716.1 | From 2860426 | to 2860821 | Unknown |
| SEQ ID NO. 2633 | LM-717.1 | From 2861424 | to 2862614 | Unknown, similar to efflux |
| | | | | protein |
| SEQ ID NO. 2634 | LM-718.1 | From 2862774 | to 2863283 | Unknown |
| SEQ ID NO. 2635 | LM-720.1 | From 2863561 | to 2864661 | Unknown, similar to |
| | | | | probable GTP-binding |
| | | | | protein |
| SEQ ID NO. 2636 | LM-721.1 | From 2864813 | to 2865121 | Unknown, similar to |
| | | | | cellobiose PTS enzyme IIA |
| SEQ ID NO. 2637 | LM-723.1 | From 2865127 | to 2867397 | Unknown, similar to beta- |
| | | | | glucosidase |
| SEQ ID NO. 2638 | LM-724.1 | From 2867432 | to 2867731 | Unknown, similar to PTS, |
| | | | | cellobiose-specific IIB |
| | | | | component |
| SEQ ID NO. 2639 | LM-725.1 | From 2867746 | to 2869041 | Unknown, similar to |
| | | | | cellobiose |
| | | | | phosphotransferase system |
| | | | | enzyme IIC |
| SEQ ID NO. 2640 | LM-726.1 | From 2869191 | to 2871107 | Unknown, simliar to |
| | | | | lichenan operon |
| | | | | transcription antiterminator |
| | | | | IicR |
| SEQ ID NO. 2641 | LM-727.1 | From 2871318 | to 2872784 | catalase |
| SEQ ID NO. 2642 | LM-728.1 | From 2872932 | to 2873915 | Unknown |
| SEQ ID NO. 2643 | LM-73.1 | From 2755580 | to 2757265 | unknown, highly similar to |
| | | | | potassium-transporting |
| | | | | atpase a chain |
| SEQ ID NO. 2644 | LM-730.1 | From 2873915 | to 2875837 | beta-glucoside-specific |
| | | | | phosphotransferase |
| | | | | enzyme II |
| SEQ ID NO. 2645 | LM-731.2 | From 2875945 | to 2876775 | transcription antiterminator |
| SEQ ID NO. 2646 | LM-733.2 | From 2877160 | to 2878011 | Partition protein ParB |
| | | | | homolg |
| SEQ ID NO. 2647 | LM-734.1 | From 2878004 | to 2878765 | Partition protein, ParA |
| | | | | homolog |
| SEQ ID NO. 2648 | LM-735.1 | From 2878989 | to 2879747 | Unknown |
| SEQ ID NO. 2649 | LM-736.1 | From 2879906 | to 2880205 | Unknown |
| SEQ ID NO. 2650 | LM-737.1 | From 2880217 | to 2881071 | Unknown, highly similar to |
| | | | | B. subtilis DNA-binding |
| | | | | protein Spo0J-like homolog |
| | | | | YyaA |
| SEQ ID NO. 2651 | LM-739.1 | From 2881243 | to 2882049 | Unknown, similar to E. coli |
| | | | | RpiR transcription regulator |
| SEQ ID NO. 2652 | LM-74.1 | From 2757606 | to 2757911 | Unknown, similar to |
| | | | | cellobiose |
| | | | | phosphotransferase |
| | | | | enzyme IIB component |
| SEQ ID NO. 2653 | LM-740.1 | From 2882033 | to 2882938 | Unknown, similar to |
| | | | | transcription regulator |
| SEQ ID NO. 2654 | LM-741.1 | From 2882963 | to 2883409 | Unknown, similar to |
| | | | | phosphotransferase system |
| | | | | mannitol-specific enzyme |
| | | | | IIA |
| SEQ ID NO. 2655 | LM-742.1 | From 2883422 | to 2884078 | Unknown, similar to |
| | | | | phosphatase |
| SEQ ID NO. 2656 | LM-744.1 | From 2884141 | to 2885547 | Unknown, similar to |
| | | | | phosphotransferase system |
| | | | | mannitol-specific enzyme |
| | | | | IIBC |
| SEQ ID NO. 2657 | LM-747.1 | From 2885581 | to 2886582 | Unknown, similar to |
| | | | | dehydrogenase |
| SEQ ID NO. 2658 | LM-748.2 | From 2886584 | to 2887297 | Unknown, similar to a |
| | | | | putative N- |
| | | | | acetylmannosamine-6- |
| | | | | phosphate epimerase |
| SEQ ID NO. 2659 | LM-749.2 | From 2536347 | to 2538581 | unknown, similar to |
| | | | | transport protein |
| SEQ ID NO. 2660 | LM-750.1 | From 2538588 | to 2539196 | Unknown, similar to |
| | | | | transcription regulator |
| SEQ ID NO. 2661 | LM-751.1 | From 2539213 | to 2539611 | Unknown |
| SEQ ID NO. 2662 | LM-752.1 | From 2539838 | to 2540173 | Unknown |
| SEQ ID NO. 2663 | LM-753.1 | From 2540421 | to 2541857 | Unknown, similar to |
| | | | | chitinase and chitin binding |
| | | | | protein |
| SEQ ID NO. 2664 | LM-754.1 | From 2542014 | to 2542610 | ATP-dependent Clp |
| | | | | protease proteolytic subunit |
| SEQ ID NO. 2665 | LM-755.1 | From 2542658 | to 2544049 | Unknown, similar to amino |
| | | | | acid transporter |
| SEQ ID NO. 2666 | LM-758.1 | From 2545487 | to 2546503 | Unknown, similar to NADH |
| | | | | oxidase |
| SEQ ID NO. 2667 | LM-759.1 | From 2546530 | to 2547501 | Unknown, conserved |
| | | | | hypothetical protein |
| SEQ ID NO. 2668 | LM-760.1 | From 2547509 | to 2548477 | unknown, conserved |
| | | | | hypothetical protein |
| SEQ ID NO. 2669 | LM-761.1 | From 2548479 | to 2549354 | unknown, conserved |
| | | | | hypothetical protein |
| SEQ ID NO. 2670 | LM-762.1 | From 2549462 | to 2551192 | Unknown, similar to |
| | | | | phosphomannomutase and |
| | | | | phosphoglucomutase |
| SEQ ID NO. 2671 | LM-763.1 | From 2551234 | to 2552295 | Unknown, similar to aldose |
| | | | | 1-epimerase (mutarotase) |
| SEQ ID NO. 2672 | LM-764.1 | From 2552312 | to 2553295 | UDP-glucose 4-epimerase |
| SEQ ID NO. 2673 | LM-765.1 | From 2553414 | to 2554373 | thioredoxin reductase |
| SEQ ID NO. 2674 | LM-766.1 | From 2554452 | to 2555927 | Unknown |
| SEQ ID NO. 2675 | LM-767.1 | From 2556013 | to 2556510 | Unknown, similar to |
| | | | | acetyltransferase |
| SEQ ID NO. 2676 | LM-768.1 | From 2556514 | to 2557167 | Unknown, similar to B. subtilis |
| | | | | P-Ser-HPr |
| | | | | phosphatase |
| SEQ ID NO. 2677 | LM-769.1 | From 2557211 | to 2558044 | unknown, highly similar to |
| | | | | prolipoprotein diacylglyceryl |
| | | | | transferase |
| SEQ ID NO. 2678 | LM-77.1 | From 2758046 | to 2759353 | Unknown, similar to |
| | | | | cellobiose |
| | | | | phosphotransferase |
| | | | | enzyme IIC component |
| SEQ ID NO. 2679 | LM-771.1 | From 2558130 | to 2559068 | HPr-P(Ser) |
| | | | | kinase/phosphatase |
| SEQ ID NO. 2680 | LM-772.1 | From 2559255 | to 2559608 | Unknown, similar to B. subtilis |
| | | | | YvlD protein |
| SEQ ID NO. 2681 | LM-774.1 | From 2559841 | to 2561052 | unknown |
| SEQ ID NO. 2682 | LM-776.1 | From 2561120 | to 2562382 | Unknown, similar to B. subtilis |
| | | | | YvlB protein |
| SEQ ID NO. 2683 | LM-778.1 | From 2562591 | to 2565461 | excinuclease ABC (subunit |
| | | | | A) |
| SEQ ID NO. 2684 | LM-78.1 | From 2759390 | to 2759692 | Unknown, similar to |
| | | | | cellobiose |
| | | | | phosphotransferase |
| | | | | enzyme IIA component |
| SEQ ID NO. 2685 | LM-780.1 | From 2565469 | to 2567445 | excinuclease ABC (subunit |
| | | | | B) |
| SEQ ID NO. 2686 | LM-781.1 | From 2567956 | to 2568603 | Unknown |
| SEQ ID NO. 2687 | LM-782.1 | From 2568625 | to 2569011 | unknown |
| SEQ ID NO. 2688 | LM-783.1 | From 2569117 | to 2569422 | Unknown, similar to |
| | | | | transcription regulator ArsR |
| | | | | family |
| SEQ ID NO. 2689 | LM-784.1 | From 2569690 | to 2570349 | Unknown, similar to |
| | | | | negative regulator of |
| | | | | phosphate regulon |
| SEQ ID NO. 2690 | LM-786.1 | From 2570362 | to 2571141 | unknown, similar to |
| | | | | phosphate ABC transporter |
| | | | | (ATP-binding protein) |
| SEQ ID NO. 2691 | LM-788.1 | From 2571156 | to 2571971 | unknown, similar to |
| | | | | phosphate ABC transporter |
| | | | | (ATP-binding protein) |
| SEQ ID NO. 2692 | LM-789.1 | From 2571999 | to 2572883 | Unknown, similar to |
| | | | | phosphate ABC transporter |
| | | | | (permease protein) |
| SEQ ID NO. 2693 | LM-791.1 | From 2572880 | to 2573803 | Unknown, similar to |
| | | | | phosphate ABC transporter |
| | | | | (permease protein) |
| SEQ ID NO. 2694 | LM-793.1 | From 2573897 | to 2574805 | Unknown, similar to |
| | | | | phosphate ABC transporter |
| | | | | (binding protein) |
| SEQ ID NO. 2695 | LM-794.1 | From 2575061 | to 2576836 | two-component sensor |
| | | | | histidine kinase |
| SEQ ID NO. 2696 | LM-795.1 | From 2576836 | to 2577546 | two-component response |
| | | | | regulator |
| SEQ ID NO. 2697 | LM-796.2 | From 2577696 | to 2578871 | Unknown |
| SEQ ID NO. 2698 | LM-797.2 | From 2578899 | to 2580347 | Unknown, similar to |
| | | | | cardiolipin synthase |
| SEQ ID NO. 2699 | LM-799.2 | From 2278639 | to 2279292 | competence negative |
| | | | | regulator mecA |
| SEQ ID NO. 2700 | LM-8.1 | From 2717422 | to 2718234 | Unknown |
| SEQ ID NO. 2701 | LM-80.1 | From 2760147 | to 2760680 | unknown |
| SEQ ID NO. 2702 | LM-801.1 | From 2277398 | to 2278513 | Unknown, similar to a |
| | | | | putative competence protein |
| | | | | from streptococcus |
| | | | | pneumoniae |
| SEQ ID NO. 2703 | LM-803.1 | From 2275520 | to 2277325 | Unknown, similar to |
| | | | | oligoendopeptidase |
| SEQ ID NO. 2704 | LM-804.1 | From 2274961 | to 2275221 | Unknown |
| SEQ ID NO. 2705 | LM-805.1 | From 2274127 | to 2274750 | Unknown |
| SEQ ID NO. 2706 | LM-806.1 | From 2272403 | to 2274112 | Unknown |
| SEQ ID NO. 2707 | LM-807.1 | From 2271444 | to 2272316 | Unknown, similar to |
| | | | | ferrichrome ABC transporter |
| | | | | (binding protein) |
| SEQ ID NO. 2708 | LM-809.1 | From 2270465 | to 2271454 | Unknown, similar to |
| | | | | ferrichrome ABC transporter |
| | | | | (permease) |
| SEQ ID NO. 2709 | LM-81.1 | From 2760845 | to 2761954 | Unknown, simlilar to cell |
| | | | | division protein FtsW |
| SEQ ID NO. 2710 | LM-810.1 | From 2269705 | to 2270484 | Unknown, similar to |
| | | | | ferrichrome ABC transporter |
| | | | | (ATP-binding protein) |
| SEQ ID NO. 2711 | LM-812.1 | From 2268961 | to 2269701 | Unknown, similar to |
| | | | | unknown protein |
| SEQ ID NO. 2712 | LM-813.2 | From 2268424 | to 2268900 | Unknown, similar to |
| | | | | unknown protein |
| SEQ ID NO. 2713 | LM-817.1 | From 2259366 | to 2259713 | Unknown, similar to |
| | | | | unknown protein |
| SEQ ID NO. 2714 | LM-818.1 | From 2258697 | to 2259260 | Unknown, similar to |
| | | | | transcriptional regulator |
| | | | | (tetR family) |
| SEQ ID NO. 2715 | LM-820.1 | From 2257754 | to 2258515 | Unknown, similar to |
| | | | | dehydrogenase |
| SEQ ID NO. 2716 | LM-821.1 | From 2256507 | to 2257580 | Unknown, similar to |
| | | | | unknown proteins |
| SEQ ID NO. 2717 | LM-823.1 | From 2255034 | to 2256401 | Unknown, similar to sigma- |
| | | | | 54-dependent |
| | | | | transcriptional activator |
| SEQ ID NO. 2718 | LM-826.1 | From 2253221 | to 2254804 | Unknown, similar to |
| | | | | propionate CoA-transferase |
| SEQ ID NO. 2719 | LM-827.1 | From 2252001 | to 2253224 | Unknown, similar to |
| | | | | antiporter proteins |
| SEQ ID NO. 2720 | LM-83.1 | From 2761951 | to 2763081 | Unknown, simlilar to cell |
| | | | | division protein FtsW |
| SEQ ID NO. 2721 | LM-830.1 | From 2251050 | to 2251979 | Unknown, similar to |
| | | | | unknown proteins |
| SEQ ID NO. 2722 | LM-831.1 | From 2250431 | to 2250820 | Unknown, similar to |
| | | | | glyoxalase I |
| SEQ ID NO. 2723 | LM-833.1 | From 2249683 | to 2250300 | Unknown, similar to |
| | | | | unknown proteins |
| SEQ ID NO. 2724 | LM-834.1 | From 2248946 | to 2249617 | Unknown |
| SEQ ID NO. 2725 | LM-835.1 | From 2247998 | to 2248693 | Unknown, similar to |
| | | | | transcription regulator |
| | | | | CRP/FNR family |
| SEQ ID NO. 2726 | LM-836.1 | From 2247050 | to 2247928 | Unknown, similar to |
| | | | | transcriptional regulator |
| | | | | (AraC/XylS family) |
| SEQ ID NO. 2727 | LM-837.1 | From 2245899 | to 2246975 | Unknown, similar to |
| | | | | oxidoreductase |
| SEQ ID NO. 2728 | LM-838.1 | From 2245143 | to 2245883 | Unknown, similar to |
| | | | | unknown proteins |
| SEQ ID NO. 2729 | LM-839.1 | From 2244419 | to 2245141 | Unknown |
| SEQ ID NO. 2730 | LM-840.1 | From 2243448 | to 2244416 | Unknown, similar to |
| | | | | unknown proteins |
| SEQ ID NO. 2731 | LM-841.1 | From 2242376 | to 2243425 | Unknown, similar to |
| | | | | oxidoreductase |
| SEQ ID NO. 2732 | LM-842.1 | From 2240068 | to 2241969 | Unknown |
| SEQ ID NO. 2733 | LM-843.1 | From 2239672 | to 2240013 | Unknown |
| SEQ ID NO. 2734 | LM-845.1 | From 2236844 | to 2239135 | Unknown, similar to |
| | | | | ribonucleoside-diphosphate |
| | | | | reductase, subunit alpha |
| SEQ ID NO. 2735 | LM-846.1 | From 2235739 | to 2236788 | Unknown, similar to |
| | | | | ribonucleoside-diphosphate |
| | | | | reductase, subunit beta |
| SEQ ID NO. 2736 | LM-847.1 | From 2235305 | to 2235742 | Unknown, similar to |
| | | | | flavodoxin |
| SEQ ID NO. 2737 | LM-848.1 | From 2234982 | to 2235308 | Unknown, similar to |
| | | | | thioredoxin |
| SEQ ID NO. 2738 | LM-849.1 | From 2234575 | to 2234874 | Unknown, similar to |
| | | | | unknown proteins |
| SEQ ID NO. 2739 | LM-85.1 | From 2763082 | to 2765652 | Unknown, highly similar to |
| | | | | Mg2+ transport ATPase |
| SEQ ID NO. 2740 | LM-850.1 | From 2233841 | to 2234158 | Unknown, similar to |
| | | | | unknown proteins |
| SEQ ID NO. 2741 | LM-851.1 | From 2233150 | to 2233791 | Unknown, similar to |
| | | | | unknown proteins |
| SEQ ID NO. 2742 | LM-853.1 | From 2232087 | to 2233094 | Unknown, similar to |
| | | | | unknown proteins |
| SEQ ID NO. 2743 | LM-854.1 | From 2231127 | to 2231981 | Unknown, similar to |
| | | | | transcription regulator LysR |
| | | | | family |
| SEQ ID NO. 2744 | LM-856.1 | From 2230042 | to 2231058 | Unknown, similar to |
| | | | | unknown protein |
| SEQ ID NO. 2745 | LM-857.1 | From 2229193 | to 2229927 | Unknown, similar to |
| | | | | transcription regulator GntR |
| | | | | family |
| SEQ ID NO. 2746 | LM-858.1 | From 2227412 | to 2229154 | Unknown, weakly similar to |
| | | | | mannose-6-phosphate |
| | | | | isomerase |
| SEQ ID NO. 2747 | LM-859.1 | From 2226533 | to 2227198 | Unknown |
| SEQ ID NO. 2748 | LM-860.1 | From 2225471 | to 2225944 | Unknown, similar to |
| | | | | unknown protein |
| SEQ ID NO. 2749 | LM-861.1 | From 2224219 | to 2225454 | Unknown, similar to ABC |
| | | | | transporter (membrane |
| | | | | protein) |
| SEQ ID NO. 2750 | LM-862.1 | From 2223324 | to 2224226 | Unknown, similar to ABC |
| | | | | transporter (ATP-binding |
| | | | | protein) |
| SEQ ID NO. 2751 | LM-863.1 | From 2221671 | to 2223128 | Unknown, similar to |
| | | | | transcription regulator |
| SEQ ID NO. 2752 | LM-865.1 | From 2221186 | to 2221659 | Unknown, similar to PTS |
| | | | | system, fructose-specific |
| | | | | enzyme IIA component |
| SEQ ID NO. 2753 | LM-866.1 | From 2220862 | to 2221173 | Unknown, similar to PTS |
| | | | | system, fructose-specific |
| | | | | enzyme IIB component |
| SEQ ID NO. 2754 | LM-867.1 | From 2219751 | to 2220845 | Unknown, similar to PTS |
| | | | | system, fructose-specific |
| | | | | enzyme IIC component |
| SEQ ID NO. 2755 | LM-869.1 | From 2218879 | to 2219733 | Unknown, similar to |
| | | | | fructose-1,6-biphosphate |
| | | | | aldolase type II |
| SEQ ID NO. 2756 | LM-87.1 | From 2766207 | to 2766773 | Unknown, similar to |
| | | | | transcription regulator, TetR |
| | | | | family |
| SEQ ID NO. 2757 | LM-870.1 | From 2217963 | to 2218862 | Unknown, similar to |
| | | | | fructose-1,6-biphosphate |
| | | | | aldolase type II |
| SEQ ID NO. 2758 | LM-871.1 | From 2217222 | to 2217953 | Unknown |
| SEQ ID NO. 2759 | LM-872.1 | From 2216054 | to 2216743 | Unknown |
| SEQ ID NO. 2760 | LM-874.2 | From 2486136 | to 2486921 | Unknown, similar to ABC |
| | | | | transporter, ATP-binding |
| | | | | protein |
| SEQ ID NO. 2761 | LM-875.1 | From 2484816 | to 2486117 | Unknown, similar to |
| | | | | aminotransferase |
| SEQ ID NO. 2762 | LM-876.1 | From 2483589 | to 2484815 | Unknown, similar to |
| | | | | aminotransferase |
| SEQ ID NO. 2763 | LM-877.1 | From 2483149 | to 2483592 | Unknown, similar to |
| | | | | conserved hypothetical |
| | | | | proteins |
| SEQ ID NO. 2764 | LM-878.1 | From 2481732 | to 2483126 | Unknown, similar to |
| | | | | conserved hypothetical |
| | | | | proteins |
| SEQ ID NO. 2765 | LM-879.1 | From 2481023 | to 2481574 | Unknown |
| SEQ ID NO. 2766 | LM-880.1 | From 2480531 | to 2480944 | Unknown |
| SEQ ID NO. 2767 | LM-881.1 | From 2479607 | to 2479921 | unknown |
| SEQ ID NO. 2768 | LM-883.1 | From 2478623 | to 2479465 | unknown, similar to B. subtilis |
| | | | | YunF protein |
| SEQ ID NO. 2769 | LM-884.1 | From 2478225 | to 2478590 | Unknown |
| SEQ ID NO. 2770 | LM-885.1 | From 2477385 | to 2478224 | Unknown, similar to |
| | | | | conserved hypothetical |
| | | | | proteins |
| SEQ ID NO. 2771 | LM-886.1 | From 2475860 | to 2477251 | Unknown, similar to B. subtilis |
| | | | | YunD protein |
| SEQ ID NO. 2772 | LM-887.1 | From 2475588 | to 2475863 | Unknown, similar to B. subtilis |
| | | | | YutD protein |
| SEQ ID NO. 2773 | LM-888.1 | From 2474801 | to 2475568 | Unknown, similar to |
| | | | | conserved hypothetical |
| | | | | protein and to B. subtilis |
| | | | | YutF protein |
| SEQ ID NO. 2774 | LM-889.1 | From 2474309 | to 2474752 | Unknown, similar to |
| | | | | acetyltransferase |
| SEQ ID NO. 2775 | LM-89.1 | From 2766935 | to 2768707 | Unknown, similar to |
| | | | | autolysin, N- |
| | | | | acetylmuramidase |
| SEQ ID NO. 2776 | LM-890.1 | From 2472963 | to 2474273 | Unknown, similar to |
| | | | | conserved hypothetical |
| | | | | proteins |
| SEQ ID NO. 2777 | LM-892.1 | From 2472438 | to 2472938 | Unknown, low temperature |
| | | | | requirement C protein, also |
| | | | | similar to B. subtilis YutG |
| | | | | protein |
| SEQ ID NO. 2778 | LM-893.1 | From 2472089 | to 2472325 | Unknown, similar to NifU |
| | | | | protein |
| SEQ ID NO. 2779 | LM-896.1 | From 2468210 | to 2468392 | Unknown, hypothetical CDS |
| SEQ ID NO. 2780 | LM-897.1 | From 2467643 | to 2467969 | Unknown, similar to B. subtilis |
| | | | | YuzD protein |
| SEQ ID NO. 2781 | LM-899.1 | From 2466713 | to 2467342 | Unknown, conserved |
| | | | | hypothetical protein similar |
| | | | | to B. subtilis YhfK protein |
| SEQ ID NO. 2782 | LM-90.1 | From 2768944 | to 2769273 | Unknown |
| SEQ ID NO. 2783 | LM-901.1 | From 2465630 | to 2466625 | Unknown, similar to |
| | | | | hypothetical thioredoxine |
| | | | | reductase |
| SEQ ID NO. 2784 | LM-903.1 | From 2463969 | to 2465180 | Unknown, similar to NADH |
| | | | | dehydrogenase |
| SEQ ID NO. 2785 | LM-904.1 | From 2463066 | to 2463884 | Unknown, similar to B. subtilis |
| | | | | YwqG protein |
| SEQ ID NO. 2786 | LM-906.1 | From 2461803 | to 2463029 | Unknown, conserved |
| | | | | hypothetical protein |
| SEQ ID NO. 2787 | LM-907.1 | From 2461219 | to 2461692 | Unknown, similar to B. subtilis |
| | | | | YuiD protein |
| SEQ ID NO. 2788 | LM-908.1 | From 2460720 | to 2461091 | Unknown, similar to B. subtilis |
| | | | | YuxO protein |
| SEQ ID NO. 2789 | LM-91.1 | From 2769381 | to 2770007 | Unknown, similar to |
| | | | | thymidylate kinase |
| SEQ ID NO. 2790 | LM-910.1 | From 2460090 | to 2460683 | unknown, similar to proteins |
| | | | | involved in resistance to |
| | | | | cholate and to NA(+) and in |
| | | | | pH homeostasis |
| SEQ ID NO. 2791 | LM-911.1 | From 2459819 | to 2460106 | unknown, similar to proteins |
| | | | | involved in resistance to |
| | | | | cholate and to NA(+) and in |
| | | | | pH homeostasis |
| SEQ ID NO. 2792 | LM-912.1 | From 2459343 | to 2459822 | unknown, similar to proteins |
| | | | | involved in resistance to |
| | | | | cholate and to NA(+) and in |
| | | | | pH homeostasis |
| SEQ ID NO. 2793 | LM-913.1 | From 2457852 | to 2459336 | unknown, similar to proteins |
| | | | | involved in resistance to |
| | | | | cholate and to NA(+) and in |
| | | | | pH homeostasis |
| SEQ ID NO. 2794 | LM-915.1 | From 2457512 | to 2457859 | unknown, similar to proteins |
| | | | | involved in resistance to |
| | | | | cholate and to NA(+) and in |
| | | | | pH homeostasis |
| SEQ ID NO. 2795 | LM-916.1 | From 2457087 | to 2457512 | unknown, similar to proteins |
| | | | | involved in resistance to |
| | | | | cholate and to NA(+) and in |
| | | | | pH homeostasis |
| SEQ ID NO. 2796 | LM-917.1 | From 2454695 | to 2457103 | unknown, similar to proteins |
| | | | | involved in resistance to |
| | | | | cholate and to NA(+) and in |
| | | | | pH homeostasis |
| SEQ ID NO. 2797 | LM-918.1 | From 2453138 | to 2454349 | Unknown, similar to multidrug |
| | | | | resistance efflux pump |
| SEQ ID NO. 2798 | LM-919.1 | From 2452322 | to 2452906 | Unknown, similar to |
| | | | | peptidyl-prolyl cis-trans |
| | | | | isomerase |
| SEQ ID NO. 2799 | LM-92.1 | From 2770060 | to 2771439 | Unknown, similar to lysine |
| | | | | decarboxylase |
| SEQ ID NO. 2800 | LM-920.1 | From 2451840 | to 2452235 | Unknown |
| SEQ ID NO. 2801 | LM-922.1 | From 2450437 | to 2451798 | Unknown, similar to |
| | | | | aspartate kinase |
| SEQ ID NO. 2802 | LM-923.1 | From 2449878 | to 2450192 | Unknown, similar to |
| | | | | phosphotransferase system |
| | | | | (PTS) beta-glucoside- |
| | | | | specific enzyme IIB |
| | | | | component |
| SEQ ID NO. 2803 | LM-924.1 | From 2449155 | to 2449835 | unknown, similar to ABC- |
| | | | | transporter ATP binding |
| | | | | proteins |
| SEQ ID NO. 2804 | LM-925.1 | From 2448052 | to 2449155 | Unknown, similar to putative |
| | | | | ABC-transporter |
| | | | | transmembrane subunit |
| SEQ ID NO. 2805 | LM-926.1 | From 2446747 | to 2447913 | Unknown, similar to |
| | | | | aminotransferase |
| SEQ ID NO. 2806 | LM-927.1 | From 2446240 | to 2446593 | Unknown, similar to B. subtilis |
| | | | | general stress |
| | | | | protein 13 containing a |
| | | | | ribosomal S1 protein |
| | | | | domain |
| SEQ ID NO. 2807 | LM-929.1 | From 2445685 | to 2446092 | Unknown |
| SEQ ID NO. 2808 | LM-930.2 | From 2444231 | to 2445583 | glucose-6-phosphate |
| | | | | isomerase |
| SEQ ID NO. 2809 | LM-931.2 | From 2443368 | to 2444126 | Unknown, similar to |
| | | | | transcription regulator DeoR |
| | | | | family |
| SEQ ID NO. 2810 | LM-935.1 | From 198201 | to 199409 | Unknown, similar to ABC |
| | | | | transporter, ATP-binding |
| | | | | protein |
| SEQ ID NO. 2811 | LM-936.1 | From 197512 | to 198204 | ABC transporter, ATP- |
| | | | | binding protein |
| SEQ ID NO. 2812 | LM-937.1 | From 196786 | to 197463 | unknown |
| SEQ ID NO. 2813 | LM-938.1 | From 195793 | to 196611 | Unknown, similar to PurR, |
| | | | | transcription repressor of |
| | | | | purine operon of B. subtilis |
| SEQ ID NO. 2814 | LM-939.1 | From 194892 | to 195629 | Unknown, similar to a |
| | | | | putative phospho-beta- |
| | | | | glucosidase |
| SEQ ID NO. 2815 | LM-94.1 | From 2771623 | to 2772612 | Unknown, similar to |
| | | | | dihydroxyacetone kinase |
| SEQ ID NO. 2816 | LM-941.1 | From 193989 | to 194870 | Unknown, similar to B. subtilis |
| | | | | YabH protein |
| SEQ ID NO. 2817 | LM-942.1 | From 193593 | to 193850 | Unknown, highly similar to |
| | | | | B. subtilis Veg protein |
| SEQ ID NO. 2818 | LM-943.1 | From 192586 | to 193473 | dimethyladenosine |
| | | | | transferase (16S rRNA |
| | | | | dimethylase) |
| SEQ ID NO. 2819 | LM-945.1 | From 192018 | to 192593 | Unknown, similar to B. subtilis |
| | | | | YabF protein |
| SEQ ID NO. 2820 | LM-946.1 | From 190690 | to 191916 | Unknown, similar to B. subtilis |
| | | | | YabE protein |
| SEQ ID NO. 2821 | LM-947.1 | From 189625 | to 190398 | Unknown, similar to |
| | | | | conserved hypothetical |
| | | | | proteins |
| SEQ ID NO. 2822 | LM-949.1 | From 187867 | to 189528 | Unknown, similar to oligo- |
| | | | | 1,6-glucosidase |
| SEQ ID NO. 2823 | LM-95.1 | From 2772634 | to 2773230 | Unknown, similar to |
| | | | | hypothetical |
| | | | | dihydroxyacetone kinase |
| SEQ ID NO. 2824 | LM-950.1 | From 185572 | to 187863 | Unknown, similar to alpha- |
| | | | | glucosidase |
| SEQ ID NO. 2825 | LM-955.1 | From 182267 | to 185569 | Unknown, similar to alpha- |
| | | | | xylosidase and alpha- |
| | | | | glucosidase |
| SEQ ID NO. 2826 | LM-958.1 | From 180928 | to 182184 | Unknown, similar to sugar |
| | | | | ABC transporter, |
| | | | | periplasmic sugar-binding |
| | | | | protein |
| SEQ ID NO. 2827 | LM-96.1 | From 2773234 | to 2773608 | unknown |
| SEQ ID NO. 2828 | LM-960.1 | From 180052 | to 180900 | Unknown, similar to sugar |
| | | | | ABC transporter, permease |
| | | | | protein |
| SEQ ID NO. 2829 | LM-961.1 | From 179174 | to 180052 | Unknown, similar to sugar |
| | | | | ABC transporters, |
| | | | | permease proteins |
| SEQ ID NO. 2830 | LM-962.1 | From 177924 | to 179138 | Unknown, similar to xylose |
| | | | | repressor |
| SEQ ID NO. 2831 | LM-964.1 | From 175766 | to 177760 | methionyl-tRNA synthetase |
| SEQ ID NO. 2832 | LM-965.1 | From 174834 | to 175694 | Unknown, similar to glucose |
| | | | | uptake protein |
| SEQ ID NO. 2833 | LM-968.1 | From 172903 | to 173208 | Unknown, similar to |
| | | | | transposase |
| SEQ ID NO. 2834 | LM-969.1 | From 172410 | to 172802 | Unknown, similar to |
| | | | | transposase (N-terminal |
| | | | | part) |
| SEQ ID NO. 2835 | LM-97.1 | From 2773714 | to 2774565 | Unknown, similar to putative |
| | | | | transcription regulator |
| SEQ ID NO. 2836 | LM-970.1 | From 172072 | to 172410 | Unknown, similar to |
| | | | | transposase C-terminal part |
| SEQ ID NO. 2837 | LM-975.1 | From 168005 | to 169270 | Unknown |
| SEQ ID NO. 2838 | LM-977.1 | From 167086 | to 167943 | Unknown, similar to a |
| | | | | glucose uptake protein |
| SEQ ID NO. 2839 | LM-978.1 | From 166687 | to 166971 | Unknown, similar to B. subtilis |
| | | | | transcription |
| | | | | regulatory protein AbrB |
| SEQ ID NO. 2840 | LM-98.1 | From 2774684 | to 2775523 | Unknown, similar to |
| | | | | conserved hypothetical |
| | | | | protein |
| SEQ ID NO. 2841 | LM-981.1 | From 165761 | to 166642 | Unknown, conserved |
| | | | | hypothetical protein |
| SEQ ID NO. 2842 | LM-982.1 | From 165492 | to 165764 | unknown, similar to B. subtilis |
| | | | | YazA protein |
| SEQ ID NO. 2843 | LM-983.1 | From 164756 | to 165508 | unknown, conserved |
| | | | | hypothetical protein |
| SEQ ID NO. 2844 | LM-984.1 | From 164309 | to 164698 | Unknown, similar to B. subtilis |
| | | | | YabA protein |
| SEQ ID NO. 2845 | LM-987.1 | From 163465 | to 164298 | Unknown |
| SEQ ID NO. 2846 | LM-988.2 | From 162467 | to 163459 | Unknown, similar to B. subtilis |
| | | | | DNA polymerase III |
| | | | | (delta subunit) |
| SEQ ID NO. 2847 | LM-990.2 | From 596580 | to 597620 | Unknown, conserved |
| | | | | hypothetical protein |
| SEQ ID NO. 2848 | LM-991.1 | From 595843 | to 596538 | Unknown, similar to |
| | | | | phosphoglycerate mutase |
| SEQ ID NO. 2849 | LM-992.1 | From 595135 | to 595842 | Unknown, similar to |
| | | | | phosphoglycerate mutase |
| SEQ ID NO. 2850 | LM-994.1 | From 593528 | to 595006 | Unknown, similar to di- |
| | | | | tripeptide ABC transporter |
| | | | | (membrane protein) |
| SEQ ID NO. 2851 | LM-995.1 | From 592257 | to 593438 | Unknown, similar to NADH- |
| | | | | dependent butanol |
| | | | | dehydrogenase |
| SEQ ID NO. 2852 | LM-996.1 | From 591412 | to 592047 | Unknown |
| SEQ ID NO. 2853 | LM-997.1 | From 590324 | to 591355 | Unknown, similar to |
| | | | | unknown protein |
| SEQ ID NO. 2854 | LM-998.1 | From 589406 | to 590248 | Unknown |

1. A nucleotide sequence of Listeria monocytogenes, characterized in that it corresponds to SEQ ID NO. 1.

2. A nucleotide sequence of Listeria monocytogenes, characterized in that it is chosen from:
a) a nucleotide sequence comprising at least 80% identity with SEQ ID NO. 1;
b) a nucleotide sequence which hybridizes, under high stringency conditions, with SEQ ID NO. 1;
c) a nucleotide sequence complementary to SEQ ED NO. 1 or complementary to a nucleotide sequence as defined in a) or b), or a nucleotide sequence of the corresponding RNA;
d) a nucleotide sequence of a representative fragment of SEQ ID NO. 1, or of a representative fragment of a nucleotide sequence as defined in a), b) or c);
e) a nucleotide sequence comprising a sequence as defined in a), b), c) or d); and
f) a modified nucleotide sequence of a nucleotide sequence as defined in a), b), c), d) or e).

3. The nucleotide sequence as claimed in claim 2, characterized in that it is a sequence derived from SEQ ID NO. 1, and in that it encodes a polypeptide chosen from the polypeptides of sequence SEQ ID NO. 1 to SEQ ID NO. 2854.

4. A nucleotide sequence, characterized in that it comprises a nucleotide sequence chosen from:
a) a nucleotide sequence as claimed in claim 3;
b) a nucleotide sequence comprising at least 80% identity with a nucleotide sequence as claimed in claim 3;
c) a nucleotide sequence which hybridizes, under high stringency conditions, with a nucleotide sequence as claimed in claim 3;
d) a complementary nucleotide sequence or an RNA sequence corresponding to a sequence as defined in a), b) or c);
e) a nucleotide sequence of a representative fragment of a sequence as defined in a), b), c) or d); and
f) a modified nucleotide sequence of a sequence as defined in a) b), c), d) or e).

5. A polypeptide encoded by a nucleotide sequence as claimed in one of claims 2 to 4.

6. The polypeptide as claimed in claim 5, characterized in that it is chosen from the sequences SEQ ID NO. 2 to SEQ ID NO. 2854.

7. A polypeptide characterized in that it comprises a polypeptide chosen from:
a) a polypeptide as claimed in either of claims 5 and 6;
b) a polypeptide which exhibits at least 80% identity with a polypeptide as claimed in either of claims 5 and 6;
c) a fragment of at least 5 amino acids of a polypeptide as claimed in either of claims 5 and 6, or as defined in b);
d) a biologically active fragment of a polypeptide as claimed in either of claims 5 and 6, or as defined in b) or c); and
e) a modified polypeptide of a polypeptide as claimed in either of claims 5 and 6, or as defined in b), c) or d).

8. A nucleotide sequence encoding a polypeptide as claimed in claim 7.

9. The nucleotide sequence as claimed in one of claims 2 to 4 and 8, characterized in that it encodes a polypeptide of Listeria monocytogenes, or a fragment thereof, involved in amino acid biosynthesis.

10. The nucleotide sequence as claimed in one of claims 2 to 4 and 8, characterized in that it encodes a polypeptide of Listeria monocytogenes, or a fragment thereof, involved in the biosynthesis of cofactors, prosthetic groups and transporters.

11. The nucleotide sequence obtained in one of claims 2 to 4 and 8, characterized in that it encodes a cell envelope polypeptide or a polypeptide located at the surface of Listeria monocytogenes, or a fragment thereof, said polypeptide preferably being SEQ ID NO. 2 to SEQ ID NO. 41.

12. The nucleotide sequence as claimed in one of claims 2 to 4 and 8, characterized in that it encodes a polypeptide of Listeria monocytogenes, or a fragment thereof, involved in the cellular machinery.

13. The nucleotide sequence as claimed in one of claims 2 to 4 and 8, characterized in that it encodes a polypeptide of Listeria monocytogenes, or a fragment thereof, involved in central intermediate metabolism.

14. The nucleotide sequence as claimed in one of claims 2 to 4 and 8, characterized in that it encodes a polypeptide of Listeria monocytogenes, or a fragment thereof, involved in energetic metabolism.

15. The nucleotide sequence as claimed in one of claims 2 to 4 and 8, characterized in that it encodes a polypeptide of Listeria monocytogenes, or a fragment thereof, involved in the metabolism of fatty acids and phospholipids.

16. The nucleotide sequence as claimed in one of claims 2 to 4 and 8, characterized in that it encodes a polypeptide of Listeria monocytogenes, or a fragment thereof, involved in the metabolism of nucleotides, purines, pyrimidines or nucleosides.

17. The nucleotide sequence as claimed in one of claims 2 to 4 and 8, characterized in that it encodes a polypeptide of Listeria monocytogenes, or a fragment thereof, involved in regulatory functions.

18. The nucleotide sequence as claimed in one of claims 2 to 4 and 8, characterized in that it encodes a polypeptide of Listeria monocytogenes, or a fragment thereof, involved in the replication process.

19. The nucleotide sequence as claimed in one of claims 2 to 4 and 8, characterized in that it encodes a polypeptide of Listeria monocytogenes, or a fragment thereof, involved in the transcription process.

20. The nucleotide sequence as claimed in one of claims 2 to 4 and 8, characterized in that it encodes a polypeptide of Listeria monocytogenes, or a fragment thereof, involved in the translation process.

21. The nucleotide sequence as claimed in one of claims 2 to 4 and 8, characterized in that it encodes a polypeptide of Listeria monocytogenes, or a fragment thereof, involved in the process of transport and binding of proteins.

22. The nucleotide sequence as claimed in one of claims 2 to 4 and 8, characterized in that it encodes a polypeptide of Listeria monocytogenes, or a fragment thereof, involved in adaptation to atypical conditions.

23. The nucleotide sequence as claimed in one of claims 2 to 4 and 8, characterized in that it encodes a polypeptide of Listeria monocytogenes, or a fragment thereof, involved in sensitivity to medicinal products and analogs.

24. A nucleotide sequence characterized in that it encodes a polypeptide of Listeria monocytogenes involved in vitamin B12 biosynthesis, said polypeptide preferably being SEQ ID NO. 42 to SEQ ID NO. 64.

25. The nucleotide sequence as claimed in one of claims 2 to 4 and 8, characterized in that it encodes a polypeptide of Listeria monocytogenes, or a fragment thereof, involved in functions relating to transposons.

26. The nucleotide sequence as claimed in one of claims 2 to 4 and 8, characterized in that it encodes a polypeptide specific for Listeria monocytogenes, or a fragment thereof.

27. The polypeptide as claimed in one of claims 5 to 7,l characterized in that it is a polypeptide of Listeria monocytogenes, or a fragment thereof, involved in amino acid biosynthesis.

28. The polypeptide as claimed in one of claims 5 to 7, characterized in that it is a polypeptide of Listeria monocytogenes, or a fragment thereof, involved in the biosynthesis of cofactors, prosthetic groups and transporters.

29. The polypeptide as claimed in one of claims 5 to 7, characterized in that it is a cell envelope polypeptide or a polypeptide located at the surface of Listeria monocytogenes, or a fragment thereof, said polypeptide preferably being SEQ ID NO. 2 to SEQ ID NO. 41.

30. The polypeptide as claimed in one of claims 5 to 7, characterized in that it is a polypeptide of Listeria monocytogenes, or a fragment thereof, involved in the cellular machinery.

31. The polypeptide as claimed in one of claims 5 to 7, characterized in that it is a polypeptide of Listeria monocytogenes, or a fragment thereof, involved in central intermediate metabolism.

32. The polypeptide as claimed in one of claims 5 to 7, characterized in that it is a polypeptide of Listeria monocytogenes, or a fragment thereof, involved in energetic metabolism.

33. The polypeptide as claimed in one of claims 5 to 7, characterized in that it is a polypeptide of Listeria monocytogenes, or a fragment thereof, involved in the metabolism of fatty acids and phospholipids.

34. The polypeptide as claimed in one of claims 5 to 7, characterized in that it is a polypeptide of Listeria monocytogenes, or a fragment thereof, involved in the metabolism of nucleotides, purines, pyrimidines or nucleosides.

35. The polypeptide as claimed in one of claims 5 to 7, characterized in that it is a polypeptide of Listeria monocytogenes, or a fragment thereof, involved in regulatory functions.

36. The polypeptide as claimed in one of claims 5 to 7, characterized in that it is a polypeptide of Listeria monocytogenes, or a fragment thereof, involved in the replication process.

37. The polypeptide as claimed in one of claims 5 to 7, characterized in that it is a polypeptide of Listeria monocytogenes, or a fragment thereof, involved in the transcription process.

38. The polypeptide as claimed in one of claims 5 to 7, characterized in that it is a polypeptide of Listeria monocytogenes, or a fragment thereof, involved in the translation process.

39. The polypeptide as claimed in one of claims 5 to 7, characterized in that it is a polypeptide of Listeria monocytogenes, or a fragment thereof, involved in the process of transport and binding of proteins.

40. The polypeptide as claimed in one of claims 5 to 7, characterized in that it is a polypeptide of Listeria monocytogenes, or a fragment thereof, involved in adaptation to atypical conditions.

41. The polypeptide as claimed in one of claims 5 to 7, characterized in that it is a polypeptide of Listeria monocytogenes, or a fragment thereof, involved in sensitivity to medicinal products and analogs.

42. The polypeptide as claimed in one of claims 5 to 7, characterized in that it is a polypeptide of Listeria monocytogenes, involved in vitamin B12 biosynthesis, said polypeptide preferably being SEQ ID NO. 42 to SEQ ID NO. 64.

43. The polypeptide as claimed in one of claims 5 to 7, characterized in that it is a polypeptide of Listeria monocytogenes, or a fragment thereof, involved in functions relating to transposons.

44. The polypeptide as claimed in one of claims 5 to 7, characterized in that it is a polypeptide specific for Listeria monocytogenes, or a fragment thereof.

45. The nucleotide sequence as claimed in one of claims 1 to 4 and 8 to 26, and/or the polypeptide sequence as claimed in one of claims 5 to 7 and 27 to 44, characterized in that the said sequence(s) is (are) recorded on a recording medium, the form and nature of which facilitate the reading, analysis and/or exploitation of said sequence(s).

46. The nucleotide sequence or the polypeptide sequence as claimed in claim 45, characterized in that the recording medium is a CD-ROM, a computer disk or a computer server.

47. A recording medium characterized in that a nucleotide or polypeptide sequence as claimed in claim 45 or 46 is recorded thereon.

48. The use of a medium as claimed in claim 47, for choosing nucleotide primers or probes for determining genes in strains close to Listeria monocytogenes.

49. The use of a medium as claimed in claim 47, for studying the genetic polymorphism of strains close to Listeria monocytogenes.

50. The use of a medium as claimed in claim 47, for studying other genomes, in particular for the automatic annotation of genes originating from other genomes.

51. A nucleotide sequence which can be used as a primer or as a probe, characterized in that said sequence is chosen from the nucleotide sequences as claimed in one of claims 2 to 4 and 8 to 26.

52. The nucleotide sequence as claimed in claim 51, characterized in that it is labeled with a radioactive compound or with a nonradioactive compound.

53. The nucleotide sequence as claimed in either of claims 51 and 52, characterized in that it is covalently or noncovalently immobilized on a support.

54. The nucleotide sequence as claimed in claim 53, characterized in that it is immobilized on a support such as a high density filter or a DNA chip.

55. The nucleotide sequence as claimed in one of claims 52 to 55, for detecting and/or amplifying nucleic acid sequences.

56. A DNA chip or filter, characterized in that it contains at least one nucleotide sequence as claimed in claim 55.

57. The DNA chip or filter as claimed in claim 56, characterized in that it also contains at least one nucleotide sequence of a microorganism other than Listeria monocytogenes, immobilized on the support of said chip.

58. The DNA chip or filter as claimed in claim 57, characterized in that the other microorganism is chosen from a microorganism associated with Listeria monocytogenes, a bacterium of the genus Listeria and a variant of Listeria monocytogenes.

59. A kit or pack for detecting and/or identifying bacteria belonging to the species belonging to the species Listeria monocytogenes, or to an associated microorganism, characterized in that it comprises a DNA chip or a filter as claimed in claim 56.

60. A kit or pack for detecting an/or identifying a microorganism, characterized in that it comprises a DNA chip or a filter as claimed in either of claims 57 and 58.

61. A kit or pack for detecting and/or quantifying the expression of at least one gene of Listeria monocytogenes, characterized in that it comprises a DNA chip or a filter as claimed in one of claims 56 to 58.

62. A cloning and/or expression vector, characterized in that it contains a nucleotide sequence as claimed in one of claims 2 to 4 and 8 to 26.

63. The cloning and/or expression vector as claimed in claim 62, characterized in that it contains a nucleotide sequence as claimed in claim 11.

64. The cloning and/or expression vector as claimed in claim 62, characterized in that it contains a nucleotide sequence as claimed in claim 14.

65. The cloning and/or expression vector as claimed in claim 62, characterized in that it contains a nucleotide sequence as claimed in claim 9, 19 or 20.

66. The cloning and/or expression vector as claimed in claim 62, characterized in that it contains a nucleotide sequence as claimed in claim 12, in particular encoding a protein involved in secretion mechanisms.

67. The cloning and/or expression vector as claimed in claim 62, characterized in that it contains a nucleotide sequence as claimed in claim 24.

68. The cloning and/or expression vector as claimed in claim 62, characterized in that it contains a nucleotide sequence encoding a protein involved in resistance and/or adaptation to stress.

69. A host cell, characterized in that it is transformed with a vector as claimed in one of claims 62 to 68.

70. The host cell as claimed in claim 69, characterized in that it is a bacterium belonging to the Listeria genus.

71. The host cell as claimed in claim 70, characterized in that it is a bacterium belonging to the species Listeria monocytogenes.

72. A plant or animal, except a human, comprising a transformed cell as claimed in one of claims 69 to 71.

73. A method for preparing a polypeptide, characterized in that a cell transformed with a vector as claimed in claim 62 is cultured under conditions which allow expression of said polypeptide, and in that said recombinant polypeptide is recovered.

74. A recombinant polypeptide which can be obtained using a method as claimed in claim 73.

75. A method for preparing a synthetic polypeptide as claimed in one of claims 5 to 7 and 27 to 44, characterized in that chemical synthesis of said polypeptide is performed.

76. A hybrid polypeptide, characterized in that it comprises at least the sequence of a polypeptide as claimed in one of claims 5 to 27, 27 to 44 and 74, and a sequence of a polypeptide capable of inducing an immune response in humans or animals.

77. A nucleotide sequence encoding a hybrid polypeptide as claimed in claim 76.

78. A vector, characterized in that it contains a nucleotide sequence as claimed in claim 77.

79. A monoclonal or polyclonal antibody, a fragment thereof or a chimeric antibody, characterized in that it is capable of specifically recognizing a polypeptide as claimed in one of claims 5 to 7, 27 to 44, 74 or 76.

80. The antibody as claimed in claim 79, characterized in that it is a labeled antibody.

81. A method for detecting and/or identifying bacteria belonging to the species Listeria monocytogenes, or to an associated microorganism, in a biological sample, characterized in that it comprises the following steps:
a) bringing the biological sample into contact with an antibody as claimed in either of claims 79 and 80;
b) demonstrating the antigen-antibody complex possibly formed.

82. A method for detecting the expression of a gene of Listeria monocytogenes , characterized in that a strain of Listeria monocytogenes is brought into contact with an antibody as claimed in claim 79 or 80, and in that the antigen/antibody complex possibly formed is detected.

83. A kit or pack for carrying out a method as claimed in claim 81 or 82, characterized in that it comprises the following elements:
a) a polyclonal or monoclonal antibody as claimed in either of claims 79 and 80;
b) optionally, the reagents for constituting the medium suitable for the immunoreaction;
c) optionally, the reagents for demonstrating the antigen-antibody complexes produced by the immunoreaction.

84. The polypeptide as claimed in one of claims 5 to 7, 27 to 44, 74 and 76, or the antibody as claimed in either of claims 79 and 80, characterized in that it is immobilized on a support, in particular a protein chip.

85. A protein chip, characterized in that it contains at least one polypeptide as claimed in one of claims 5 to 7, 27 to 44, 74 and 76, or at least one antibody as claimed in either of claims 79 and 80, immobilized on the support of said chip.

86. The protein chip as claimed in claim 85, characterized in that it also contains at least one polypeptide of a microorganism other than Listeria monocytogenes, or at least one antibody directed against a compound of a microorganism other than Listeria monocytogenes, immobilized on the support of said chip.

87. A kit or pack for detecting and/or identifying bacteria belonging to the species Listeria monocytogenes, or to an associated microorganism, characterized in that it comprises a protein chip as claimed in either of claims 85 and 86.

88. A kit or pack for detecting and/or identifying a microorganism, characterized in that in that it comprises a protein chip as claimed in claim 86.

89. A method for detecting and/or identifying bacteria belonging to the species Listeria monocytogenes, or to an associated microorganism, in a biological sample, characterized in that it uses a nucleotide sequence as claimed in one of claims 1 to 4, 8 to 26, 51 to 55 and 77.

90. The method as claimed in claim 89, characterized in that it comprises the following steps:
a) optionally isolating the DNA from the biological sample to be analyzed, or obtaining a cDNA from the RNA of the biological sample;
b) specifically amplifying the DNA of bacteria belonging to the species Listeria monocytogenes, or to an associated microorganism, using at least one primer as claimed in one of claims 51 to 55;
c) demonstrating the amplification products.

91. The method as claimed in claim 89, characterized in that it comprises the following steps:
a) bringing a nucleotide probe as claimed in one of claims 51 to 55 into contact with a biological sample, the nucleic acid contained in the biological sample having, where appropriate, been previously made accessible to hybridization, under conditions which allow hybridization of the probe to the nucleic acid of a bacterium belonging to the species Listeria monocytogenes, or to an associated microorganism;
b) demonstrating the hybrid possibly formed between the nucleotide probe and the nucleic acid of the biological sample.

92. The method as claimed in claim 89, characterized in that it comprises the following steps:
a) bringing a nucleotide probe immobilized on a support as claimed in claim 53 into contact with a biological sample, the nucleic acid of the sample having, where appropriate, previously been made accessible to hybridization, under conditions which allow hybridization of the probe to the nucleic acid of a bacterium belonging to the species Listeria monocytogenes, or to an associated microorganism;
b) bringing the hybrid formed between the nucleotide probe immobilized on a support and the nucleic acid contained in the biological sample, where appropriate after removing the nucleic acid of the biological sample which has not hybridized with the probe, into contact with a labeled nucleotide probe as claimed in claim 52;
c) demonstrating the new hybrid formed in step b).

93. The method as claimed in claim 92, characterized in that, prior to step a), the DNA of the biological sample, or the cDNA optionally obtained by reverse transcription of the RNA of the sample, is amplified using at least one primer as claimed in one of claims 51 to 55.

94. A kit or pack for detecting and/or identifying bacteria belonging to the species Listeria monocytogenes, or to an associated microorganism, characterized in that it comprises the following elements:
a) a nucleotide probe as claimed in one of claims 51 to 55;
b) optionally, the reagents required for carrying out a hybridization reaction;
c) optionally, at least one primer as claimed in one of claims 51 to 55 and also the reagents required for a DNA amplification reaction.

95. A kit or pack for detecting and/or identifying bacteria belonging to the species Listeria monocytogenes, or to an associated microorganism, characterized in that it comprises the following elements:
a) a nucleotide probe, termed capture probe, as claimed in claim 53;
b) an oligonucleotide probe, termed detection probe, as claimed in claim 52;
c) optionally, at least one primer as claimed in one of claims 51 to 55 and also the reagents required for a DNA amplification reaction.

96. A kit or pack for detecting and/or identifying bacteria belonging to the species Listeria monocytogenes, or to an associated microorganism, characterized in that it comprises the following elements:
a) at least one primer as claimed in one of claims 51 to 55;
b) optionally, the reagents required for carrying out a DNA amplification reaction;
c) optionally, a component for verifying the sequence of the amplified fragment, more particularly an oligonucleotide probe as claimed in one of claims 51 to 55.

97. A method as claimed in claims 81, 82 and 89 to 93, or a kit or pack as claimed in claims 83, 87, 88 and 94 to 96, for detecting and/or identifying bacteria belonging to the species Listeria monocytogenes, characterized in that said primer and/or said probe are chosen from the nucleotide sequences as claimed in one of claims 2 to 4, 8 to 26, 51 to 55 and 77, specific for the species Listeria monocytogenes, and in that said polypeptides are chosen from the polypeptides as claimed in one of claims 5 to 7, 27 to 44 and 74 or 76, specific for the species Listeria monocytogenes, and in that said antibodies are chosen from the antibodies as claimed in either of claims 79 and 80, directed against polypeptides chosen from the polypeptides as claimed in one of claims 5 to 7, 26 to 44, 74 or 76, specific for the species Listeria monocytogenes.

98. A method or a kit or pack as claimed in claim 97, characterized in that said primer and/or said probe are chosen from the nucleotide sequences encoding a secreted protein, in that said polypeptides are chosen from secreted polypeptides, and in that said antibodies are chosen from the antibodies as claimed in either of claims 79 and 80, directed against secreted or surface polypeptides of Listeria monocytogenes, said polypeptides preferably being SEQ ID NO. 2 to SEQ ID NO. 41.

99. A strain of Listeria monocytogenes, characterized in that it contains at least one mutation in at least one nucleotide sequence as claimed in one of claims 2 to 4 or 8 to 26.

100. The strain of Listeria monocytogenes as claimed in claim 99, characterized in that it contains a mutation in a nucleotide sequence as claimed in claim 13.

101. The strain of Listeria monocytogenes as claimed in claim 99, characterized in that it contains a mutation in a nucleotide sequence as claimed in claim 14.

102. The strain of Listeria monocytogenes as claimed in claim 99, characterized in that it contains a mutation in a nucleotide sequence as claimed in claim 9, 19 or 20.

103. The strain of Listeria monocytogenes as claimed in claim 99, characterized in that it contains a mutation in a nucleotide sequence as claimed in claim 12, in particular encoding a protein involved in secretion mechanisms.

104. The strain of Listeria monocytogenes as claimed in claim 99, characterized in that it contains a mutation in a nucleotide sequence encoding a protein involved in resistance and/or adaptation to stress.

105. The strain of Listeria monocytogenes as claimed in claim 99, characterized in that it contains a mutation in a nucleotide sequence encoding a protein involved in vitamin B 12 biosynthesis.

106. The strain of Listeria monocytogenes as claimed in one of claims 99 to 105, characterized in that the mutation leads to inactivation of the gene.

107. The strain of Listeria monocytogenes as claimed in one of claims 99 to 105, characterized in that the mutation leads to overexpression of the gene.

108. The use of a polypeptide as claimed in one of claims 5 to 7, 27 to 44, 74 and 76, of a transformed cell as claimed in one of claims 69 to 71, of a strain as claimed in claims 99 to 107 and/or of an animal as claimed in claim 72, for the biosynthesis or biodegradation of a compound of interest.

109. A method of biosynthesis or of biodegradation of a compound of interest, characterized in that it uses a polypeptide as claimed in one of claims 5 to 7, 26 to 44, 74 and 76, a transformed cell as claimed in one of claims 69 to 71, a strain as claimed in one of claims 99 to 107 and/or an animal as claimed in claim 72.

110. The use as claimed in claim 108 or the method as claimed in claim 109, characterized in that the compound of interest is vitamin B12.

111. The use of a nucleotide sequence as claimed in one of claims 2 to 4 and 8 to 26, of a polypeptide as claimed in one of claims 5 to 7, 27 to 44, 74 and 76, of an antibody as claimed in either of claims 79 and 80, of a cell as claimed in one of claims 69 to 71 and/or of a transformed animal as claimed in claim 72, for selecting an organic or inorganic compound capable of modulating, regulating, inducing or inhibiting gene expression, and/or of modifying cell replication in eukaryotic or prokaryotic cells, or capable of inducing, inhibiting or worsening, in an animal or human organism, pathological conditions linked to an infection with Listeria monocytogenes or with an associated microorganism.

112. A method for selecting a compound capable of binding to a polypeptide as claimed in one of claims 5 to 7, 27 to 44, 74 and 76, capable of binding to a nucleotide sequence as claimed in one of claims 2 to 4 and 8 to 26, or capable of recognizing an antibody as claimed in either of claims 79 and 80, and/or capable of modulating, regulating, inducing or inhibiting gene expression, and/or of modifying cell replication in eukaryotic or prokaryotic cells, or capable of inducing, inhibiting or worsening, in an animal or human organism, pathological conditions linked to an infection with Listeria monocytogenes, characterized in that it comprises the following steps:
a) bringing said compound into contact with said polypeptide or said nucleotide sequence or with a transformed cell as claimed in one of claims 69 to 71 and/or administering said compound to a transformed animal as claimed in claim 72;
b) determining the ability of said compound to bind to said polypeptide or said nucleotide sequence, or to modulate, regulate, induce or inhibit gene expression, or to modulate the cell growth or replication, or to induce, inhibit or worsen, in said animal or human organism, pathological conditions linked to an infection with Listeria monocytogenes or with an associated microorganism.

113. A compound which can be selected using a method as claimed in claim 112.

114. A pharmaceutical composition comprising a compound chosen from the following compounds:
a) a nucleotide sequence as claimed in one of claims 2 to 4 and 8 to 26;
b) a polypeptide as claimed in one of claims 5 to 7, 27 to 44, 74 and 76;
c) a vector as claimed in one of claims 62 to 68 and 78;
d) an antibody as claimed in claim 79 or 80; and
e) a compound as claimed in claim 113.

115. A composition as claimed in claim 114, optionally in combination with a pharmaceutically acceptable vehicle.

116. The pharmaceutical composition as claimed in either of claims 114 and 115, for preventing or treating an infection with a bacterium belonging to the species Listeria monocytogenes, or with an associated microorganism.

117. A vaccinal composition, characterized in that it comprises one or more polypeptides as claimed in one of claims 5 to 7, 27 to 44 and 74, and/or one or more hybrid polypeptides as claimed in claim 76.

118. The use of a cell as claimed in one of claims 62 to 68, for preparing a vaccinal composition.

119. A vaccinal composition, characterized in that it contains a vector as claimed in one of claims 62 to 68 and 78, and/or a cell as claimed in one of claims 69 to 71.

120. A vaccinal composition capable of inducing a cellular or humoral response, for preventing or treating an infection with a bacterium belonging to the species Listeria monocytogenes, or with an associated microorganism, characterized in that it comprises an immunogenic composition as claimed in either of claims 117 and 119, in combination with a pharmaceutically acceptable vehicle and, optionally, one or more suitable adjuvants of immunity.

121. A genomic DNA library of a bacterium of the Listeria genus, characterized in that said DNA library is cloned in bacterial artificial chromosomes (BACs).

122. The genomic DNA library as claimed in claim 121, characterized in that said bacterium is Listeria monocytogenes.

123. The genomic DNA library as claimed in claim 121, characterized in that said bacterium is the EGD-e strain of Listeria monocytogenes.

124. The library as claimed in claim 122, characterized in that it is the LM-baclib library deposited with the CNCM on Apr. 11, 2000, under the number I-2439.

125. A method for isolating a polynucleotide of interest present in a strain of Listeria and absent from another strain, characterized in that it uses at least one library as claimed in either of claims 121 and 124.

126. The method as claimed in claim 125, characterized in that it comprises the following steps:
a) isolating at least one polynucleotide contained in a clone from the DNA library based on a BAC, of listeria origin;
b) isolating:
at least one genomic polynucleotide or cDNA of a listeria, said listeria belonging to a strain which is different from the strain used to construct the BAC DNA library of step a) or, alternatively,
at least one polynucleotide contained in a clone from a DNA library based on a BAC prepared from the genome of a Listeria which is different from the listeria used to construct the DNA library based on the BAC of step a);
c) hybridizing the polynucleotide of step a) to the polynucleotide of step b);
d) selecting the polynucleotides of step a) which have not formed a hybridization complex with the polynucleotides of step b);
e) characterizing the polynucleotide selected.

127. A genomic library of a bacterium of the Listeria genus.

128. The genomic library as claimed in claim 127, characterized in that the bacterium is Listeria monocytogenes.
